(19) United States
(12) Patent Application Publication
(10) Pub. No.: US 2025/0098537 A1
(43) Pub. Date: Mar. 20, 2025

(54) ELECTRONIC BARRIER MATERIAL AND ORGANIC SEMICONDUCTOR ELEMENT

(71) Applicant: KYULUX, INC., Fukuoka-shi, Fukuoka (JP)

(72) Inventors: Hiroaki OZAWA, Fukuoka-shi, Fukuoka (JP); Takahiro KASHIWAZAKI, Fukuoka-shi, Fukuoka (JP); Aiko GOTO, Fukuoka-shi, Fukuoka (JP); Tomoki YUKAWA, Fukuoka-shi, Fukuoka (JP); Momoko MORIO, Fukuoka-shi, Fukuoka (JP); Songhye HWANG, Fukuoka-shi, Fukuoka (JP); Makoto YOSHIZAKI, Fukuoka-shi, Fukuoka (JP); Ayataka ENDO, Fukuoka-shi, Fukuoka (JP)

(21) Appl. No.: 18/722,157

(22) PCT Filed: Nov. 29, 2022
(86) PCT No.: PCT/JP2022/044014

§ 371 Date: Jun. 20, 2024

(30) Foreign Application Priority Data
Dec. 23, 2021 (JP) 2021-209760
Jun. 23, 2022 (WO) PCT/JP2022/025151
Oct. 11, 2022 (JP) 2022-163580

(51) Int. Cl. H10K 85/60; H10K 50/10; H10K 50/81; H10K 50/82

(52) U.S. Cl. CPC H10K 85/658; H10K 50/10; H10K 50/81; H10K 50/82; H10K 85/657

(57) ABSTRACT

A compound represented by the following general formula is useful as an electron barrier material. R¹ to R²¹ each are H, a deuterium atom, or a substituent except a cyano group; X represents O or S.

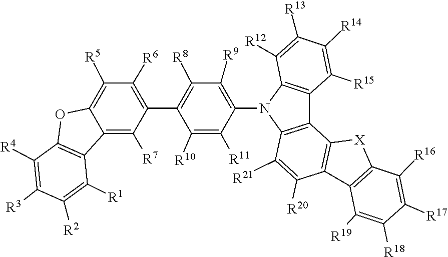

### TECHNICAL FIELD

The present invention relates to a compound useful as an electron barrier material, and to an organic semiconductor device using the compound.

### BACKGROUND ART

Studies for enhancing the performance of organic semiconductor devices such as organic electroluminescent devices (organic EL devices) are being made actively. For example, for improving the device lifetime and the drive voltage of an organic electroluminescent device, it is desirable to improve the functions of the materials participating in charge transportation, such as an electron transport material, a hole transport material, an electron barrier material, and a hole barrier material, and therefore development and improvement of these materials are also being promoted.

For example, an electron barrier material is a material of an electron barrier layer that is arranged between a light emitting layer and a hole transport layer, and has a function of blocking the electrons existing in the light emitting layer from passing from the light emitting layer to the hole transport layer, and of transporting the holes from the hole transport layer to the light emitting layer. When an excellent electron barrier material is used, the recombination probability of electrons and holes in the light emitting layer is improved, and as a result, the lifetime of the device is prolonged. Heretofore, various compounds have been proposed for electron barrier materials, and for example, PTL 1 uses a compound having the following structure.

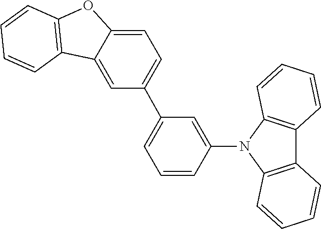 [SMILES: c1cc(-c2ccc3oc4ccccc4c3c2)cc(-n2c3ccccc3c3ccccc32)c1]

### CITATION LIST

### Patent Literature

PTL 1: WO2022/168956A

### SUMMARY OF INVENTION

### Technical Problem

However, an organic electroluminescent device using the above-mentioned compound as an electron barrier material has room for further improvement in drive voltage and device life. Consequently, the present inventors have conducted intensive studies on an object of providing an electron barrier material which, when used in an organic electroluminescent device, can lower the drive voltage and can prolong the device lifetime.

### Solution to Problem

As a result of promoting intensive studies, the present inventors have found that a compound having a specific structure can function as an excellent electron barrier material. The present invention has been provided based on these findings, and specifically has the following configuration.

[1] An electron barrier material containing a compound represented by the following general formula (1).

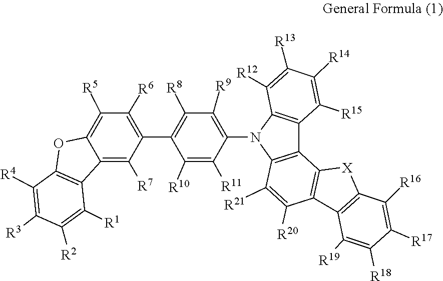

In the formula, R¹ to R²¹ each independently represent a hydrogen atom, a deuterium atom, or a substituent not including a cyano group. One combination of R¹² and R¹³, R³ and R¹⁴, and R¹⁴ and R¹⁵ can bond to each other to form a benzofuro skeleton or a benzothieno skeleton. R¹ to R¹¹, and R¹⁶ to R²¹ do not bond to the other R¹ to R¹¹, R¹⁶ to R²¹ or R¹² to R¹⁵ to form a cyclic structure. X represents an oxygen atom or a sulfur atom.

[2] The electron barrier material according to [1], wherein R¹ to R²¹ do not bond to the other R¹ to R²¹ to form a cyclic structure.

[3] The electron barrier material according to [1] or [2], wherein R¹ to R²¹ each independently represent a hydrogen atom, a deuterium atom, an optionally-deuterated alkyl group, or an optionally-deuterated phenyl group.

[4] The electron barrier material according to any one of [1] to [3], wherein R¹ to R¹¹, R²⁰ and R²¹ each independently represent a hydrogen atom or a deuterium atom.

[5] The electron barrier material according to any one of [1] to [4], wherein R¹² to R¹⁵ each independently represent a hydrogen atom or a deuterium atom.

[6] The electron barrier material according to any one of [1] to [5], wherein R¹⁶ to R¹⁹ each independently represent a hydrogen atom or a deuterium atom.

[7] The electron barrier material according to any one of [1] to [6], wherein X is an oxygen atom.

[8] The electron barrier material according to any one of [1] to [7], which is used in combination with a compound represented by the following general formula (G).

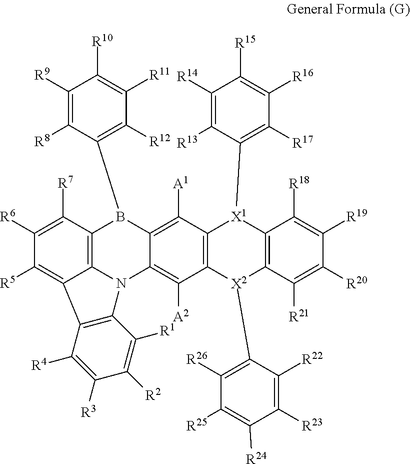

In the general formula (G), one of X¹ and X² is a nitrogen atom, and the other is a boron atom. R¹ to R²⁶, A¹ and A² each independently represent a hydrogen atom, a deuterium atom, or a substituent. R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ can bond to each other to form a cyclic structure. However, when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring.

In one aspect of the present invention, in the case where X¹ is a nitrogen atom, and where R⁷ and R⁸ and R²¹ and R²² each bond to each other via a nitrogen atom to form a 6-membered ring, and R¹⁷ and R¹⁸ bond to each other to form a single bond, at least one of R¹ to R⁶ is a substituted or unsubstituted aryl group, or any of R¹ and R², R² and R³, R³ and R⁴, R⁴ and R⁵, and R⁵ and R⁶ bond to each other to form an aromatic ring or a heteroaromatic ring. In one aspect of the present invention, in the case where X¹ is a boron atom, X² is a nitrogen atom, and R⁷ and R⁸, and R¹⁷ and R¹⁸ each bond to each other to form a boron atom-containing cyclic structure, the cyclic structure is a 5 to 7-membered ring, and in the case of a 6-membered ring, R⁷ and R⁸, and R¹⁷ and R¹⁸ each bond to each other to form —B(R³²)—, —CO—, —CS— or —N(R²⁷)—. R²⁷ represents a hydrogen atom, a deuterium atom or a substituent.

[9] An organic semiconductor device containing the electron barrier material according to any one of [1] to [7].

[10] The organic semiconductor device according to [9], wherein the organic semiconductor device is an organic electroluminescent device having an anode, a cathode, and at least two organic layers containing an electron barrier layer that contains the above electron barrier material and a light emitting layer, between the anode and the cathode.

[11] The organic semiconductor device according to [10], wherein the light emitting layer contains a host material and a delayed fluorescent material.

[12] The organic semiconductor device according to [10], wherein the light emitting layer contains a host material, a delayed florescent material and a fluorescence emitting material, and the amount of light emitted from the fluorescence emitting material is the largest among the light from the device.

[13] The organic semiconductor device according to any one of [10] to [12], wherein the light emitting layer is adjacent to the electron barrier layer.

[14] The organic semiconductor device according to any one of [10] to [13], wherein the light emitting layer contains the compound represented by the above general formula (G).

### Advantageous Effects of Invention

The compound represented by the general formula (1) is useful as an electron barrier material, and can be effectively used in an organic semiconductor device. For example, by using the compound of the present invention as an electron barrier layer of an organic electroluminescent device, the drive voltage can be lowered and the device lifetime can be prolonged.

### DESCRIPTION OF EMBODIMENTS

Hereinafter, the contents of the present invention will be described in detail. The constituent elements can be described below with reference to representative embodiments and specific examples of the present invention, but the present invention is not limited to the embodiments and the examples. In this application, a numerical range expressed as “to” means a range which includes the numerical values described before and after “to” as the lower limit value and the upper limit value. Further, in this application, “consisting of” means that it contains only what is described before “consisting of” and does not contain anything else. Furthermore, some or all of the hydrogen atoms that are present in the compounds used in the present invention can be substituted with deuterium atoms (²H, deuterium D). In the chemical structural formula of the present description, the hydrogen atom is indicated by H, or the indication thereof is omitted. For example, when the indication of an atom bonding to a ring skeleton forming carbon atom of a benzene ring is omitted, it is assumed that, at a location where the indication is omitted, H bonds to the ring skeleton forming carbon atom. In the present description, the term of “substituent” means an atom or a group of atoms other than a hydrogen atom and a deuterium atom. Meanwhile, the expression of “substituted or unsubstituted” or “optionally substituted” means that a hydrogen atom can be substituted with a deuterium atom or a substituent. “Transparent” in the present invention means that the visible light transmittance is 50% or more, preferably 80% or more, more preferably 90% or more, further preferably 99% or more. The visible light transmittance can be measured with a UV/visible light spectrophotometer.

### [Compound Represented by General Formula (1)]

In the present invention, a compound represented by the following general formula (1) is used.

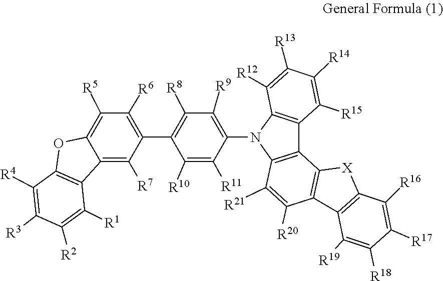

In the general formula (1), R¹ to R²¹ each independently represent a hydrogen atom, a deuterium atom, or a substituent not including a cyano group.

In one aspect of the present invention, the substituent of R¹ to R²¹ are each independently a substituent having a Hammett's σp value falling within a range of −0.3 to 0.3. In one preferred aspect of the present invention, the substituent of R¹ to R²¹ are each independently a substituent having a Hammett's σp value falling within a range of −0.2 to 0.2. In one preferred aspect of the present invention, the substituent of R¹ to R²¹ are each independently a substituent having a Hammett's σp value falling within a range of −0.1 to 0.1. In one aspect of the present invention, the substituent of R¹ to R²¹ are each independently a substituent having a Hammett's σp value falling within a range of larger than 0 and 0.3 or less. In one aspect of the present invention, the substituent of R¹ to R²¹ are each independently a substituent having a Hammett's σp value falling within a range of −0.3 or more and less than 0.

Here, the “Hammett's σp value”, which is proposed by L. P. Hammett, indicates the quantified effect of a substituent on the reaction rate or equilibrium of a para-substituted benzene derivative. Specifically, the value is a constant (σp) peculiar to the substituent in the following equation that is established between a substituent and a reaction rate constant or an equilibrium constant in a para-substituted benzene derivative.

$$log ( k / {k}_{0} ) = ρσ p$$

$$or$$

$$log ( K / {K}_{0} ) = ρσ p$$

In the equation, k₀ represents a rate constant of a benzene derivative having no substituent, k represents a rate constant of a benzene derivative substituted with a substituent, K₀ represents an equilibrium constant of a benzene derivative having no substituent, K represents an equilibrium constant of a benzene derivative substituted with a substituent, and p represents a reaction constant determined by the type and condition of the reaction. In relation to descriptions on “the Hammett's σp value” and the numerical value of each substituent in the present invention, the description on the σp value can be referred to in Hansch, C., et. al., Chem. Rev., 91, 165-195(1991). A group having a negative Hammett's σp value tends to exhibit electron-donating performance (donor-like performance) and a group having a positive Hammett's σp value tends to exhibit electron-accepting performance (acceptor-like performance).

In one aspect of the present invention, R¹ to R²¹ are each independently a substituent not having an unshared electron pair. In one aspect of the present invention, R¹ to R²¹ are each independently a substituent not having a π electron.

In one aspect of the present invention, R¹ to R²¹ are each independently a hydrogen atom, or selected from the group consisting of a deuterium atom, an alkyl group, an aryl group, and a group of a combination of these. In one preferred aspect of the present invention, R¹ to R²¹ are each independently a hydrogen atom, a deuterium atom, an optionally-deuterated alkyl group, or a phenyl group optionally substituted with a deuterium atom. In one aspect of the present invention, R¹ to R²¹ are each independently a hydrogen atom, a deuterium atom, or a phenyl group optionally substituted with a deuterium atom. In one aspect of the present invention, R¹ to R²¹ are each independently a hydrogen atom, a deuterium atom, or an optionally-deuterated alkyl group. In one aspect of the present invention, R¹ to R¹¹, R²⁰ and R²¹ are each independently a hydrogen atom or a deuterium atom. In one aspect of the present invention, R¹² to R¹⁵ are each independently a hydrogen atom or a deuterium atom. In one aspect of the present invention, R¹⁶ to R¹⁹ are each independently a hydrogen atom or a deuterium atom. In one aspect of the present invention, R¹ to R²¹ are each independently a hydrogen atom or a deuterium atom.

In this application, “alkyl group” can be linear, branched or cyclic. Further, two or more types of the linear portion, the cyclic portion, and the branched portion can be mixed. The number of carbon atoms of the alkyl group can be, for example, one or more, two or more, or four or more. Further, the number of carbon atoms can be 30 or less, 20 or less, 10 or less, 6 or less, or 4 or less. Specific examples of the alkyl group include a methyl group, an ethyl group, an n-propyl group, an isopropyl group, an n-butyl group, an isobutyl group, a tert-butyl group, an n-pentyl group, an isopentyl group, an n-hexyl group, a cyclopentyl group, a cyclohexyl group, and a cycloheptyl group. In one aspect of the present invention, the carbon number of the alkyl group is 1 to 4. In one aspect of the present invention, the alkyl group is a methyl group. In one aspect of the present invention, the alkyl group is an isopropyl group. In one aspect of the present invention, the alkyl group is a tert-butyl group. In the case where plural alkyl groups exist in the molecule represented by the general formula (1), these alkyl groups can be the same as or different from each other. In one aspect of the present invention, the alkyl groups in the molecule represented by the general formula (1) are all the same. The number of the alkyl groups in the molecule represented by the general formula (1) can be 0 or more, 1 or more, 2 or more, 4 or more, or 8 or more. The number of the alkyl groups in the molecule represented by the general formula (1) can be 20 or less, 10 or less, 5 or less, or 3 or less. The number of the alkyl groups in the molecule represented by the general formula (1) can be 0.

In this application, “aryl group” can be a monocycle, or can be a fused ring in which two or more rings are fused. In the case of the fused ring, the number of rings to be fused is preferably 2 to 6, and, for example, can be selected from 2 to 4. Specific examples of the ring include a benzene ring, a naphthalene ring, and an anthracene ring. Preferred are a benzene ring and a naphthalene ring, and especially preferred is a benzene ring. Specific examples of the aryl group include a phenyl group, a 1-naphthyl group, and a 2-naphthyl group, and preferred is a phenyl group. A preferred aryl group can be substituted with a substituent selected from the group consisting of a deuterium atom, an alkyl group, an aryl group, and a group of a combination of these. An unsubstituted aryl group, especially an unsubstituted phenyl group is also preferred. In one aspect of the present invention, the aryl groups in the molecule represented by the general formula (1) are all the same. The number of the aryl groups in the molecule represented by the general formula (1) can be 0 or more, 1 or more, 2 or more, or 4 or more. The number of the aryl groups in the molecule represented by the general formula (1) can be 10 or less, 5 or less, 3 or less, 2 or less, or 1 or less. The number of the aryl groups in the molecule represented by the general formula (1) can be 0.

One combination of R¹² and R¹³, R¹³ and R¹⁴, and R¹⁴ and R¹⁵ can bond to each other to form a benzofuro skeleton or a benzothieno skeleton. Any further ring is not fused with the benzofuro skeleton and the benzothieno skeleton referred to herein. In one aspect of the present invention, R¹² and R¹³ bond to each other to form a benzofuro skeleton or a benzothieno skeleton. In one aspect of the present invention, R¹³ and R¹⁴ bond to each other to form a benzofuro skeleton or a benzothieno skeleton. In one aspect of the present invention, R¹⁴ and R¹⁵ bond to each other to form a benzofuro skeleton or a benzothieno skeleton. In one aspect of the present invention, R¹² and R¹³, R¹³ and R¹⁴, and R¹⁴ and R¹⁵ all do not bond to each other to form a cyclic structure.

R¹ to R¹¹, and R¹⁶ to R²¹ do not bond to any of the other R¹ to R²¹ to form a cyclic structure. For example, R¹ does not bond to any of R² to R²¹ to form a cyclic structure. The compound represented by the general formula (1) tends to be superior to compounds in which at least one of R¹ to R¹¹ and R¹⁶ to R²¹ bonds to any of the other R¹ to R²¹ to form a cyclic structure.

In the general formula (1), X represents an oxygen atom or a sulfur atom. In one aspect of the present invention, X is a sulfur atom. In one preferred aspect of the present invention, X is an oxygen atom.

Specific examples of the group bonding to the phenylene group substituted with R⁸ to R¹¹ from the right side thereof (5-membered structure substituted with R¹² to R²¹) in the general formula (1) are shown below. However, the structures which can be adopted in this invention are not construed as limiting to these specific examples. In this application, * indicates a bonding site.

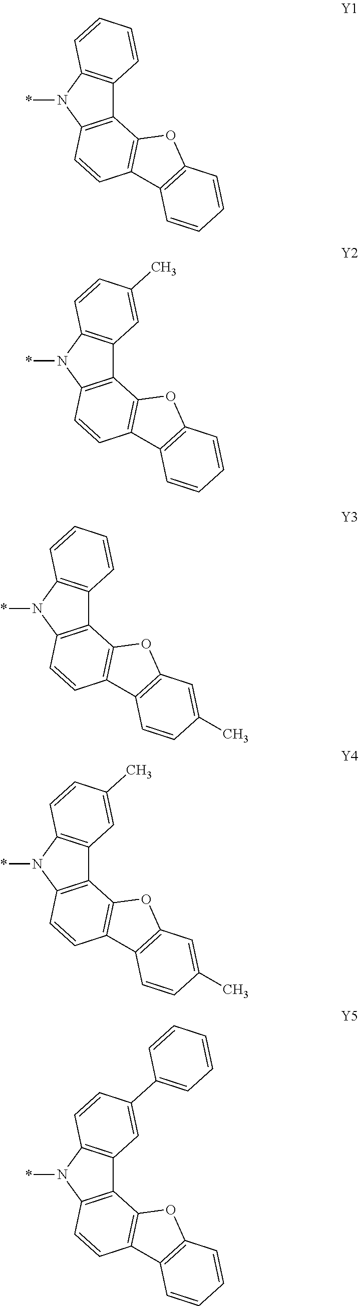

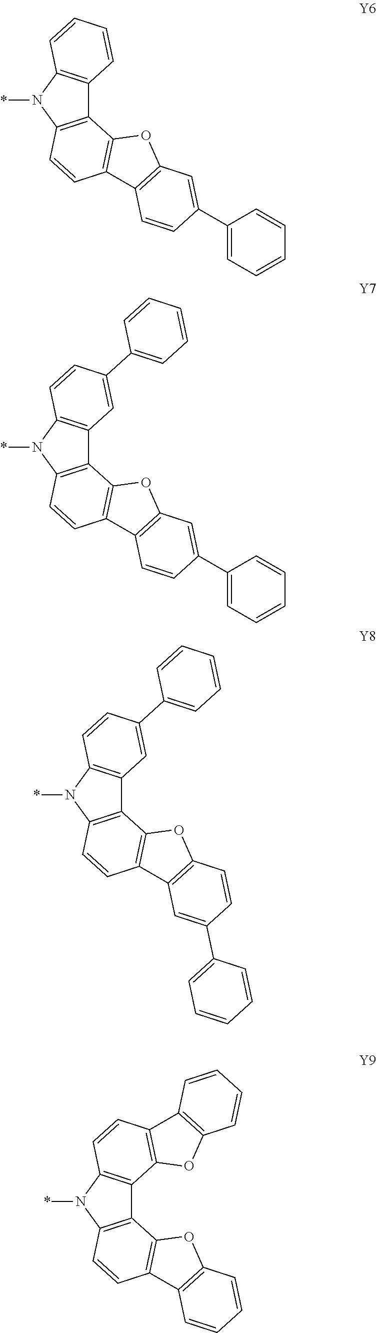

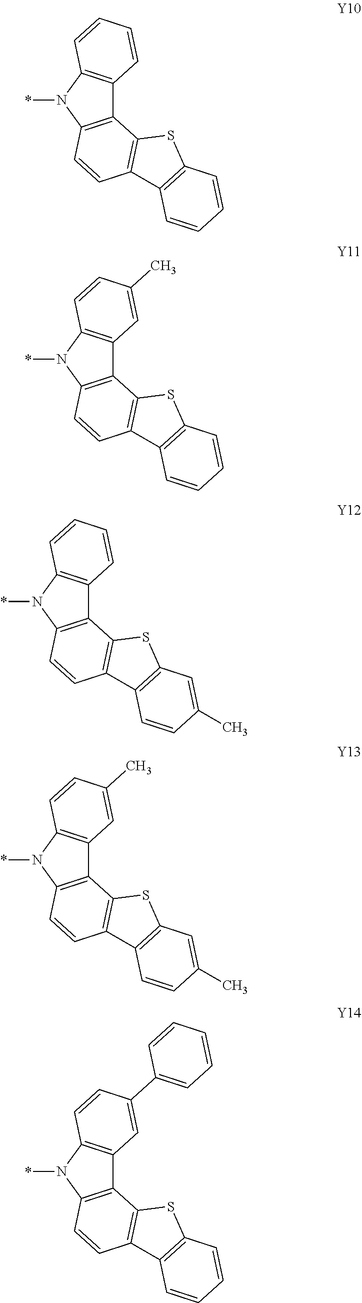

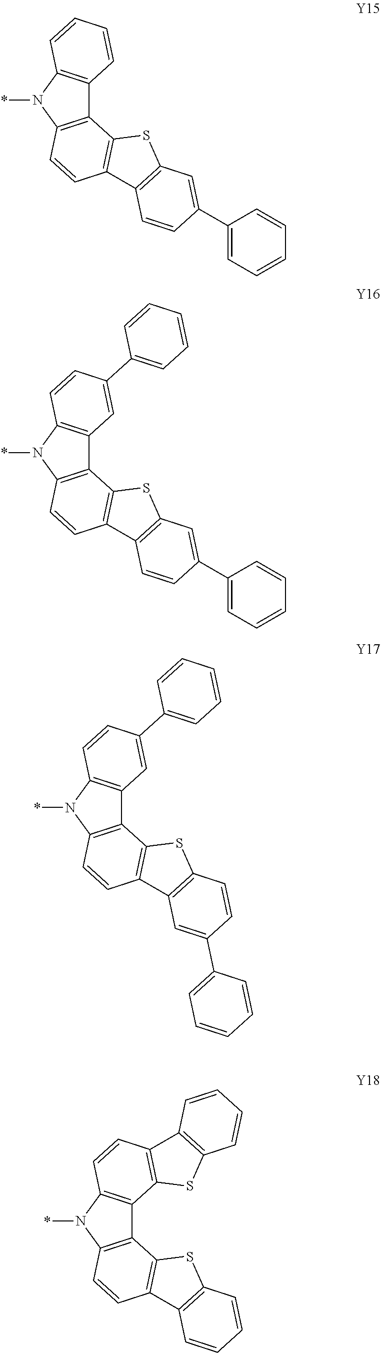

Those produced by substituting all hydrogen atoms in the above Y1 to Y18 with deuterium atoms are exemplified here as Y19 to Y36. Those produced by deuterating all hydrogen atoms of the methyl group (CH₃) existing in the above Y2 to Y8, and Y11 to Y17, or all hydrogen atoms of the phenyl group (C₆H₅) therein are exemplified here as Y37 to Y50. In one aspect of the present invention, the group is selected from Y1 to Y50. In one aspect of the present invention, the group is selected from Y1 to Y9, Y19 to Y27, and Y37 to Y43. In one aspect of the present invention, the group is selected from Y10 to Y18, Y28 to Y36, and Y44 to Y50. In one aspect of the present invention, the group is selected from Y1, Y9, Y10, Y18, Y19, Y27, Y28, and Y36. In one aspect of the present invention, the group is selected from Y2 to Y4, Y11 to Y13, Y20 to Y22, Y29 to Y31, Y37 to Y39, and Y44 to Y46. In one aspect of the present invention, the group is selected from Y5 to Y8, Y14 to Y17, Y23 to Y26, Y32 to Y35, Y40 to Y43, and Y47 to Y50. In one aspect of the present invention, the group is selected from Y9, Y18, Y27, and Y36.

The phenylene group substituted with R⁸ to R¹¹ in the general formula (1) is preferably a phenylene group optionally substituted with a deuterium atom. Examples thereof include an unsubstituted phenylene group, and a phenylene group with R⁸ to R¹¹ of deuterium atoms.

Specific examples of the group bonding to the phenylene group substituted with R⁸ to R¹¹ from the right side thereof (the dibenzofuryl group substituted with R¹ to R⁷) in the general formula (1) are shown below. However, the structures which can be adopted in this invention are not construed as limiting to these specific examples. In this application, * indicates a bonding site, and D represents a deuterium atom.

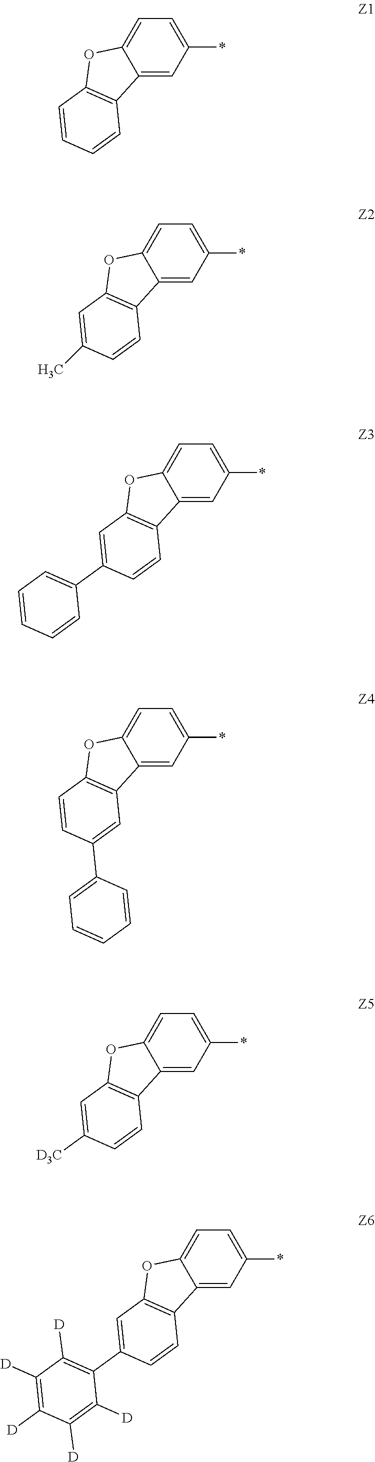

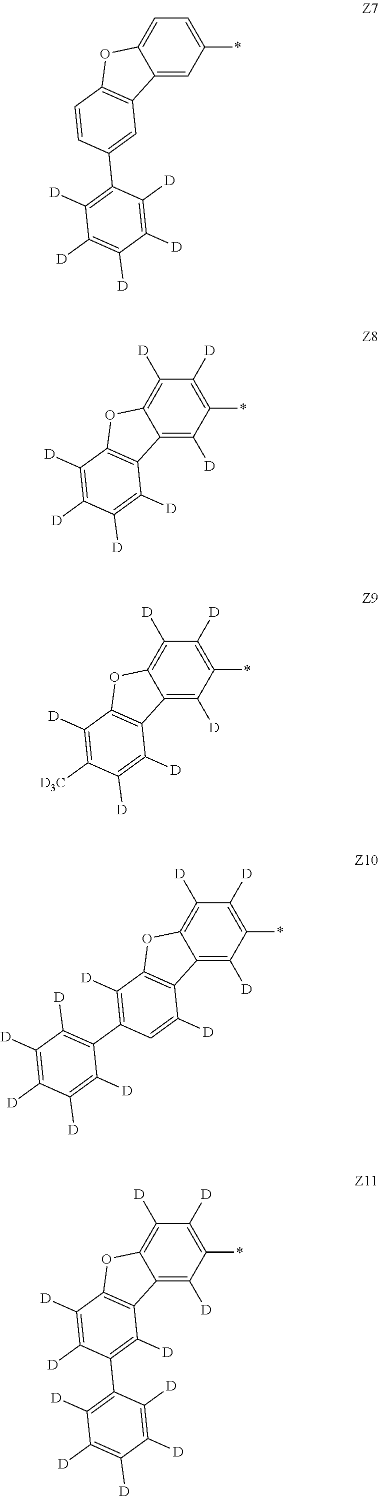

In one aspect of the present invention, the group is selected from Z1 to Z11. In one aspect of the present invention, the group is Z1 or Z8. In one aspect of the present invention, the group is selected from Z2, Z5, and Z9. In one aspect of the present invention, the group is selected from Z4, Z7, and Z11. In one aspect of the present invention, the group is selected from Z3, Z4, Z6, Z7, Z10, and Z11.

The molecular weight of the compound represented by the general formula (1) is preferably 1500 or less, more preferably 1200 or less, further preferably 1000 or less, still further preferably 900 or less, for example, when there is an intention to form and use a film of an organic layer containing the compound represented by the general formula (1) through a vapor deposition method. The lower limit value of the molecular weight is the molecular weight of the smallest compound in the compound group represented by the general formula (1).

The compound represented by the general formula (1) can be formed into a film by a coating method regardless of the molecular weight. When the coating method is used, even a compound having a relatively large molecular weight can be formed into a film. The compound represented by the general formula (1) has an advantage of being easily dissolved in an organic solvent. For this reason, the compound represented by the general formula (1) is easily applicable to a coating method and is easily purified to increase its purity.

It is preferable that the compound represented by the general formula (1) does not include a metal atom and a boron atom. For example, as the compound represented by the general formula (1), a compound including an atom selected from the group consisting of a carbon atom, a hydrogen atom, a deuterium atom, a nitrogen atom, an oxygen atom, and a sulfur atom can be selected. For example, as the compound represented by the general formula (1), a compound including an atom selected from the group consisting of a carbon atom, a hydrogen atom, a deuterium atom, a nitrogen atom, and an oxygen atom can be selected. For example, as the compound represented by the general formula (1), a compound including an atom selected from the group consisting of a carbon atom, a hydrogen atom, a nitrogen atom, and an oxygen atom can be selected.

Hereinafter, specific examples of the compound represented by the general formula (1) will be given. However, the group represented by the general formula (1) that can be adopted in the present invention is not construed as limiting to these specific examples.

First, specific examples of the compound having a structure represented by the following general formula (1a) are shown below. In Table 1, the structures of Compounds 1 to 352 are specified by specifying the groups of Z and Y of the compounds.

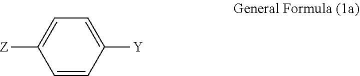

TABLE 1

| No. | Z | Y |
|---|---|---|
| 1 | Z1 | Y1 |
| 2 | Z1 | Y2 |
| 3 | Z1 | Y3 |
| 4 | Z1 | Y4 |
| 5 | Z1 | Y5 |
| 6 | Z1 | Y6 |
| 7 | Z1 | Y7 |
| 8 | Z1 | Y8 |
| 9 | Z1 | Y9 |
| 10 | Z1 | Y10 |
| 11 | Z1 | Y11 |
| 12 | Z1 | Y12 |
| 13 | Z1 | Y13 |
| 14 | Z1 | Y14 |
| 15 | Z1 | Y15 |
| 16 | Z1 | Y16 |
| 17 | Z1 | Y17 |
| 18 | Z1 | Y18 |
| 19 | Z1 | Y19 |
| 20 | Z1 | Y20 |
| 21 | Z1 | Y21 |
| 22 | Z1 | Y22 |
| 23 | Z1 | Y23 |
| 24 | Z1 | Y24 |
| 25 | Z1 | Y25 |
| 26 | Z1 | Y26 |
| 27 | Z1 | Y27 |
| 28 | Z1 | Y28 |
| 29 | Z1 | Y29 |
| 30 | Z1 | Y30 |
| 31 | Z1 | Y31 |
| 32 | Z1 | Y32 |
| 33 | Z1 | Y33 |
| 34 | Z1 | Y34 |
| 35 | Z1 | Y35 |
| 36 | Z1 | Y36 |
| 37 | Z1 | Y37 |
| 38 | Z1 | Y38 |
| 39 | Z1 | Y39 |
| 40 | Z1 | Y40 |
| 41 | Z1 | Y41 |
| 42 | Z1 | Y42 |
| 43 | Z1 | Y43 |
| 44 | Z1 | Y44 |
| 45 | Z1 | Y45 |
| 46 | Z1 | Y46 |
| 47 | Z1 | Y47 |
| 48 | Z1 | Y48 |
| 49 | Z1 | Y49 |
| 50 | Z1 | Y50 |
| 51 | Z2 | Y1 |
| 52 | Z2 | Y2 |
| 53 | Z2 | Y3 |
| 54 | Z2 | Y4 |
| 55 | Z2 | Y5 |
| 56 | Z2 | Y6 |
| 57 | Z2 | Y7 |
| 58 | Z2 | Y8 |
| 59 | Z2 | Y9 |
| 60 | Z2 | Y10 |
| 61 | Z2 | Y11 |
| 62 | Z2 | Y12 |
| 63 | Z2 | Y13 |
| 64 | Z2 | Y14 |
| 65 | Z2 | Y15 |
| 66 | Z2 | Y16 |
| 67 | Z2 | Y17 |
| 68 | Z2 | Y18 |
| 69 | Z2 | Y19 |
| 70 | Z2 | Y20 |
| 71 | Z2 | Y21 |
| 72 | Z2 | Y22 |
| 73 | Z2 | Y23 |
| 74 | Z2 | Y24 |
| 75 | Z2 | Y25 |
| 76 | Z2 | Y26 |
| 77 | Z2 | Y27 |
| 78 | Z2 | Y28 |
| 79 | Z2 | Y29 |
| 80 | Z2 | Y30 |
| 81 | Z2 | Y31 |
| 82 | Z2 | Y32 |
| 83 | Z2 | Y33 |
| 84 | Z2 | Y34 |
| 85 | Z2 | Y35 |
| 86 | Z2 | Y36 |
| 87 | Z2 | Y37 |
| 88 | Z2 | Y38 |
| 89 | Z2 | Y39 |
| 90 | Z2 | Y40 |
| 91 | Z2 | Y41 |
| 92 | Z2 | Y42 |
| 93 | Z2 | Y43 |
| 94 | Z2 | Y44 |
| 95 | Z2 | Y45 |
| 96 | Z2 | Y46 |
| 97 | Z2 | Y47 |
| 98 | Z2 | Y48 |
| 99 | Z2 | Y49 |
| 100 | Z2 | Y50 |
| 101 | Z3 | Y1 |
| 102 | Z3 | Y2 |
| 103 | Z3 | Y3 |
| 104 | Z3 | Y4 |
| 105 | Z3 | Y5 |
| 106 | Z3 | Y6 |
| 107 | Z3 | Y7 |
| 108 | Z3 | Y8 |
| 109 | Z3 | Y9 |
| 110 | Z3 | Y10 |
| 111 | Z3 | Y11 |
| 112 | Z3 | Y12 |
| 113 | Z3 | Y13 |
| 114 | Z3 | Y14 |
| 115 | Z3 | Y15 |
| 116 | Z3 | Y16 |
| 117 | Z3 | Y17 |
| 118 | Z3 | Y18 |
| 119 | Z3 | Y19 |
| 120 | Z3 | Y20 |
| 121 | Z3 | Y21 |
| 122 | Z3 | Y22 |
| 123 | Z3 | Y23 |
| 124 | Z3 | Y24 |
| 125 | Z3 | Y25 |
| 126 | Z3 | Y26 |
| 127 | Z3 | Y27 |
| 128 | Z3 | Y28 |
| 129 | Z3 | Y29 |
| 130 | Z3 | Y30 |
| 131 | Z3 | Y31 |
| 132 | Z3 | Y32 |
| 133 | Z3 | Y33 |
| 134 | Z3 | Y34 |
| 135 | Z3 | Y35 |
| 136 | Z3 | Y36 |
| 137 | Z3 | Y37 |
| 138 | Z3 | Y38 |
| 139 | Z3 | Y39 |
| 140 | Z3 | Y40 |
| 141 | Z3 | Y41 |
| 142 | Z3 | Y42 |
| 143 | Z3 | Y43 |
| 144 | Z3 | Y44 |
| 145 | Z3 | Y45 |
| 146 | Z3 | Y46 |
| 147 | Z3 | Y47 |
| 148 | Z3 | Y48 |
| 149 | Z3 | Y49 |
| 150 | Z3 | Y50 |
| 151 | Z4 | Y1 |
| 152 | Z4 | Y2 |
| 153 | Z4 | Y3 |
| 154 | Z4 | Y4 |
| 155 | Z4 | Y5 |
| 156 | Z4 | Y6 |
| 157 | Z4 | Y7 |
| 158 | Z4 | Y8 |
| 159 | Z4 | Y9 |
| 160 | Z4 | Y10 |
| 161 | Z4 | Y11 |
| 162 | Z4 | Y12 |
| 163 | Z4 | Y13 |
| 164 | Z4 | Y14 |
| 165 | Z4 | Y15 |
| 166 | Z4 | Y16 |
| 167 | Z4 | Y17 |
| 168 | Z4 | Y18 |
| 169 | Z4 | Y19 |
| 170 | Z4 | Y20 |
| 171 | Z4 | Y21 |
| 172 | Z4 | Y22 |
| 173 | Z4 | Y23 |
| 174 | Z4 | Y24 |
| 175 | Z4 | Y25 |
| 176 | Z4 | Y26 |
| 177 | Z4 | Y27 |
| 178 | Z4 | Y28 |
| 179 | Z4 | Y29 |
| 180 | Z4 | Y30 |
| 181 | Z4 | Y31 |
| 182 | Z4 | Y32 |
| 183 | Z4 | Y33 |
| 184 | Z4 | Y34 |
| 185 | Z4 | Y35 |
| 186 | Z4 | Y36 |
| 187 | Z4 | Y37 |
| 188 | Z4 | Y38 |
| 189 | Z4 | Y39 |
| 190 | Z4 | Y40 |
| 191 | Z4 | Y41 |
| 192 | Z4 | Y42 |
| 193 | Z4 | Y43 |
| 194 | Z4 | Y44 |
| 195 | Z4 | Y45 |
| 196 | Z4 | Y46 |
| 197 | Z4 | Y47 |
| 198 | Z4 | Y48 |
| 199 | Z4 | Y49 |
| 200 | Z4 | Y50 |
| 201 | Z5 | Y1 |
| 202 | Z5 | Y2 |
| 203 | Z5 | Y3 |
| 204 | Z5 | Y4 |
| 205 | Z5 | Y5 |
| 206 | Z5 | Y6 |
| 207 | Z5 | Y7 |
| 208 | Z5 | Y8 |
| 209 | Z5 | Y9 |
| 210 | Z5 | Y10 |
| 211 | Z5 | Y11 |
| 212 | Z5 | Y12 |
| 213 | Z5 | Y13 |
| 214 | Z5 | Y14 |
| 215 | Z5 | Y15 |
| 216 | Z5 | Y16 |
| 217 | Z5 | Y17 |
| 218 | Z5 | Y18 |
| 219 | Z5 | Y19 |
| 220 | Z5 | Y20 |
| 221 | Z5 | Y21 |
| 222 | Z5 | Y22 |
| 223 | Z5 | Y23 |
| 224 | Z5 | Y24 |
| 225 | Z5 | Y25 |
| 226 | Z5 | Y26 |
| 227 | Z5 | Y27 |
| 228 | Z5 | Y28 |
| 229 | Z5 | Y29 |
| 230 | Z5 | Y30 |
| 231 | Z5 | Y31 |
| 232 | Z5 | Y32 |
| 233 | Z5 | Y33 |
| 234 | Z5 | Y34 |
| 235 | Z5 | Y35 |
| 236 | Z5 | Y36 |
| 237 | Z5 | Y37 |
| 238 | Z5 | Y38 |
| 239 | Z5 | Y39 |
| 240 | Z5 | Y40 |
| 241 | Z5 | Y41 |
| 242 | Z5 | Y42 |
| 243 | Z5 | Y43 |
| 244 | Z5 | Y44 |
| 245 | Z5 | Y45 |
| 246 | Z5 | Y46 |
| 247 | Z5 | Y47 |
| 248 | Z5 | Y48 |
| 249 | Z5 | Y49 |
| 250 | Z5 | Y50 |
| 251 | Z6 | Y1 |
| 252 | Z6 | Y2 |
| 253 | Z6 | Y3 |
| 254 | Z6 | Y4 |
| 255 | Z6 | Y5 |
| 256 | Z6 | Y6 |
| 257 | Z6 | Y7 |
| 258 | Z6 | Y8 |
| 259 | Z6 | Y9 |
| 260 | Z6 | Y10 |
| 261 | Z6 | Y11 |
| 262 | Z6 | Y12 |
| 263 | Z6 | Y13 |
| 264 | Z6 | Y14 |
| 265 | Z6 | Y15 |
| 266 | Z6 | Y16 |
| 267 | Z6 | Y17 |
| 268 | Z6 | Y18 |
| 269 | Z6 | Y19 |
| 270 | Z6 | Y20 |
| 271 | Z6 | Y21 |
| 272 | Z6 | Y22 |
| 273 | Z6 | Y23 |
| 274 | Z6 | Y24 |
| 275 | Z6 | Y25 |
| 276 | Z6 | Y26 |
| 277 | Z6 | Y27 |
| 278 | Z6 | Y28 |
| 279 | Z6 | Y29 |
| 280 | Z6 | Y30 |
| 281 | Z6 | Y31 |
| 282 | Z6 | Y32 |
| 283 | Z6 | Y33 |
| 284 | Z6 | Y34 |
| 285 | Z6 | Y35 |
| 286 | Z6 | Y36 |
| 287 | Z6 | Y37 |
| 288 | Z6 | Y38 |
| 289 | Z6 | Y39 |
| 290 | Z6 | Y40 |
| 291 | Z6 | Y41 |
| 292 | Z6 | Y42 |
| 293 | Z6 | Y43 |
| 294 | Z6 | Y44 |
| 295 | Z6 | Y45 |
| 296 | Z6 | Y46 |
| 297 | Z6 | Y47 |
| 298 | Z6 | Y48 |
| 299 | Z6 | Y49 |
| 300 | Z6 | Y50 |
| 301 | Z7 | Y1 |
| 302 | Z7 | Y2 |
| 303 | Z7 | Y3 |
| 304 | Z7 | Y4 |
| 305 | Z7 | Y5 |
| 306 | Z7 | Y6 |
| 307 | Z7 | Y7 |
| 308 | Z7 | Y8 |
| 309 | Z7 | Y9 |
| 310 | Z7 | Y10 |
| 311 | Z7 | Y11 |
| 312 | Z7 | Y12 |
| 313 | Z7 | Y13 |
| 314 | Z7 | Y14 |
| 315 | Z7 | Y15 |
| 316 | Z7 | Y16 |
| 317 | Z7 | Y17 |
| 318 | Z7 | Y18 |
| 319 | Z7 | Y19 |
| 320 | Z7 | Y20 |
| 321 | Z7 | Y21 |
| 322 | Z7 | Y22 |
| 323 | Z7 | Y23 |
| 324 | Z7 | Y24 |
| 325 | Z7 | Y25 |
| 326 | Z7 | Y26 |
| 327 | Z7 | Y27 |
| 328 | Z7 | Y28 |
| 329 | Z7 | Y29 |
| 330 | Z7 | Y30 |
| 331 | Z7 | Y31 |
| 332 | Z7 | Y32 |
| 333 | Z7 | Y33 |
| 334 | Z7 | Y34 |
| 335 | Z7 | Y35 |
| 336 | Z7 | Y36 |
| 337 | Z7 | Y37 |
| 338 | Z7 | Y38 |
| 339 | Z7 | Y39 |
| 340 | Z7 | Y40 |
| 341 | Z7 | Y41 |
| 342 | Z7 | Y42 |
| 343 | Z7 | Y43 |
| 344 | Z7 | Y44 |
| 345 | Z7 | Y45 |
| 346 | Z7 | Y46 |
| 347 | Z7 | Y47 |
| 348 | Z7 | Y48 |
| 349 | Z7 | Y49 |
| 350 | Z7 | Y50 |
| 351 | Z8 | Y1 |
| 352 | Z8 | Y2 |
| 353 | Z8 | Y3 |
| 354 | Z8 | Y4 |
| 355 | Z8 | Y5 |
| 356 | Z8 | Y6 |
| 357 | Z8 | Y7 |
| 358 | Z8 | Y8 |
| 359 | Z8 | Y9 |
| 360 | Z8 | Y10 |
| 361 | Z8 | Y11 |
| 362 | Z8 | Y12 |
| 363 | Z8 | Y13 |
| 364 | Z8 | Y14 |
| 365 | Z8 | Y15 |
| 366 | Z8 | Y16 |
| 367 | Z8 | Y17 |
| 368 | Z8 | Y18 |
| 369 | Z8 | Y19 |
| 370 | Z8 | Y20 |
| 371 | Z8 | Y21 |
| 372 | Z8 | Y22 |
| 373 | Z8 | Y23 |
| 374 | Z8 | Y24 |
| 375 | Z8 | Y25 |
| 376 | Z8 | Y26 |
| 377 | Z8 | Y27 |
| 378 | Z8 | Y28 |
| 379 | Z8 | Y29 |
| 380 | Z8 | Y30 |
| 381 | Z8 | Y31 |
| 382 | Z8 | Y32 |
| 383 | Z8 | Y33 |
| 384 | Z8 | Y34 |
| 385 | Z8 | Y35 |
| 386 | Z8 | Y36 |
| 387 | Z8 | Y37 |
| 388 | Z8 | Y38 |
| 389 | Z8 | Y39 |
| 390 | Z8 | Y40 |
| 391 | Z8 | Y41 |
| 392 | Z8 | Y42 |
| 393 | Z8 | Y43 |
| 394 | Z8 | Y44 |
| 395 | Z8 | Y45 |
| 396 | Z8 | Y46 |
| 397 | Z8 | Y47 |
| 398 | Z8 | Y48 |
| 399 | Z8 | Y49 |
| 400 | Z8 | Y50 |
| 401 | Z9 | Y1 |
| 402 | Z9 | Y2 |
| 403 | Z9 | Y3 |
| 404 | Z9 | Y4 |
| 405 | Z9 | Y5 |
| 406 | Z9 | Y6 |
| 407 | Z9 | Y7 |
| 408 | Z9 | Y8 |
| 409 | Z9 | Y9 |
| 410 | Z9 | Y10 |
| 411 | Z9 | Y11 |
| 412 | Z9 | Y12 |
| 413 | Z9 | Y13 |
| 414 | Z9 | Y14 |
| 415 | Z9 | Y15 |
| 416 | Z9 | Y16 |
| 417 | Z9 | Y17 |
| 418 | Z9 | Y18 |
| 419 | Z9 | Y19 |
| 420 | Z9 | Y20 |
| 421 | Z9 | Y21 |
| 422 | Z9 | Y22 |
| 423 | Z9 | Y23 |
| 424 | Z9 | Y24 |
| 425 | Z9 | Y25 |
| 426 | Z9 | Y26 |
| 427 | Z9 | Y27 |
| 428 | Z9 | Y28 |
| 429 | Z9 | Y29 |
| 430 | Z9 | Y30 |
| 431 | Z9 | Y31 |
| 432 | Z9 | Y32 |
| 433 | Z9 | Y33 |
| 434 | Z9 | Y34 |
| 435 | Z9 | Y35 |
| 436 | Z9 | Y36 |
| 437 | Z9 | Y37 |
| 438 | Z9 | Y38 |
| 439 | Z9 | Y39 |
| 440 | Z9 | Y40 |
| 441 | Z9 | Y41 |
| 442 | Z9 | Y42 |
| 443 | Z9 | Y43 |
| 444 | Z9 | Y44 |
| 445 | Z9 | Y45 |
| 446 | Z9 | Y46 |
| 447 | Z9 | Y47 |
| 448 | Z9 | Y48 |
| 449 | Z9 | Y49 |
| 450 | Z9 | Y50 |
| 451 | Z10 | Y1 |
| 452 | Z10 | Y2 |
| 453 | Z10 | Y3 |
| 454 | Z10 | Y4 |
| 455 | Z10 | Y5 |
| 456 | Z10 | Y6 |
| 457 | Z10 | Y7 |
| 458 | Z10 | Y8 |
| 459 | Z10 | Y9 |
| 460 | Z10 | Y10 |
| 461 | Z10 | Y11 |
| 462 | Z10 | Y12 |
| 463 | Z10 | Y13 |
| 464 | Z10 | Y14 |
| 465 | Z10 | Y15 |
| 466 | Z10 | Y16 |
| 467 | Z10 | Y17 |
| 468 | Z10 | Y18 |
| 469 | Z10 | Y19 |
| 470 | Z10 | Y20 |
| 471 | Z10 | Y21 |
| 472 | Z10 | Y22 |
| 473 | Z10 | Y23 |
| 474 | Z10 | Y24 |
| 475 | Z10 | Y25 |
| 476 | Z10 | Y26 |
| 477 | Z10 | Y27 |
| 478 | Z10 | Y28 |
| 479 | Z10 | Y29 |
| 480 | Z10 | Y30 |
| 481 | Z10 | Y31 |
| 482 | Z10 | Y32 |
| 483 | Z10 | Y33 |
| 484 | Z10 | Y34 |
| 485 | Z10 | Y35 |
| 486 | Z10 | Y36 |
| 487 | Z10 | Y37 |
| 488 | Z10 | Y38 |
| 489 | Z10 | Y39 |
| 490 | Z10 | Y40 |
| 491 | Z10 | Y41 |
| 492 | Z10 | Y42 |
| 493 | Z10 | Y43 |
| 494 | Z10 | Y44 |
| 495 | Z10 | Y45 |
| 496 | Z10 | Y46 |
| 497 | Z10 | Y47 |
| 498 | Z10 | Y48 |
| 499 | Z10 | Y49 |
| 500 | Z10 | Y50 |
| 501 | Z11 | Y1 |
| 502 | Z11 | Y2 |
| 503 | Z11 | Y3 |
| 504 | Z11 | Y4 |
| 505 | Z11 | Y5 |
| 506 | Z11 | Y6 |
| 507 | Z11 | Y7 |
| 508 | Z11 | Y8 |
| 509 | Z11 | Y9 |
| 510 | Z11 | Y10 |
| 511 | Z11 | Y11 |
| 512 | Z11 | Y12 |
| 513 | Z11 | Y13 |
| 514 | Z11 | Y14 |
| 515 | Z11 | Y15 |
| 516 | Z11 | Y16 |
| 517 | Z11 | Y17 |
| 518 | Z11 | Y18 |
| 519 | Z11 | Y19 |
| 520 | Z11 | Y20 |
| 521 | Z11 | Y21 |
| 522 | Z11 | Y22 |
| 523 | Z11 | Y23 |
| 524 | Z11 | Y24 |
| 525 | Z11 | Y25 |
| 526 | Z11 | Y26 |
| 527 | Z11 | Y27 |
| 528 | Z11 | Y28 |
| 529 | Z11 | Y29 |
| 530 | Z11 | Y30 |
| 531 | Z11 | Y31 |
| 532 | Z11 | Y32 |
| 533 | Z11 | Y33 |
| 534 | Z11 | Y34 |
| 535 | Z11 | Y35 |
| 536 | Z11 | Y36 |
| 537 | Z11 | Y37 |
| 538 | Z11 | Y38 |
| 539 | Z11 | Y39 |
| 540 | Z11 | Y40 |
| 541 | Z11 | Y41 |
| 542 | Z11 | Y42 |
| 543 | Z11 | Y43 |
| 544 | Z11 | Y44 |
| 545 | Z11 | Y45 |
| 546 | Z11 | Y46 |
| 547 | Z11 | Y47 |
| 548 | Z11 | Y48 |
| 549 | Z11 | Y49 |
| 550 | Z11 | Y50 |

Those produced by substituting all hydrogen atoms in Compounds 1 to 550 with deuterium atoms are exemplified here as Compounds 551 to 1100.

In one aspect of the present invention, the compound represented by the general formula (1) is selected from Compounds 1 to 1100. In one aspect of the present invention, the compound is selected from Compounds 1 to 50, and 551 to 600. In one aspect of the present invention, the compound is selected from Compounds 51 to 100, 201 to 250, 401 to 450, 601 to 650, 751 to 800, and 951 to 1000. In one aspect of the present invention, the compound is selected from Compounds 101 to 200, 251 to 350, 451 to 550, 651 to 750, 801 to 900, and 1001 to 1100. In one aspect of the present invention, the compound is selected from Compounds 151 to 200, 301 to 350, 501 to 550, 701 to 750, 851 to 900, and 1051 to 1100.

In the compound represented by the general formula (1), Z and Y bond via the para-position of the benzene ring, as shown in the general formula (1a). The compound represented by the general formula (1) tends to be superior to the compound where Z and Y bond via the meta-position.

In the compound represented by the general formula (1), Z of the general formula (1a) is a substituted or unsubstituted dibenzofuryl group bonding at the 2-position. The compound represented by the general formula (1) tends to be superior to the compound where Z is a substituted or unsubstituted dibenzofuryl group bonding to the other position (for example, the 4-position).

In the compound represented by the general formula (1), Y of the general formula (1a) is a group fused with a benzofuro structure or a benzothieno structure at the specific position of the carbazole ring. The compound represented by the general formula (1) tends to be superior to the compound where Y is a group fused with a benzofuro structure or a benzothieno structure at a different position of the carbazole ring.

The compound represented by the general formula (1) can be synthesized using a known synthesis method. For example, the compound represented by the general formula (1a) can be readily synthesized by coupling Z—C₆H₅Br and H—Y according to the following reaction formula. Specifically, the compound can be synthesized by reacting Z—C₆H₅Br and an equimolar amount of H—Y, for example, in the presence of tris(dibenzylideneacetone)dipalladium(0), tri-tert-butylphosphonium tetrafluoroborate and sodium tert-butoxide. As the solvent, for example, toluene can be used, and the reaction can be promoted by refluxing for one day. The resultant product is extracted with an organic solvent, and purified by silica gel column chromatography and recrystallization to give the intended compound having a high purity.

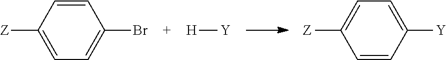

### [Organic Semiconductor Device]

The compound represented by the general formula (1) can be favorably applied to an organic semiconductor device. For example, a CMOS (complementary metal-oxide film semiconductor) or the like using the compound represented by the general formula (1) can be produced. In some embodiments of the present disclosure, an organic optical device such as an organic electroluminescent device or a solid-state imaging device (for example, a CMOS image sensor) can be produced by using the compound represented by the general formula (1). Above all, the compound represented by the general formula (1) can be used for an organic light emitting device such as an organic electroluminescent device (organic EL device). In particular, the compound represented by the general formula (1) of the present invention can be effectively used as an electron barrier material for an organic light emitting device. In particular, by using the compound represented by the general formula (1) of the present invention in an electron barrier layer, the device life can be prolonged.

The organic electroluminescent device has a structure in which at least an anode, a cathode, and an organic layer between the anode and the cathode are formed. The organic layer includes at least a light emitting layer, and preferably has at least one organic layer (especially electron barrier layer) in addition to the light emitting layer. The organic layer to constitute the organic electroluminescent device includes a hole transport layer, a hole injection layer, an electron barrier layer, a hole barrier layer, an electron injection layer, an electron transport layer, an exciton barrier layer, an underlayer for the light emitting layer, and the like. The hole transport layer can be a hole injection transport layer having a hole injection function, and the electron transport layer can be an electron injection transport layer having an electron injection function.

In the following, the constituent members and layers of the organic electroluminescent device are described. The description of the substrate and the light emitting layer can apply also to the substrate and the light emitting layer of an organic photoluminescent device.

### (Electron Barrier Layer)

In one preferred aspect of the present invention, the compound represented by the general formula (1) is used for the electron barrier layer of an organic electroluminescent device. The electron barrier layer can contain only the compound represented by the general formula (1), or can additionally contain any other compound than the compound represented by the general formula (1). The concentration of the compound represented by the general formula (1) in the electron barrier layer is preferably 50% by weight or more, more preferably 90% by weight or more, and can be, for example, 99% by weight or more, and can be 99.9% by weight or more. The thickness of the electron barrier layer is preferably 1 nm or more, more preferably 3 nm or more, and for example, can be 5 nm or more, or can be, for example, 10 nm or more. The thickness of the electron barrier layer is preferably less than 30 nm, more preferably less than 20 nm, and for example, can be 15 nm. The thickness of the electron barrier layer is preferably smaller than the thickness of the light emitting layer. The thickness of the electron barrier layer is preferably one-second of the thickness of the light emitting layer or less, more preferably one-third or less, and for example can be one-fourth or less. In addition, it is preferably one-twentieth or more, and for example can be one-tenth or more, or for example can be one-sixth or more.

The electron barrier layer containing the compound represented by the general formula (1) is preferably arranged between the light emitting layer and the anode. In one aspect of the present invention, the light emitting layer and the electron barrier layer are laminated so as to be in direct contact with each other.

In one aspect of the present invention, the device includes a laminate structure of an electron barrier layer containing the compound represented by the general formula (1), an underlayer, and a light emitting layer laminated in that order from the anode side. The electron barrier layer and the underlayer are laminated so as to be in direct contact with each other, and the underlayer and the light emitting layer are laminated so as to be in direct contact with each other, but the electron barrier layer and the light emitting layer are not in contact with each other.

### (Underlayer)

The underlayer is formed for the purpose of improving the orientation of the light emitting layer and the like, and is a layer containing a hole transporting material. In one aspect of the present invention, the underlayer contains a compound having a partial structure common to the compound contained in the light emitting layer. The term “common partial structure” as used herein means that a partial structure composed of 12 or more atoms other than a hydrogen atom and a deuterium atom is in common, and a partial structure composed of 16 or more atoms other than a hydrogen atom and a deuterium atom is preferably in common, and for example, a partial structure composed of 20 or more atoms other than a hydrogen atom and a deuterium atom can be in common. In one aspect of the present invention, the underlayer contains a compound that is the same as the compound contained in the light emitting layer. In one aspect of the present invention, the underlayer contains only a compound that is the same as the compound contained in the light emitting layer. In one aspect of the present invention, the underlayer contains a compound that is the same as the host material contained in the light emitting layer. The thickness of the underlayer is preferably 1 nm or more, more preferably 3 nm or more, and for example, can be 5 nm or more. The thickness of the adjacent layer is preferably less than 30 nm, more preferably less than 20 nm, and for example, can be 10 nm or less, or can be 7 nm or less. The thickness of the underlayer is preferably smaller than the thickness of the light emitting layer. The thickness of the underlayer is preferably one-second of the thickness of the light emitting layer or less, more preferably one-third or less, and for example, can be one-fourth or less. In addition, it is preferably one-twentieth or more, and for example, can be one-tenth or more. The thickness of the underlayer is preferably smaller than the thickness of the electron barrier layer. The thickness of the underlayer can be, for example, three-fourth of the thickness of the electron barrier layer or less, can be, for example, two-third or less, or can be, for example, one-second or less. In addition, it is preferably one-twentieth or more, and for example, can be one-tenth or more, or for example, can be one-fourth or more.

### (Light Emitting Layer)

The light emitting layer is a layer where holes and electrons injected from the anode and the cathode, respectively, are recombined to form excitons, and then emit light. The light emitting layer contains at least a light emitting material.

In order that an organic electroluminescent device can express a high light emission efficiency, it is important that the singlet excitons and the triplet excitons in the light emitting material are confined in the light emitting material. Accordingly, it is preferable to use a host material in addition to the light emitting material in the light emitting layer. As the host material, usable is an organic compound having a higher excited singlet energy than that of the light emitting material in the present invention, and preferably used here is an organic compound whose excited singlet energy and excited triplet energy are both higher than those of the light emitting material. Using a host material, the singlet excitons and the triplet excitons formed in the light emitting material can be confined in the molecule of the light emitting material, and light emission efficiency can be sufficiently expressed. Naturally, even if the singlet excitons and the triplet excitons could not be sufficiently confined, a high light emission efficiency can be attained in some cases, and therefore, a host material capable of expressing a high light emission efficiency can be used in the present invention with no specific limitation. In the organic electroluminescent device of the present invention, the maximum amount of light emitted from the device is light emitted from the light emitting material contained in the light emitting layer. The light emission includes fluorescent light emission and can contain delayed fluorescence. However, the host material can partly or partially emit light.

In the case of using a host material, the concentration of the light emitting material in the light emitting layer is preferably 0.1% by weight or more, more preferably 1% by weight or more, and is preferably 50% by weight or less, more preferably 20% by weight or less, further preferably 10% by weight or less.

An assist dopant can be used in the light emitting layer. In that case, the light emitting layer is composed of a host material, an assist dopant and a light emitting material. Here, as the host material, used is one having a higher lowest excited singlet energy than that of the assist dopant, and as the light emitting material, used is one having a lower lowest excited singlet energy than that of the assist dopant. In the present invention, it is especially preferable to use a delayed fluorescent material as the assist dopant. Delayed fluorescence means fluorescence which a compound having been in an excited state emits after the compound has undergone reverse intersystem crossing from an excited triplet state to an excited singlet state and when it returns back from the excited singlet state to a ground state, and is fluorescence observed later than fluorescence (instantaneous fluorescence) from the excited singlet state that has directly transitioned from the ground state. In the present invention, in the case where a transient decay curve of light emission of a thin film containing a targeted compound is measured at 300K, when a light emission component having a long light emission lifetime (delayed fluorescence) is observed apart from a light emission component having a short light emission lifetime (instantaneous fluorescence), that targeted compound is a delayed fluorescent material. The delayed fluorescent material is preferably a thermal activation-type delayed fluorescent material that can undergo reverse intersystem crossing by absorption of thermal energy. The fact that the fluorescent material is a thermal activation-type delayed fluorescent material can be confirmed by the fact that the light emission lifetime of the material to be determined by measurement of the transient decay curve of light emission thereof becomes long depending on the measurement temperature. Using a delayed fluorescent material as an assist dopant, the energy of the excited singlet state formed by direct transition from the ground state of the assist dopant and the excited singlet energy by reverse intersystem crossing thereof can efficiently move to a light emitting material to thereby effectively assist the light emission of the light emitting material.

In the case where the light emitting layer is composed of a host material, an assist dopant and a light emitting material, the concentration of the assist dopant in the light emitting layer is preferably smaller than the content of the host material therein. Specifically, when the total weight of the content of the host material, the content of the assist dopant, and the content of the light emitting layer is 100% by weight, the content of the host material is preferably 15% by weight or more and 99.9% by weight or less, the content of the assist dopant is preferably 5.0% by weight or more and 50% by weight or less, and the content of the light emitting material is preferably 0.5% by weight or more and 5.0% by weight or less.

In one aspect of the present invention, the light emitting layer does not contain an inorganic compound. Also in one aspect of the present invention, the light emitting layer does not contain a metal atom. In one aspect of the present invention, phosphorescence is not observed from the light emitting layer at 300K.

The host material used in the light emitting layer is preferably an organic compound having a hole transporting ability and an electron transporting ability, preventing the light emission from being a longer wavelength, and having a high glass transition temperature. In one aspect of the present invention, a compound containing a carbazole structure is preferably selected as the host material. In one preferred aspect of the present invention, a compound containing at least two structures selected from the group consisting of a carbazole structure, a dibenzofuran structure and a dibenzothiophene structure, for example, containing two such structures, or containing three such structures can be selected as the host material. In one preferred aspect of the present invention, a compound containing a 1,3-phenylene structure can be selected as the host material. In one preferred aspect of the present invention, a compound containing a biphenylene structure can be selected as the host material. In one preferred aspect of the present invention, a compound having 5 to 8 benzene rings in the molecule can be selected as the host material, and for example, a compound having 5 benzene rings can be selected, a compound having 6 benzene rings can be selected, or a compound having 7 benzene rings can be selected.

Compounds preferably usable as the host material are shown below, but the host material that can be adopted in the present invention is not construed as limiting to the following specific examples.

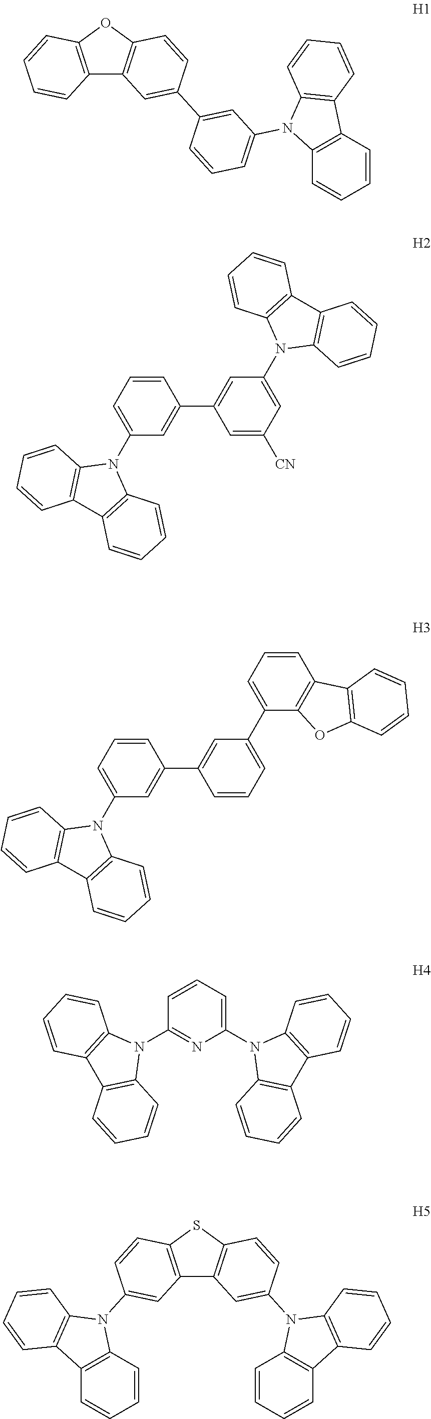 [SMILES: c1cc(-c2ccc3oc4ccccc4c3c2)cc(-n2c3ccccc3c3ccccc32)c1 ; N#Cc1cc(-c2cccc(-n3c4ccccc4c4ccccc43)c2)cc(-n2c3ccccc3c3ccccc32)c1 ; c1cc(-c2cccc(-n3c4ccccc4c4ccccc43)c2)cc(-c2cccc3c2oc2ccccc23)c1 ; c1cc(-n2c3ccccc3c3ccccc32)nc(-n2c3ccccc3c3ccccc32)c1 ; c1ccc2c(c1)c1ccccc1n2-c1ccc2sc3ccc(-n4c5ccccc5c5ccccc54)cc3c2c1]

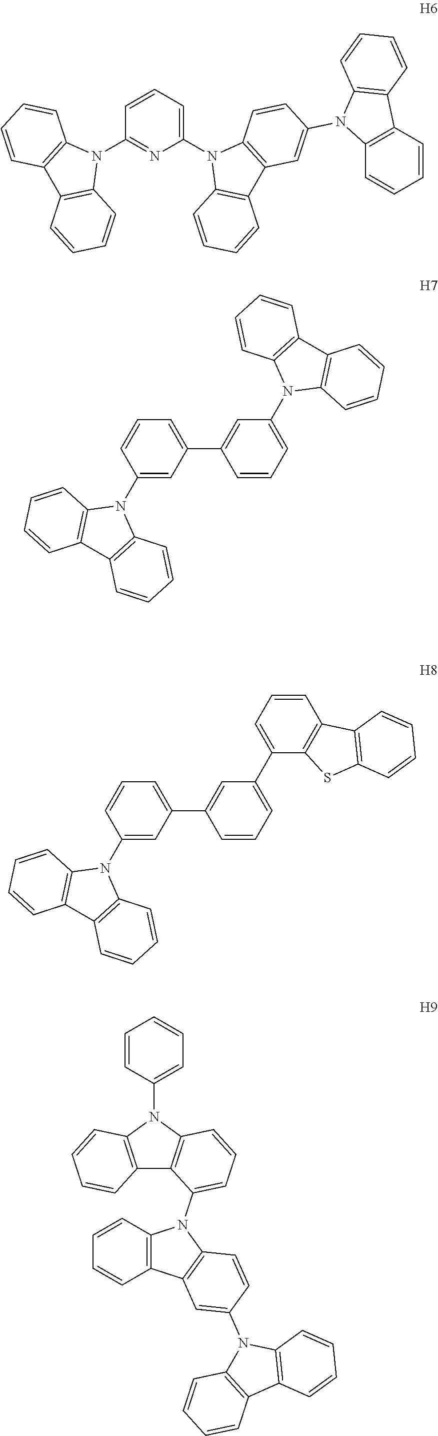 [SMILES: c1cc(-n2c3ccccc3c3ccccc32)nc(-n2c3ccccc3c3cc(-n4c5ccccc5c5ccccc54)ccc32)c1 ; c1cc(-c2cccc(-n3c4ccccc4c4ccccc43)c2)cc(-n2c3ccccc3c3ccccc32)c1 ; c1cc(-c2cccc(-n3c4ccccc4c4ccccc43)c2)cc(-c2cccc3c2sc2ccccc23)c1 ; c1ccc(-n2c3ccccc3c3c(-n4c5ccccc5c5cc(-n6c7ccccc7c7ccccc76)ccc54)cccc32)cc1]

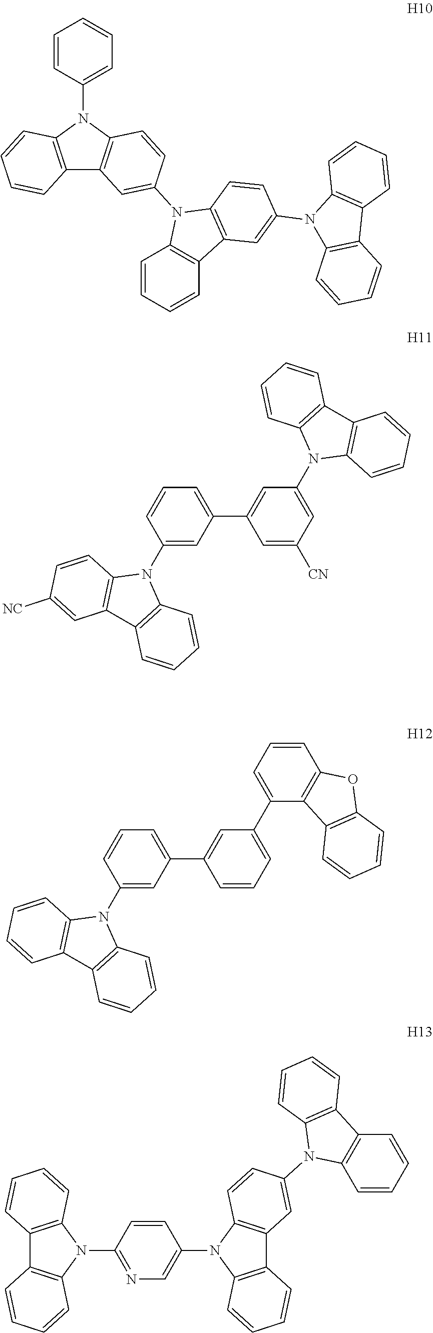 [SMILES: c1ccc(-n2c3ccccc3c3cc(-n4c5ccccc5c5cc(-n6c7ccccc7c7ccccc76)ccc54)ccc32)cc1 ; N#Cc1cc(-c2cccc(-n3c4ccccc4c4cc(C#N)ccc43)c2)cc(-n2c3ccccc3c3ccccc32)c1 ; c1cc(-c2cccc(-n3c4ccccc4c4ccccc43)c2)cc(-c2cccc3oc4ccccc4c23)c1 ; c1ccc2c(c1)c1ccccc1n2-c1ccc2c(c1)c1ccccc1n2-c1ccc(-n2c3ccccc3c3ccccc32)nc1]

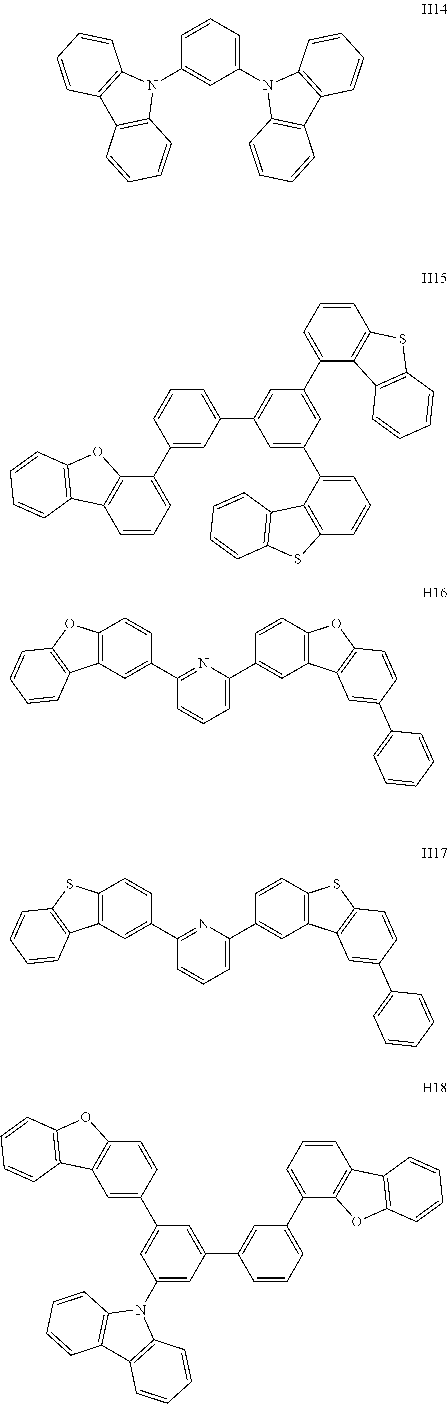 [SMILES: c1cc(-n2c3ccccc3c3ccccc32)cc(-n2c3ccccc3c3ccccc32)c1 ; c1cc(-c2cc(-c3cccc4sc5ccccc5c34)cc(-c3cccc4sc5ccccc5c34)c2)cc(-c2cccc3c2oc2ccccc23)c1 ; c1ccc(-c2ccc3oc4ccc(-c5cccc(-c6ccc7oc8ccccc8c7c6)n5)cc4c3c2)cc1 ; c1ccc(-c2ccc3sc4ccc(-c5cccc(-c6ccc7sc8ccccc8c7c6)n5)cc4c3c2)cc1 ; c1cc(-c2cc(-c3ccc4oc5ccccc5c4c3)cc(-n3c4ccccc4c4ccccc43)c2)cc(-c2cccc3c2oc2ccccc23)c1]

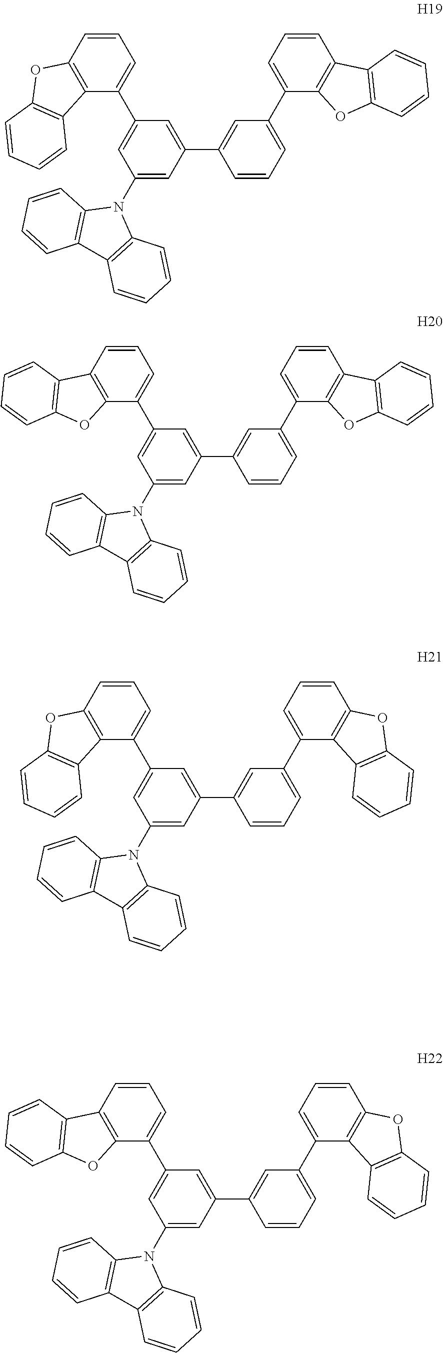

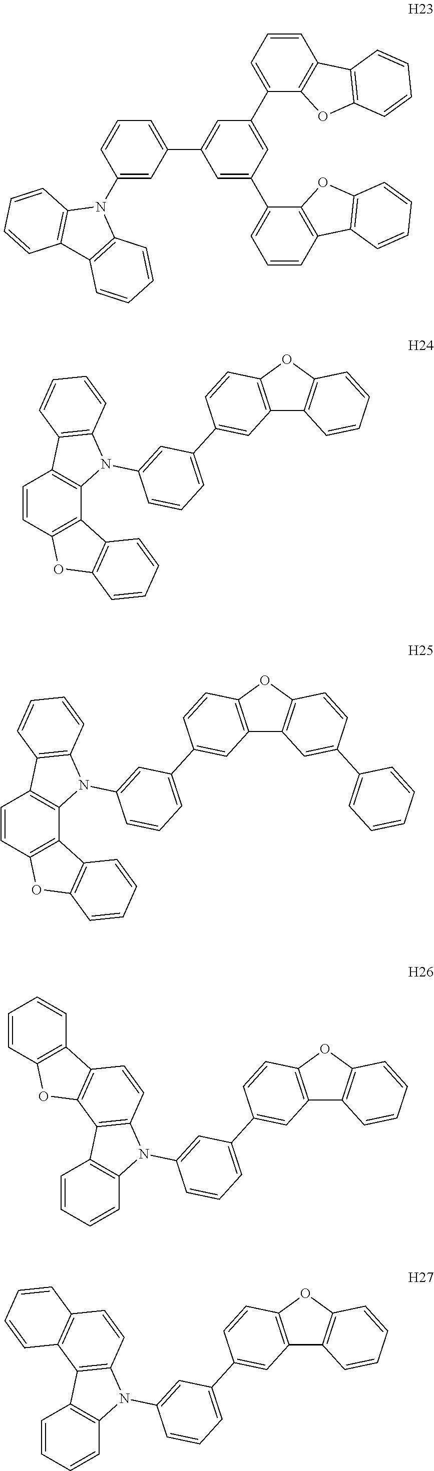 [SMILES: c1cc(-c2cc(-c3cccc4c3oc3ccccc34)cc(-c3cccc4c3oc3ccccc34)c2)cc(-n2c3ccccc3c3ccccc32)c1 ; c1cc(-c2ccc3oc4ccccc4c3c2)cc(-n2c3ccccc3c3ccc4oc5ccccc5c4c32)c1 ; c1ccc(-c2ccc3oc4ccc(-c5cccc(-n6c7ccccc7c7ccc8oc9ccccc9c8c76)c5)cc4c3c2)cc1 ; c1cc(-c2ccc3oc4ccccc4c3c2)cc(-n2c3ccccc3c3c4oc5ccccc5c4ccc32)c1 ; c1cc(-c2ccc3oc4ccccc4c3c2)cc(-n2c3ccccc3c3c4ccccc4ccc32)c1]

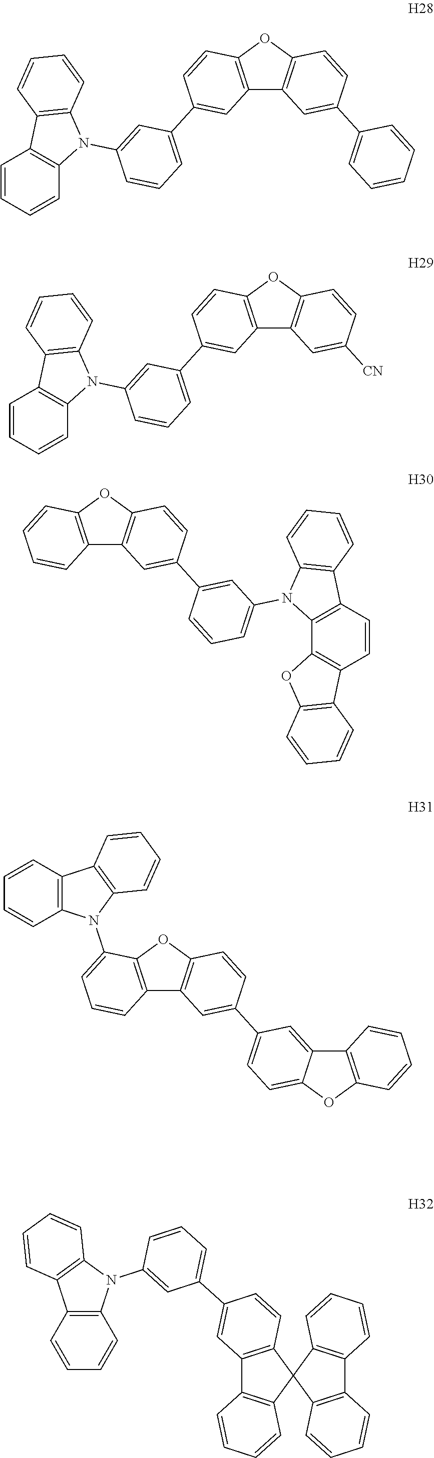 [SMILES: c1ccc(-c2ccc3oc4ccc(-c5cccc(-n6c7ccccc7c7ccccc76)c5)cc4c3c2)cc1 ; N#Cc1ccc2oc3ccc(-c4cccc(-n5c6ccccc6c6ccccc65)c4)cc3c2c1 ; c1cc(-c2ccc3oc4ccccc4c3c2)cc(-n2c3ccccc3c3ccc4c5ccccc5oc4c32)c1 ; c1ccc2c(c1)oc1ccc(-c3ccc4oc5c(-n6c7ccccc7c7ccccc76)cccc5c4c3)cc12 ; c1cc(-c2ccc3c(c2)-c2ccccc2C32c3ccccc3-c3ccccc32)cc(-n2c3ccccc3c3ccccc32)c1]

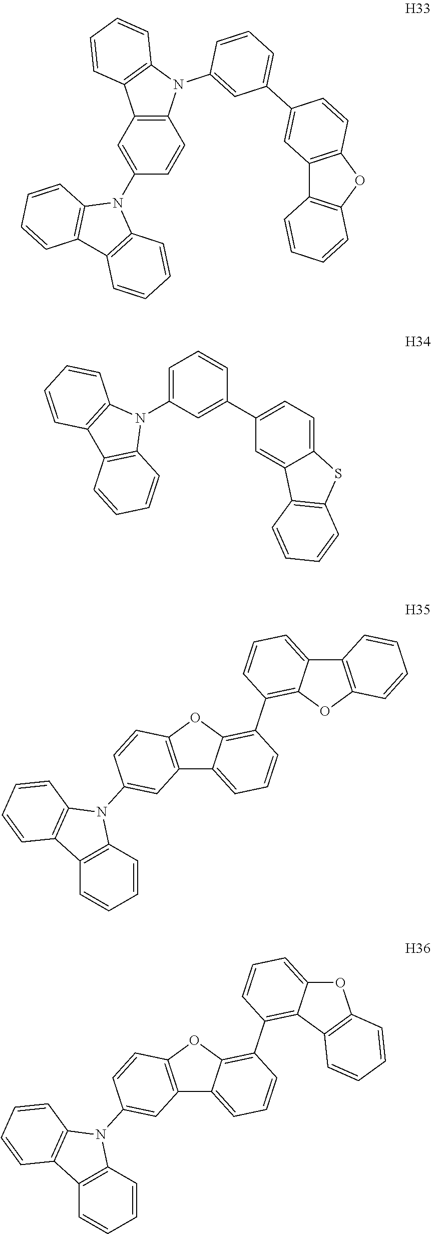 [SMILES: c1cc(-c2ccc3oc4ccccc4c3c2)cc(-n2c3ccccc3c3cc(-n4c5ccccc5c5ccccc54)ccc32)c1 ; c1cc(-c2ccc3sc4ccccc4c3c2)cc(-n2c3ccccc3c3ccccc32)c1 ; c1ccc2c(c1)oc1c(-c3cccc4c3oc3ccc(-n5c6ccccc6c6ccccc65)cc34)cccc12 ; c1ccc2c(c1)oc1cccc(-c3cccc4c3oc3ccc(-n5c6ccccc6c6ccccc65)cc34)c12]

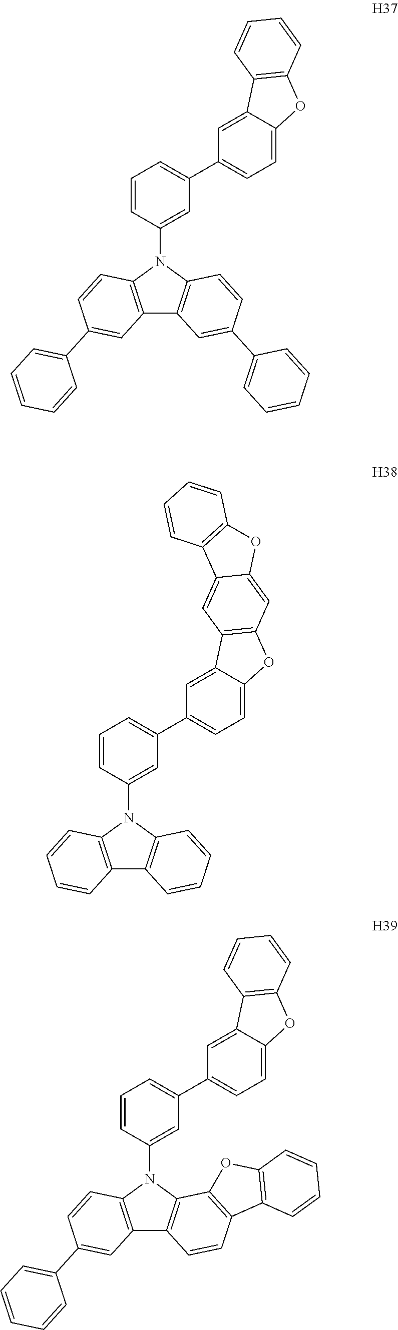 [SMILES: c1ccc(-c2ccc3c(c2)c2cc(-c4ccccc4)ccc2n3-c2cccc(-c3ccc4oc5ccccc5c4c3)c2)cc1 ; c1cc(-c2ccc3oc4cc5oc6ccccc6c5cc4c3c2)cc(-n2c3ccccc3c3ccccc32)c1 ; c1ccc(-c2ccc3c(c2)c2ccc4c5ccccc5oc4c2n3-c2cccc(-c3ccc4oc5ccccc5c4c3)c2)cc1]

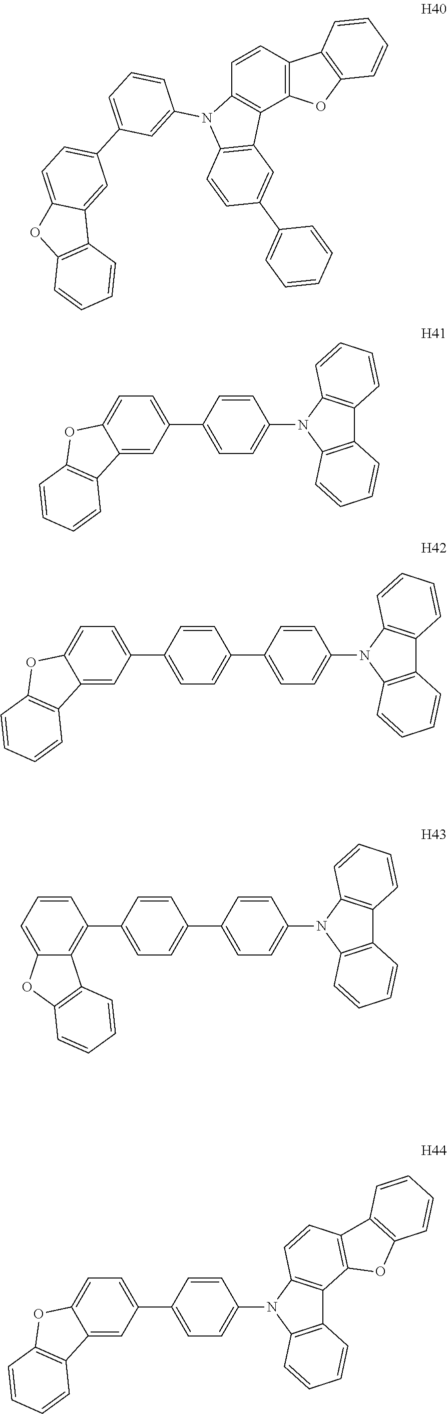 [SMILES: c1ccc(-c2ccc3c(c2)c2c4oc5ccccc5c4ccc2n3-c2cccc(-c3ccc4oc5ccccc5c4c3)c2)cc1 ; c1ccc2c(c1)oc1ccc(-c3ccc(-n4c5ccccc5c5ccccc54)cc3)cc12 ; c1ccc2c(c1)oc1ccc(-c3ccc(-c4ccc(-n5c6ccccc6c6ccccc65)cc4)cc3)cc12 ; c1ccc2c(c1)oc1cccc(-c3ccc(-c4ccc(-n5c6ccccc6c6ccccc65)cc4)cc3)c12 ; c1ccc2c(c1)oc1ccc(-c3ccc(-n4c5ccccc5c5c6oc7ccccc7c6ccc54)cc3)cc12]

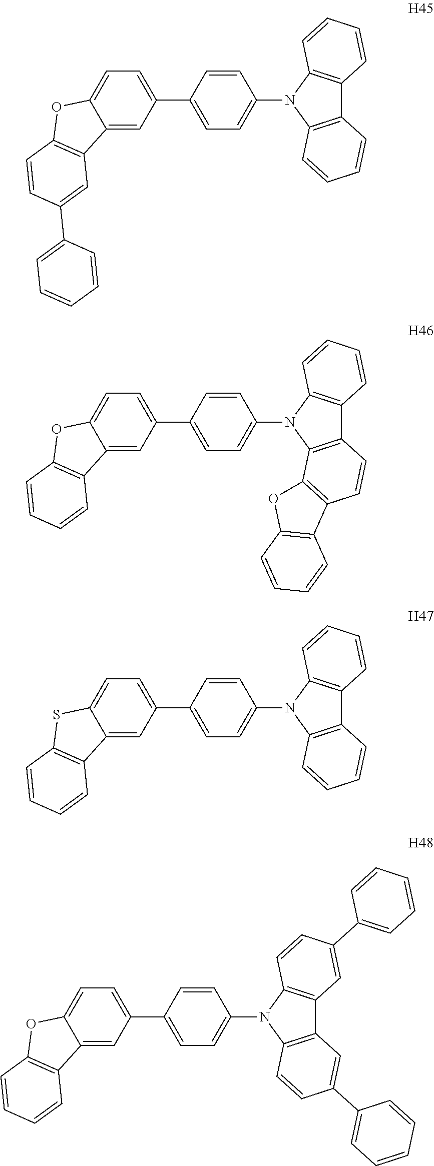

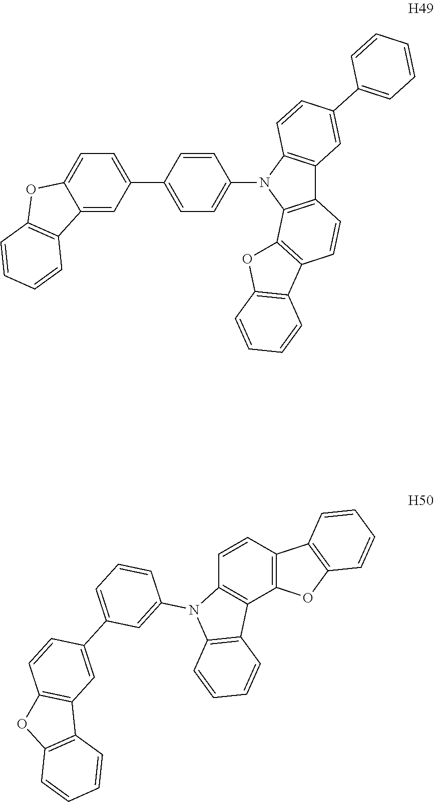 [SMILES: c1ccc(-c2ccc3c(c2)c2ccc4c5ccccc5oc4c2n3-c2ccc(-c3ccc4oc5ccccc5c4c3)cc2)cc1 ; c1cc(-c2ccc3oc4ccccc4c3c2)cc(-n2c3ccccc3c3c4oc5ccccc5c4ccc32)c1]

In the light emitting layer, a delayed fluorescent material can be used as the light emitting material or an assist dopant. For the light emitting material and the assist dopant, different delayed fluorescent materials can be used. The delayed fluorescent material generally gives fluorescence that has an emission lifetime of 100 ns (nanoseconds) or longer, when the emission lifetime thereof is measured with a fluorescence lifetime measuring system (for example, a streak camera system by Hamamatsu Photonics K.K.). The delayed fluorescent material is preferably such that the difference ΔE_{ST} between the lowest excited singlet energy and the lowest excited triplet energy at 77K is 0.3 eV or less, more preferably 0.25 eV or less, further preferably 0.2 eV or less, still further preferably 0.15 eV or less, still further more preferably 0.1 eV or less, still further more preferably 0.07 eV or less, still further more preferably 0.05 eV or less, still further more preferably 0.03 eV or less, particularly preferably 0.01 eV or less. When ΔE_{ST} is small, reverse intersystem crossing from an excited triplet state to an excited singlet state can readily occur through thermal energy absorption, and therefore the compound of the type can function as a thermal activation type delayed fluorescent material. A thermal activation type delayed fluorescent material can absorb heat generated by a device to relatively readily undergo reverse intersystem crossing from an excited triplet state to an excited singlet state, and can make the excited triplet energy efficiently contribute toward light emission.

In the present invention, the lowest excited singlet energy (E_{S1}) and the lowest excited triplet energy (E_{T1}) of a compound are determined according to the following process. ΔE_{ST} is a value determined by calculating E_{S1}−E_{T1}.

### (1) Lowest Excited Singlet Energy (E_{S1})

A thin film or a toluene solution (concentration: 10⁻⁵ mol/L) of the targeted compound is prepared as a measurement sample. The fluorescent spectrum of the sample is measured at room temperature (300 K). For the fluorescent spectrum, the emission intensity is on the vertical axis and the wavelength is on the horizontal axis. A tangent line is drawn to the rising of the emission spectrum on the short wavelength side, and the wavelength value λedge [nm] at the intersection between the tangent line and the horizontal axis is read. The wavelength value is converted into an energy value according to the following conversion expression to calculate E_{S1}.

Conversion Expression: E_{S1} [eV]=1239.85/λedge

For the measurement of the emission spectrum in Examples given below, an LED light source (by Thorlabs Corporation, M300L4) was used as an excitation light source along with a detector (by Hamamatsu Photonics K.K., PMA-12 Multichannel Spectroscope C10027-01).

### (2) Lowest Excited Triplet Energy (E_{T1})

The same sample as that for measurement of the lowest excited singlet energy (E_{S1}) is cooled to 77 [K] with liquid nitrogen, and the sample for phosphorescence measurement is irradiated with excitation light (300 nm), and using the detector, the phosphorescence thereof is measured. The light emission after 100 milliseconds from irradiation with the excitation light is drawn as a phosphorescent spectrum. A tangent line is drawn to the rising of the phosphorescent spectrum on the short wavelength side, and the wavelength value λedge [nm] at the intersection between the tangent line and the horizontal axis is read. The wavelength value is converted into an energy value according to the following conversion expression to calculate E_{T1}.

Conversion Expression: E_{T1} [eV]=1239.85/λedge

The tangent line to the rising of the phosphorescent spectrum on the short wavelength side is drawn as follows. While moving on the spectral curve from the short wavelength side of the phosphorescent spectrum toward the local maximum value on the shortest wavelength side among the local maximum values of the spectrum, a tangent line at each point on the curve toward the long wavelength side is taken into consideration. With rising thereof(that is, with increase in the vertical axis), the inclination of the tangent line increases. The tangent line drawn at the point at which the inclination value has a local maximum value is referred to as the tangent line to the rising on the short wavelength side of the phosphorescent spectrum.

The local maximum point having a peak intensity of 10% or less of the maximum peak intensity of the spectrum is not included in the local maximum value on the above-mentioned shortest wavelength side, and the tangent line drawn at the point which is closest to the local maximum value on the shortest wavelength side and at which the inclination value has a local maximum value is referred to as the tangent line to the rising on the short wavelength side of the phosphorescent spectrum.

Preferably, the delayed fluorescent material does not contain a metal atom. For example, as the delayed fluorescent material, a compound including an atom selected from the group consisting of a carbon atom, a hydrogen atom, a deuterium atom, a nitrogen atom, an oxygen atom, and a sulfur atom can be selected. For example, as the delayed fluorescent material, a compound composed of a carbon atom, a hydrogen atom and a nitrogen atom can be selected.

A typical delayed fluorescent material includes a compound having a structure in which 1 or 2 acceptor groups and at least one donor group bond to a benzene ring. Preferred examples of the acceptor group include a cyano group, and a group that contains a heteroaryl ring containing a nitrogen atom as a ring skeleton-constituting atom such as a triazinyl ring. Preferred examples of the donor group include a substituted or unsubstituted carbazol-9-yl group. Examples thereof include a compound in which at least three substituted or unsubstituted carbazol-9-yl groups bond to a benzene ring, and a compound in which a 5-membered ring moiety of a substituted or unsubstituted benzofuran ring, a substituted or unsubstituted benzothiophene ring, a substituted or unsubstituted indole ring, a substituted or unsubstituted indene ring, or a substituted or unsubstituted silaindene ring is fused to at least one of the two benzene rings constituting a carbazol-9-yl group.

In one preferred aspect of the present invention, a compound represented by the following general formula (4) is used as the delayed fluorescent material.

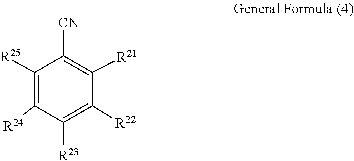

In the general formula (4), one of R²¹ to R²³ represents a cyano group or a group represented by the following general formula (5), the remaining two of R²¹ to R²³ and at least one of R²⁴ and R²⁵ each represent a group represented by the following general formula (6), the remaining R²¹ to R²⁵ each represent a hydrogen atom or a substituent, provided that the substituent referred to here is not a cyano group, the group represented by the following general formula (5) and the group represented by the following general formula (6).

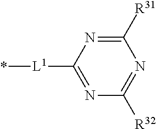

In the general formula (5), L¹ represents a single bond or a divalent linking group, R³¹ and R³² each independently represents a hydrogen atom or a substituent, * indicates a bonding site.

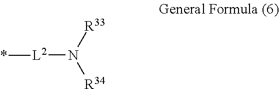

In the general formula (6), L² represents a single bond or a divalent linking group, R³³ and R³⁴ each independently represents a hydrogen atom or a substituent, * indicates a bonding site.

In one preferred aspect of the present invention, R²² is a cyano group. In one preferred aspect of the present invention, R²² is a group represented by the general formula (5). In one aspect of the present invention, R²¹ is a cyano group, or a group represented by the general formula (5). In one aspect of the present invention, R²³ is a cyano group, or a group represented by the general formula (5). In one aspect of the present invention, one of R²¹ to R²³ is a cyano group. In one aspect of the present invention, one of R²¹ to R²³ is a group represented by the general formula (5).

In one preferred aspect of the present invention, L¹ in the general formula (5) is a single bond. In one aspect of the present invention, L¹ is a divalent linking group, and is preferably a substituted or unsubstituted arylene group or a substituted or unsubstituted heteroarylene group, more preferably a substituted or unsubstituted arylene group, further preferably a substituted or unsubstituted 1,4-phenylene group (in which the substituent is, for example, an alkyl group having 1 to 3 carbon atoms).

In one aspect of the present invention, R³¹ and R³² in the general formula (5) are each independently one group selected from the group consisting of an alkyl group (for example, having 1 to 40 carbon atoms), an aryl group (for example, having 6 to 30 carbon atoms), a heteroaryl group (for example, having 5 to 30 ring skeleton-constituting atoms), an alkenyl group (for example, having 2 to 40 carbon atoms) and an alkynyl group (for example, having 2 to 40 carbon atoms), or a group formed by combining at least two such groups (hereinunder these groups are referred to as “groups of Substituent Group A”). In one preferred aspect of the present invention, R³¹ and R³² are each independently a substituted or unsubstituted aryl group (for example, having 6 to 30 carbon atoms), and the substituent for the aryl group includes the groups of Substituent Group A. In one preferred aspect of the present invention, R³¹ and R³² are the same.

In one preferred aspect of the present invention, L² in the general formula (6) is a single bond. In one aspect of the present invention, L² is a divalent linking group, and is preferably a substituted or unsubstituted arylene group or a substituted or unsubstituted heteroarylene group, more preferably a substituted or unsubstituted arylene group, further preferably a substituted or unsubstituted 1,4-phenylene group (in which the substituent is, for example, an alkyl group having 1 to 3 carbon atoms).

In one aspect of the present invention, R³³ and R³⁴ in the general formula (6) are each independently a substituted or unsubstituted alkyl group (for example, having 1 to 40 carbon atoms), a substituted or unsubstituted alkenyl group (for example, having 2 to 40 carbon atoms), a substituted or unsubstituted aryl group (for example, having 6 to 30 carbon atoms), or a substituted or unsubstituted heteroaryl group (for example, having 5 to 30 carbon atoms). The substituent for the alkyl group, the alkenyl group, the aryl group and the heteroaryl group as referred to herein includes one group selected from the group consisting of a hydroxy group, a halogen atom (for example, a fluorine atom, a chlorine atom, a bromine atom, an iodine atom), an alkyl group (for example, having 1 to 40 carbon atoms), an alkoxy group (for example, having 1 to 40 carbon atoms), an alkylthio group (for example, having 1 to 40 carbon atoms), an aryl group (for example, having 6 to 30 carbon atoms), an aryloxy group (for example, having 6 to 30 carbon atoms), an arylthio group (for example, having 6 to 30 carbon atoms), a heteroaryl group (for example, having 5 to 30 ring skeleton-constituting atoms), a heteroaryloxy group (for example, having 5 to 30 ring skeleton-constituting atoms), a heteroarylthio group (for example, having 5 to 30 ring skeleton-constituting atoms), an acyl group (for example, having 2 to 40 carbon atoms), an alkenyl group (for example, having 2 to 40 carbon atoms), an alkynyl group (for example, having 2 to 40 carbon atoms), an alkoxycarbonyl group (for example, having 2 to 40 carbon atoms), an aryloxycarbonyl group (for example, having 7 to 40 carbon atoms), a heteroaryloxycarbonyl group (for example, having 7 to 40 carbon atoms), a silyl group (for example, a trialkylsilyl group having 3 to 40 carbon atoms), a nitro group and a cyano group, or a group formed by combining at least two such groups (hereinunder these groups are referred to as “groups of Substituent Group B”).

R³³ and R³⁴ can bond to each other via a single bond or a linking group to form a cyclic structure. In particular, in the case were R³³ and R³⁴ are aryl groups, preferably, they bond to each other via a single bond or a linking group to form a cyclic structure. The linking group as referred to herein includes —O—, —S—, —N(R³⁵)—, —C(R³⁶)(R³⁷)—, and —C(═O)—, preferably —O—, —S—, —N(R³⁵)—, and —C(R³⁶)(R³⁷)—, more preferably —O—, —S—, and —N(R³⁵)—. R³⁵ to R³⁷ each independently represent a hydrogen atom or a substituent. For the substituent, the groups of the above Substituent Group A can be selected, or the groups of the above Substituent Group B can be selected, and preferably, the substituent is one group selected from the group consisting of an alkyl group having 1 to 10 carbon atoms and an aryl group having 6 to 14 carbon atoms, or a group formed by combining at least two such groups.

The group represented by the general formula (6) is preferably a group represented by the following general formula (7).

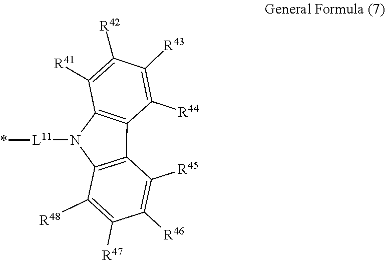

In the general formula (7), L¹¹ represents a single bond or a divalent linking group. Regarding the description and the preferred range of L¹¹, reference can be made to the description and the preferred range of L² described hereinabove.

In the general formula (7), R⁴¹ to R⁴⁸ each independently represent a hydrogen atom or a substituent. R⁴¹ and R⁴², R⁴² and R⁴³, R⁴³ and R⁴⁴, R⁴⁴ and R⁴⁵, R⁴⁵ and R⁴⁶, R⁴⁶ and R⁴⁷, and R⁴⁷ and R⁴⁸, each can bond to each other to form a cyclic structure. The cyclic structure to be formed by bonding to each other can be an aromatic ring or an aliphatic ring, or can contain a hetero atom, and further, the cyclic structure can also be a fused ring of two or more rings. Here the hetero atom is preferably selected from the group consisting of a nitrogen atom, an oxygen atom and a sulfur atom. Examples of the cyclic structure to be formed include a benzene ring, a naphthalene ring, a pyridine ring, a pyridazine ring, a pyrimidine ring, a pyrazine ring, a pyrrole ring, an imidazole ring, a pyrazole ring, an imidazoline ring, an oxazole ring, an isoxazole ring, a thiazole ring, an isothiazole ring, a cyclohexadiene ring, a cyclohexene ring, a cyclopentaene ring, a cycloheptatriene ring, a cycloheptadiene ring, a cycloheptaene ring, a furan ring, a thiophene ring, a naphthyridine ring, a quinoxaline ring, and a quinoline ring. Many rings can be fused to form a ring such as a phenanthrene ring or a triphenylene ring. The number of the rings contained in the group represented by the general formula (7) can be selected from the range of 3 to 5, or can be selected from the range of 5 to 7.

The substituent which R⁴¹ to R⁴⁸ can take includes the groups of the above-mentioned Substituent Group B, and is preferably an unsubstituted alkyl group having 1 to 10 carbon atoms, or an aryl group having 6 to 10 carbon atoms optionally substituted with an unsubstituted alkyl group having 1 to 10 carbon atoms. In one preferred aspect of the present invention, R⁴¹ to R⁴⁸ each are a hydrogen atom or an unsubstituted alkyl group having 1 to 10 carbon atoms. In one preferred aspect of the present invention, R⁴¹ to R⁴⁸ each are a hydrogen atom or an unsubstituted aryl group having 6 to 10 carbon atoms. In one preferred aspect of the present invention, R⁴¹ to R⁴⁸ are all hydrogen atoms.

In the general formula (7), * indicates a bonding site.

In one preferred aspect of the present invention, an azabenzene derivative is used as the delayed fluorescent material. In one preferred aspect of the present invention, the azabenzene derivative has an azabenzene structure in which three ring skeleton-constituting carbon atoms of the benzene ring are substituted with nitrogen atoms. For example, an azabenzene derivative having a 1,3,5-triazine structure can be preferably selected. In one preferred aspect of the present invention, the azabenzene derivative has an azabenzene structure in which two ring skeleton-constituting carbon atoms of the benzene ring are substituted with nitrogen atoms. For example, it includes an azabenzene derivative having a pyridazine structure, a pyrimidine structure, or a pyrazine structure, and an azabenzene derivative having a pyrimidine structure can be preferably selected. In one aspect of the present invention, the azabenzene derivative has a pyridine structure in which one ring skeleton-constituting carbon atom of the benzene ring is substituted with a nitrogen atom.

In one preferred aspect of the present invention, a compound represented by the following general formula (8) is used as the delayed fluorescent material.

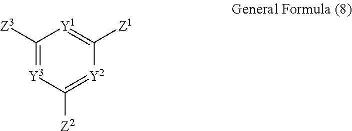

In the general formula (8), at least one of Y¹, Y² and Y³ is a nitrogen atom and the remainder represents a methine group. In one aspect of the present invention, Y¹ is a nitrogen atom, and Y² and Y³ are methine groups. Preferably, Y¹ and Y² are nitrogen atoms, and Y³ is a methine group. More preferably, Y¹ to Y³ are all nitrogen atoms.

In the general formula (8), Z¹ to Z³ each independently represent a hydrogen atom or a substituent, but at least one is a donor substituent. The donor substituent means a group having a negative Hammett's σp value. Preferably, at least one of Z¹ to Z³ is a group containing a diarylamino structure (in which the two aryl groups bonding to the nitrogen atom can bond to each other), and is more preferably a group represented by the above general formula (6), for example, a group represented by the above general formula (7). In one aspect of the present invention, only one of Z¹ to Z³ is a group represented by the general formula (6) or (7). In one aspect of the present invention, only two of Z¹ to Z³ are each independently a group represented by the general formula (6) or (7). In one aspect of the present invention, all of Z¹ to Z³ are each independently a group represented by the general formula (6) or (7). For details and preferable ranges of the general formula (6) and the general formula (7), the corresponding descriptions given above can be referred to. The remaining Z¹ to Z³ that are not the groups represented by the general formula (6) and the general formula (7) each are preferably a substituted or unsubstituted aryl group (for example, having 6 to 40 carbon atoms, preferably 6 to 20 carbon atoms), and examples of the substituent for the aryl group as referred to herein include one group selected from the group consisting of an aryl group (for example, having 6 to 20 carbon atoms, preferably 6 to 14 carbon atoms) and an alkyl group (for example, having 1 to 20 carbon atoms, preferably 1 to 6 carbon atoms), and a group formed by combining at least two such groups. In one aspect of the present invention, the general formula (8) does not include a cyano group.

In one preferred aspect of the present invention, a compound represented by the following general formula (9) is used as the delayed fluorescent material.

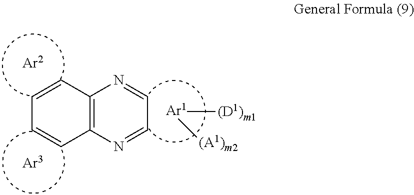

In the general formula (9), Ar¹ forms a cyclic structure optionally substituted with the following A¹ and D¹, and represents a benzene ring, a naphthalene ring, an anthracene ring or a phenanthrene ring. Ar² and Ar³ each can form a cyclic structure, and in the case of forming a cyclic structure, they represent a benzene ring, a naphthalene ring, a pyridine ring, or a benzene ring substituted with a cyano group. m1 represents an integer of any of 0 to 2, and m2 represents an integer of any of 0 to 1. A¹ represents a cyano group, a phenyl group, a pyrimidyl group, a triazyl group, or a benzonitrile group. D¹ represents a substituted or unsubstituted 5H-indolo[3,2,1-de]phenazin-5-yl group, or a substituted or unsubstituted hetero ring-fused carbazolyl group not containing a naphthalene structure, and in the case where the general formula (9) has plural D¹'s, they can be the same or different. The substituents for D¹ can bond to each other to form a cyclic structure.

Compounds represented by the following general formula (E1) are further preferred delayed fluorescent materials.

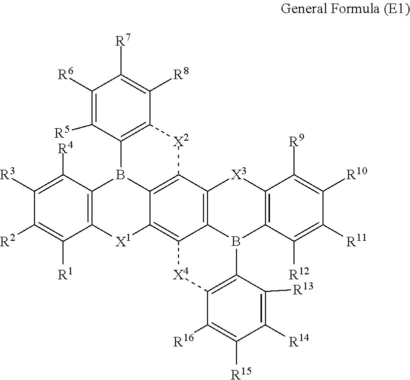

In the general formula (E1), R¹, and R³ to R¹⁶ each independently represent a hydrogen atom, a deuterium atom or a substituent. R² represents an acceptor group, or R¹ and R² bond to each other to form an acceptor group, or R² and R³ bond to each other to form an acceptor group. R³ and R⁴, R⁴ and R⁵, R⁵ and R⁶, R⁶ and R⁷, R⁷ and R⁸, R⁹ and R¹⁰, R¹⁰ and R¹¹, R¹¹ and R¹², R¹² and R¹³, R¹³ and R¹⁴, R¹⁴ and R¹⁵, and R¹⁵ and R¹⁶ each can bond to each other to form a cyclic structure. X¹ represents O or NR, and R represents a substituent. Of X² to X⁴, at least one of X³ and X⁴ is O or NR, and the remainder can be O or R, or unlinked. When not linked, both ends each independently represent a hydrogen atom, a deuterium atom or a substituent. In the general formula (E1), C—R¹, C—R³, C—R⁴, C—R⁵, C—R⁶, C—R⁷, C—R⁸, C—R⁹, C—R¹⁰, C—R¹¹, C—R¹², C—R¹³, C—R¹⁴, C—R¹⁵, and C—R¹⁶ can be substituted with N.

Compounds represented by the following general formula (E2) are further preferred delayed fluorescent materials.

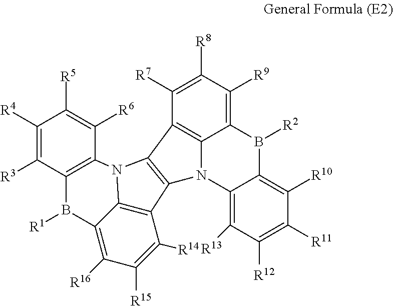

In the general formula (E2), R¹ and R² each independently represent a substituted or unsubstituted alkyl group, a substituted or unsubstituted aryl group, or a substituted or unsubstituted heteroaryl group, R³ to R¹⁶ each independently represent a hydrogen atom, a deuterium atom or a substituent. R¹ and R³, R³ and R⁴, R⁴ and R⁵, R⁵ and R⁶, R⁶ and R⁷, R⁷ and R⁸, R⁸ and R⁹, R⁹ and R², R² and R¹⁰, R¹⁰ and R¹¹, R¹¹ and R¹², R¹² and R¹³, R¹³ and R¹⁴, R¹⁴ and R¹⁵, R¹⁵ and R¹⁶, and R¹⁶ and R¹ each can bond to each other to form a cyclic structure. In the general formula (E2), C—R³, C—R⁴, C—R⁵, C—R⁶, C—R⁷, C—R⁸, C—R⁹, C—R¹⁰, C—R¹¹, C—R¹², C—R¹³, C—R¹⁴, C—R¹⁵, and C—R¹⁶ can be substituted with N.

Compounds represented by the following general formula (E3) are further preferred delayed fluorescent materials.

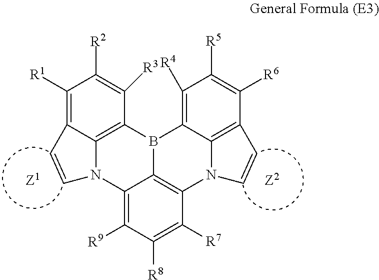

In the general formula (E3), Z¹ and Z² each independently represent a substituted or unsubstituted aromatic ring, or a substituted or unsubstituted heteroaromatic ring, and R¹ to R⁹ each independently represent a hydrogen atom, a deuterium atom or a substituent. R¹ and R², R² and R³, R³ and R⁴, R⁴ and R⁵, R⁵ and R⁶, R⁷ and R⁸ and R⁸ and R⁹ each can bond to each other to form a cyclic structure. However, at least one of the ring formed by Z¹, Z², or R¹ and R² bonding to each other, the ring formed by R² and R³ bonding to each other, the ring formed by R⁴ and R⁵ bonding to each other, and the ring formed by R⁵ and R⁶ bonding to each other is a furan ring of a substituted or unsubstituted benzofuran, a thiophene ring of a substituted or unsubstituted benzothiophene, or a pyrrole ring of a substituted or unsubstituted indole, and at least one of R¹ to R⁹ is a substituted or unsubstituted aryl group or an acceptor group, or at least one of Z¹ and Z² is a ring having an aryl group or an acceptor group as a substituent. Of the benzene ring skeleton-constituting carbon atoms to constitute the benzofuran ring, the benzothiophene ring, and the indole ring, a substitutable carbon atom can be substituted with a nitrogen atom. In the general formula (E3), C—R¹, C—R², C—R³, C—R⁴, C—R⁵, C—R⁶, C—R⁷, C—R⁸, and C—R⁹ can be substituted with N.

Compounds represented by the following general formula (E4) are further preferred delayed fluorescent materials.

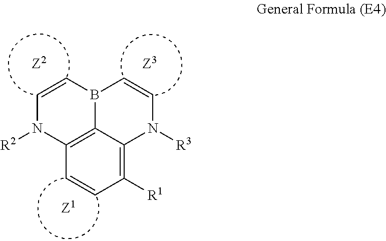

In the general formula (E4), Z¹ represents a furan ring fused with a substituted or unsubstituted benzene ring, a thiophene ring fused with a substituted or unsubstituted benzene ring, or an N-substituted pyrrole ring fused with a substituted or unsubstituted benzene ring, Z² and Z³ each independently represent a substituted or unsubstituted aromatic ring, or a substituted or unsubstituted heteroaromatic ring, R¹ represents a hydrogen atom, a deuterium atom, or a substituent, R² and R³ each independently represent a substituted or unsubstituted aryl group, or a substituted or unsubstituted heteroaryl group. Z¹ and R¹, R² and Z², Z² and Z³, and Z³ and R³ each can bond to each other to form a cyclic structure. However, at least one combination of R² and Z², Z² and Z³, and Z³ and R³ bonds to each other to form a cyclic structure.

Compounds represented by the following general formula (E5) are further preferred delayed fluorescent materials.

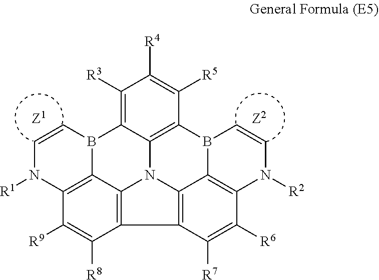

In the general formula (E5), R¹ and R² each independently represent a substituted or unsubstituted alkyl group, a substituted or unsubstituted aryl group, or a substituted or unsubstituted heteroaryl group, Z¹ and Z² each independently represent a substituted or unsubstituted aromatic ring, or a substituted or unsubstituted heteroaromatic ring, R³ to R⁹ each independently represent a hydrogen atom, a deuterium atom or a substituent. However, at least one of R¹, R², Z¹ and Z² includes a substituted or unsubstituted benzofuran ring, a substituted or unsubstituted benzothiophene ring, or a substituted or unsubstituted indole ring. R¹ and Z¹, Z¹ and R³, R³ and R⁴, R⁴ and R⁵, R⁵ and Z², Z² and R², R² and R⁶, R⁶ and R⁷, R⁷ and R⁸, R⁸ and R⁹, and R⁹ and R¹ each can bond to each other to form a cyclic structure. Of the benzene ring skeleton-constituting carbon atoms to constitute the benzofuran ring, the benzothiophene ring, and the indole ring, a substitutable carbon atom can be substituted with a nitrogen atom. In the general formula (E5), C—R³, C—R⁴, C—R⁵, C—R⁶, C—R⁷, C—R⁸, and C—R⁹ can be substituted with N.

Compounds represented by the following general formula (E6) are further preferred delayed fluorescent materials.

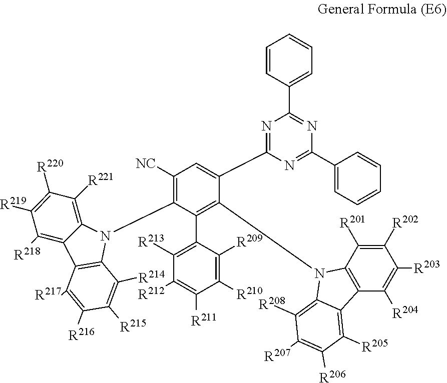

In the general formula (E6), R²⁰¹ to R²²¹ each independently represent a hydrogen atom, a deuterium atom or a substituent, preferably a hydrogen atom, a deuterium atom, an alkyl group, an aryl group, or a group formed by combining an alkyl group and an aryl group. At least one combination of R²⁰¹ and R²⁰², R²⁰² and R²⁰³, R²⁰³ and R²⁰⁴, R²⁰⁵ and R²⁰⁶, R²⁰⁶ and R²⁰⁷, R²⁰⁷ and R²⁰⁸, R²¹⁴ and R²¹⁵, R²¹⁵ and R²¹⁶, R²¹⁶ and R²¹⁷, R²¹⁸ and R²¹⁹, R²¹⁹ and R²²⁰, and R²²⁰ and R²²¹ each bond to each other to form a benzofuro structure or a benzothieno structure. Preferably, one or two combinations of R²⁰¹ and R²⁰², R²⁰² and R²⁰³, R²⁰³ and R²⁰⁴, R²⁰⁵ and R²⁰⁶, R²⁰⁶ and R²⁰⁷ and R²⁰⁷ and R²⁰⁸, and one or two combinations of R²¹⁴ and R²¹⁵, R²¹⁵ and R²¹⁶, R²¹⁶ and R²¹⁷, R²¹⁸ and R²¹⁹, R²¹⁹ and R²²⁰ and R²²⁰ and R²²¹ bond to each other to form a benzofuro structure or a benzothieno structure. Further preferably, R²⁰³ and R²⁰⁴ bond to each other to form a benzofuro structure or a benzothieno structure, further preferably, R²⁰³ and R²⁰⁴, and R²¹⁶ and R²¹⁷ each bond to each other to form a benzofuro structure or a benzothieno structure. Especially preferably, R²⁰³ and R²⁰⁴, and R²¹⁶ and R²¹⁷ each bond to each other to form a benzofuro structure or a benzothieno structure, and R²⁰⁶ and R²¹⁹ each represent a substituted or unsubstituted aryl group (preferably, a substituted or unsubstituted phenyl group, more preferably an unsubstituted phenyl group).

In the general formula (E6), R²⁰¹ to R²⁰⁸, and R²¹⁴ to R²²¹ can be each independently a deuterium atom, but contain a structure not a hydrogen atom (¹H). Specifically, in the case where R²⁰¹ to R²⁰⁸, and R²¹⁴ to R²²¹ contain an atom having one proton, the atom contains a structure limited to a deuterium atom.

Further, compounds represented by the general formulae (1) described in Japanese Patent Application Nos. 2021-103698, 2021-103699, 2021-103700, 2021-081332, 2021-103701, 2021-151805, and 2021-188860 can be used as delayed fluorescent materials. Descriptions of these general formulae (1) and specific compounds are hereby incorporated by reference as a part of this description.

Preferred compounds usable as a delayed fluorescent material are shown below. In the structural formulae of the following exemplary compounds, t-Bu represents a tertiary butyl group (tert-butyl group).

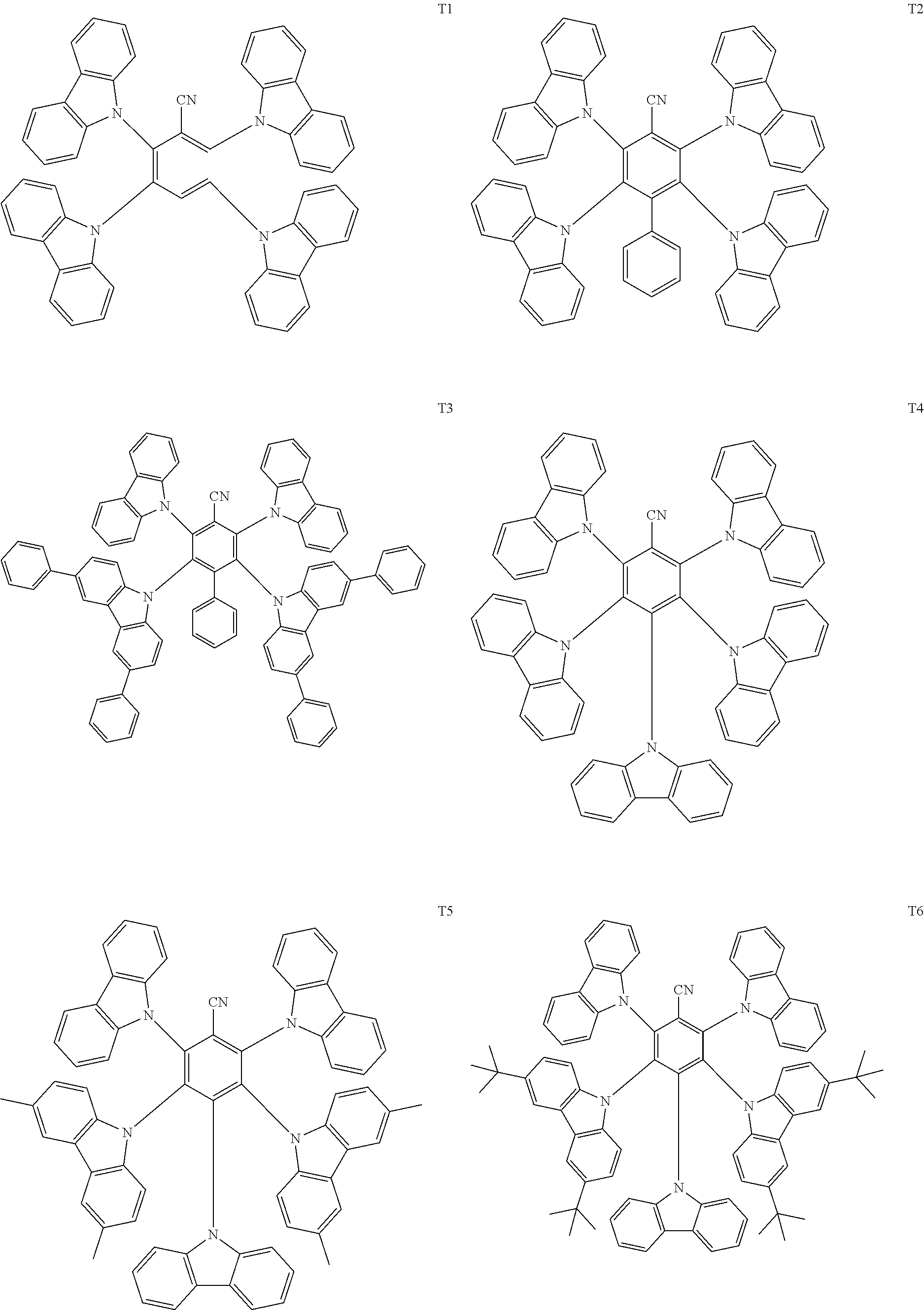

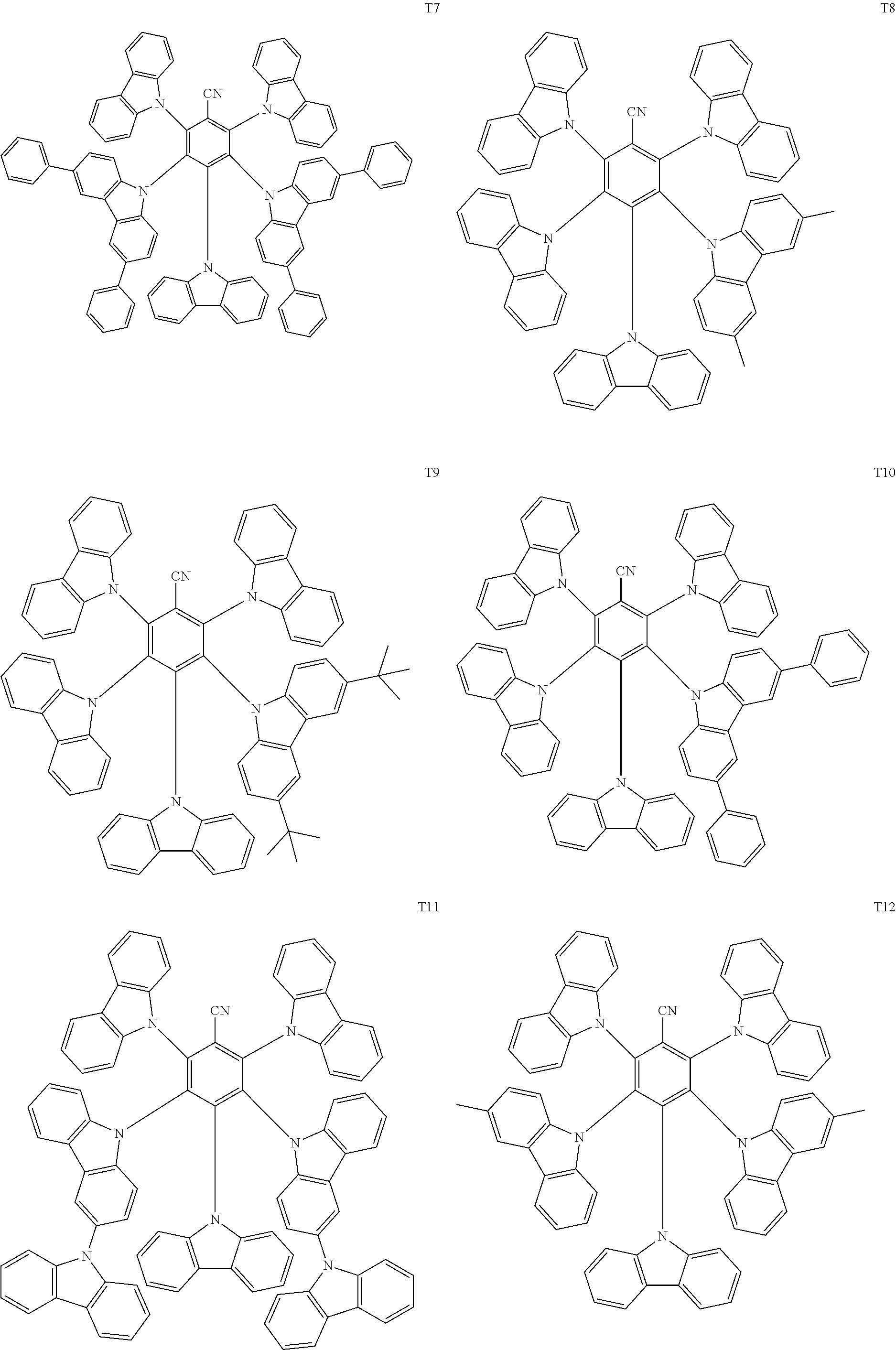

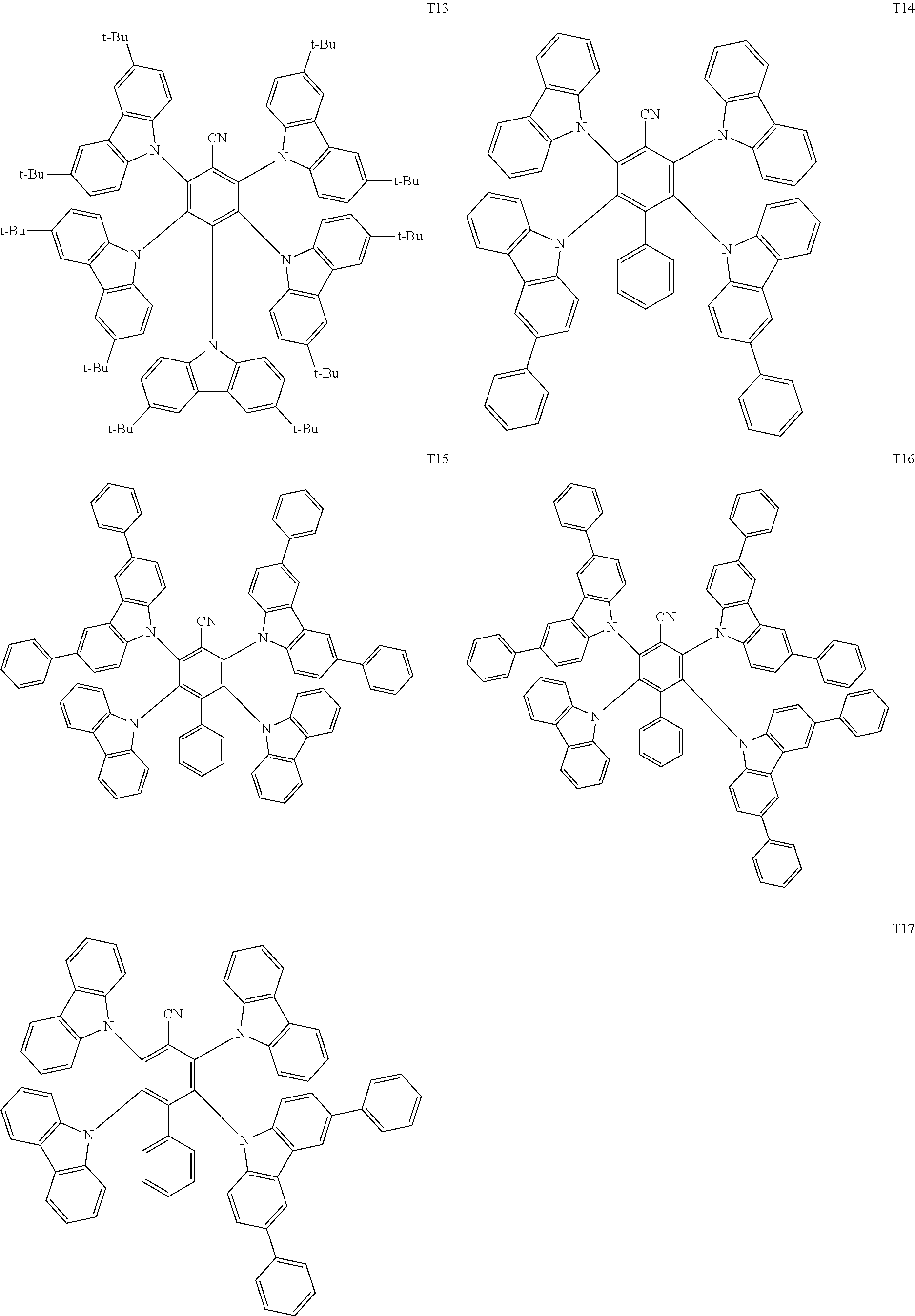

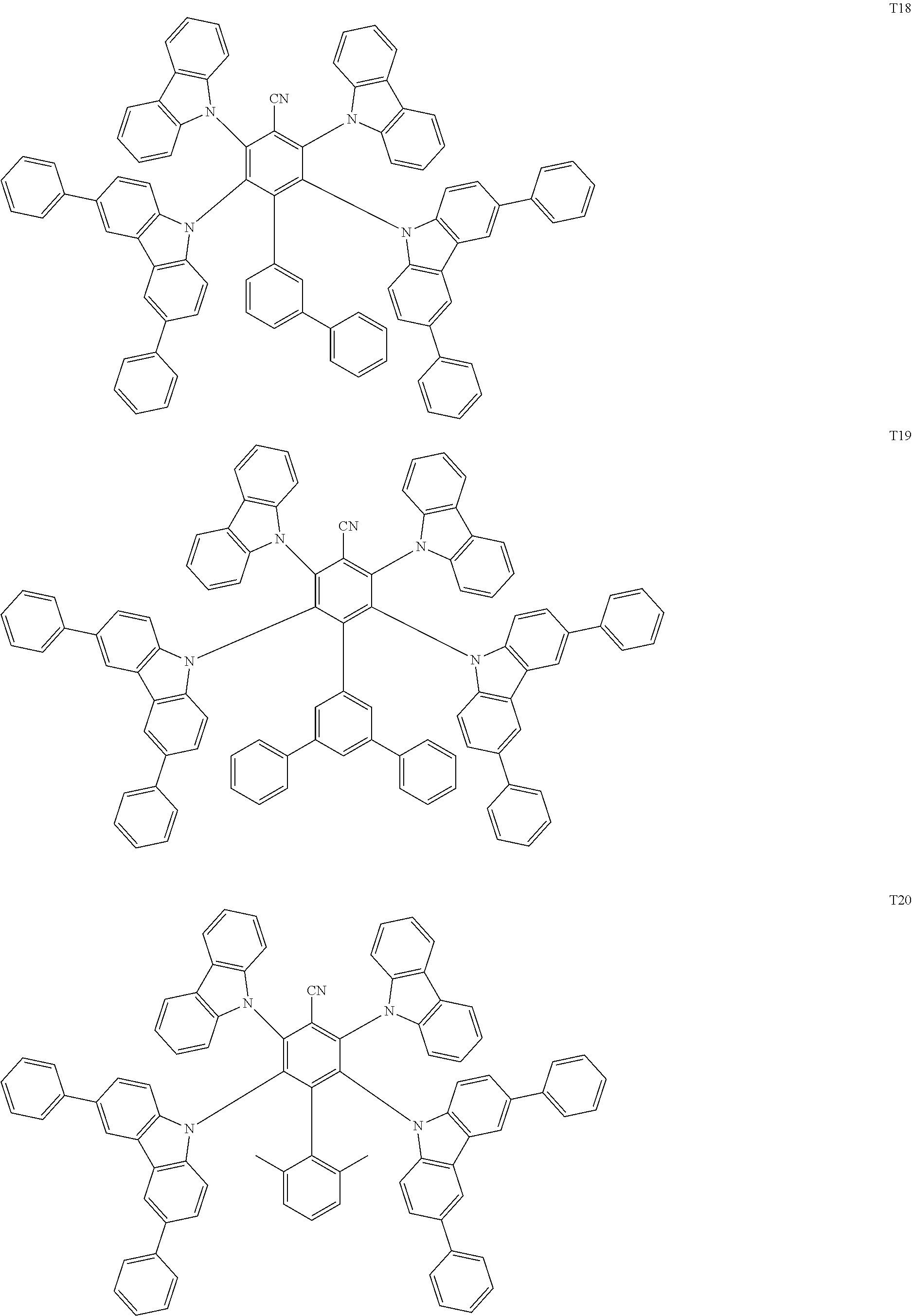

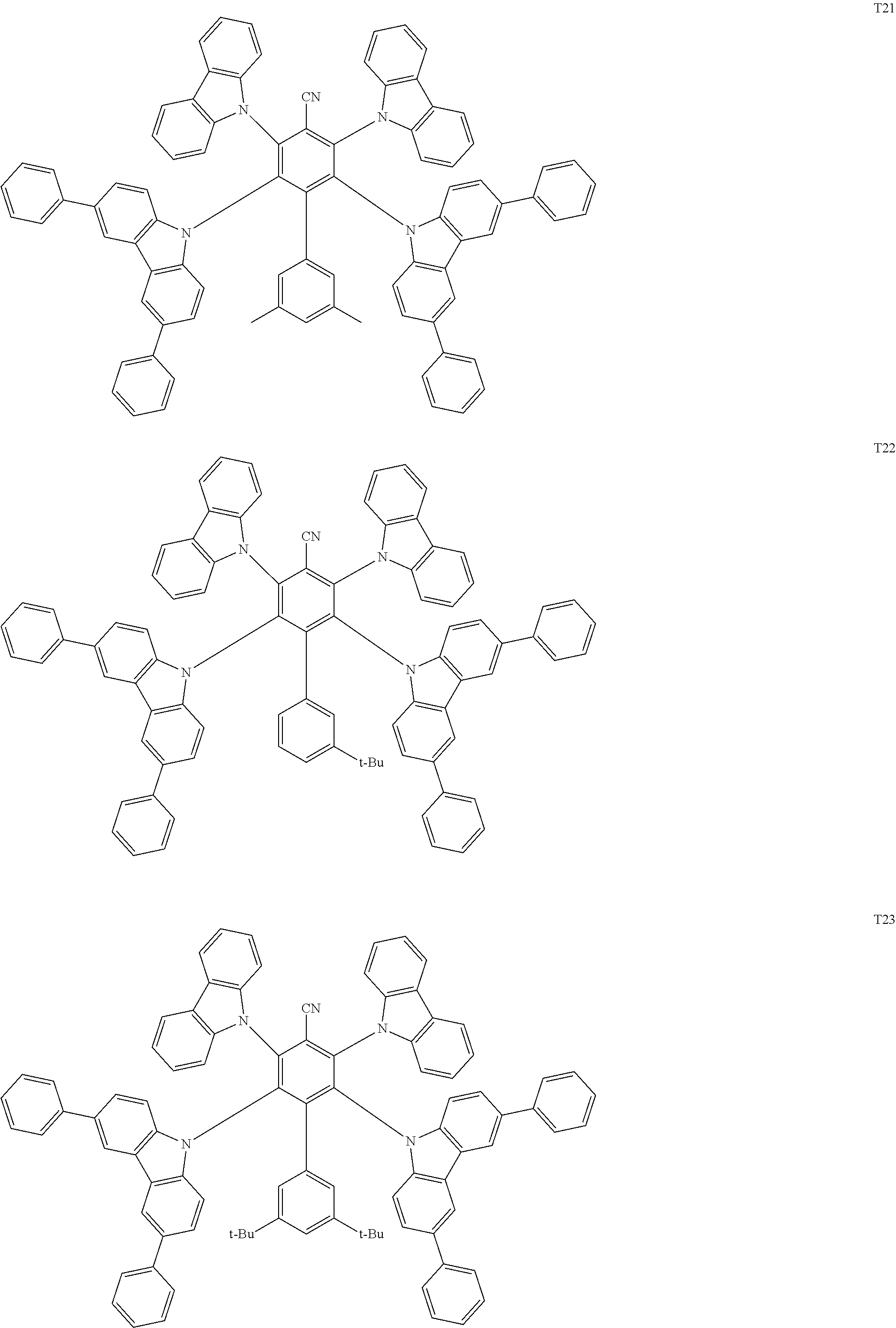 [SMILES: Cc1cc(C)cc(-c2c(-n3c4ccc(-c5ccccc5)cc4c4cc(-c5ccccc5)ccc43)c(-n3c4ccccc4c4ccccc43)c(C#N)c(-n3c4ccccc4c4ccccc43)c2-n2c3ccc(-c4ccccc4)cc3c3cc(-c4ccccc4)ccc32)c1 ; CC(C)(C)c1cccc(-c2c(-n3c4ccc(-c5ccccc5)cc4c4cc(-c5ccccc5)ccc43)c(-n3c4ccccc4c4ccccc43)c(C#N)c(-n3c4ccccc4c4ccccc43)c2-n2c3ccc(-c4ccccc4)cc3c3cc(-c4ccccc4)ccc32)c1 ; CC(C)(C)c1cc(-c2c(-n3c4ccc(-c5ccccc5)cc4c4cc(-c5ccccc5)ccc43)c(-n3c4ccccc4c4ccccc43)c(C#N)c(-n3c4ccccc4c4ccccc43)c2-n2c3ccc(-c4ccccc4)cc3c3cc(-c4ccccc4)ccc32)cc(C(C)(C)C)c1]

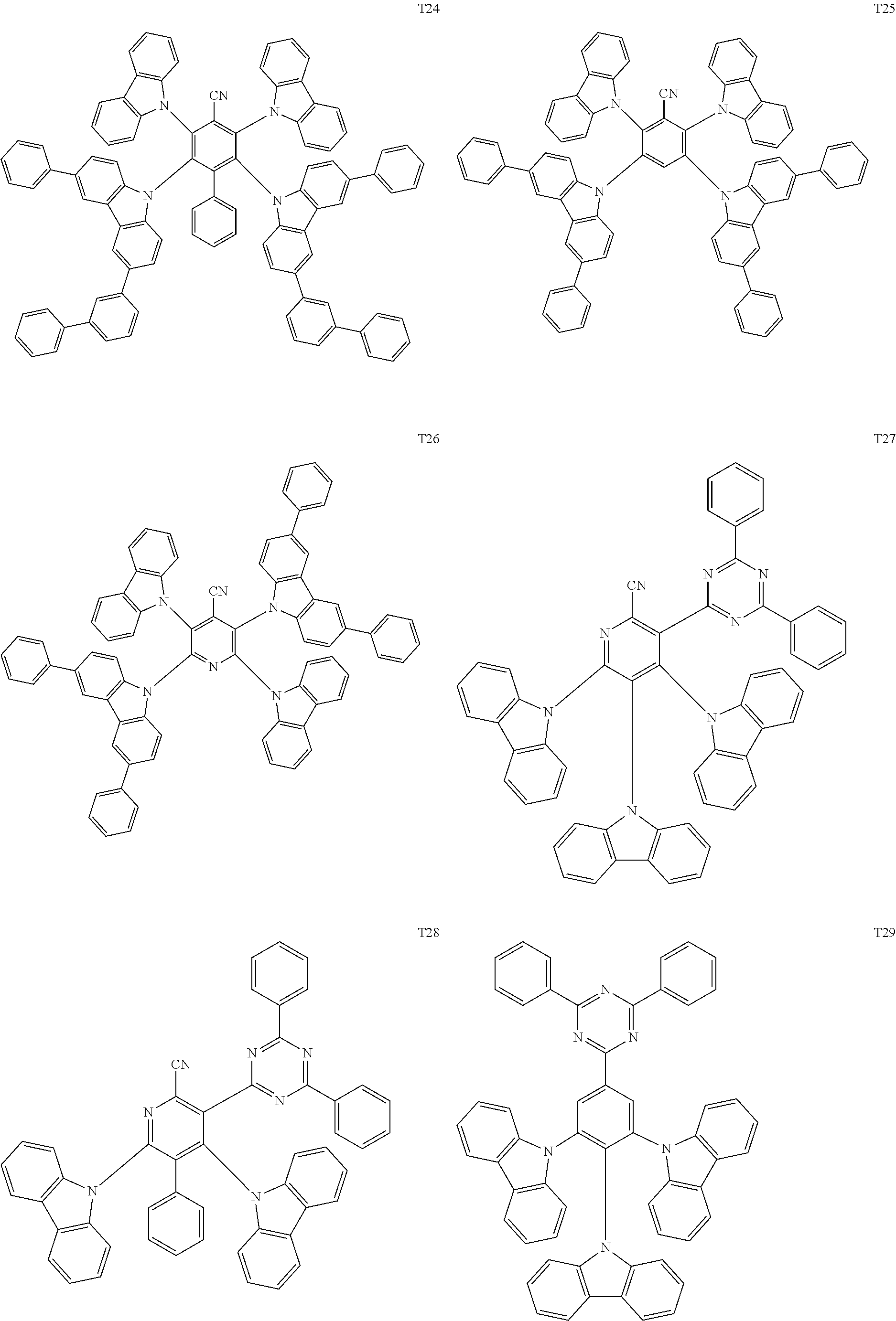

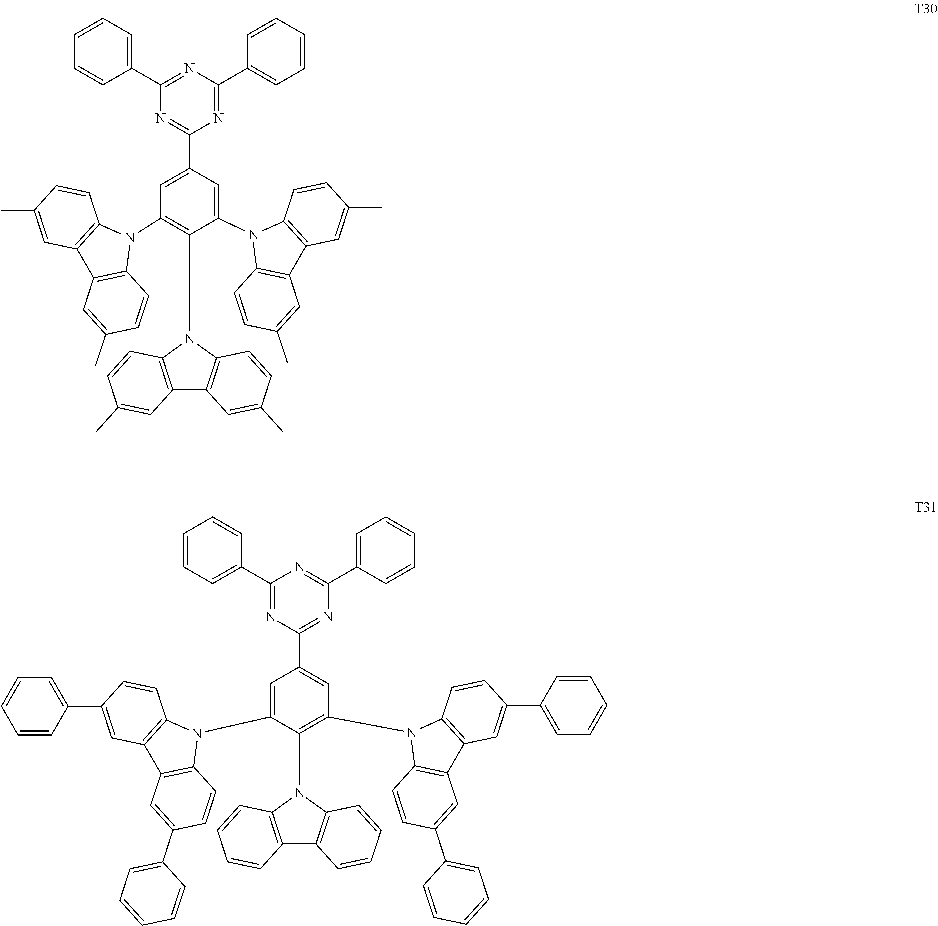

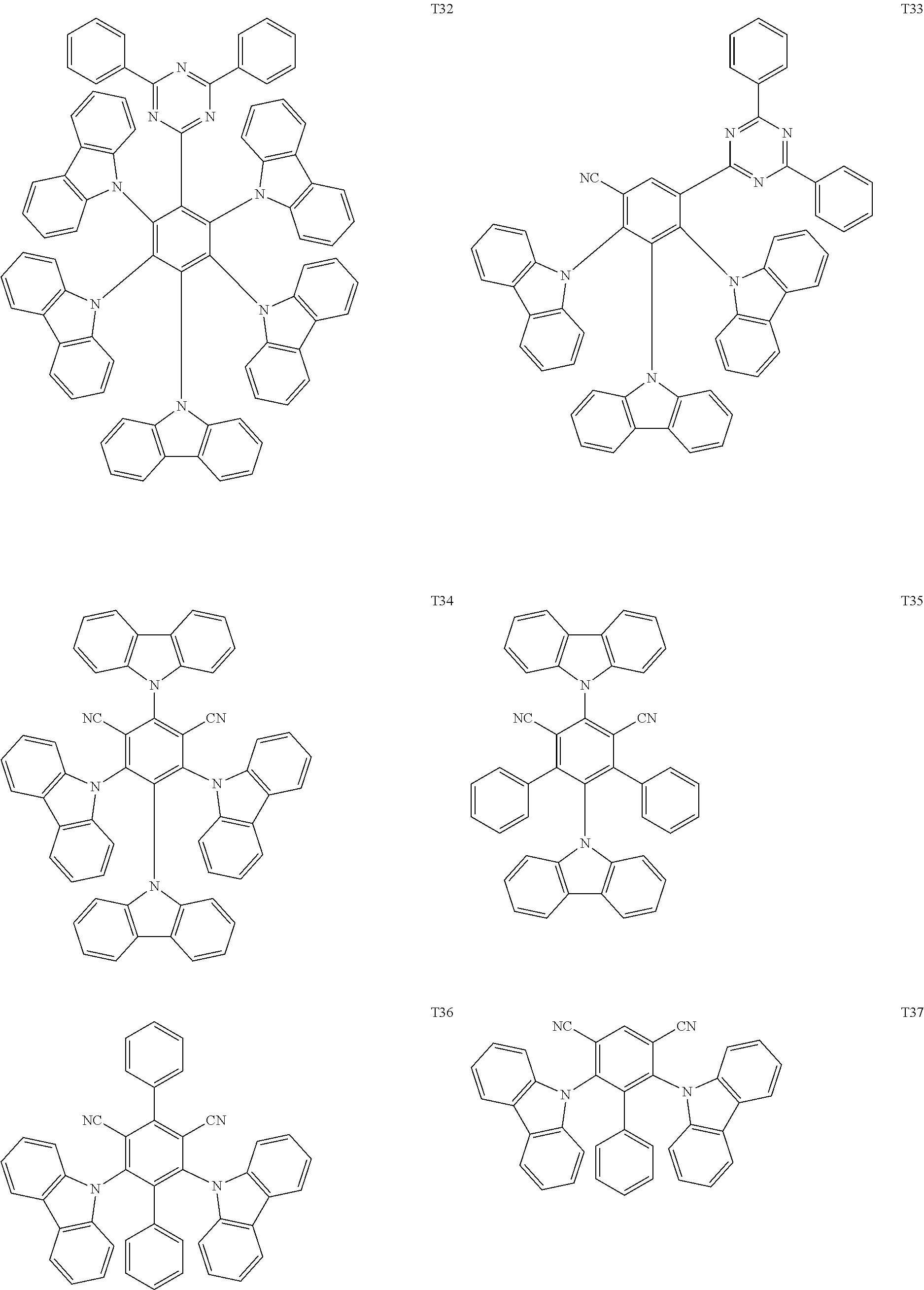

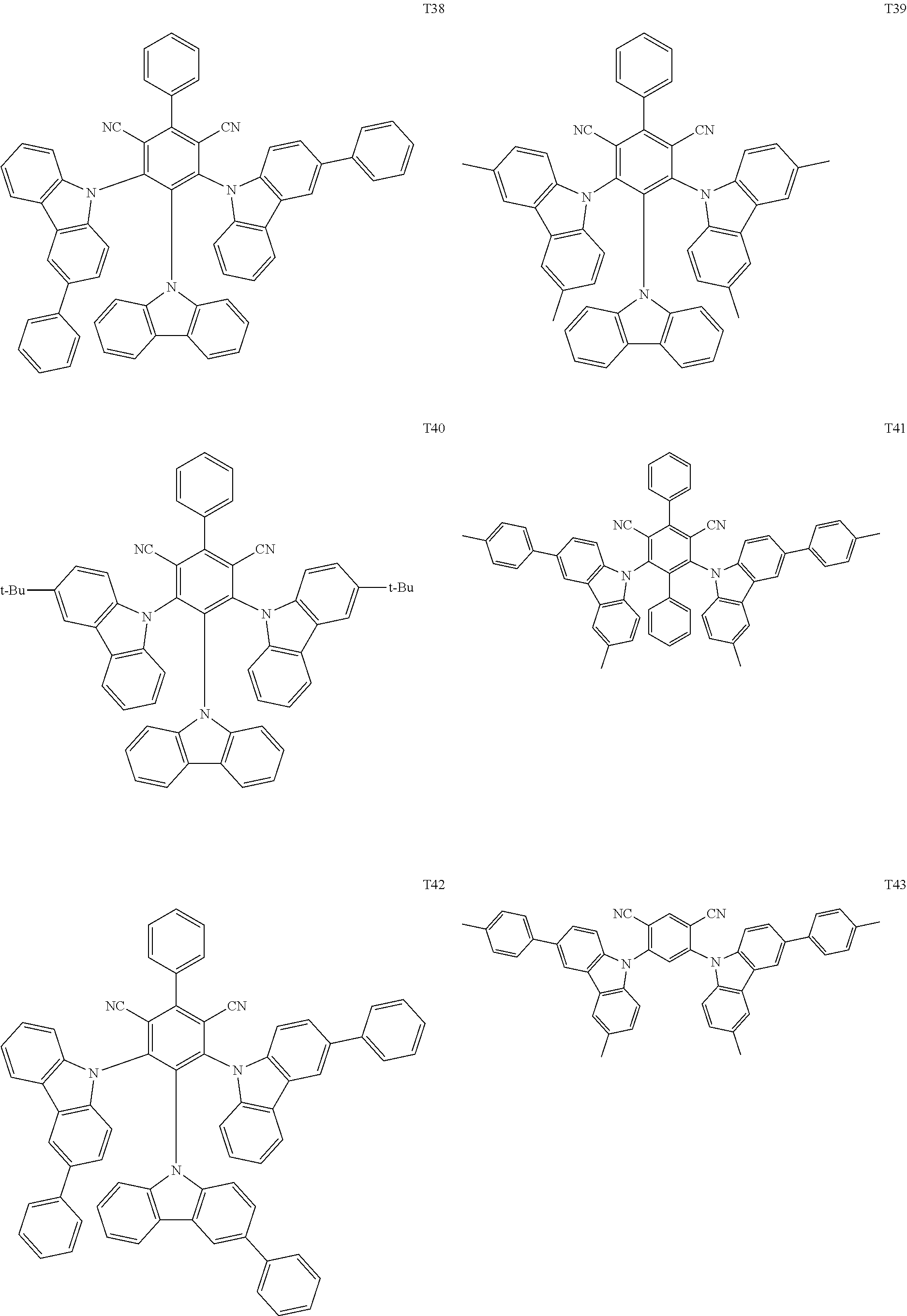

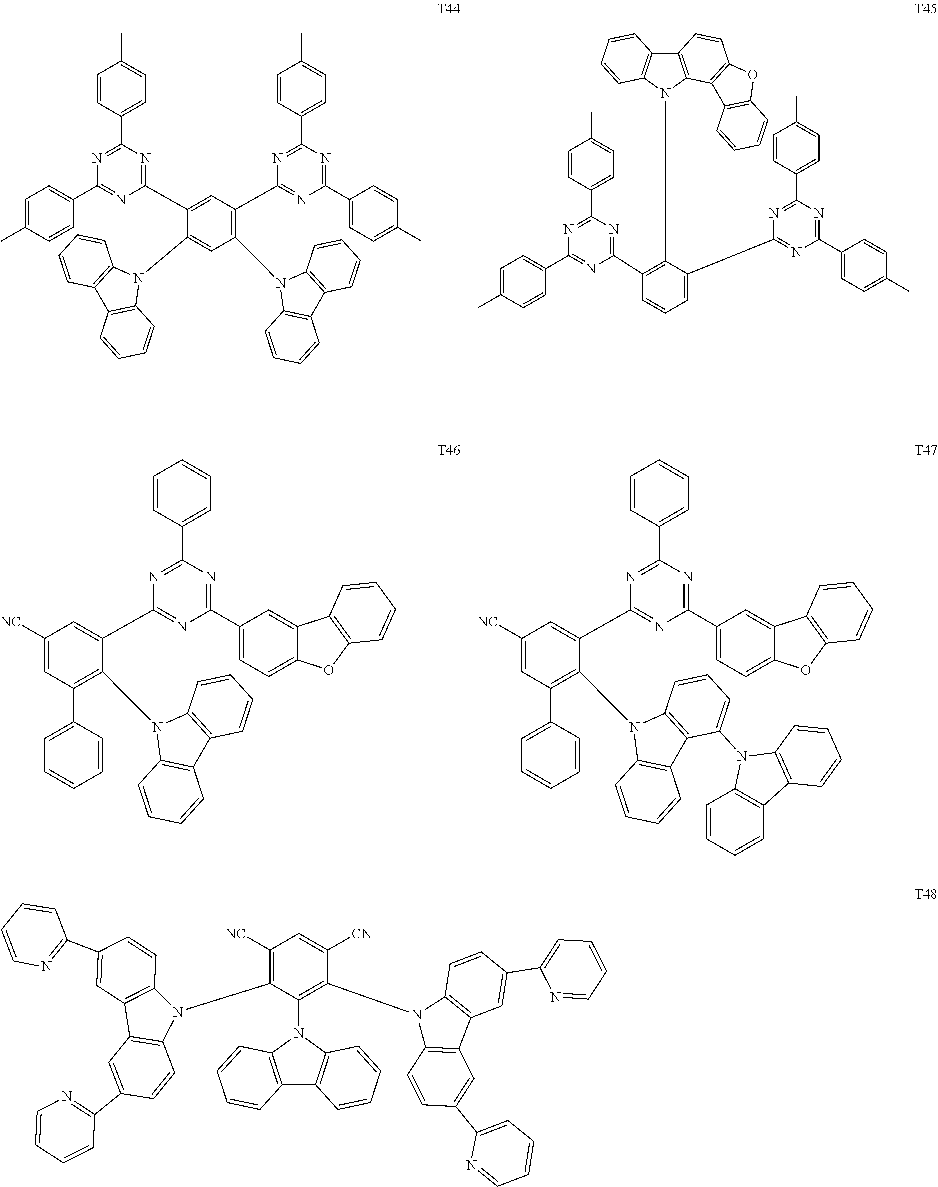

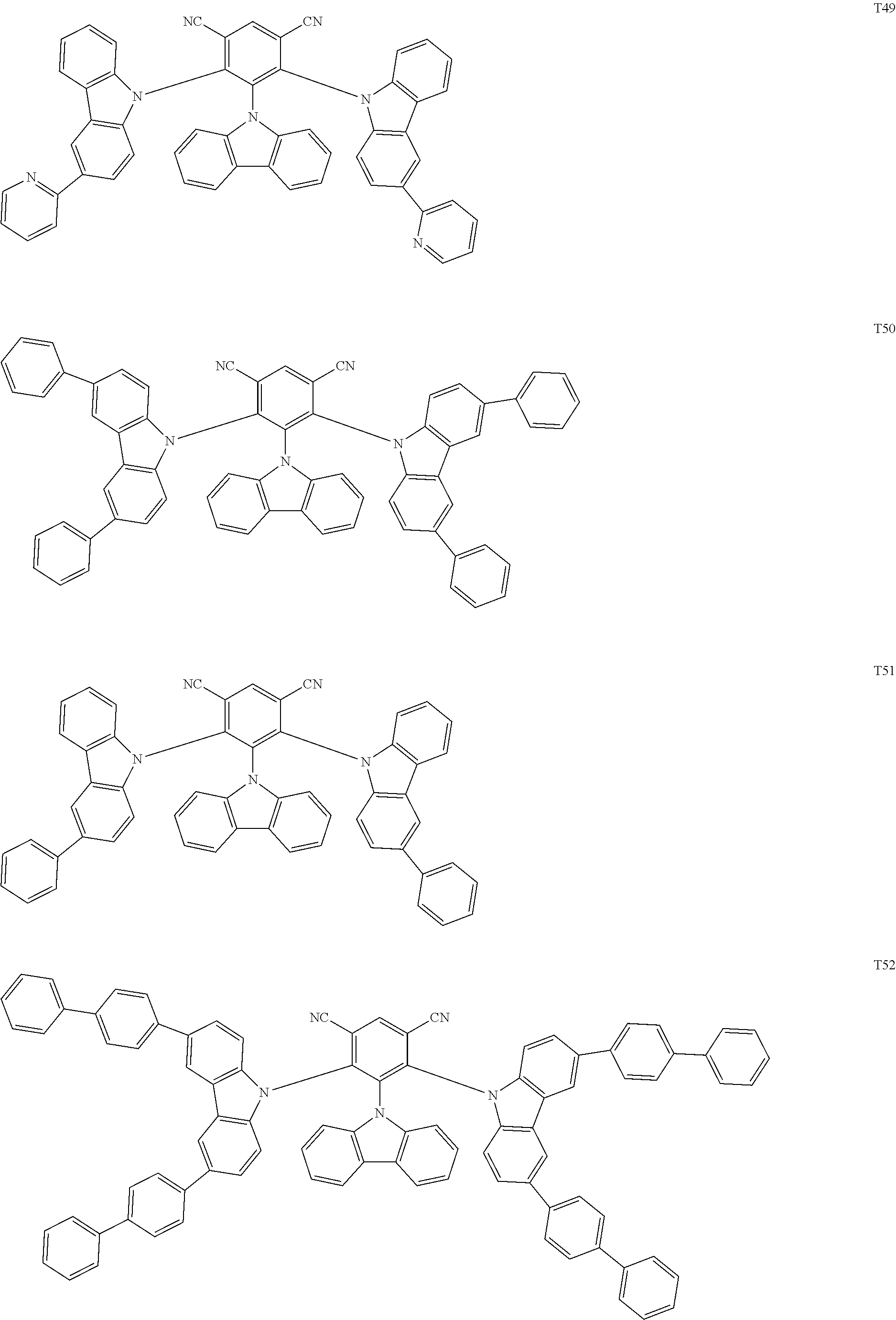

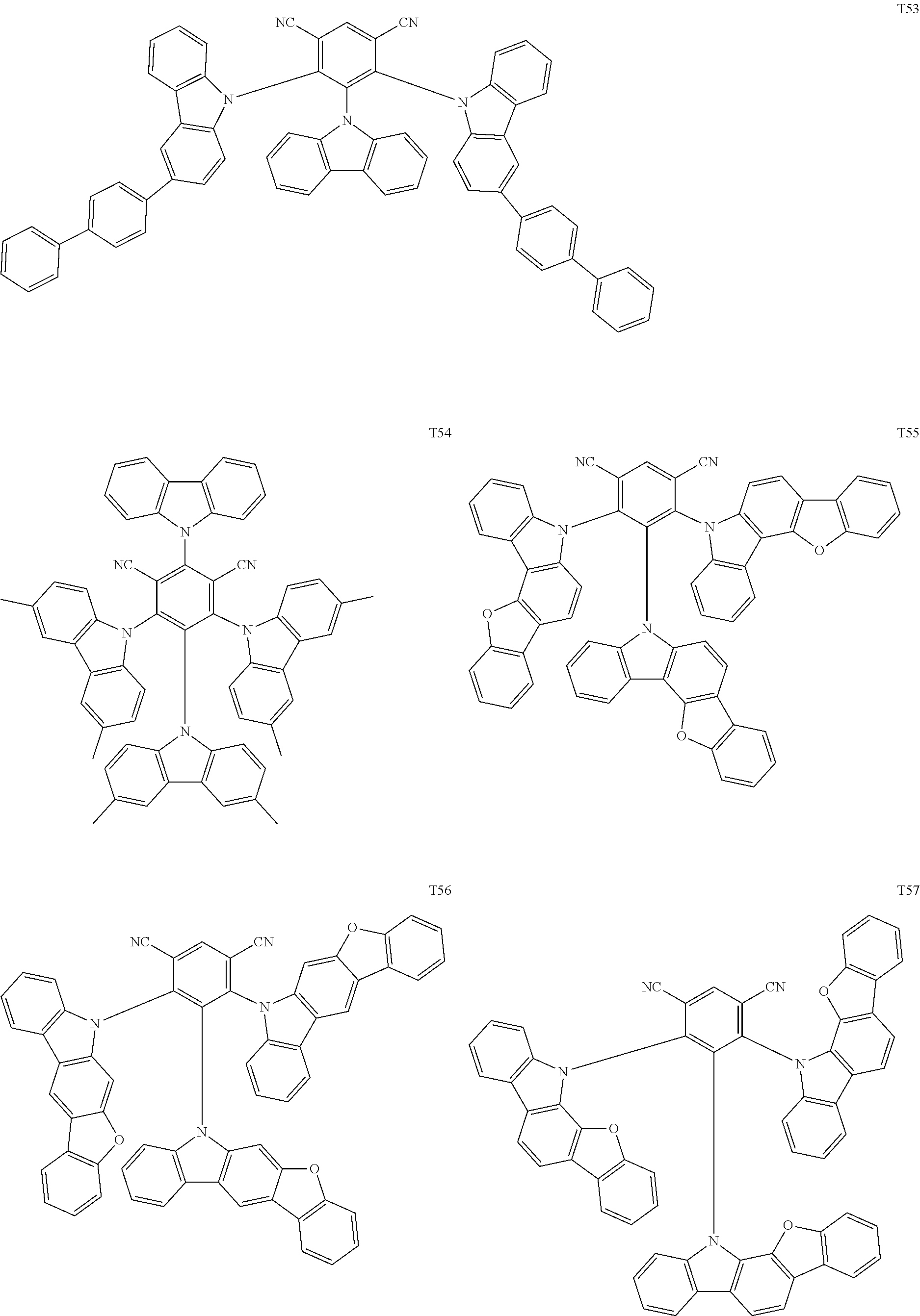

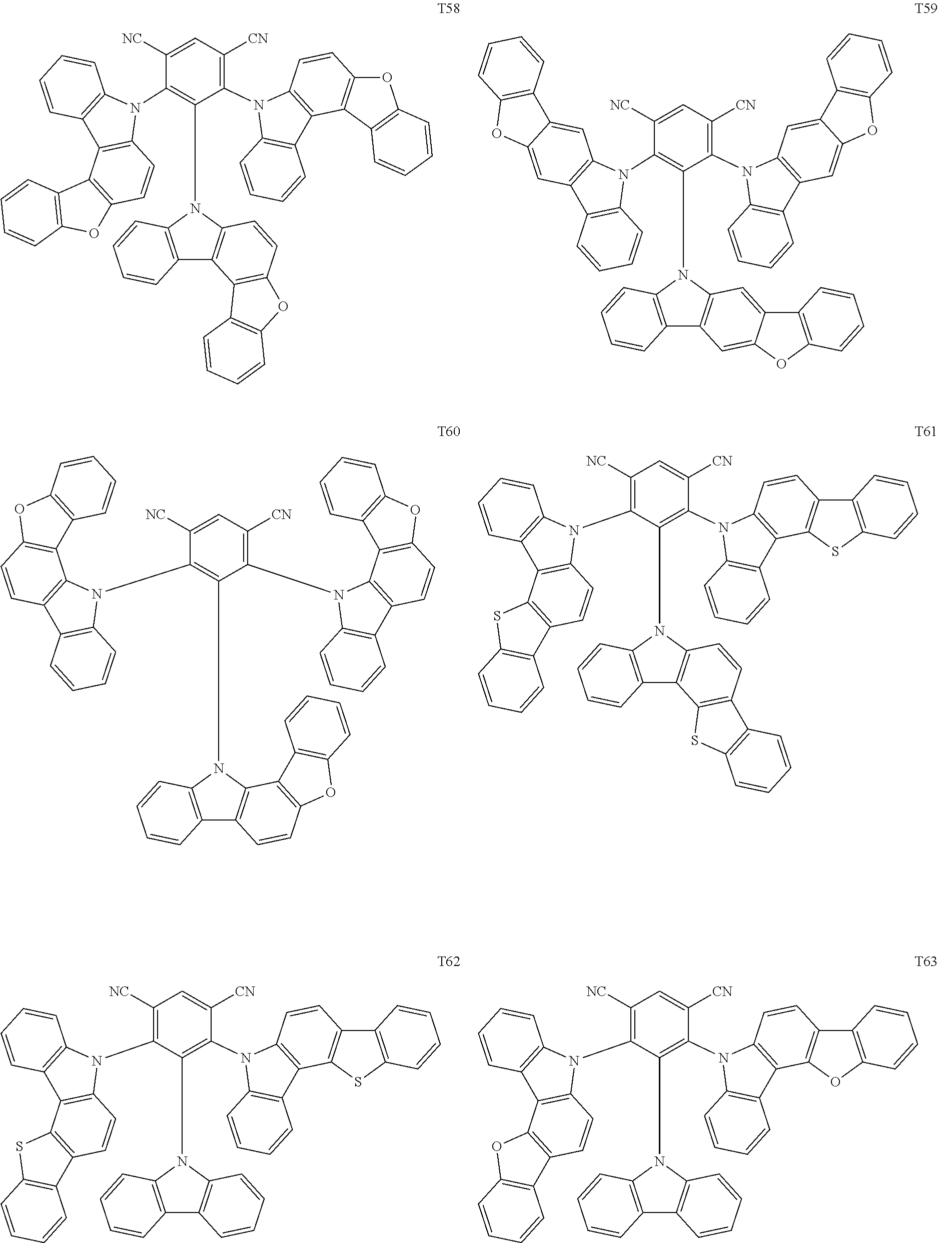

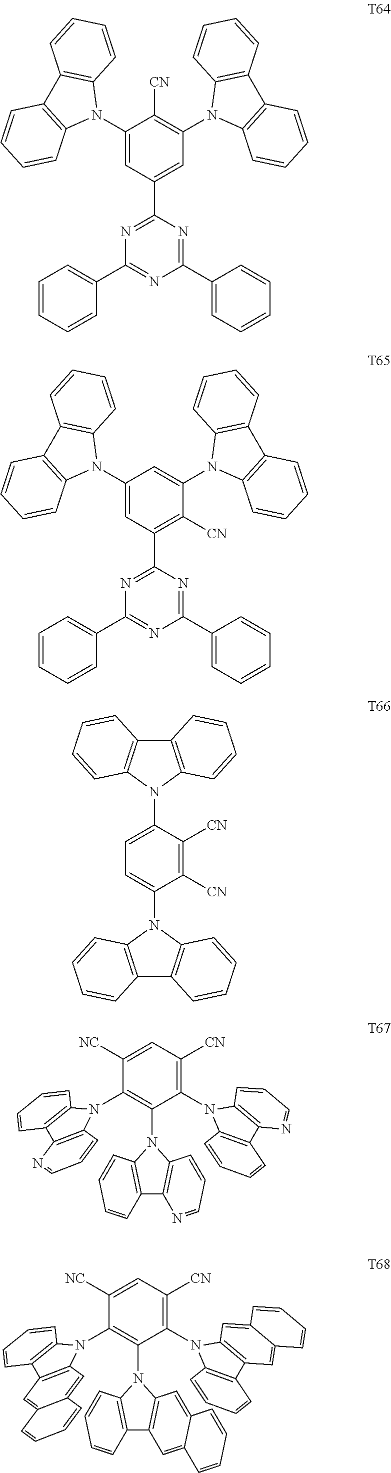

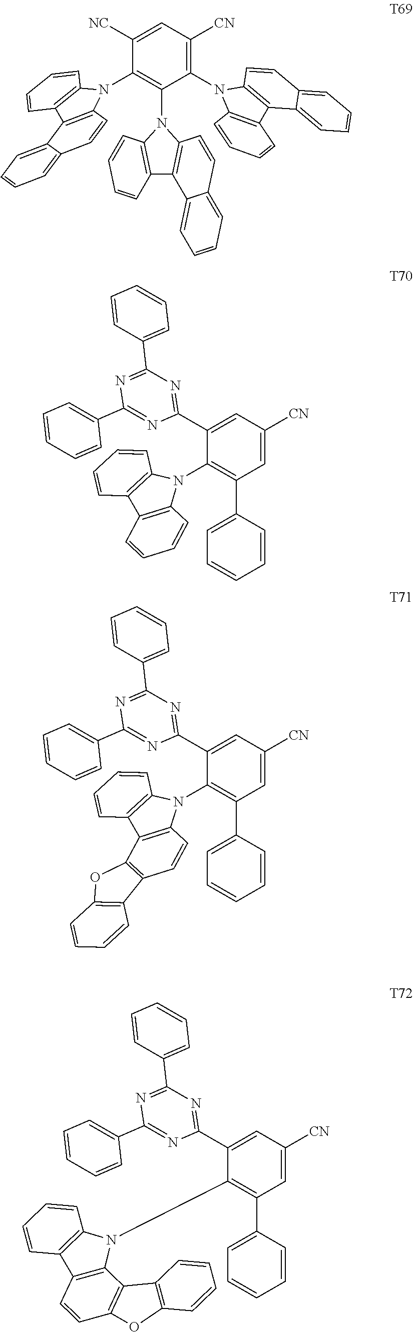

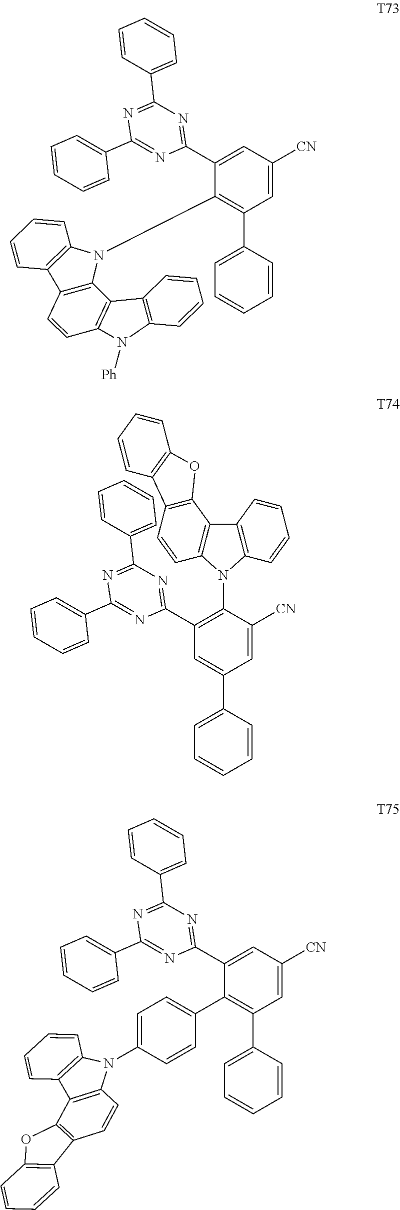

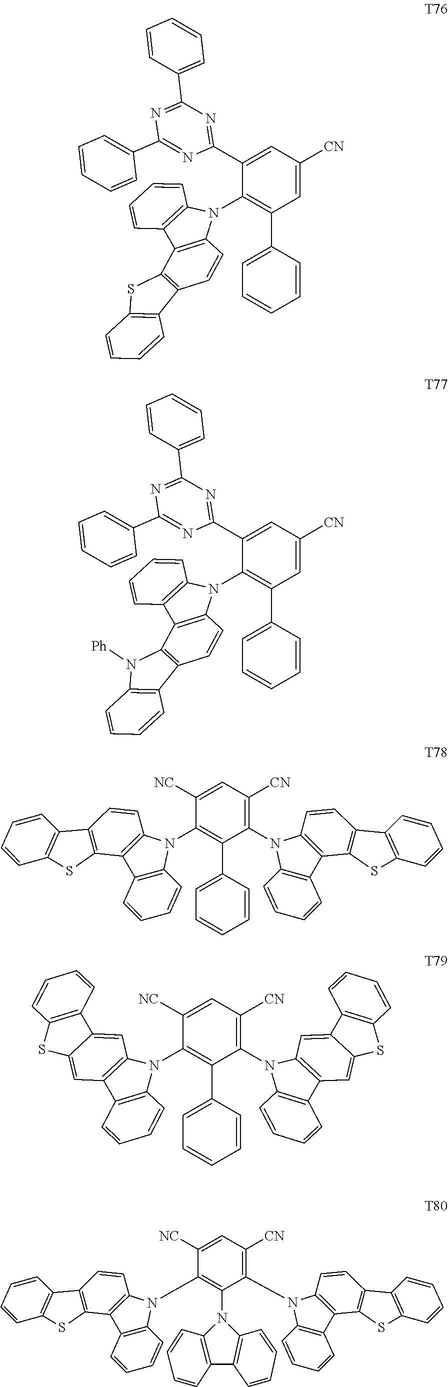 [SMILES: N#Cc1cc(-c2ccccc2)c(-n2c3ccccc3c3c4sc5ccccc5c4ccc32)c(-c2nc(-c3ccccc3)nc(-c3ccccc3)n2)c1 ; N#Cc1cc(-c2ccccc2)c(-n2c3ccccc3c3c2ccc2c4ccccc4n(-c4ccccc4)c23)c(-c2nc(-c3ccccc3)nc(-c3ccccc3)n2)c1 ; N#Cc1cc(C#N)c(-n2c3ccccc3c3c4sc5ccccc5c4ccc32)c(-c2ccccc2)c1-n1c2ccccc2c2c3sc4ccccc4c3ccc21 ; N#Cc1cc(C#N)c(-n2c3ccccc3c3cc4sc5ccccc5c4cc32)c(-c2ccccc2)c1-n1c2ccccc2c2cc3sc4ccccc4c3cc21 ; N#Cc1cc(C#N)c(-n2c3ccccc3c3c4sc5ccccc5c4ccc32)c(-n2c3ccccc3c3ccccc32)c1-n1c2ccccc2c2c3sc4ccccc4c3ccc21]

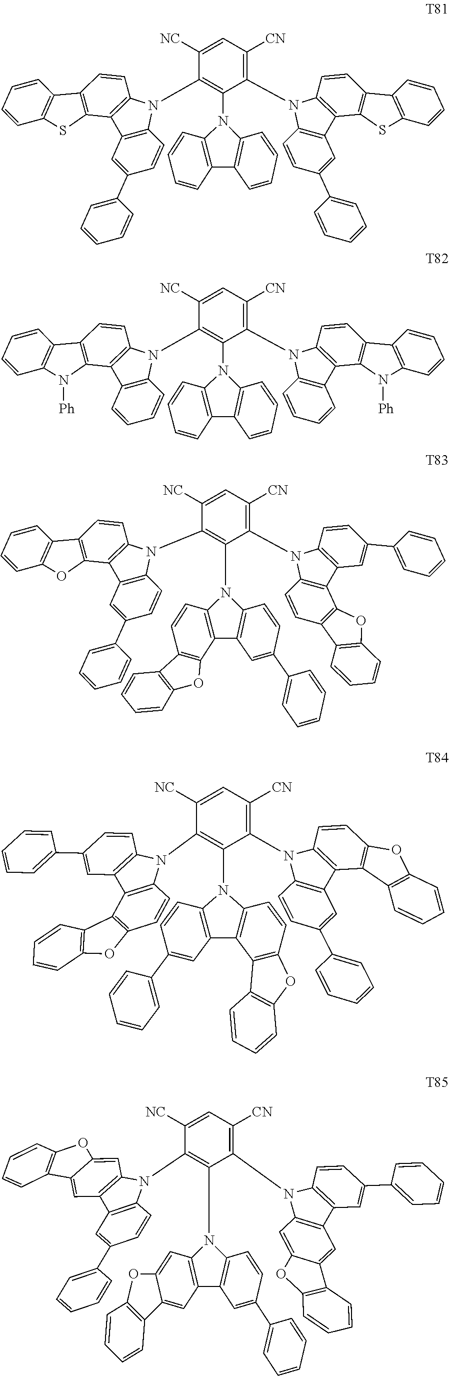

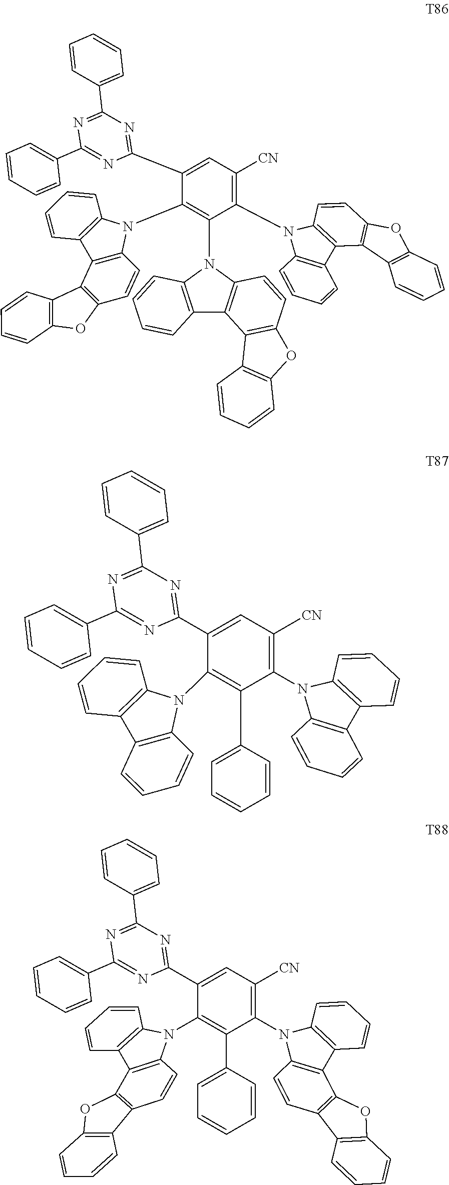

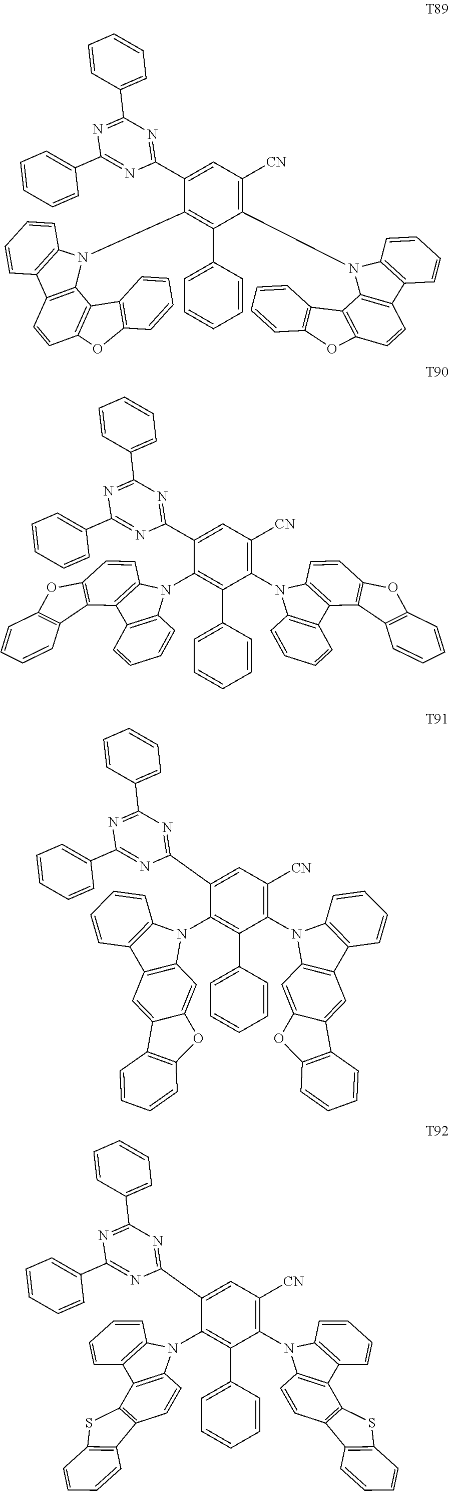

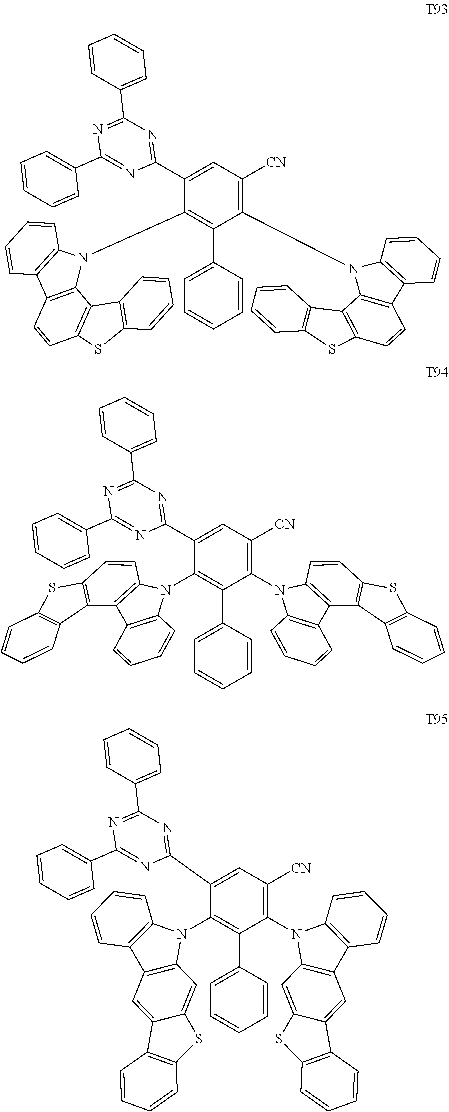

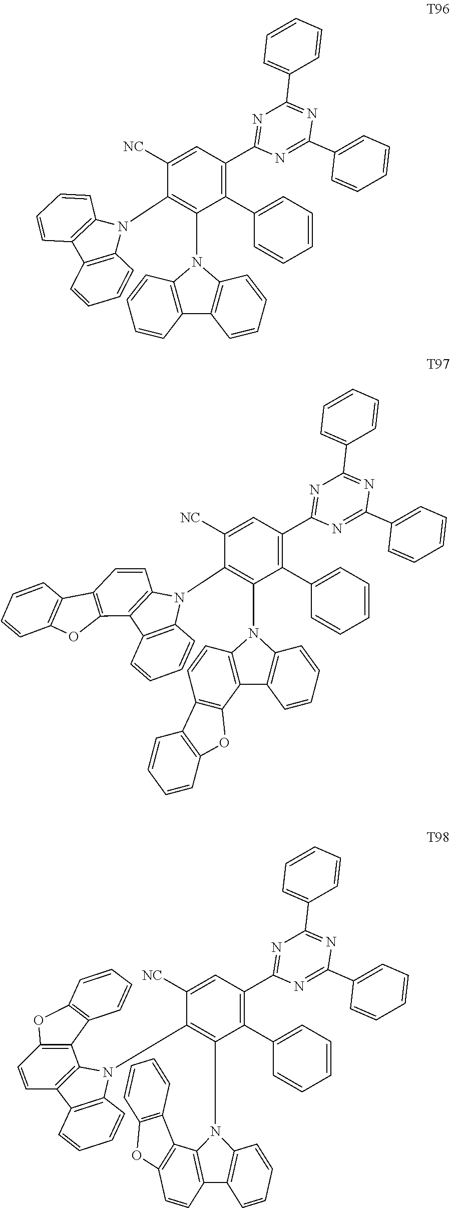

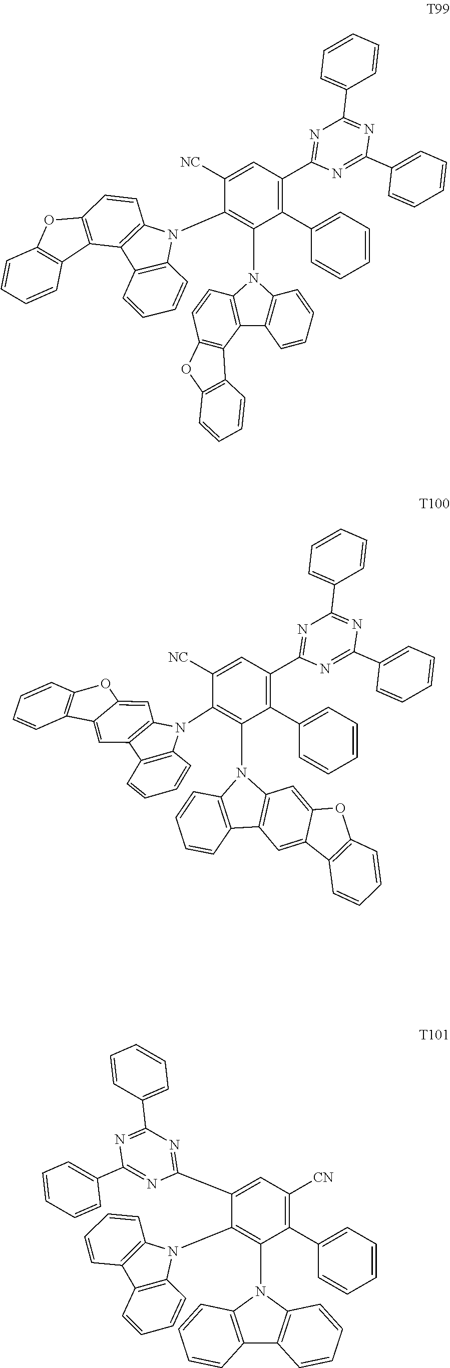

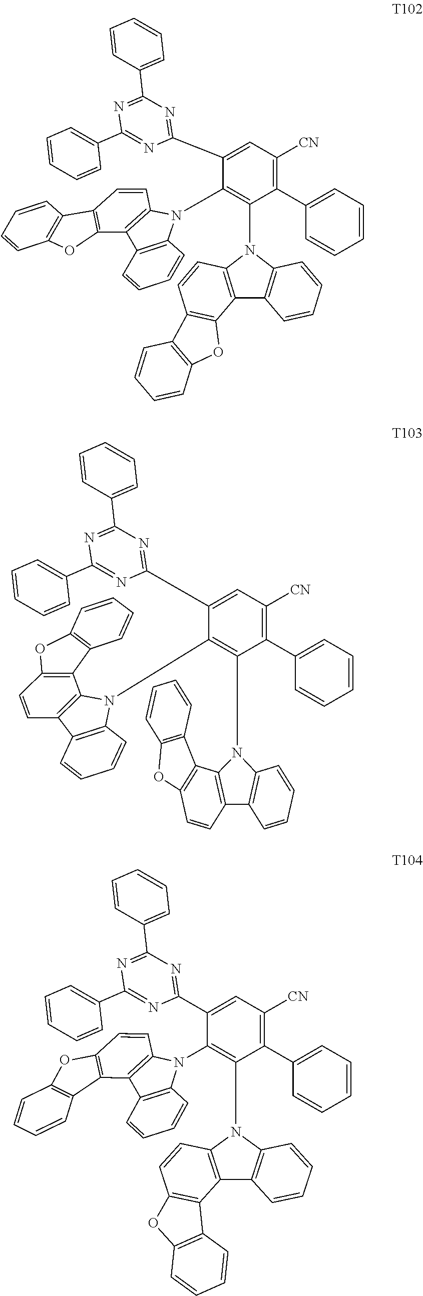 [SMILES: N#Cc1cc(-c2nc(-c3ccccc3)nc(-c3ccccc3)n2)c(-n2c3ccccc3c3c4oc5ccccc5c4ccc32)c(-n2c3ccccc3c3c4oc5ccccc5c4ccc32)c1-c1ccccc1 ; N#Cc1cc(-c2nc(-c3ccccc3)nc(-c3ccccc3)n2)c(-n2c3ccccc3c3ccc4oc5ccccc5c4c32)c(-n2c3ccccc3c3ccc4oc5ccccc5c4c32)c1-c1ccccc1 ; N#Cc1cc(-c2nc(-c3ccccc3)nc(-c3ccccc3)n2)c(-n2c3ccccc3c3c4c(ccc32)oc2ccccc24)c(-n2c3ccccc3c3c4c(ccc32)oc2ccccc24)c1-c1ccccc1]

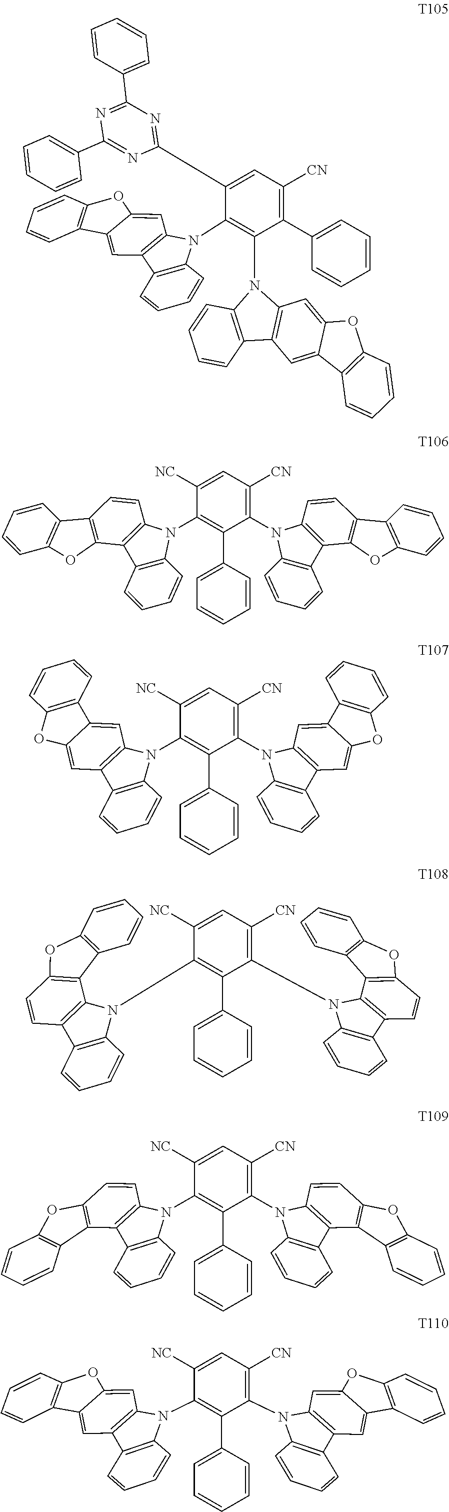

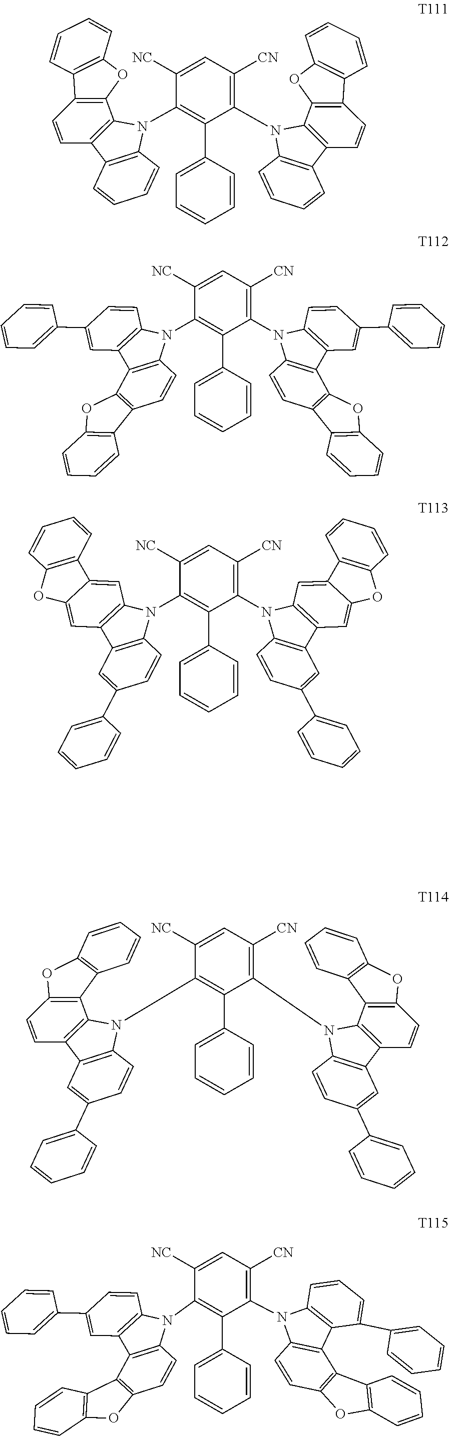

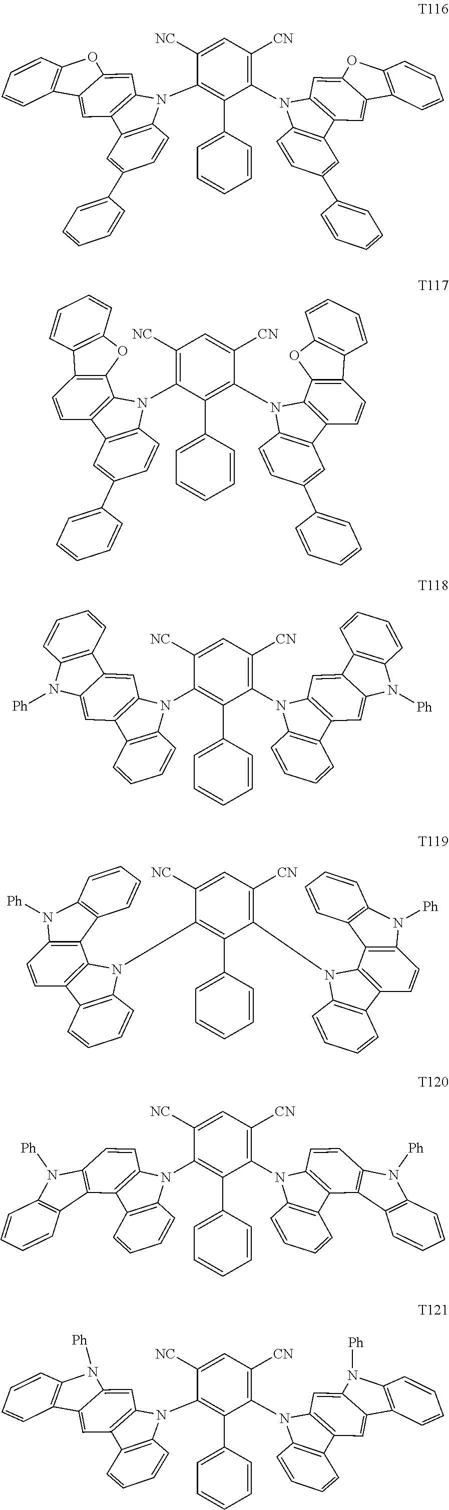

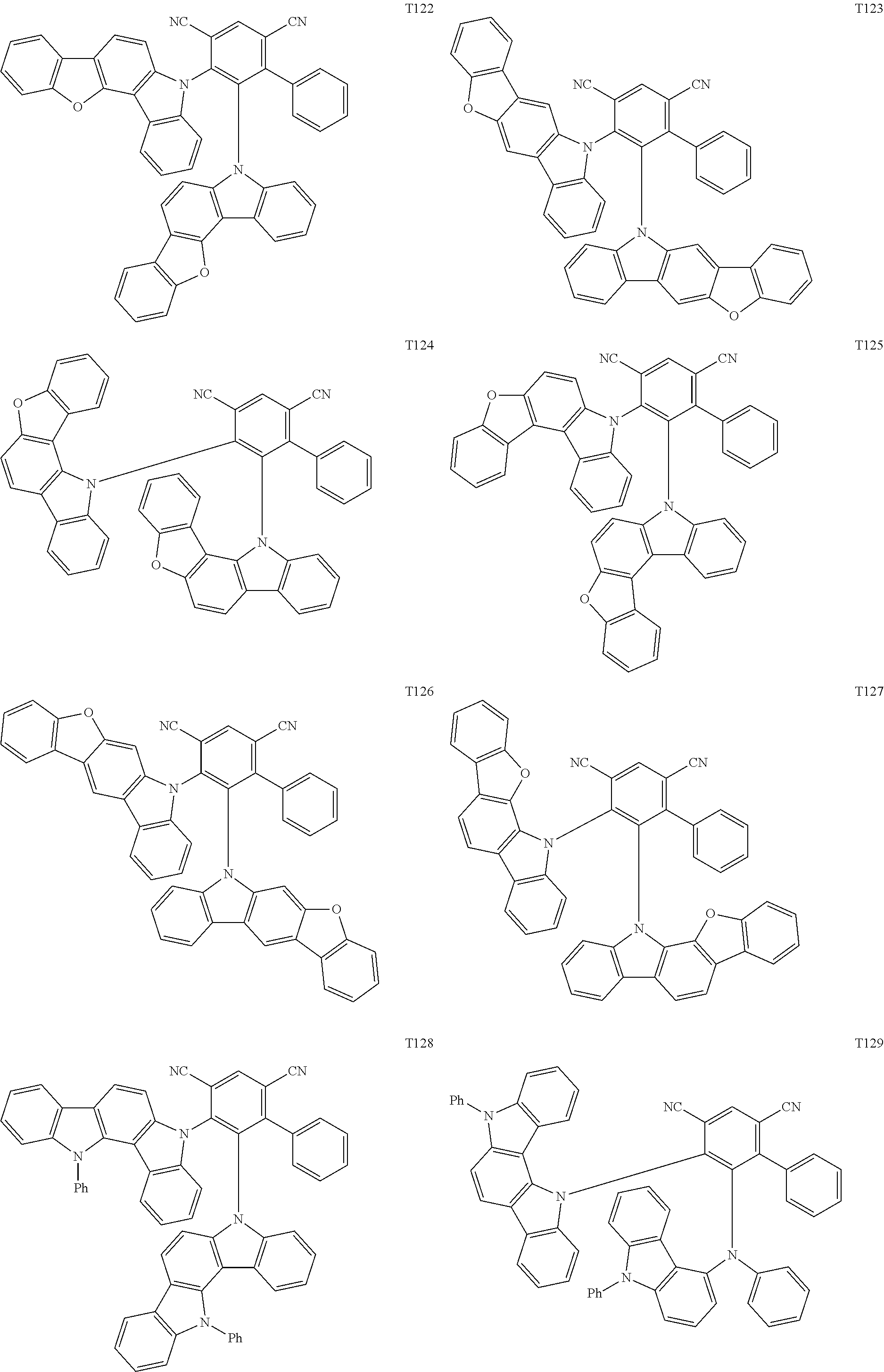

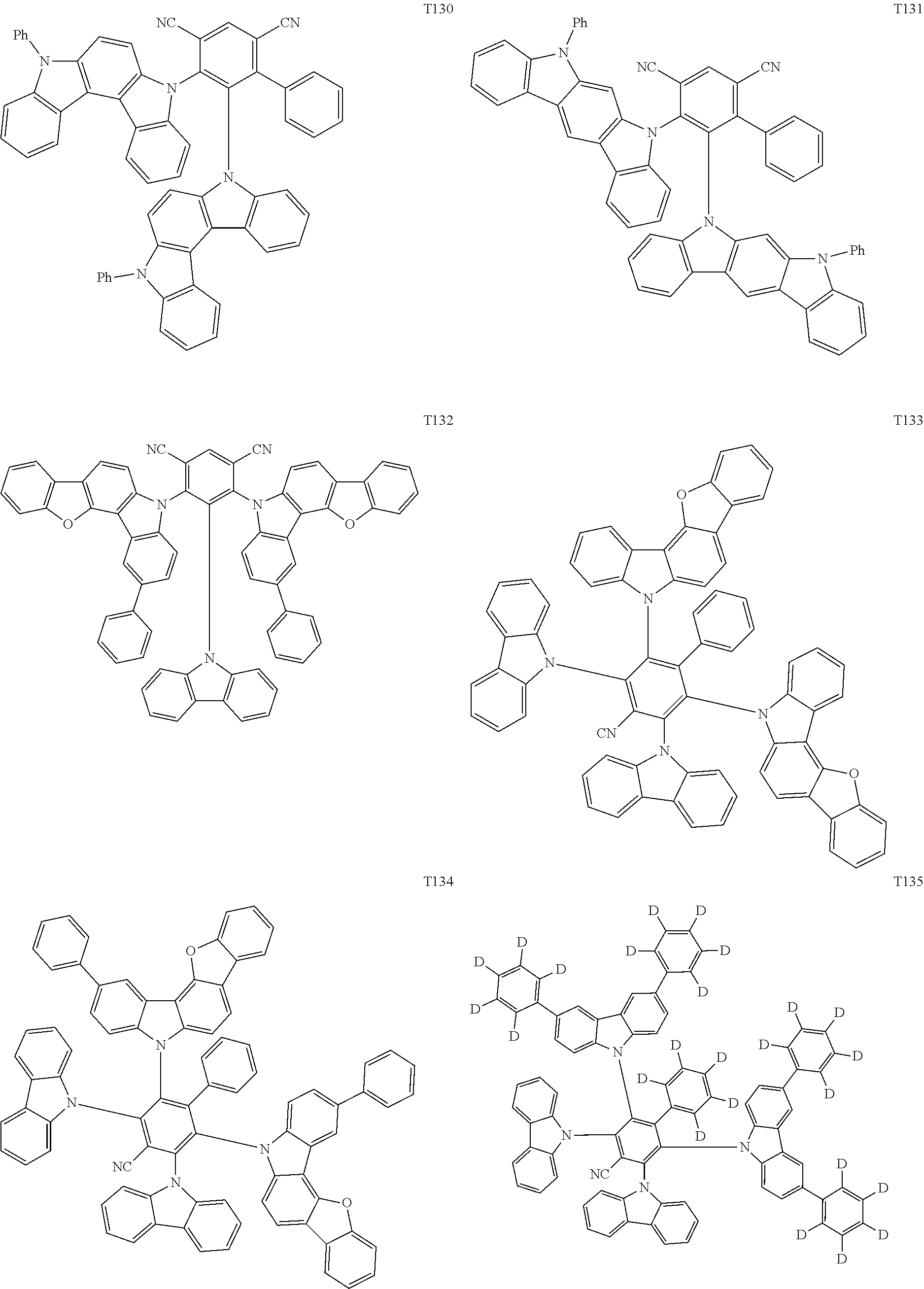

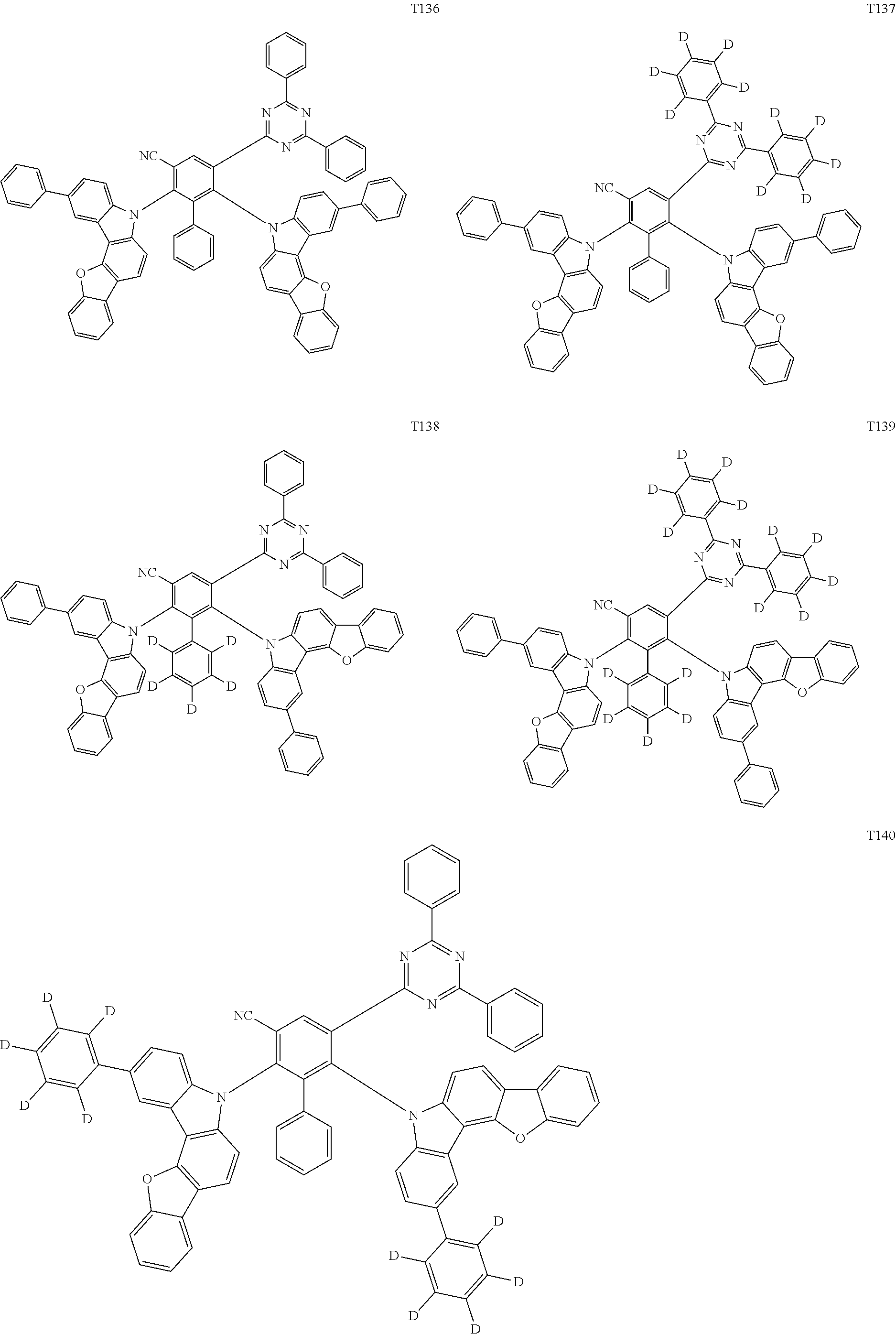

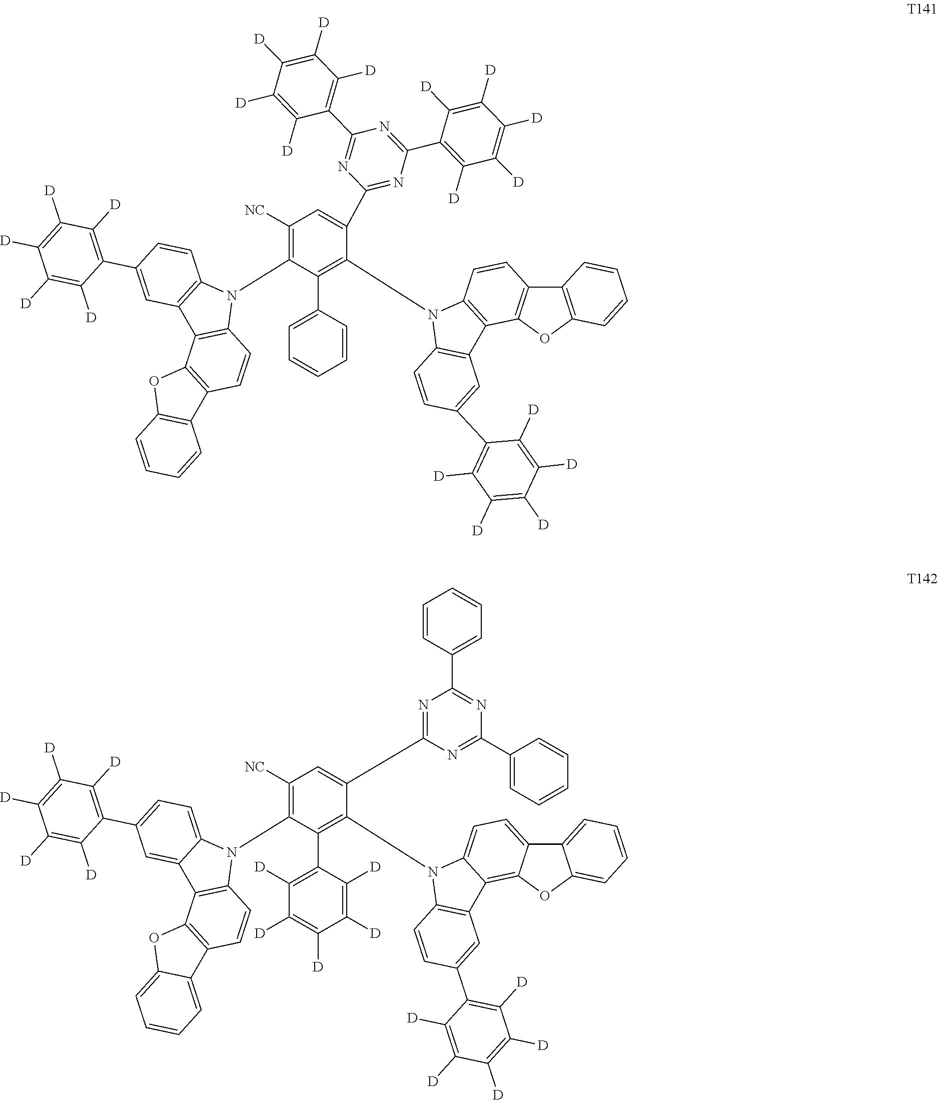 [SMILES: [2H]c1c([2H])c([2H])c(-c2ccc3c(c2)c2c4oc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5c([2H])c([2H])c([2H])c([2H])c5[2H])cc4c4c5oc6ccccc6c5ccc43)c2-c2ccccc2)c([2H])c1[2H] ; [2H]c1c([2H])c([2H])c(-c2ccc3c(c2)c2c4oc5ccccc5c4ccc2n3-c2c(C#N)cc(-c3nc(-c4ccccc4)nc(-c4ccccc4)n3)c(-n3c4ccc(-c5c([2H])c([2H])c([2H])c([2H])c5[2H])cc4c4c5oc6ccccc6c5ccc43)c2-c2c([2H])c([2H])c([2H])c([2H])c2[2H])c([2H])c1[2H]]

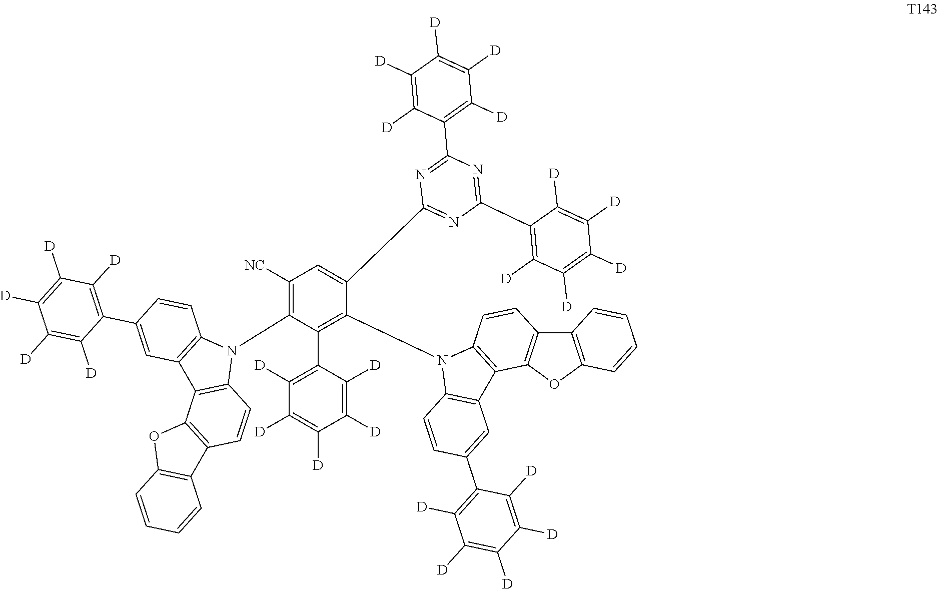 [SMILES: [2H]c1c([2H])c([2H])c(-c2ccc3c(c2)c2c4oc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5c([2H])c([2H])c([2H])c([2H])c5[2H])cc4c4c5oc6ccccc6c5ccc43)c2-c2c([2H])c([2H])c([2H])c([2H])c2[2H])c([2H])c1[2H]]

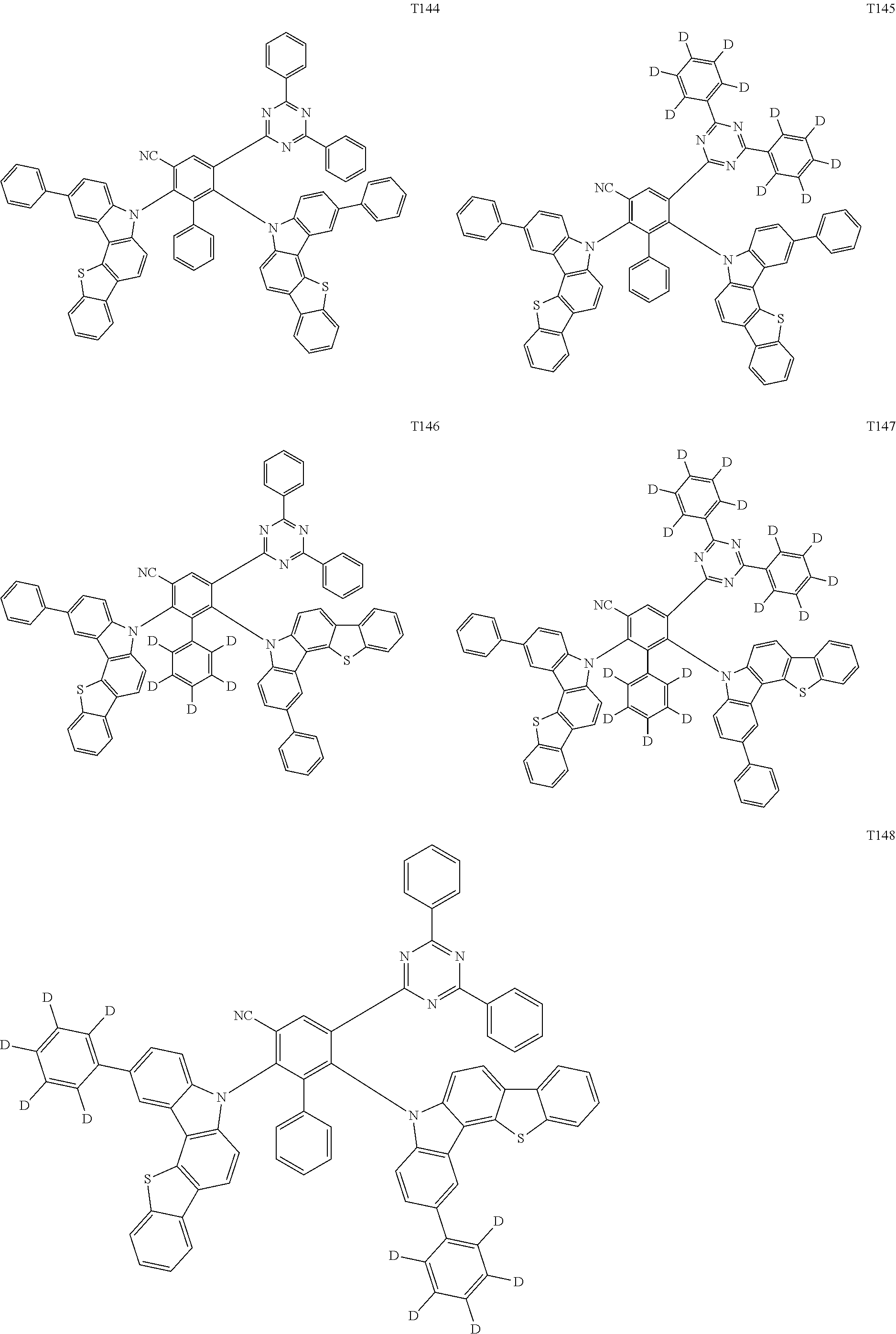

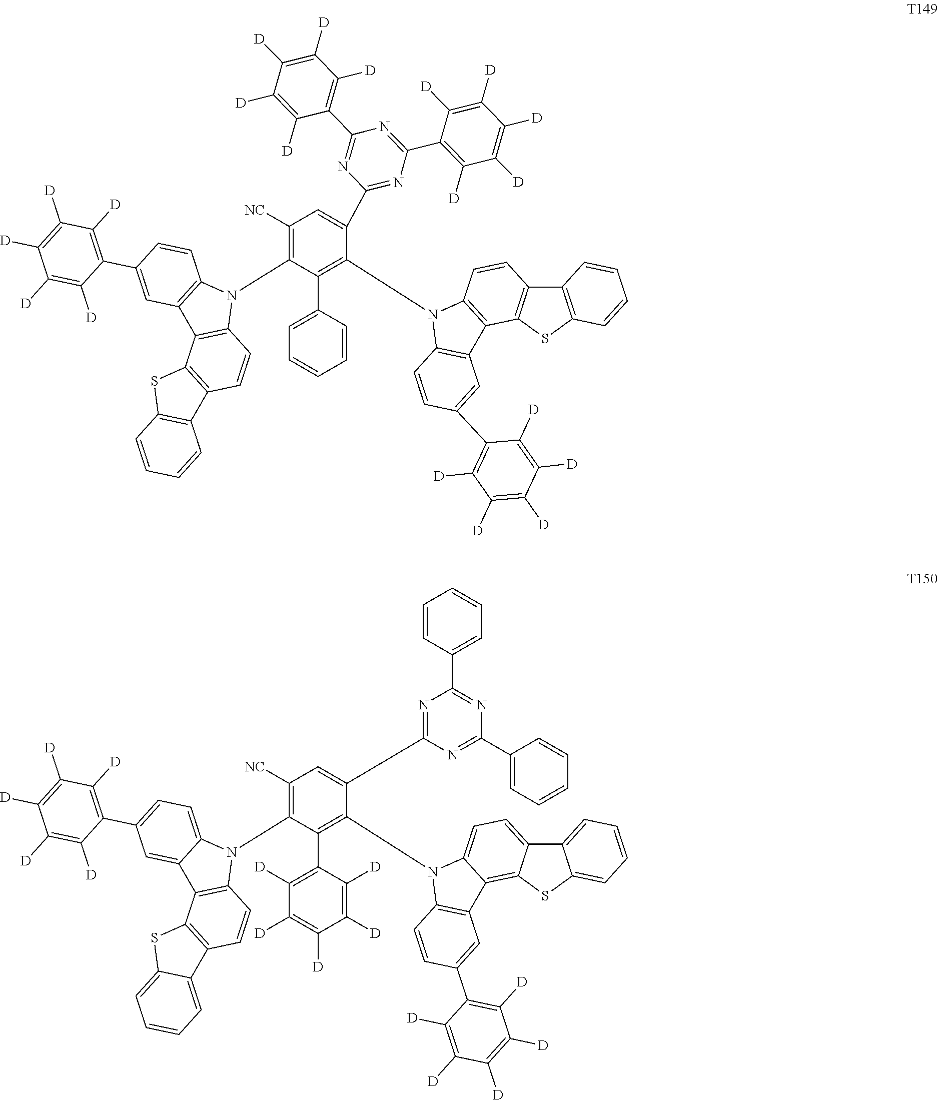 [SMILES: [2H]c1c([2H])c([2H])c(-c2ccc3c(c2)c2c4sc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5c([2H])c([2H])c([2H])c([2H])c5[2H])cc4c4c5sc6ccccc6c5ccc43)c2-c2ccccc2)c([2H])c1[2H] ; [2H]c1c([2H])c([2H])c(-c2ccc3c(c2)c2c4sc5ccccc5c4ccc2n3-c2c(C#N)cc(-c3nc(-c4ccccc4)nc(-c4ccccc4)n3)c(-n3c4ccc(-c5c([2H])c([2H])c([2H])c([2H])c5[2H])cc4c4c5sc6ccccc6c5ccc43)c2-c2c([2H])c([2H])c([2H])c([2H])c2[2H])c([2H])c1[2H]]

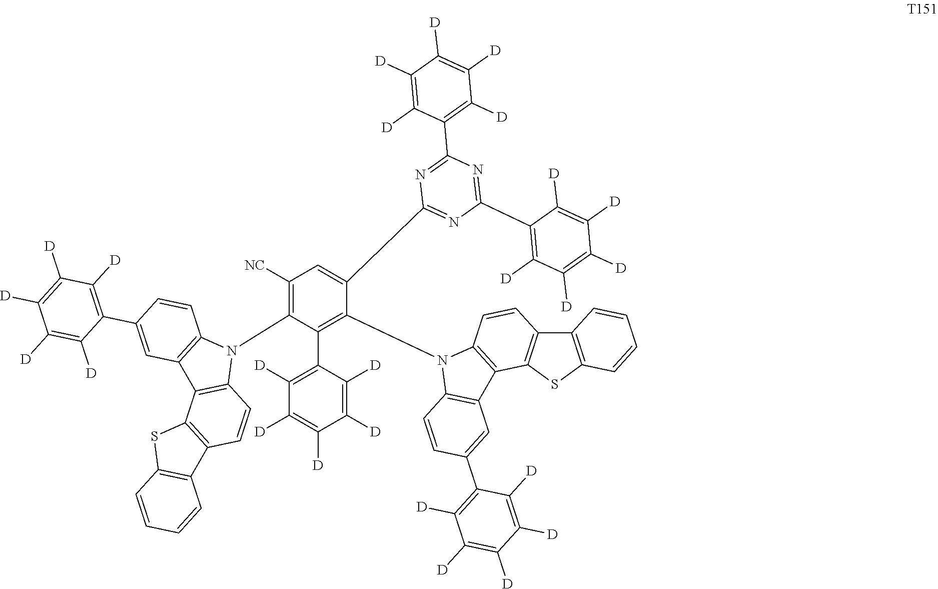 [SMILES: [2H]c1c([2H])c([2H])c(-c2ccc3c(c2)c2c4sc5ccccc5c4ccc2n3-c2c(C#N)cc(-c3nc(-c4c([2H])c([2H])c([2H])c([2H])c4[2H])nc(-c4c([2H])c([2H])c([2H])c([2H])c4[2H])n3)c(-n3c4ccc(-c5c([2H])c([2H])c([2H])c([2H])c5[2H])cc4c4c5sc6ccccc6c5ccc43)c2-c2c([2H])c([2H])c([2H])c([2H])c2[2H])c([2H])c1[2H]]

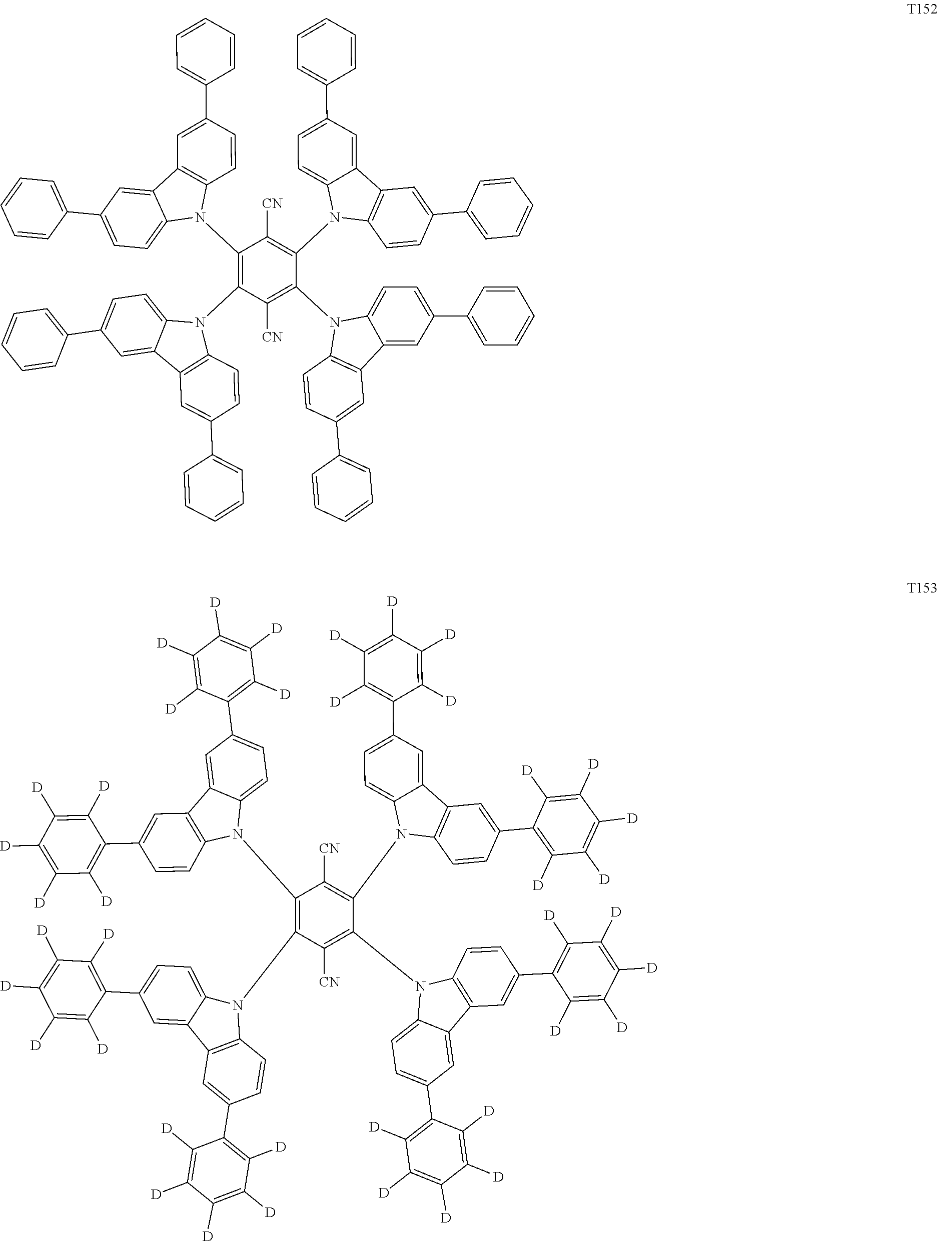

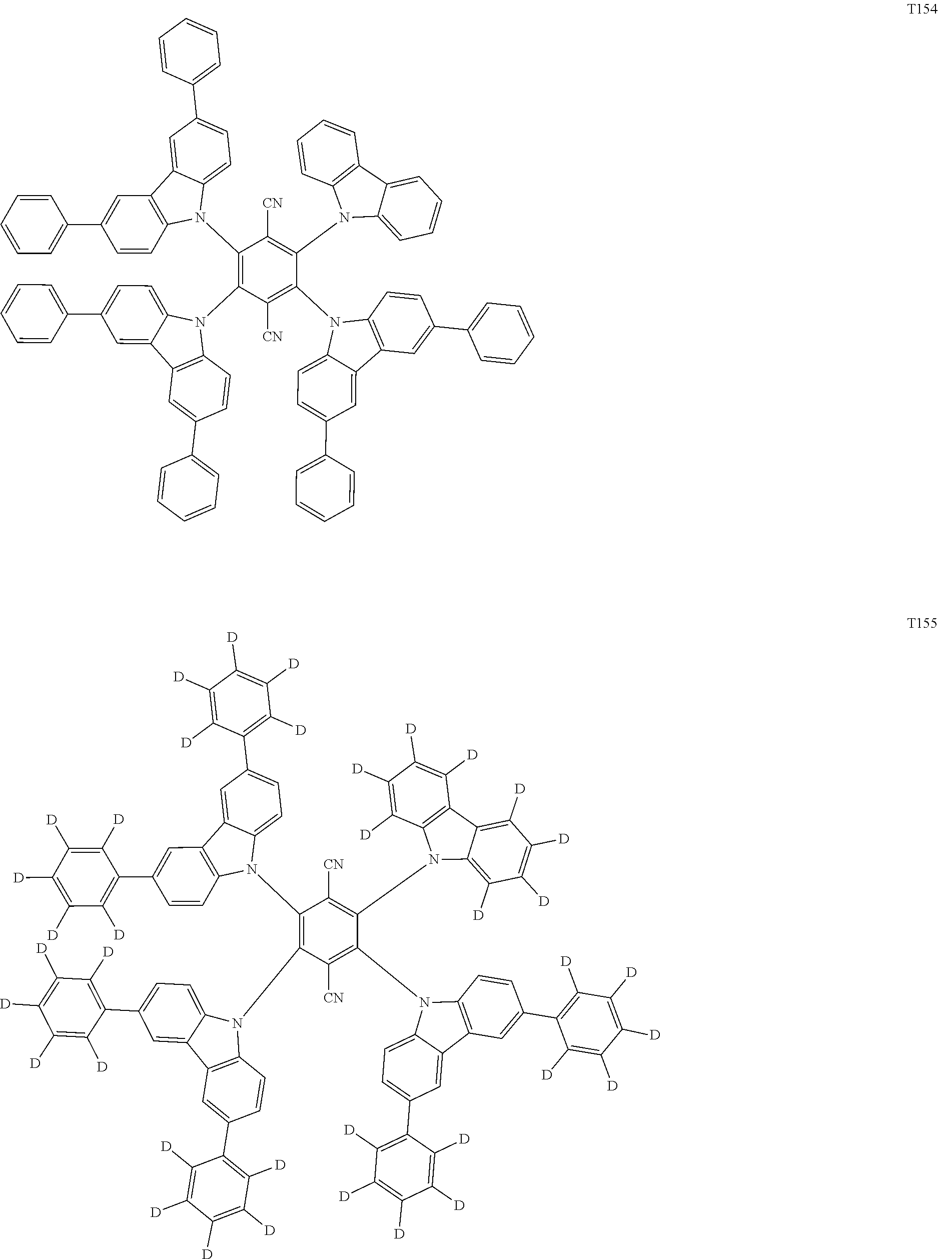

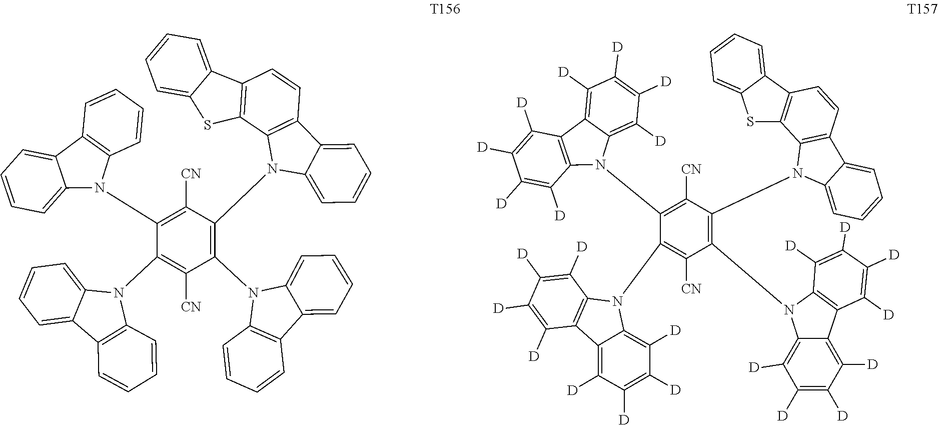 [SMILES: N#Cc1c(-n2c3ccccc3c3ccccc32)c(-n2c3ccccc3c3ccccc32)c(C#N)c(-n2c3ccccc3c3ccc4c5ccccc5sc4c32)c1-n1c2ccccc2c2ccccc21 ; [2H]c1c([2H])c([2H])c2c(c1[2H])c1c([2H])c([2H])c([2H])c([2H])c1n2-c1c(C#N)c(-n2c3c([2H])c([2H])c([2H])c([2H])c3c3c([2H])c([2H])c([2H])c([2H])c32)c(-n2c3ccccc3c3ccc4c5ccccc5sc4c32)c(C#N)c1-n1c2c([2H])c([2H])c([2H])c([2H])c2c2c([2H])c([2H])c([2H])c([2H])c21]

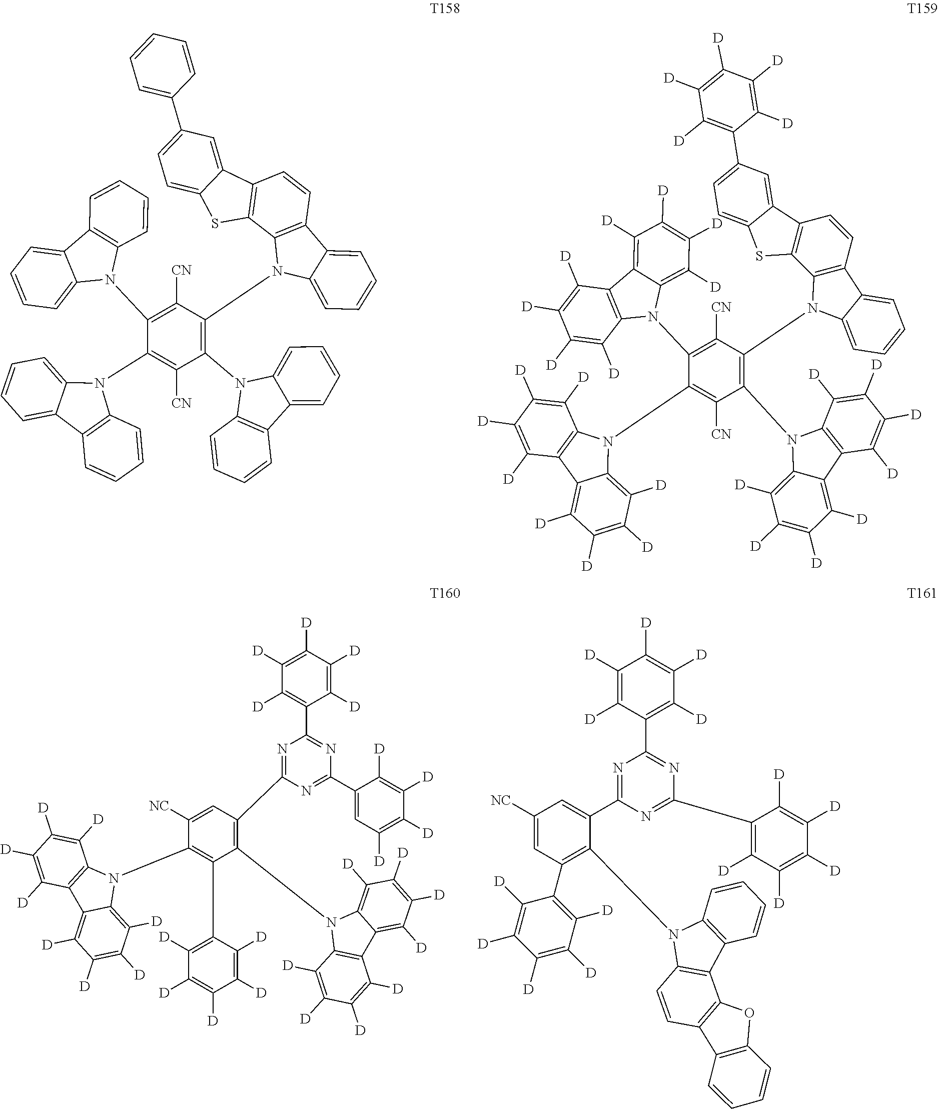

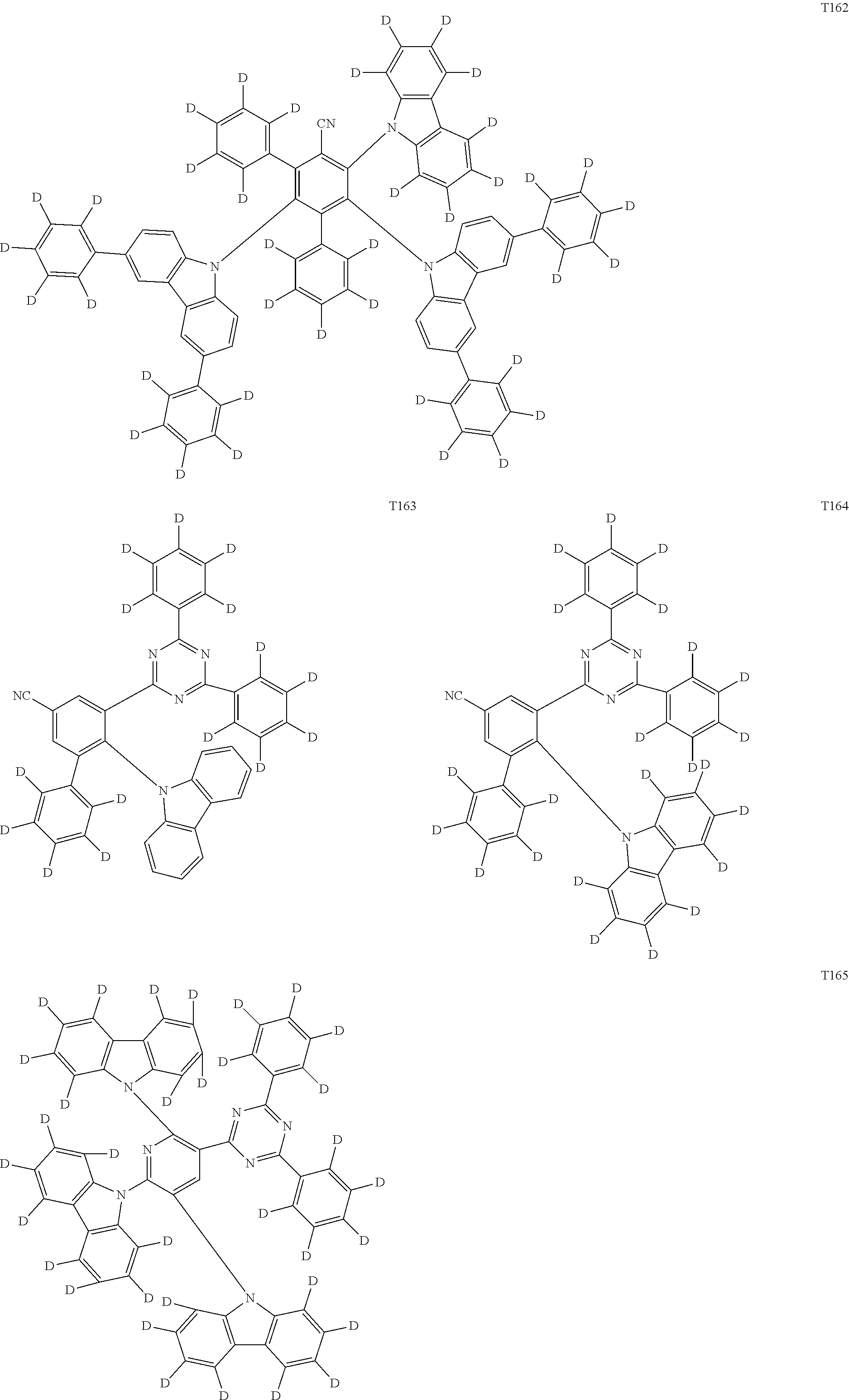

Those produced by substituting all hydrogen atoms in the above Compounds T1 to T165 with deuterium atoms are exemplified here as T1(D) to T165(D). Those produced by substituting all hydrogen atoms in the substituted or unsubstituted carbazol-9-yl group (including those further fused with a ring) present in the above Compounds T1 to T165 with deuterium atoms are exemplified here as T1(d) to T165(d).

Any other known delayed fluorescent materials than the above can be appropriately combined and used. In addition, unknown delayed fluorescent materials can also be used.

As delayed fluorescent materials, there can be mentioned compounds included in the general formulae described in WO2013/154064, paragraphs 0008 to 0048 and 0095 to 0133; WO2013/011954, paragraphs 0007 to 0047 and 0073 to 0085; WO2013/011955, paragraphs 0007 to 0033 and 0059 to 0066; WO2013/081088, paragraphs 0008 to 0071 and 0118 to 0133; JP 2013-256490 A, paragraphs 0009 to 0046 and 0093 to 0134; JP 2013-116975 A, paragraphs 0008 to 0020 and 0038 to 0040; WO2013/133359, paragraphs 0007 to 0032 and 0079 to 0084; WO2013/161437, paragraphs 0008 to 0054 and 0101 to 0121; JP 2014-9352 A, paragraphs 0007 to 0041 and 0060 to 0069; JP 2014-9224 A, paragraphs 0008 to 0048 and 0067 to 0076; JP 2017-119663 A, paragraphs 0013 to 0025; JP 2017-119664 A, paragraphs 0013 to 0026; JP 2017-222623 A, paragraphs 0012 to 0025; JP 2017-226838 A, paragraphs 0010 to 0050; JP 2018-100411 A, paragraphs 0012 to 0043; and WO2018/047853, paragraphs 0016 to 0044; and especially, exemplary compounds therein capable of emitting delayed fluorescence. In addition, also employable here are light emitting materials capable of emitting delayed fluorescence, as described in JP 2013-253121 A, WO2013/133359, WO2014/034535, WO2014/115743, WO2014/122895, WO2014/126200, WO2014/136758, WO2014/133121, WO2014/136860, WO2014/196585, WO2014/189122, WO2014/168101, WO2015/008580, WO2014/203840, WO2015/002213, WO2015/016200, WO2015/019725, WO2015/072470, WO2015/108049, WO2015/080182, WO2015/072537, WO2015/080183, JP 2015-129240 A, WO2015/129714, WO2015/129715, WO2015/133501, WO2015/136880, WO2015/137244, WO2015/137202, WO2015/137136, WO2015/146541 and WO2015/159541. These patent publications described in these paragraphs are hereby incorporated as a part of this description by reference.

In the case where a delayed fluorescent material is used as an assist dopant in the light emitting layer, a compound having a smaller lowest excited singlet energy than the assist dopant is used as the light emitting material. Examples of the light emitting material that is used in combination with an assist dopant include compounds of a boron atom and a nitrogen atom having a multiple resonance effect, and compounds containing a fused aromatic ring structure such as anthracene, pyrene and perylene. In addition, delayed fluorescent materials exemplified hereinabove can also be used.

In one preferred aspect of the present invention, a compound represented by the following general formula (F1) is used as the light emitting material to be used in combination with an assist dopant.

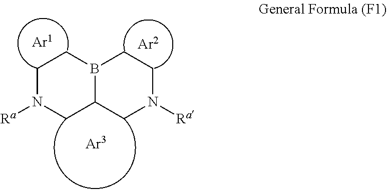

In the above general formula (F1), Ar¹ to Ar³ are each independently an aryl ring or a heteroaryl ring, and at least one hydrogen atom in these rings can be substituted or can be fused with a ring. In the case where the hydrogen atom is substituted, preferably, it is substituted with one group selected from the group consisting of a deuterium atom, an aryl group, a heteroaryl group and an alkyl group, or a group formed by combining at least two such groups. In the case where a ring is fused, preferably, a benzene ring or a heteroaromatic ring (for example, a furan ring, a thiophene ring, and a pyrrole ring) is fused. R^{a} and R^{a′} each independently represent a substituent, preferably one group selected from the group consisting of a deuterium atom, an aryl group, a heteroaryl group and an alkyl group, or a group formed by combining at least two such groups. R^{a} and Ar¹, Ar¹ and Ar², Ar² and R^{a′}, R^{a′} and Ar³, and Ar³ and R^{a} each can bond to each other to form a cyclic structure.

Preferably, the compound represented by the general formula (F1) contains at least one carbazole structure. For example, one benzene ring constituting the carbazole structure can be a ring represented by Ar¹, one benzene ring constituting the carbazole structure can be a ring represented by Ar², and one benzene ring constituting the carbazole structure can be a ring represented by Ar³. Also, a carbazolyl group can bond to at least any one of Ar¹ to Ar³. For example, a substituted or unsubstituted carbazol-9-yl group can bond to the ring represented by Ar³.

A fused aromatic ring structure such as anthracene, pyrene or perylene can bond to Ar¹ to Ar³. Also, the ring represented by Ar¹ to Ar³ can be one ring constituting a fused aromatic ring structure. Further, at least one of R^{a} and R^{a′} can be a group having a fused aromatic ring structure.

The compound can have plural skeletons represented by the general formula (F1). For example, the compound can have a structure where skeletons represented by the general formula (F1) bond to each other via a single bond or a linking group. Also, a structure that exhibits a multiple resonance effect formed by linking benzene rings with a boron atom, a nitrogen atom, an oxygen atom or a sulfur atom can be added to the skeleton represented by the general formula (F1).

In one preferred aspect of the present invention, a compound having a BODIPY (4,4-difluoro-4-bora-3a,4a-diaza-s-indacene) structure is used as the light emitting material to be used in combination with an assist dopant. For example, a compound represented by the following general formula (F2) is used.

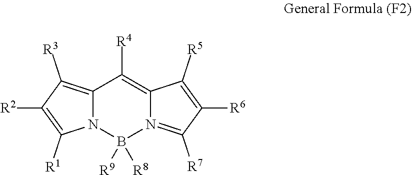

In the general formula (F2), R¹ to R⁷ are each independently a hydrogen atom, a deuterium atom, or a substituent. At least one of R¹ to R⁷ is preferably a group represented by the following general formula (F3).

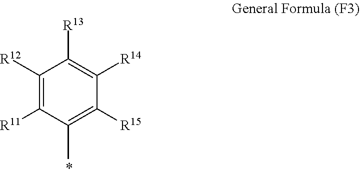

In the general formula (F3), R¹¹ to R¹⁵ each independently represent a hydrogen atom, a deuterium atom or a substituent, and * indicates a bonding site.

The group represented by the general formula (F3) can be one of R¹ to R⁷ in the general formula (F2), or can be two thereof, or can be three thereof. Also, they can be at least four, and for example, four or five. In one preferred aspect of the present invention, one of R¹ to R⁷ is a group represented by the general formula (F3). In one preferred aspect of the present invention, at least R¹, R³, R⁵ and R⁷ each are a group represented by the general formula (F3). In one preferred aspect of the present invention, only R¹, R³, R⁴, R⁵, and R⁷ are groups represented by the general formula (F3). In one preferred aspect of the present invention, R¹, R³, R⁴, R⁵, and R⁷ are groups represented by the general formula (F3), and R² and R⁴ each are a hydrogen atom, a deuterium atom, an unsubstituted alkyl group (for example, having 1 to 10 carbon atoms), or an unsubstituted aryl group (for example, having 6 to 14 carbon atoms). In one aspect of the present invention, all R¹ to R⁷ are groups represented by the general formula (F3).

In one preferred aspect of the present invention, R¹ and R⁷ are the same. In one preferred aspect of the present invention, R³ and R⁵ are the same. In one preferred aspect of the present invention, R² and R⁶ are the same. In one preferred aspect of the present invention, R¹ and R⁷ are the same, R³ and R⁵ are the same, and R¹ and R³ differ from each other. In one preferred aspect of the present invention, R¹, R³, R⁵ and R⁷ are the same. In one preferred aspect of the present invention, R¹, R⁴ and R⁷ are the same, and differ from R³ and R⁵. In one preferred aspect of the present invention, R³, R⁴ and R⁵ are the same, and differ from R¹ and R⁷. In one preferred aspect of the present invention, R¹, R³, R⁵ and R⁷ all differ from R⁴.

The substituent that R¹¹ to R¹⁵ in the general formula (F3) can take can be selected, for example, from the above Substituent Group A, or from the above Substituent Group B, or from the following Substituent Group C, or from the following Substituent Group D. In the case where a substituted amino group is selected for the substituent, it is preferably a di-substituted amino group, and the two substituents of the amino group are each independently preferably a substituted or unsubstituted aryl group, a substituted or unsubstituted heteroaryl group, or a substituted or unsubstituted alkyl group, and especially preferably a substituted or unsubstituted aryl group (a diarylamino group). The substituent that the two aryl groups of the diarylamino group can take can be selected, for example, from the above Substituent Group A, or from the above Substituent Group B, or from the following Substituent Group C, or from the following Substituent Group D. The two aryl groups of the diarylamino group can bond to each other via a single bond or a linking group, and for the linking group as referred to here, reference can be made to the description of the linking group in R³³ and R³⁴. Specific examples of the diarylamino group include a substituted or unsubstituted carbazol-9-yl group. Examples of the substituted or unsubstituted carbazol-9-yl group include a group of the general formula (9) where L¹¹ is a single bond.

In one preferred aspect of the present invention, only R¹³ in the general formula (F3) is a substituent, and R¹¹, R¹², R¹⁴ and R¹⁵ therein are hydrogen atoms. In one preferred aspect of the present invention, only R¹¹ in the general formula (F3) is a substituent, and R¹², R¹³, R¹⁴ and R¹⁵ therein are hydrogen atoms. In one preferred aspect of the present invention, only R¹¹ and R¹³ in the general formula (F3) are substituents, and R¹², R¹⁴ and R¹⁵ therein are hydrogen atoms.

R¹ to R⁷ in the general formula (F2) can include a group of the general formula (F3) where R¹¹ to R¹⁵ are all hydrogen atoms (namely, a phenyl group). For example, R², R⁴, and R⁶ can be phenyl groups.

In the general formula (F2), preferably, R⁸ and R⁹ are each independently one group selected from the group consisting of a hydrogen atom, a deuterium atom, a halogen atom, an alkyl group (for example, having 1 to 40 carbon atoms), an alkoxy group (for example, having 1 to 40 carbon atoms), an aryloxy group (for example, having 6 to 30 carbon atoms) and a cyano group, or a group formed by combining at least two such groups. In one preferred aspect of the present invention, R⁸ and R⁹ are the same. In one preferred aspect of the present invention, R⁸ and R⁹ are halogen atoms, especially preferably fluorine atoms.

In one aspect of the present invention, the number of the substituted or unsubstituted alkoxy group, the substituted or unsubstituted aryloxy group and the substituted or unsubstituted amino group existing in R¹ to R⁹ in the general formula (F2) is preferably at least three in total, and a compound in which the total number is three can be employed, or a compound in which the total number is four can be employed. More preferably, the total number of the substituted or unsubstituted alkoxy group, the substituted or unsubstituted aryloxy group and the substituted or unsubstituted amino group existing in R¹ to R⁷ in the general formula (F2) is preferably three or more in total, and for example, a compound in which the total number is three can be employed, or a compound in which the total number is four can be employed. In that case, an alkoxy group, an aryloxy group and an amino group may not exist in R⁸ and R⁹. Further preferably, the number of the substituted or unsubstituted alkoxy group, the substituted or unsubstituted aryloxy group and the substituted or unsubstituted amino group existing in R¹, R³, R⁴, R⁵ and R⁷ in the general formula (F2) is preferably three or more in total, and for example, a compound in which the total number is three can be employed, or a compound in which the total number is four can be employed. In that case, an alkoxy group, an aryloxy group and an amino group may not exist in R², R⁶, R⁸ and R⁹. In one preferred aspect of the present invention, the compound has at least three substituted or unsubstituted alkoxy groups. In one preferred aspect of the present invention, the compound has at least four substituted or unsubstituted alkoxy groups. In one preferred aspect of the present invention, the compound has at least one substituted or unsubstituted alkoxy group, and at least two substituted or unsubstituted aryloxy groups. In one preferred aspect of the present invention, the compound has at least two substituted or unsubstituted alkoxy groups, and at least one substituted or unsubstituted amino group. In one preferred aspect of the present invention, R¹, R⁴ and R⁷ each have a substituted or unsubstituted alkoxy group or a substituted or unsubstituted aryloxy group. In one preferred aspect of the present invention, R¹, R⁴ and R⁷ each have a substituted or unsubstituted alkoxy group.

In one aspect of the present invention, the number of the substituent having a Hammett's σp value of less than −0.2 existing in R¹ to R⁹ in the general formula (F2) is three or more in total. Examples of the substituent having a Hammett's σp value of less than −0.2 include a methoxy group (−0.27), an ethoxy group (−0.24), an n-propoxy group (−0.25), an isopropoxy group (−0.45), and an n-butoxy group (−0.32). On the other hand, a fluorine atom (0.06), a methyl group (−0.17), an ethyl group (−0.15), a tert-butyl group (−0.20), an n-hexyl group (−0.15), and a cyclohexyl group (−0.15) are not substituents having a Hammett's σp value of less than −0.2.

In one aspect of the present invention, a compound having three substituents each having a Hammett's σp value of less than −0.2 in R¹ to R⁹ in the general formula (F2) can be employed, or a compound having four such substituents can be employed. More preferably, the number of the substituents having a Hammett's σp value of less than −0.2 in R¹ to R⁷ in the general formula (F2) is three or more, and for example, a compound having three such substituents can be employed, or a compound having four such substituents can be employed. In that case, a substituent having a Hammett's σp value of less than −0.2 may not exist in R⁸ and R⁹. Further preferably, the number of the substituents having a Hammett's σp value of less than −0.2 in R¹, R³, R⁴, R⁵ and R⁷ in the general formula (F2) is preferably three or more, and for example, a compound having three such substituents can be employed, or a compound having four such substituents can be employed. In that case, a substituent having a Hammett's σp value of less than −0.2 may not exist in R², R⁶, R⁸ and R⁹. In one preferred aspect of the present invention, R¹, R⁴ and R⁷ each have a substituent having a Hammett's σp value of less than −0.2.

In the present invention, a compound containing a carbazole structure can be selected for the light emitting material to be used in combination with an assist dopant. A compound not containing any of a carbazole structure, a dibenzofuran structure and a dibenzothiophene structure can be selected for the light emitting material to be used in combination with an assist dopant.

Preferred compounds for use as the light emitting material for use in combination with an assist dopant are shown below. However, the light emitting material usable in combination with an assist dopant in the present invention is not construed as limiting to the following specific examples. In the structural formulae of the following exemplary compounds, t-Bu represents a tertiary butyl group (tert-butyl group).

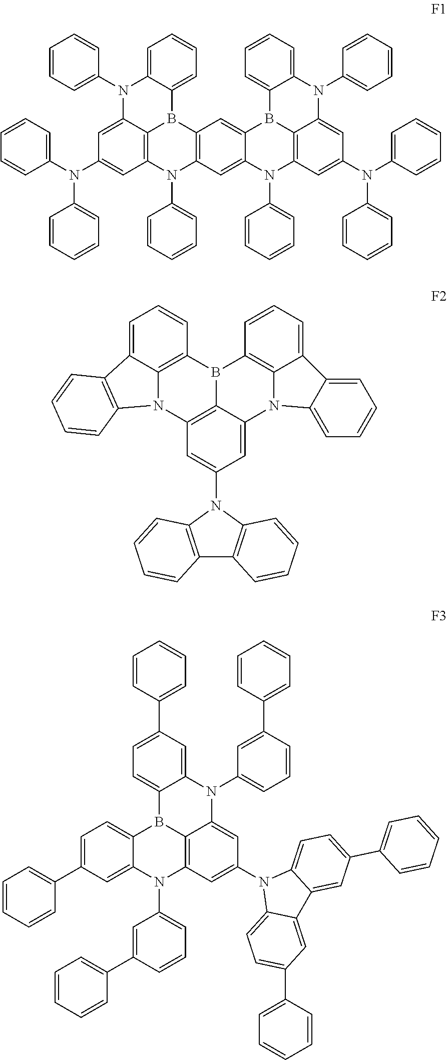 [SMILES: c1ccc(N(c2ccccc2)c2cc3c4c(c2)N(c2ccccc2)c2cc5c(cc2B4c2ccccc2N3c2ccccc2)B2c3ccccc3N(c3ccccc3)c3cc(N(c4ccccc4)c4ccccc4)cc(c32)N5c2ccccc2)cc1 ; c1ccc2c(c1)c1ccccc1n2-c1cc2c3c(c1)-n1c4ccccc4c4cccc(c41)B3c1cccc3c4ccccc4n-2c13 ; c1ccc(-c2cccc(N3c4cc(-c5ccccc5)ccc4B4c5ccc(-c6ccccc6)cc5N(c5cccc(-c6ccccc6)c5)c5cc(-n6c7ccc(-c8ccccc8)cc7c7cc(-c8ccccc8)ccc76)cc3c54)c2)cc1]

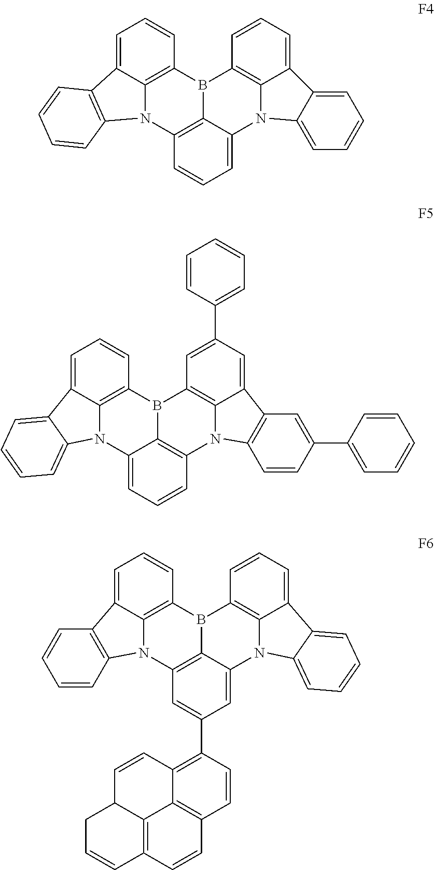 [SMILES: c1cc2c3c(c1)-n1c4ccccc4c4cccc(c41)B3c1cccc3c4ccccc4n-2c13 ; c1ccc(-c2ccc3c(c2)c2cc(-c4ccccc4)cc4c2n3-c2cccc3c2B4c2cccc4c5ccccc5n-3c24)cc1 ; C1=Cc2ccc3ccc(-c4cc5c6c(c4)-n4c7ccccc7c7cccc(c74)B6c4cccc6c7ccccc7n-5c46)c4c3c2C(C=C4)C1]

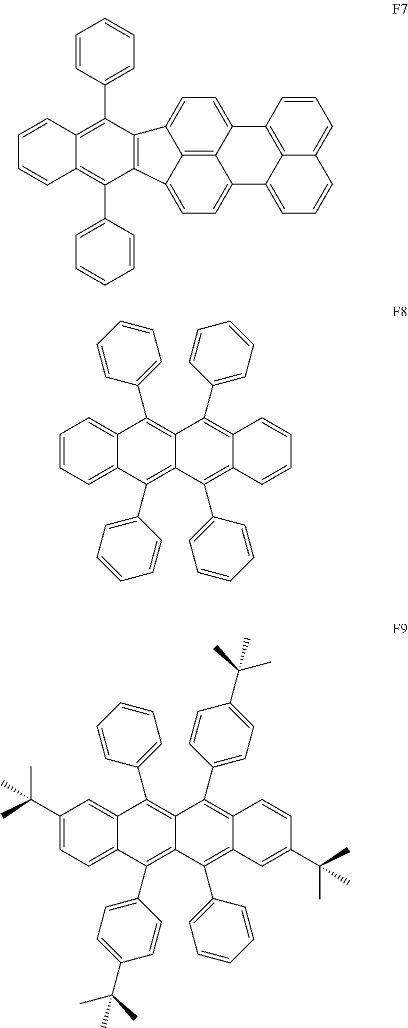

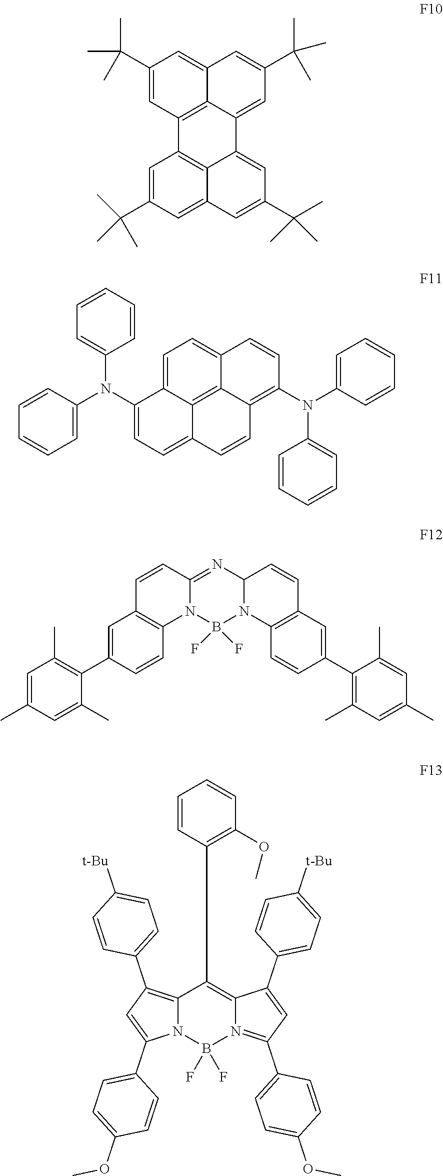

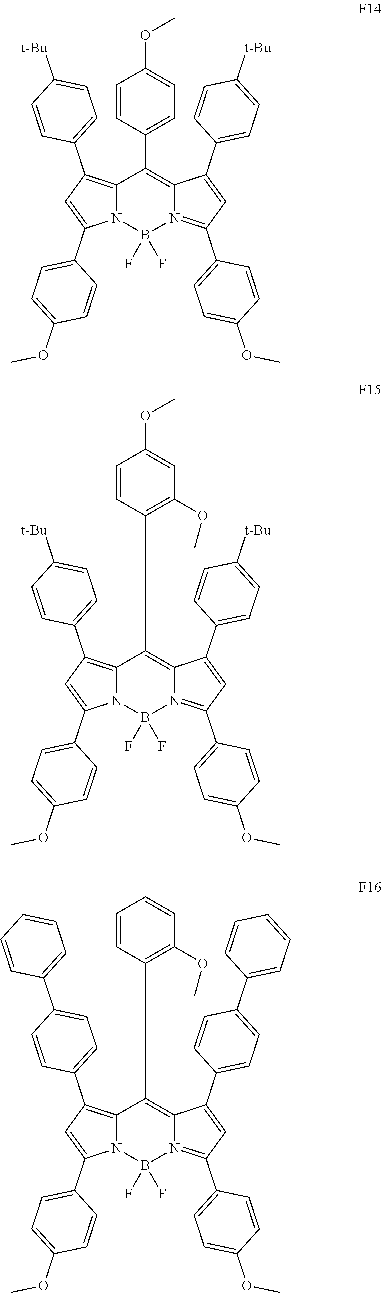

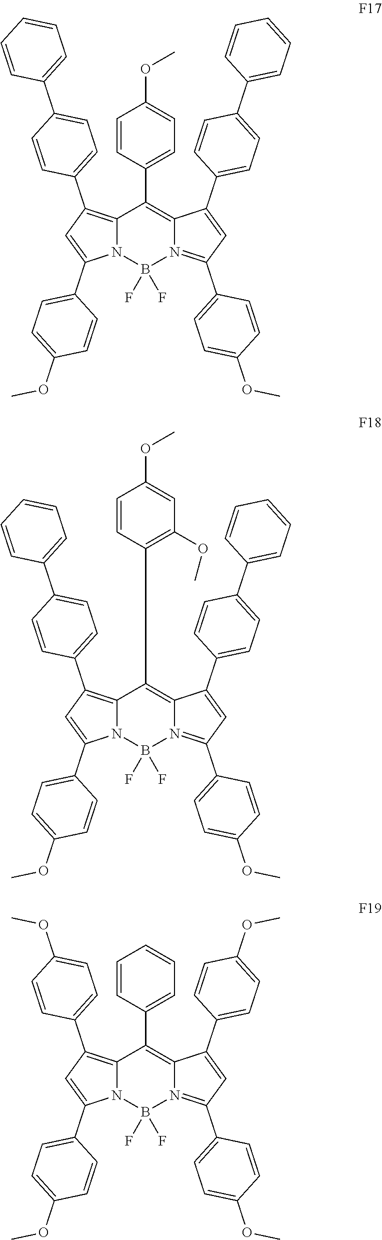

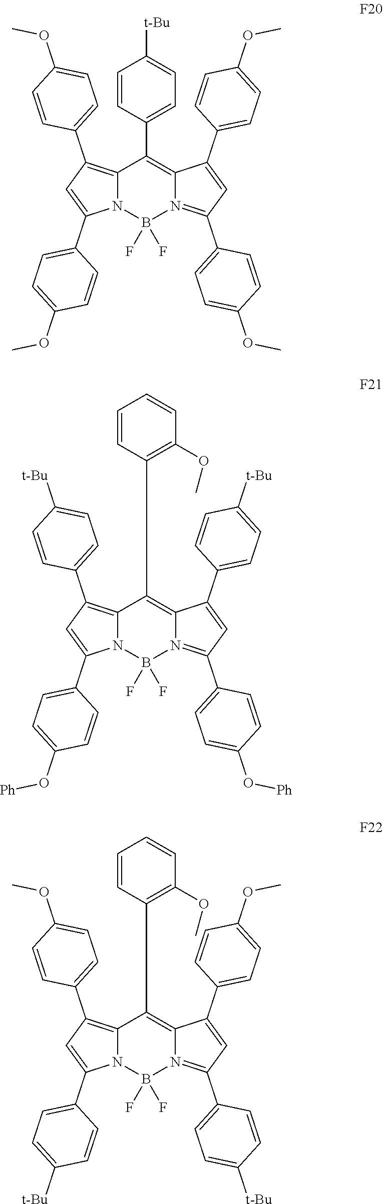

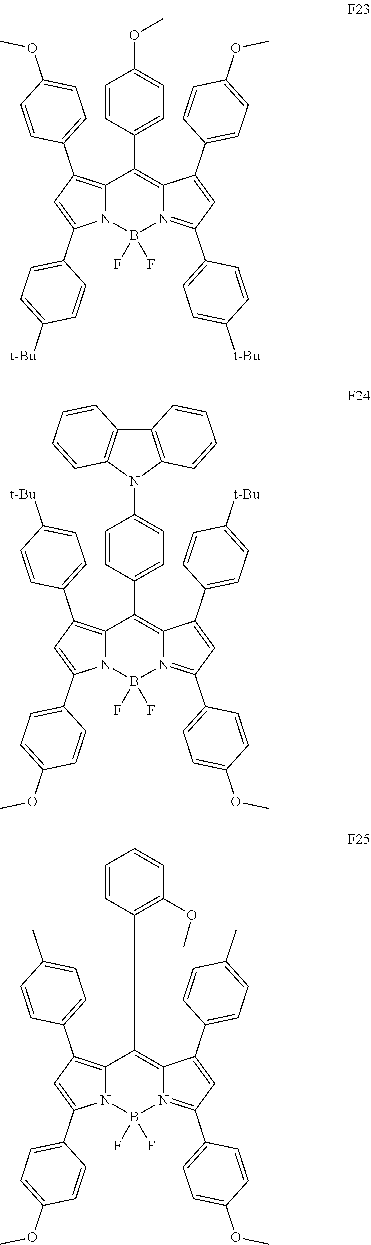

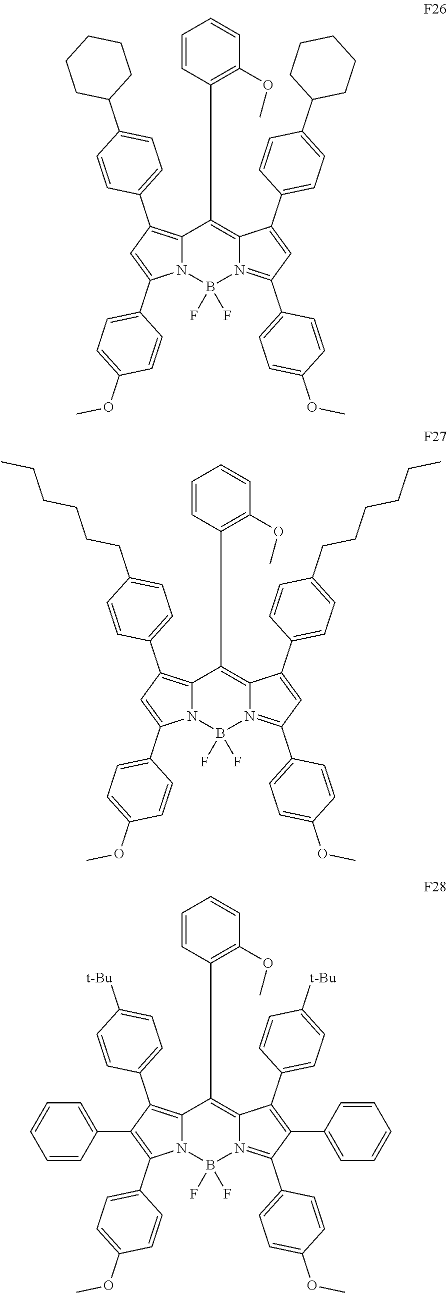

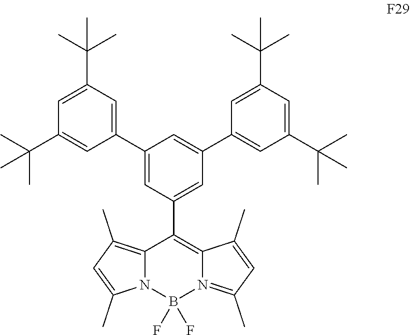

Derivatives of the above exemplary compounds include compounds thereof in which at least one hydrogen atom is substituted with a deuterium atom, an alkyl group, an aryl group, a heteroaryl group, or a diarylamino group.

In addition, compounds described in WO2015/022974, paragraphs 0220 to 0239 are also favorably employable as the light emitting material for use in combination with an assist dopant.

In one preferred aspect of the present invention, a compound represented by the following general formula (G) is used in the light emitting layer. Preferably, the compound represented by the general formula (G) is employed as the light emitting material for use in combination with an assist dopant. The compound represented by the general formula (G) can be employed also as an assist dopant.

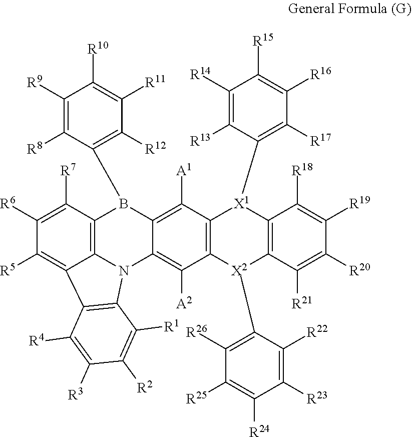

In the general formula (G), one of X¹ and X² is a nitrogen atom, and the other is a boron atom. In one aspect of the present invention, X¹ is a nitrogen atom, and X² is a boron atom. In that case, R¹⁷ and R¹⁸ bond to each other to form a single bond so as to form a pyrrole ring. In another aspect of the present invention, X¹ is a boron atom, and X² is a nitrogen atom. In that case, R²¹ and R²² bond to each other to form a single bond so as to form a pyrrole ring.

In the general formula (G), R¹ to R²⁶, A¹, and A² each independently represent a hydrogen atom, a deuterium atom, or a substituent.

R¹ and R², R² and R³, R³ and R⁴, R⁴ and R⁵, R⁶ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ can bond to each other to form a cyclic structure.

The cyclic structure formed by bonding R⁷ and R⁸ to each other includes a boron atom and four carbon atoms as ring skeleton-constituting atoms. The cyclic structure formed by bonding R¹⁷ and R¹⁸ to each other includes a boron atom and four carbon atoms as ring skeleton-constituting atoms when X¹ is a boron atom. When X¹ is a nitrogen atom, the cyclic structure is limited to a pyrrole ring. The cyclic structure formed by bonding R²¹ and R²² to each other includes a boron atom and four carbon atoms as ring skeleton-constituting atoms when X² is a boron atom. When X² is a nitrogen atom, the cyclic structure is limited to a pyrrole ring. When R⁷ and R⁸, R¹⁷ and R¹⁸, and R²¹ and R²² bond to each other to form boron atom-containing cyclic structures, the cyclic structure is preferably a 5 to 7-membered ring, more preferably a 5 or 6-membered ring, further preferably a 6-membered ring. When R⁷ and R⁸, R¹⁷ and R¹⁸, and R²¹ and R²² bond to each other, these preferably form a single bond, —O—, —S—, —N(R²⁷)—, —C(R²⁸)(R²⁹)—, —Si(R³⁰)(R³¹)—, —B(R³²)—, —CO—, or —CS— by bonding to each other, more preferably form —O—, —S— or —N(R²⁷)—, further preferably form —N(R²⁷)—. Here, each of R²⁷ to R³² independently represents a hydrogen atom, a deuterium atom, or a substituent. As the substituent, a group selected from any of substituent groups A to E to be described below can be employed, but a substituted or unsubstituted alkyl group, a substituted or unsubstituted aryl group, or a substituted or unsubstituted heteroaryl group is preferable. In particular, R²⁷ is preferably a substituted or unsubstituted aryl group. When R²⁷ to R³² are substituents, R²⁷ to R³² in the ring formed by bonding R⁷ and R⁸ to each other may further form a cyclic structure by bonding to at least one of R⁶ and R⁹, R²⁷ to R³² in the ring formed by bonding R¹⁷ and R¹⁸ to each other may further form a cyclic structure by bonding to at least one of R¹⁶ and R¹⁹, and R²⁷ to R³² in the ring formed by bonding R²¹ and R²² to each other may further form a cyclic structure by bonding to at least one of R²⁰ and R²³. In one aspect of the present invention, in only one combination among R⁷ and R⁸, R¹⁷ and R¹⁸, and R²¹ and R²², these bond to each other. In one aspect of the present invention, only two combinations of R⁷ and R⁸, R¹⁷ and R¹⁸, and R²¹ and R²² bond to each other. In one aspect of the present invention, all of R⁷ and R⁸, R¹⁷ and R¹⁸, and R²¹ and R²² bond to each other.

The cyclic structure formed by bonding R¹ and R², R² and R³, R³ and R⁴, R⁴ and R⁵, R⁵ and R⁶, R⁶ and R⁷, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁸ and R¹⁹, R¹⁹ and R²⁰, R²⁰ and R²¹, R²² and R²³, R²³ and R²⁴, R²⁴ and R²⁵, and R²⁵ and R²⁶ to each other can be an aromatic ring or an aliphatic ring, or can contain a hetero atom, and further can be fused with at least one other ring. Here the hetero atom is preferably selected from the group consisting of a nitrogen atom, an oxygen atom and a sulfur atom. Examples of the cyclic structure to be formed include a benzene ring, a pyridine ring, a pyridazine ring, a pyrimidine ring, a pyrazine ring, a pyrrole ring, an imidazole ring, a pyrazole ring, a triazole ring, an imidazoline ring, a furan ring, a thiophene ring, an oxazole ring, an isoxazole ring, a thiazole ring, an isothiazole ring, a cyclohexadiene ring, a cyclohexene ring, a cyclopentene ring, a cycloheptatriene ring, a cycloheptadiene ring, a cycloheptene ring, and a ring in which one or more rings selected from the group consisting of these rings are further fused. In one preferred aspect of the present invention, the cyclic structure is a substituted or unsubstituted benzene ring (further, a ring can be fused), and is for example, a benzene ring which can be substituted with an alkyl group or an aryl group. In one preferred aspect of the present invention, the cyclic structure is a substituted or unsubstituted heteroaromatic ring, preferably a furan ring of benzofuran, or a thiophene ring of benzothiophene. Among R¹ and R², R² and R³, R³ and R⁴, R⁴ and R⁵, R⁴ and R⁶, R⁶ and R⁷, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁸ and R¹⁹, R¹⁹ and R²⁰, R²⁰ and R²¹, R²² and R²³, R²³ and R²⁴, R²⁴ and R²⁵, and R²⁵ and R²⁶, the number of combinations that bond to each other to form cyclic structures can be 0, or can be, for example, any one of 1 to 6. For example, it can be any one of 1 to 4, 1 can be selected, 2 can be selected, or 3 or 4 can be selected. In one aspect of the present invention, in one combination selected from R¹ and R², R² and R³, and R³ and R⁴, a cyclic structure is formed through bonding to each other. In one aspect of the present invention, R⁵ and R⁶ bond to each other to form a cyclic structure. In one aspect of the present invention, in one combination selected from R⁹ and R¹⁰, R¹⁰ and R¹¹, and R¹¹ and R¹², a cyclic structure is formed through bonding to each other. In one aspect of the present invention, in both of R¹ and R², and R¹³ and R¹⁴, cyclic structures are formed through bonding to each other. In one aspect of the present invention, in one combination selected from R¹ and R², R² and R³, and R³ and R⁴, a cyclic structure is formed through bonding to each other, and moreover R⁵ and R⁶ bond to each other to form a cyclic structure. In one aspect of the present invention, in both of R⁵ and R⁶, and R¹⁹ and R²⁰, cyclic structures are formed through bonding to each other.

R¹ to R²⁶ which do not bond to adjacent Rn (n=1 to 26) are hydrogen atoms, deuterium atoms, or substituents. As the substituent, a group selected from any of substituent groups A to E to be described below can be employed.

Preferable substituents which R¹ to R²⁶ can have include a substituted or unsubstituted alkyl group, a substituted or unsubstituted aryl group, and a substituted or unsubstituted heteroaryl group. For example, the substituent can be a substituted or unsubstituted aryl group, and for example the substituent can be a substituted or unsubstituted alkyl group. As the substituent for the alkyl group, the aryl group, or the heteroaryl group mentioned herein, a group selected from any of substituent groups A to E can be employed, but the substituent is preferably at least one group selected from the group consisting of an alkyl group, an aryl group and a heteroaryl group, more preferably a group of Substituent Group E, and the groups can be unsubstituted. In one preferred aspect of the present invention, at least one of R¹ to R⁶ is a substituent, preferably a group of Substituent Group E. For example, at least one of R² to R⁶ is a substituent, preferably a group of Substituent Group E. For example, at least one of R⁵ and R⁶ is a substituent, preferably a group of Substituent Group E. In one preferred aspect of the present invention, at least one of R³ and R⁶ is a substituent, more preferably both are substituents, and a group of Substituent Group E is preferred. In one preferred aspect of the present invention, when X¹ is a nitrogen atom, at least one of R¹⁵ and R²⁰ is a substituent, more preferably both are substituents, and a group of Substituent Group E is preferred. Here, R¹⁷ and R¹⁸ bond to each other to form a single bond. In one preferred aspect of the present invention, when X² is a nitrogen atom, at least one of R¹⁹ and R²⁴ is a substituent, more preferably both are substituents, and a group of Substituent Group E is preferred. Here, R²¹ and R²² bond to each other to form a single bond. In one aspect of the present invention, at least one of R⁸ and R¹² is a substituent, and preferably both are substituents. In one aspect of the present invention, R⁸, R¹⁰ and R¹² are substituents. As for the substituent of R⁸ to R¹², an unsubstituted alkyl group is preferable. In particular, the case where R⁸ and R¹² are alkyl groups having 2 or more carbon atoms (preferably alkyl groups having 3 or more carbon atoms, more preferably alkyl groups having 3 to 8 carbon atoms, further preferably alkyl groups having 3 or 4 carbon atoms) is preferable because orientation becomes high when a film is formed. Among them, particularly preferred is a case where R⁸ and R¹² are substituents (preferably alkyl groups, more preferably alkyl groups having 2 or more carbon atoms, further preferably alkyl groups having 3 or more carbon atoms, still further preferably alkyl groups having 3 to 8 carbon atoms, particularly preferably alkyl groups having 3 or 4 carbon atoms), and moreover, at least one of R¹ to R⁶ is a substituent (preferably a group of Substituent Group E). When X¹ is a boron atom, at least one of R¹³ and R¹⁷ is a substituent, and preferably both are substituents. In one aspect of the present invention, when X¹ is a boron atom, R¹³, R¹⁵ and R¹⁷ are substituents. When X¹ is a boron atom, as for the substituent of R¹³ to R¹⁷, an unsubstituted alkyl group is preferable. When X² is a boron atom, at least one of R²² and R²⁶ is a substituent, and preferably both are substituents. In one aspect of the present invention, when X² is a boron atom, R²², R²⁴ and R²⁶ are substituents. When X² is a boron atom, as for the substituent of R²² to R²⁶, an unsubstituted alkyl group is preferable. Specific examples of the group that bonds to the boron atom represented by B in the general formula (G) or the boron atom represented by X¹ or X² will be given below. Meanwhile, groups bonded to the boron atom, which can be adopted in the present invention, are not construed as limiting to the following specific examples. In the present description, indication of CH₃ is omitted for a methyl group. * indicates a bonding site.

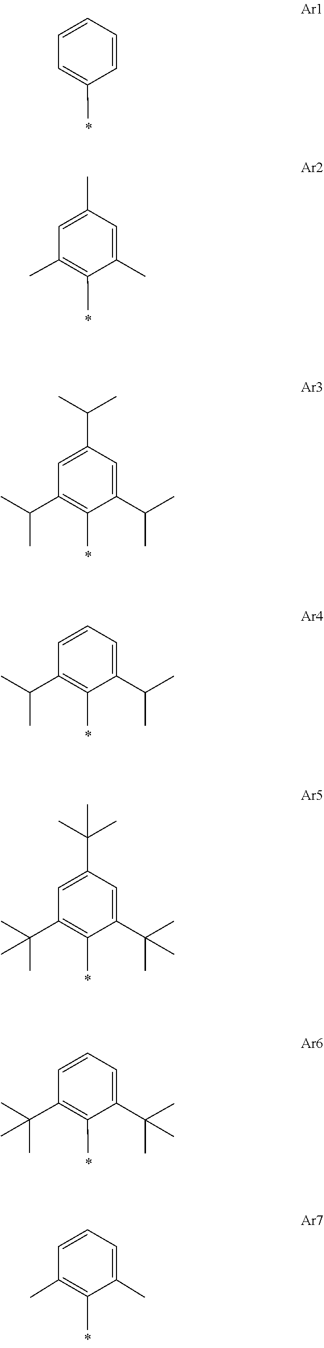

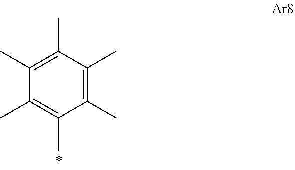

Hereinafter, specific examples of R¹ to R²⁶ in the general formula (G) will be given. G1 to G9 are preferable as R¹ to R⁷, as R¹³ to R²¹ when X¹ is a nitrogen atom, and as R¹⁸ to R²⁶ when X² is a nitrogen atom, G1 to G7 are preferable as R⁸ to R¹², as R²² to R²⁶ when X¹ is a nitrogen atom, and as R¹³ to R¹⁷ when X² is a nitrogen atom. Meanwhile, groups bonded to the boron atom, which can be adopted in the present invention, are not construed as limiting to the following specific examples. D represents a deuterium atom. * indicates a bonding site.

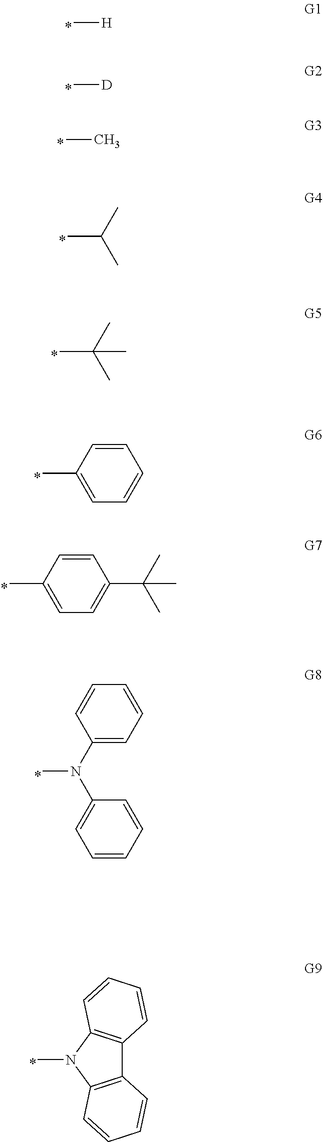

A¹ and A² are hydrogen atoms, deuterium atoms, or substituents. As the substituent, a group selected from any of substituent groups A to E to be described below can be employed.

In one preferred aspect of the present invention, each of A¹ and A² is independently a hydrogen atom or a deuterium atom. For example, A¹ and A² are hydrogen atoms. For example, A¹ and A² are deuterium atoms.
One of A¹ and A² can be a substituent. Further, each of A¹ and A² can be independently a substituent. A preferable substituent which A¹ and A² can have is an acceptor group. The acceptor group is a group having a positive Hammett's σp value.

The acceptor group which A¹ and A² can have is more preferably a group having a Hammett's σp value greater than 0.2. Examples of the group having a Hammett's op value greater than 0.2 include a cyano group, an aryl group substituted with at least a cyano group, a fluorine atom-containing group, and a substituted or unsubstituted heteroaryl group containing a nitrogen atom as a ring skeleton-constituting atom. The aryl group substituted with at least a cyano group, which is mentioned herein, can be substituted with a substituent other than the cyano group (for example, an alkyl group or an aryl group), but can be an aryl group substituted with only a cyano group. The aryl group substituted with at least a cyano group is preferably a phenyl group substituted with at least a cyano group. The number of substitutions of the cyano group is preferably one or two, and, for example, can be one, or can be two. As the fluorine atom-containing group, a fluorine atom, a fluoroalkyl group, and an aryl group substituted with at least a fluorine atom or a fluoroalkyl group can be mentioned. The fluoroalkyl group is preferably a perfluoroalkyl group, and the number of carbon atoms thereof is preferably 1 to 6, more preferably 1 to 3. Further, the heteroaryl group containing a nitrogen atom as a ring skeleton-constituting atom can be a monocycle, or can be a fused ring in which two or more rings are fused. In the case of a fused ring, the number of rings after fusing is preferably two to six, and, for example, can be selected from two to four, or can be two. Specific examples of the ring constituting the heteroaryl group include a pyridine ring, a pyrimidine ring, a pyrazine ring, a triazine ring, a quinoline ring, an isoquinoline ring, a quinazoline ring, a quinoxaline ring, and a naphthyridine ring other than the quinazoline ring or the quinoxaline ring. The ring constituting the heteroaryl group can be substituted with a deuterium atom or a substituent, and as for the substituent, for example, one group selected from the group consisting of an alkyl group, an aryl group and a heteroaryl group or a group formed by combining two or more thereof can be mentioned. As the acceptor group that A¹ and A² can have, a cyano group is particularly preferable.

In one aspect of the present invention, at least one of A¹ and A² is an acceptor group. In one aspect of the present invention, only one of A¹ and A² is an acceptor group. In one aspect of the present invention, both A¹ and A² are the same acceptor groups. In one aspect of the present invention, A¹ and A² are different acceptor groups. In one aspect of the present invention, A¹ and A² are cyano groups. In one aspect of the present invention, A¹ and A² are halogen atoms, for example, bromine atoms.

Hereinafter, specific examples of the acceptor group that can be adopted in the present invention will be illustrated. However, the acceptor group that can be used in the present invention is not construed as limiting to the following specific examples. In the present description, indication of CH₃ is omitted for a methyl group. Thus, for example, A15 indicates a group including two 4-methylphenyl groups. Further, “D” represents a deuterium atom. * indicates a bonding site.

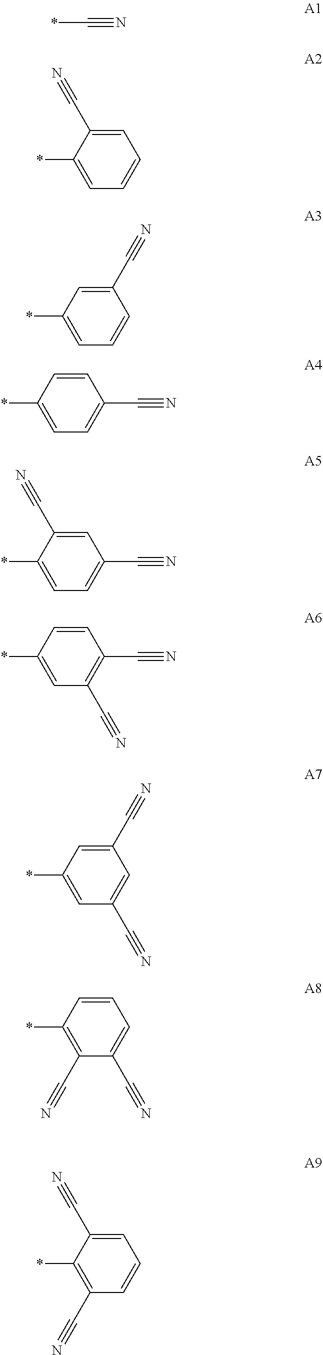

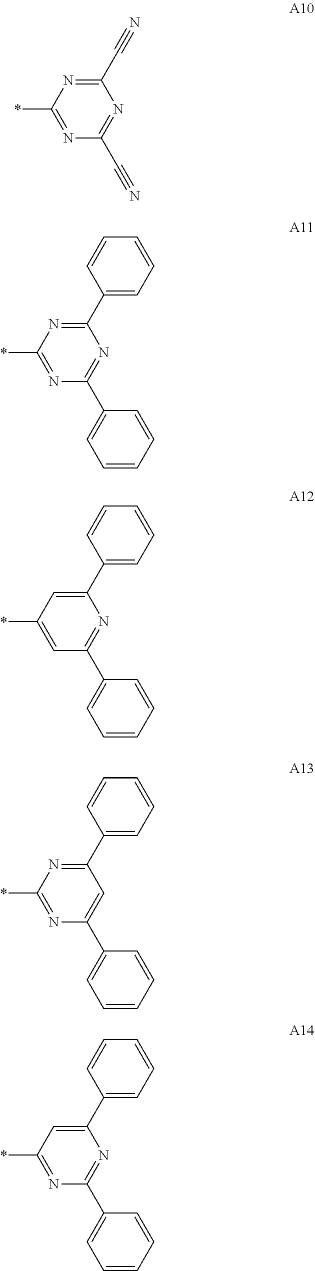

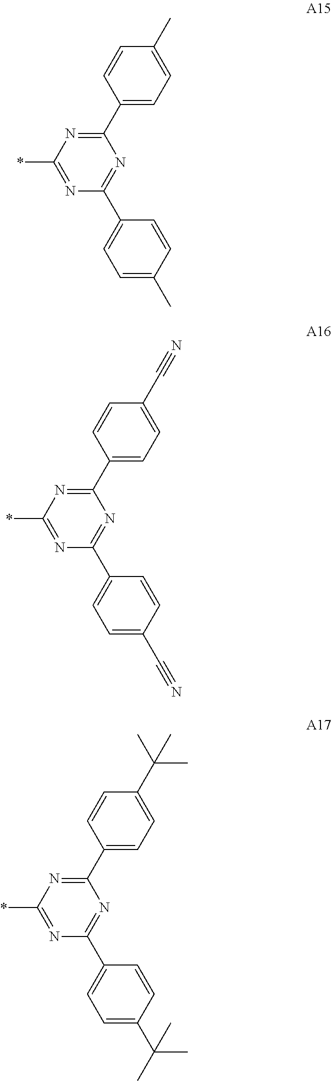

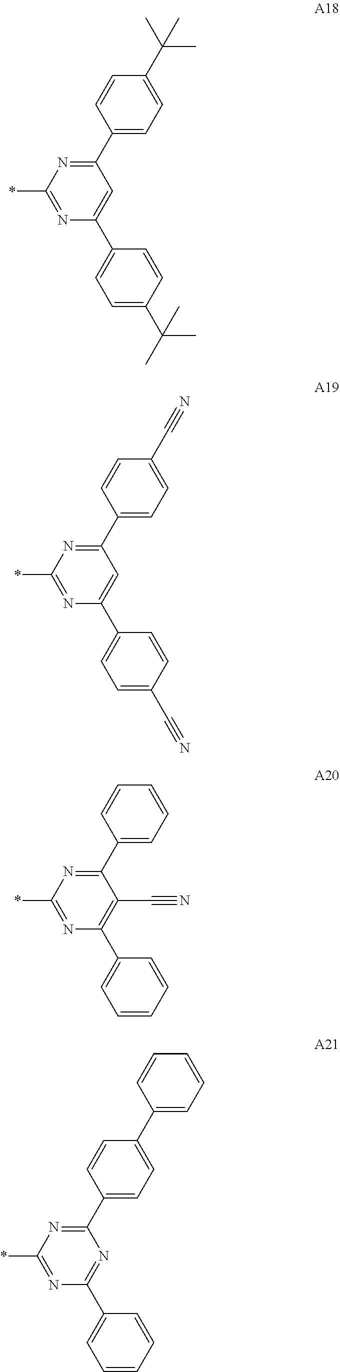

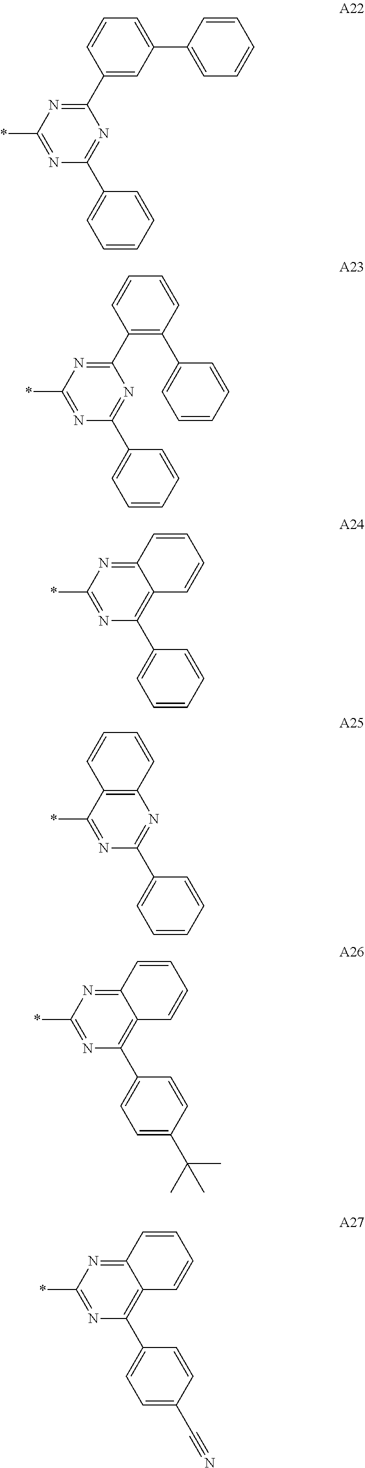

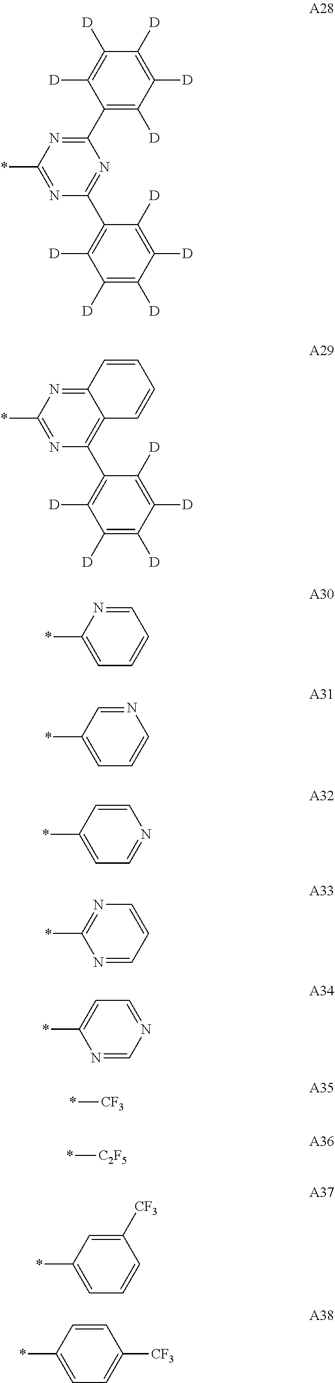

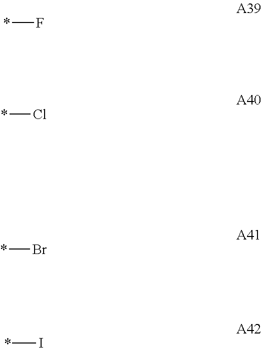

When X¹ is a nitrogen atom, R⁷ and R⁸ bond via a nitrogen atom to form a 6-membered ring, R²¹ and R²² bond via a nitrogen atom to form a 6-membered ring, and R¹⁷ and R¹⁸ bond to each other to form a single bond, at least one of R¹ to R⁶ is a substituted or unsubstituted aryl group, or any of R¹ and R², R² and R³, R³ and R⁴, R⁴ and R⁵, and R⁵ and R⁶ bond to each other to form an aromatic ring (a substituted or unsubstituted benzene ring which can be fused) or a heteroaromatic ring (preferably a substituted or unsubstituted furan ring of benzofuran which can be fused, or a substituted or unsubstituted thiophene ring of benzothiophene which can be fused).

Further, when X¹ is a boron atom, X² is a nitrogen atom, and R⁷ and R⁸, and R¹⁷ and R¹⁸ bond to each other to form boron atom-containing cyclic structures, the cyclic structure is a 5 to 7-membered ring, and in the case of a 6-membered ring, R⁷ and R⁸, and R¹⁷ and R¹⁸ bond to each other to form —B(R³²)—, —CO—, —CS— or —N(R²⁷)—. R²⁷ preferably represents a hydrogen atom, a deuterium atom, or a substituent.

When X¹ in the general formula (G) is a nitrogen atom, the compound of the present invention has the following skeleton (1a). When X² in the general formula (G) is a nitrogen atom, the compound of the present invention has the following skeleton (1b).

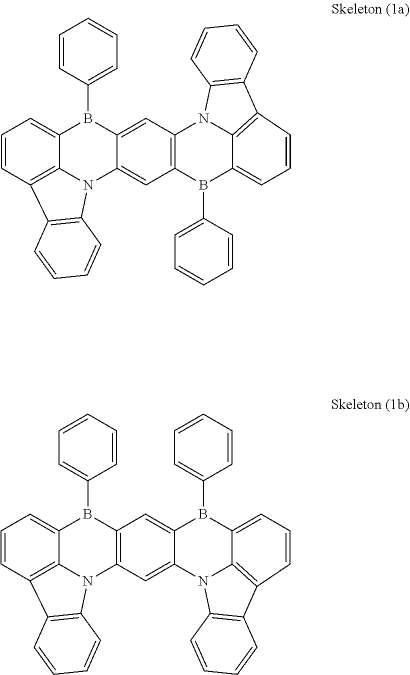

In the skeletons (1a) and (1b), each hydrogen atom can be substituted with a deuterium atom or a substituent. Further, it can be substituted with a linking group together with an adjacent hydrogen atom to form a cyclic structure. For details, corresponding descriptions on R¹ to R²⁶, A¹, and A² in the general formula (G) can be referred to. Compounds, in which all phenyl groups bonding to boron atoms in the skeletons (1a) and (1b) are substituted with mesityl groups, 2,6-diisopropylphenyl groups or 2,4,6-triisopropylphenyl groups, can be exemplified. In one aspect of the present invention, each hydrogen atom in the skeletons (1a) and (1b) is not substituted with a linking group together with an adjacent hydrogen atom to form a cyclic structure.

As one preferable group of compounds having the skeleton (1a), compounds represented by the following general formula (1a) can be exemplified.

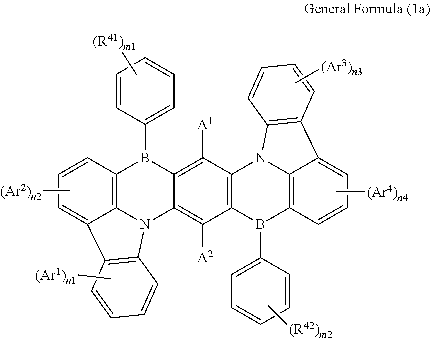

In the general formula (1a), Ar¹ to Ar⁴ each independently represent a substituted or unsubstituted aryl group, a substituted or unsubstituted heteroaryl group, or a substituted or unsubstituted alkyl group, and, for example, a substituted or unsubstituted aryl group can be preferably selected. R⁴¹ and R⁴² each independently represent a substituted or unsubstituted alkyl group. m1 and m2 each independently represent an integer of 0 to 5, n1 and n3 each independently represent an integer of 0 to 4, and n2 and n4 each independently represent an integer of 0 to 3. Each of A¹ and A² independently represents a hydrogen atom, a deuterium atom, or a substituent. It is preferable that at least one of n1 to n4 is 1 or more, and each of m1 and m2 is independently any integer of 1 to 5.

In one aspect of the present invention, n1 to n4 each independently represent an integer of 0 to 2. In one preferred aspect of the present invention, at least one of n1 to n4 is 1 or more. Preferably, at least one of n1 and n2 is 1 or more, and at least one of n3 and n4 is 1 or more. In one aspect of the present invention, each of n1 and n3 is independently 1 or 2, and n2 and n4 are 0. In one aspect of the present invention, each of n2 and n4 is independently 1 or 2, and n1 and n3 are 0. In one aspect of the present invention, each of n1 to n4 is independently 1 or 2. In one aspect of the present invention, n1 and n3 are the same, and n2 and n4 are the same. In one aspect of the present invention, n1 and n3 are 1, and n2 and n4 are 0. In one aspect of the present invention, n1 and n3 are 0, and n2 and n4 are 1. In one aspect of the present invention, n1 to n4 are all 1. The bonding sites of Ar¹ to Ar⁴ can be at least one of 3 and 6 positions in the carbazole ring, can be at least one of 2 and 7 positions, can be at least one of 1 and 8 positions, or can be at least one of 4 and 5 positions. The bonding sites of Ar¹ to Ar⁴ can be both of 3 and 6 positions in the carbazole ring, can be both of 2 and 7 positions, can be both of 1 and 8 positions, or can be both of 4 and 5 positions. For example, at least one of 3 and 6 positions can be preferably selected, or both of 3 and 6 positions can be further preferably selected. In one preferred aspect of the present invention, Ar¹ to Ar⁴ are all the same groups. In one preferred aspect of the present invention, each of Ar¹ to Ar⁴ is independently a substituted or unsubstituted aryl group, more preferably a substituted or unsubstituted phenyl group or naphthyl group, further preferably a substituted or unsubstituted phenyl group. As the substituent, a group selected from any of Substituent Groups A to E to be described below can be mentioned, but an unsubstituted phenyl group is also preferable. Specific preferable examples of Ar¹ to Ar⁴ include a phenyl group, an o-biphenyl group, a m-biphenyl group, a p-biphenyl group, and a terphenyl group.

In one aspect of the present invention, each of m1 and m2 is independently 0. In one aspect of the present invention, each of m1 and m2 is independently any integer of 1 to 5. In one aspect of the present invention, m1 and m2 are the same. In one aspect of the present invention, R⁴¹ and R⁴² are alkyl groups having 1 to 6 carbon atoms and can be selected from, for example, alkyl groups having 1 to 3 carbon atoms, or a methyl group can be selected. When a carbon atom bonded to a boron atom is the 1-position, as the substitution position of the alkyl group, only the 2-position, only the 3-position, only the 4-position, the 3 and 5 positions, the 2 and 4 positions, the 2 and 6 positions, the 2, 4, and 6 positions, and the like can be exemplified. At least the 2-position is preferable, and at least 2 and 6 positions are more preferable.

For descriptions and preferable ranges of A¹ and A², corresponding descriptions on the general formula (G) can be referred to.

Hereinafter, specific examples of the compound represented by the general formula (1a) will be given. Compounds of the general formula (1a) which can be used in the present invention are not construed as limiting to specific examples in the following group. For example, as one preferable group, a group including all the following compounds, except for the compound at the center in the fourth row and the compound at the center in the eighth row, can be mentioned.

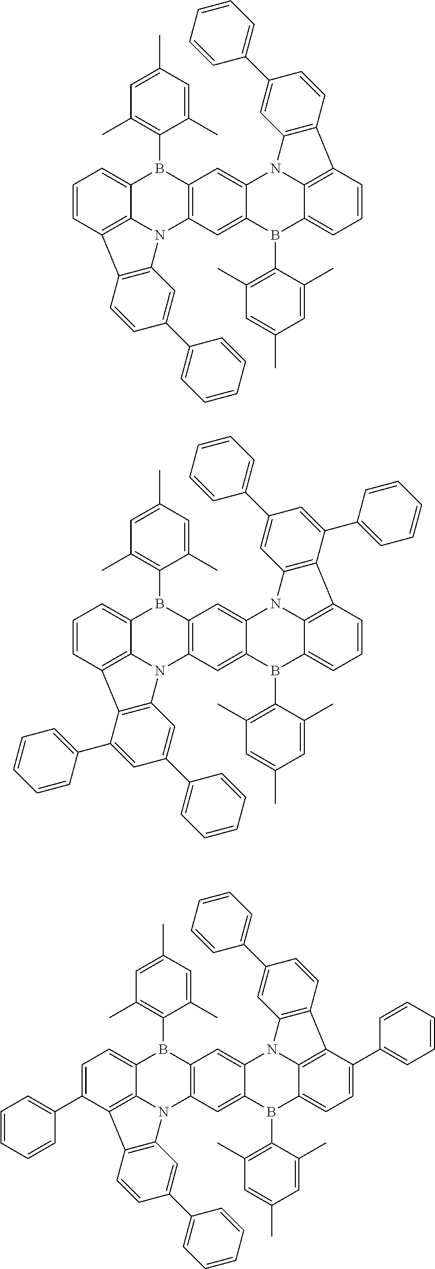

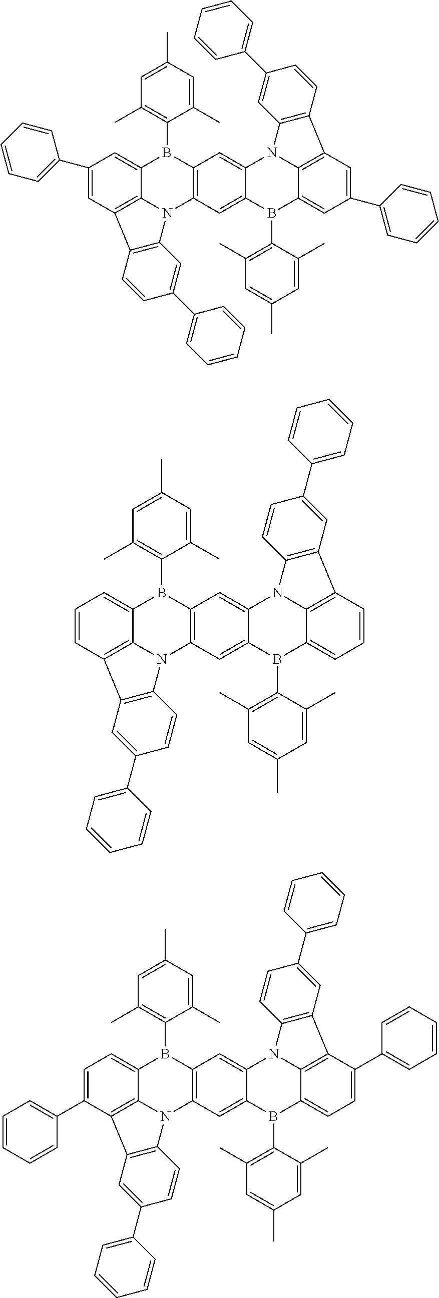 [SMILES: Cc1cc(C)c(B2c3cc4c(cc3-n3c5cc(-c6ccccc6)ccc5c5cc(-c6ccccc6)cc2c53)B(c2c(C)cc(C)cc2C)c2cc(-c3ccccc3)cc3c5ccc(-c6ccccc6)cc5n-4c23)c(C)c1 ; Cc1cc(C)c(B2c3cc4c(cc3-n3c5ccc(-c6ccccc6)cc5c5cccc2c53)B(c2c(C)cc(C)cc2C)c2cccc3c5cc(-c6ccccc6)ccc5n-4c23)c(C)c1 ; Cc1cc(C)c(B2c3cc4c(cc3-n3c5ccc(-c6ccccc6)cc5c5c(-c6ccccc6)ccc2c53)B(c2c(C)cc(C)cc2C)c2ccc(-c3ccccc3)c3c5cc(-c6ccccc6)ccc5n-4c23)c(C)c1]

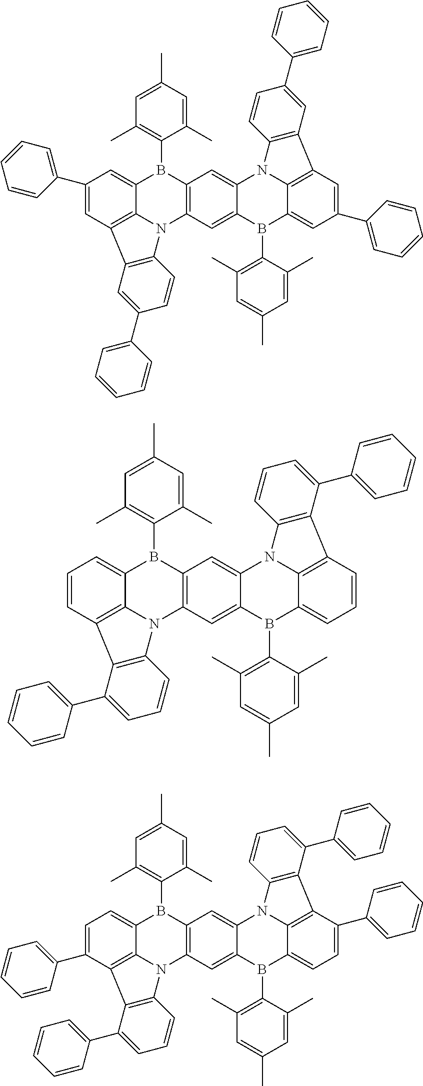

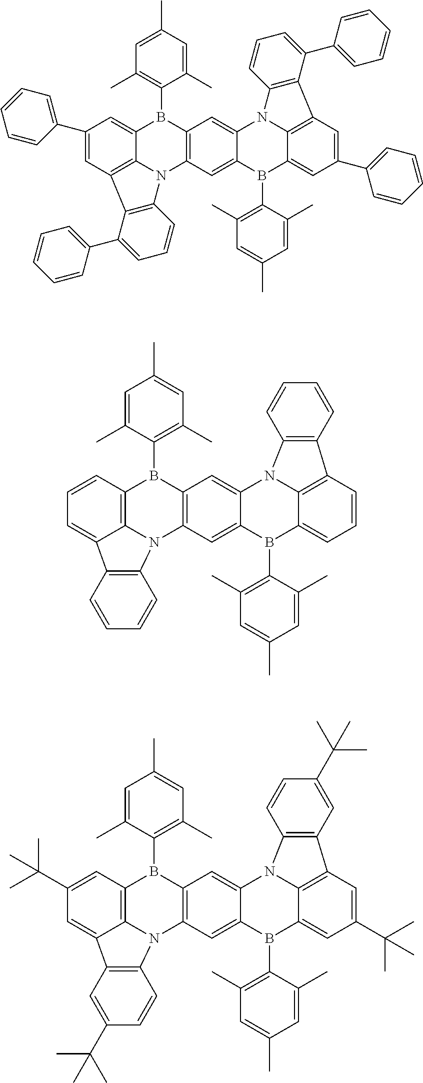 [SMILES: Cc1cc(C)c(B2c3cc4c(cc3-n3c5cccc(-c6ccccc6)c5c5cc(-c6ccccc6)cc2c53)B(c2c(C)cc(C)cc2C)c2cc(-c3ccccc3)cc3c5c(-c6ccccc6)cccc5n-4c23)c(C)c1 ; Cc1cc(C)c(B2c3cc4c(cc3-n3c5ccccc5c5cccc2c53)B(c2c(C)cc(C)cc2C)c2cccc3c5ccccc5n-4c23)c(C)c1 ; Cc1cc(C)c(B2c3cc4c(cc3-n3c5ccc(C(C)(C)C)cc5c5cc(C(C)(C)C)cc2c53)B(c2c(C)cc(C)cc2C)c2cc(C(C)(C)C)cc3c5cc(C(C)(C)C)ccc5n-4c23)c(C)c1]

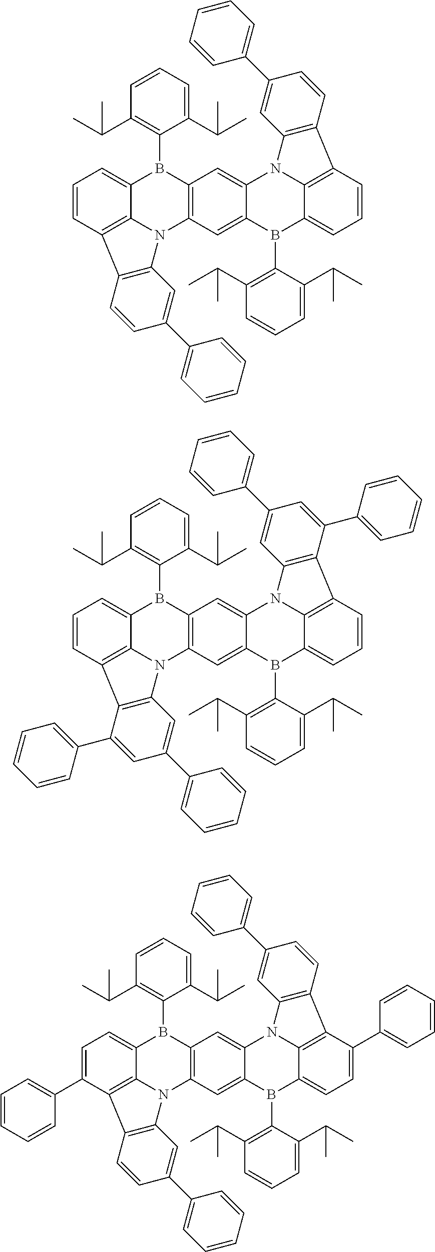

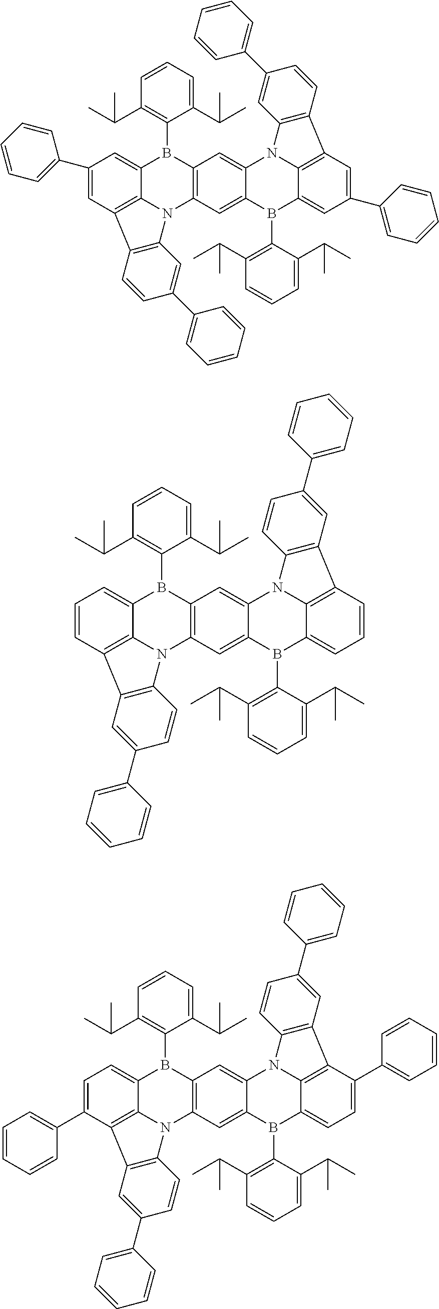 [SMILES: CC(C)c1cccc(C(C)C)c1B1c2cc3c(cc2-n2c4cc(-c5ccccc5)ccc4c4cc(-c5ccccc5)cc1c42)B(c1c(C(C)C)cccc1C(C)C)c1cc(-c2ccccc2)cc2c4ccc(-c5ccccc5)cc4n-3c12 ; CC(C)c1cccc(C(C)C)c1B1c2cc3c(cc2-n2c4ccc(-c5ccccc5)cc4c4cccc1c42)B(c1c(C(C)C)cccc1C(C)C)c1cccc2c4cc(-c5ccccc5)ccc4n-3c12 ; CC(C)c1cccc(C(C)C)c1B1c2cc3c(cc2-n2c4ccc(-c5ccccc5)cc4c4c(-c5ccccc5)ccc1c42)B(c1c(C(C)C)cccc1C(C)C)c1ccc(-c2ccccc2)c2c4cc(-c5ccccc5)ccc4n-3c12]

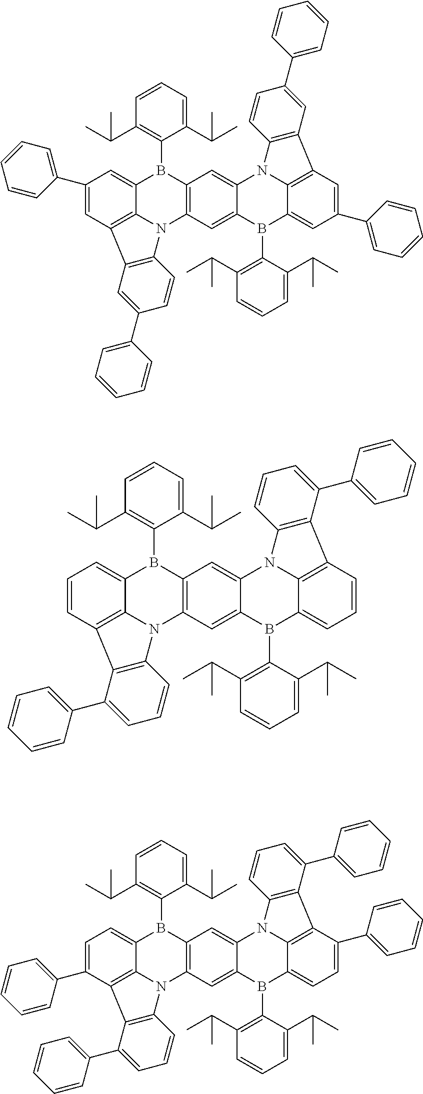 [SMILES: CC(C)c1cccc(C(C)C)c1B1c2cc3c(cc2-n2c4ccc(-c5ccccc5)cc4c4cc(-c5ccccc5)cc1c42)B(c1c(C(C)C)cccc1C(C)C)c1cc(-c2ccccc2)cc2c4cc(-c5ccccc5)ccc4n-3c12 ; CC(C)c1cccc(C(C)C)c1B1c2cc3c(cc2-n2c4cccc(-c5ccccc5)c4c4cccc1c42)B(c1c(C(C)C)cccc1C(C)C)c1cccc2c4c(-c5ccccc5)cccc4n-3c12 ; CC(C)c1cccc(C(C)C)c1B1c2cc3c(cc2-n2c4cccc(-c5ccccc5)c4c4c(-c5ccccc5)ccc1c42)B(c1c(C(C)C)cccc1C(C)C)c1ccc(-c2ccccc2)c2c4c(-c5ccccc5)cccc4n-3c12]

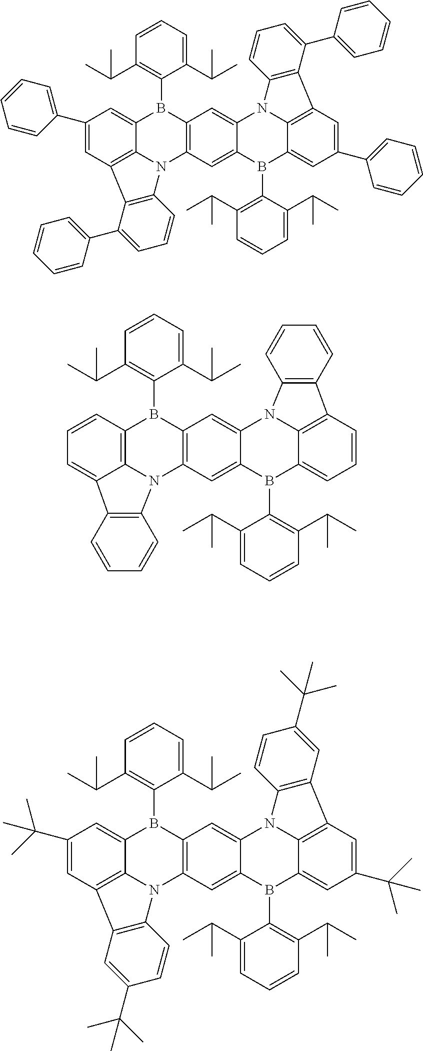

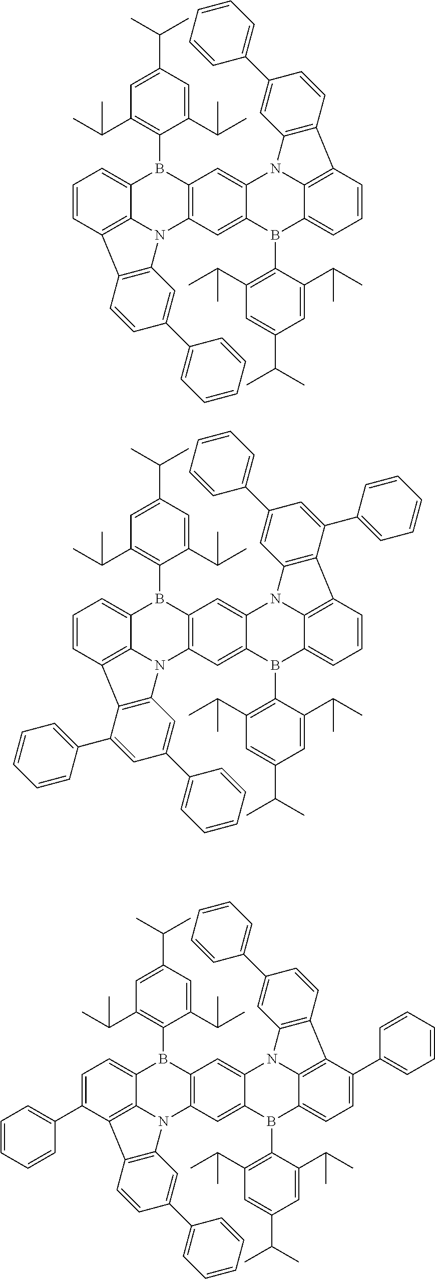

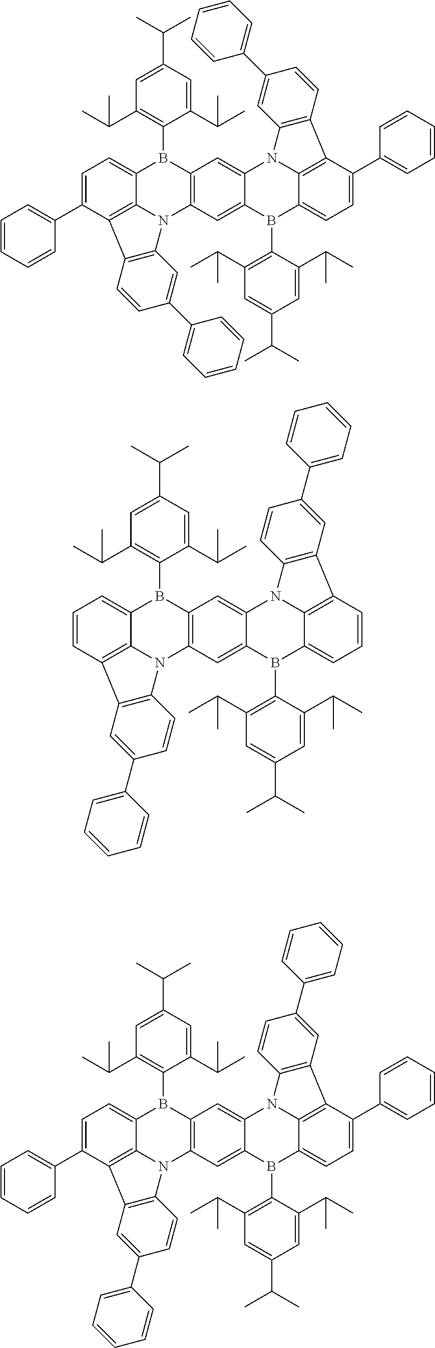 [SMILES: CC(C)c1cc(C(C)C)c(B2c3cc4c(cc3-n3c5cc(-c6ccccc6)ccc5c5c(-c6ccccc6)ccc2c53)B(c2c(C(C)C)cc(C(C)C)cc2C(C)C)c2ccc(-c3ccccc3)c3c5ccc(-c6ccccc6)cc5n-4c23)c(C(C)C)c1 ; CC(C)c1cc(C(C)C)c(B2c3cc4c(cc3-n3c5ccc(-c6ccccc6)cc5c5cccc2c53)B(c2c(C(C)C)cc(C(C)C)cc2C(C)C)c2cccc3c5cc(-c6ccccc6)ccc5n-4c23)c(C(C)C)c1 ; CC(C)c1cc(C(C)C)c(B2c3cc4c(cc3-n3c5ccc(-c6ccccc6)cc5c5c(-c6ccccc6)ccc2c53)B(c2c(C(C)C)cc(C(C)C)cc2C(C)C)c2ccc(-c3ccccc3)c3c5cc(-c6ccccc6)ccc5n-4c23)c(C(C)C)c1]

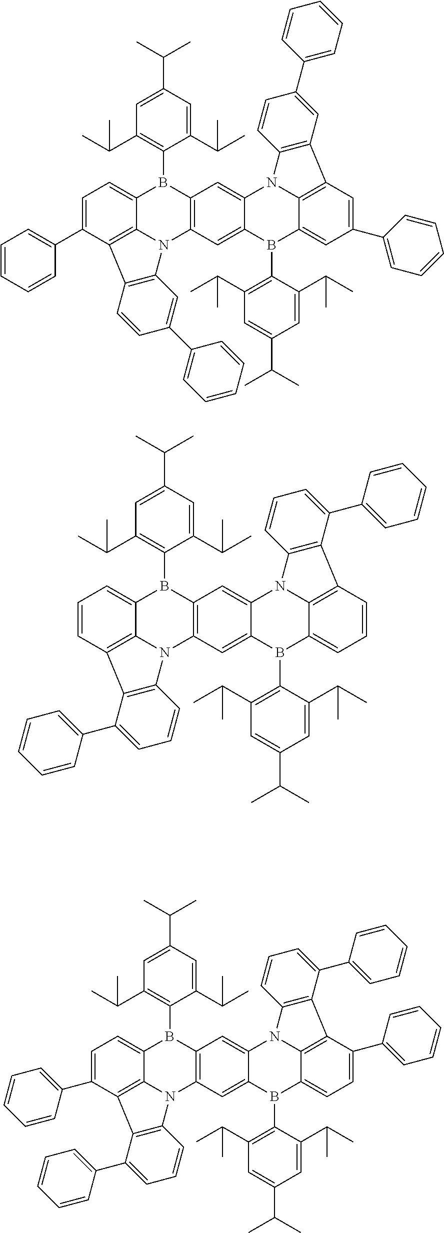

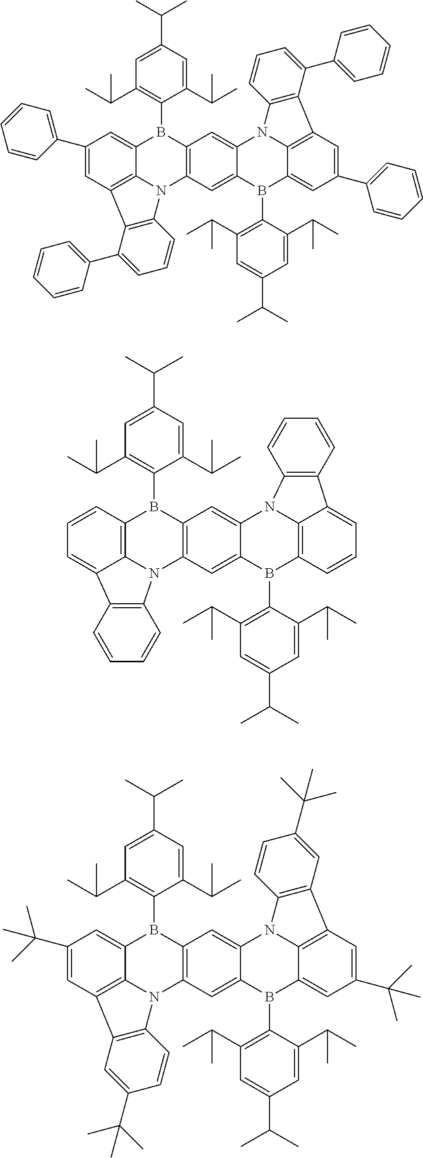

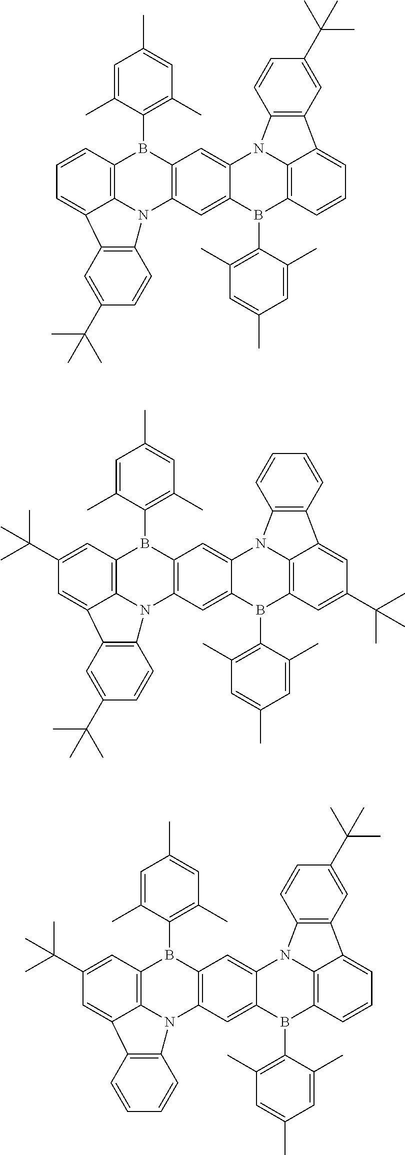

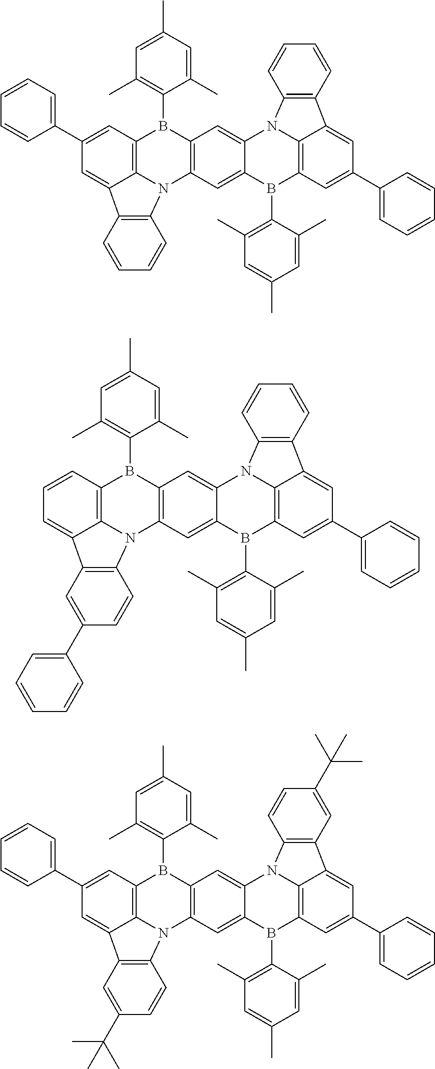 [SMILES: Cc1cc(C)c(B2c3cc4c(cc3-n3c5ccccc5c5cc(-c6ccccc6)cc2c53)B(c2c(C)cc(C)cc2C)c2cc(-c3ccccc3)cc3c5ccccc5n-4c23)c(C)c1 ; Cc1cc(C)c(B2c3cc4c(cc3-n3c5ccc(-c6ccccc6)cc5c5cccc2c53)B(c2c(C)cc(C)cc2C)c2cc(-c3ccccc3)cc3c5ccccc5n-4c23)c(C)c1 ; Cc1cc(C)c(B2c3cc4c(cc3-n3c5ccc(C(C)(C)C)cc5c5cc(-c6ccccc6)cc2c53)B(c2c(C)cc(C)cc2C)c2cc(-c3ccccc3)cc3c5cc(C(C)(C)C)ccc5n-4c23)c(C)c1]

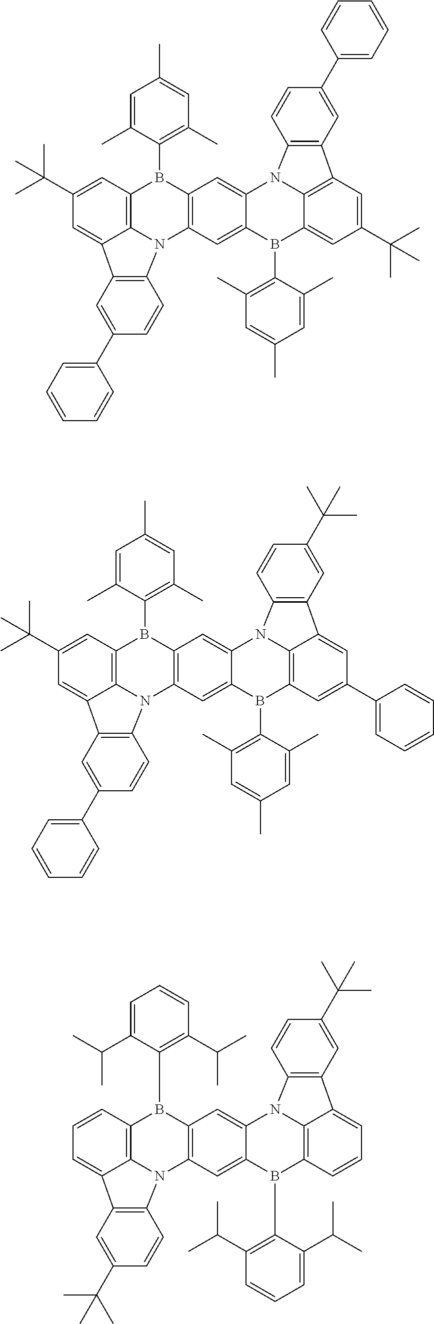

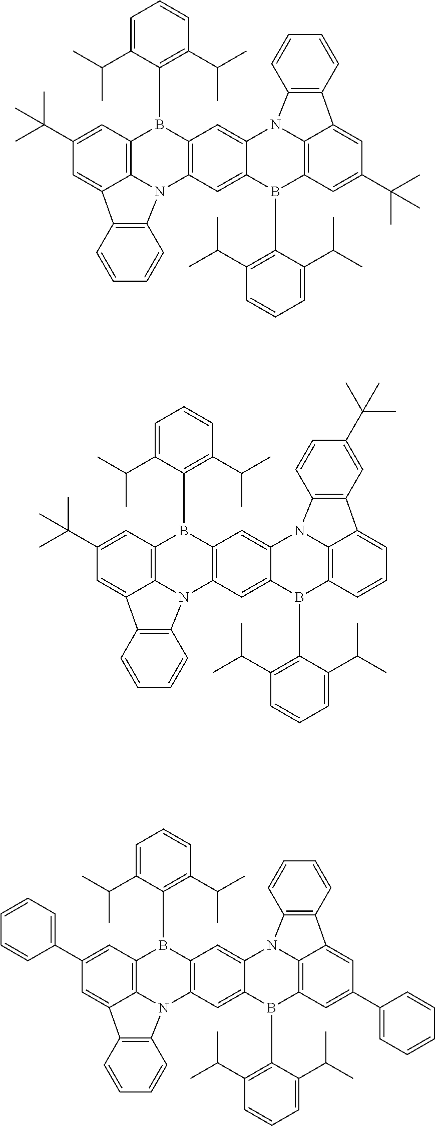

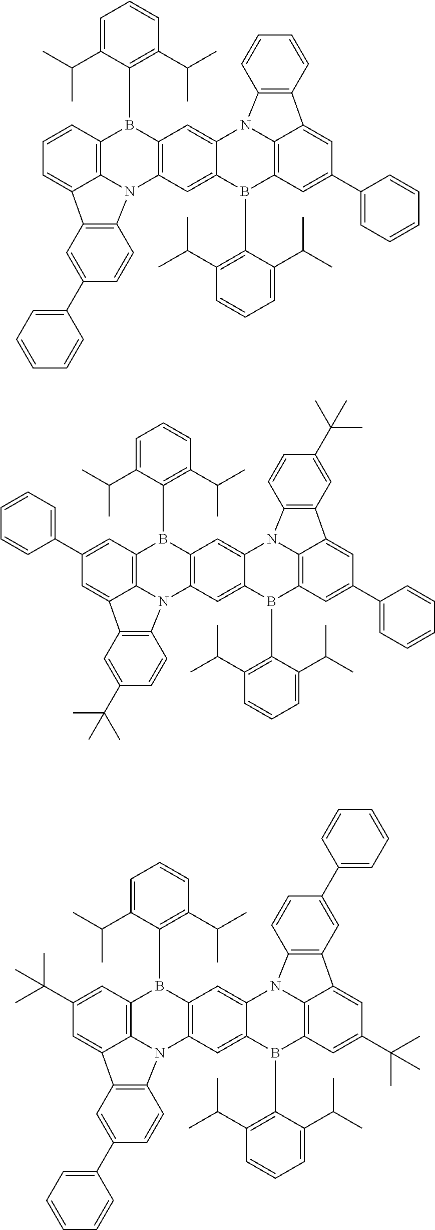 [SMILES: CC(C)c1cccc(C(C)C)c1B1c2cc3c(cc2-n2c4ccc(-c5ccccc5)cc4c4cccc1c42)B(c1c(C(C)C)cccc1C(C)C)c1cc(-c2ccccc2)cc2c4ccccc4n-3c12 ; CC(C)c1cccc(C(C)C)c1B1c2cc3c(cc2-n2c4ccc(C(C)(C)C)cc4c4cc(-c5ccccc5)cc1c42)B(c1c(C(C)C)cccc1C(C)C)c1cc(-c2ccccc2)cc2c4cc(C(C)(C)C)ccc4n-3c12 ; CC(C)c1cccc(C(C)C)c1B1c2cc3c(cc2-n2c4ccc(-c5ccccc5)cc4c4cc(C(C)(C)C)cc1c42)B(c1c(C(C)C)cccc1C(C)C)c1cc(C(C)(C)C)cc2c4cc(-c5ccccc5)ccc4n-3c12]

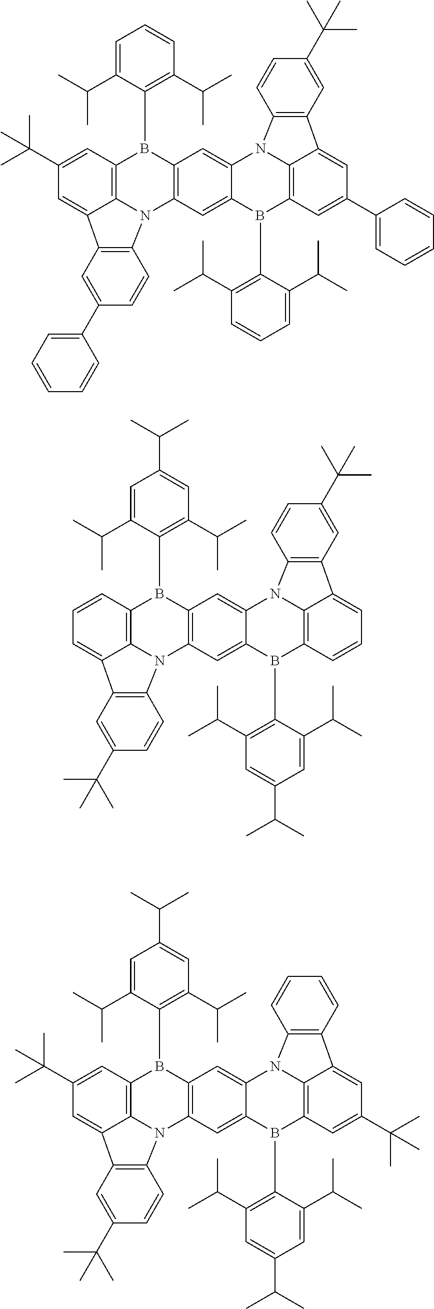

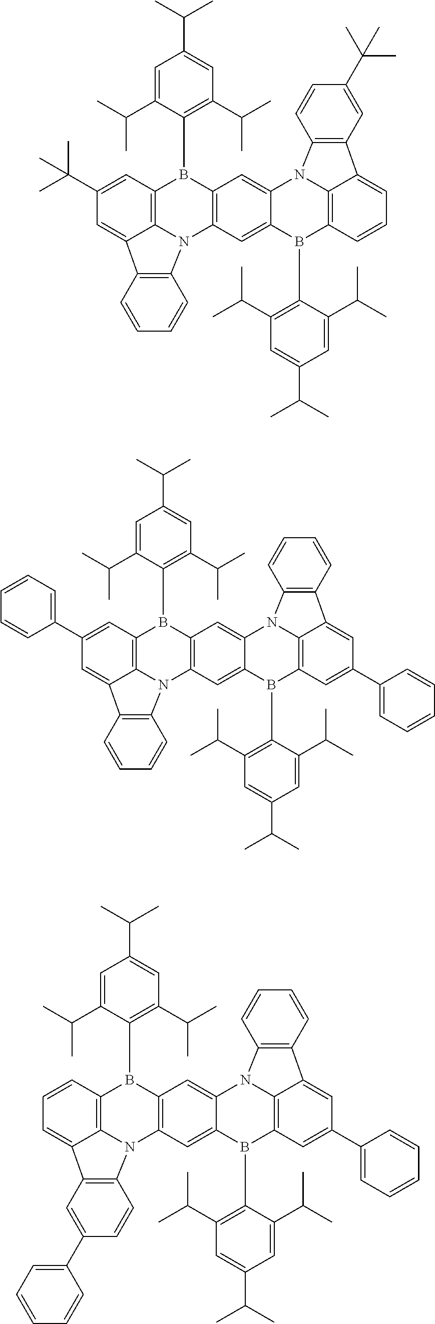 [SMILES: CC(C)c1cc(C(C)C)c(B2c3cc4c(cc3-n3c5ccc(C(C)(C)C)cc5c5cccc2c53)B(c2c(C(C)C)cc(C(C)C)cc2C(C)C)c2cc(C(C)(C)C)cc3c5ccccc5n-4c23)c(C(C)C)c1 ; CC(C)c1cc(C(C)C)c(B2c3cc4c(cc3-n3c5ccccc5c5cc(-c6ccccc6)cc2c53)B(c2c(C(C)C)cc(C(C)C)cc2C(C)C)c2cc(-c3ccccc3)cc3c5ccccc5n-4c23)c(C(C)C)c1 ; CC(C)c1cc(C(C)C)c(B2c3cc4c(cc3-n3c5ccc(-c6ccccc6)cc5c5cccc2c53)B(c2c(C(C)C)cc(C(C)C)cc2C(C)C)c2cc(-c3ccccc3)cc3c5ccccc5n-4c23)c(C(C)C)c1]

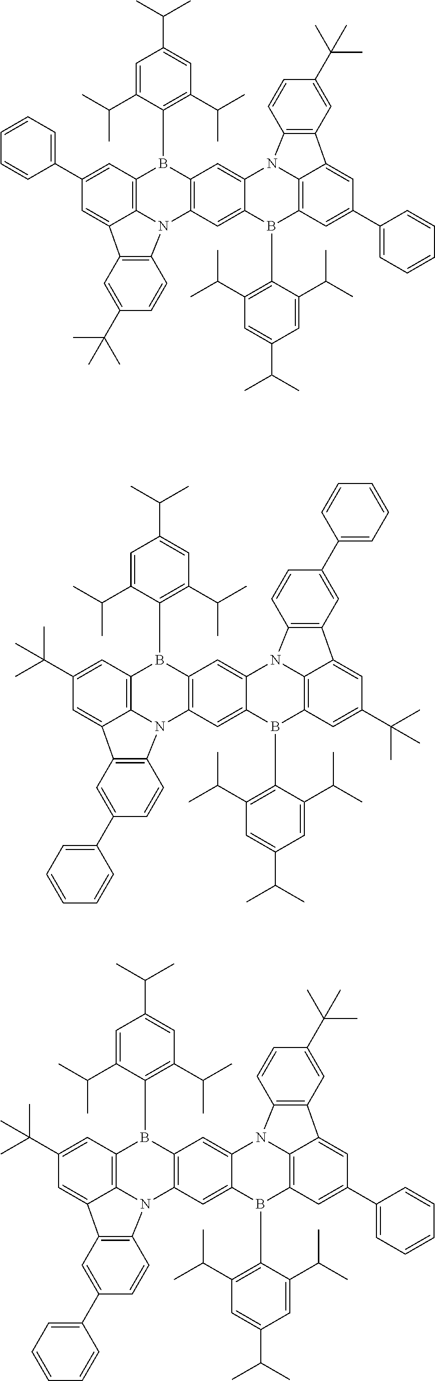

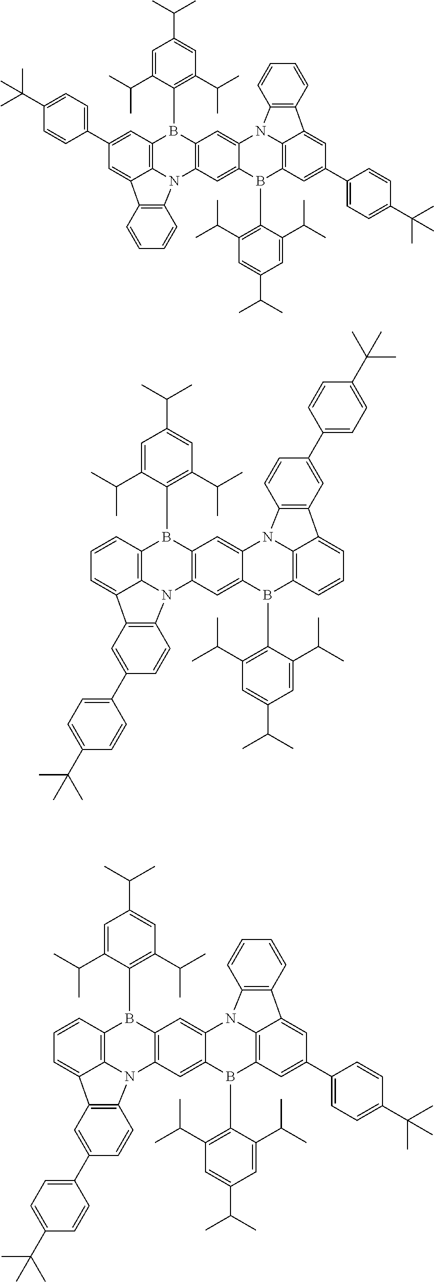

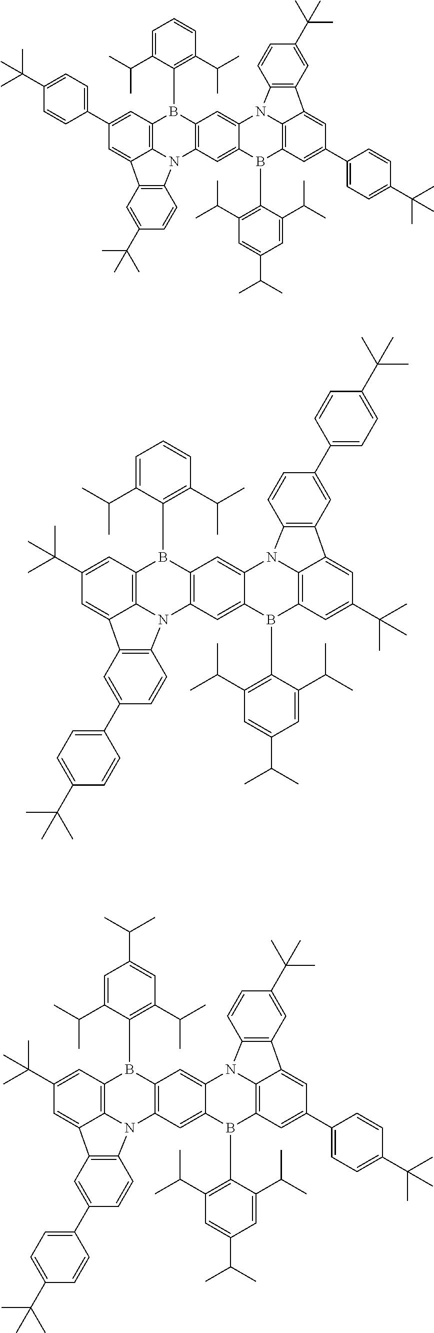

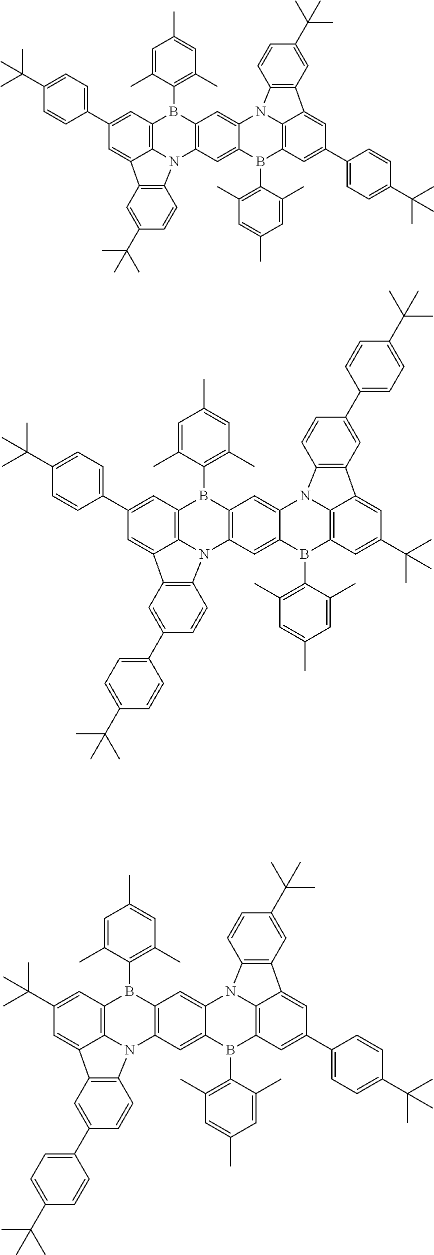

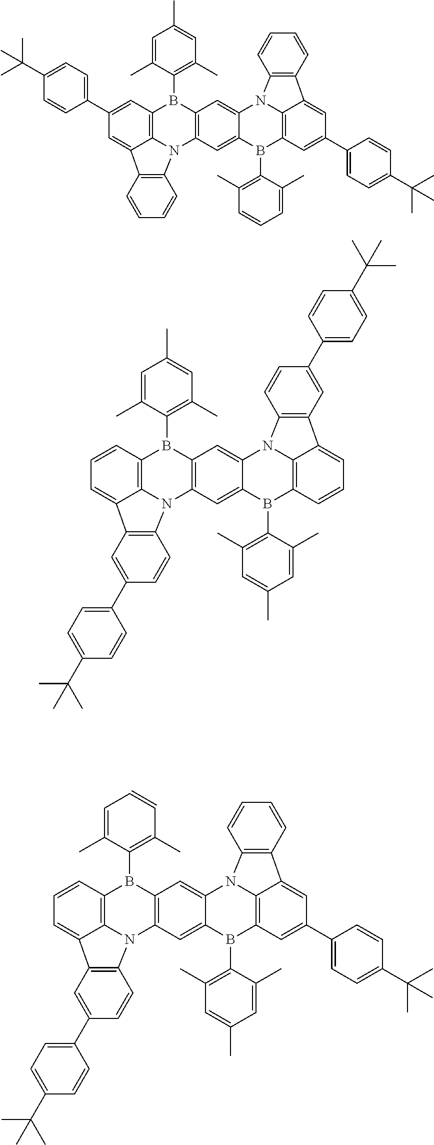

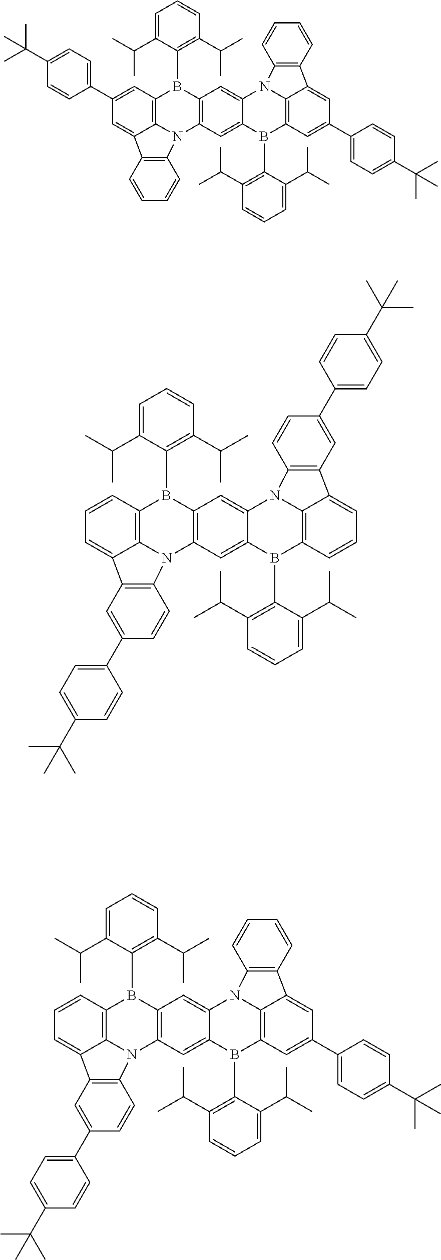

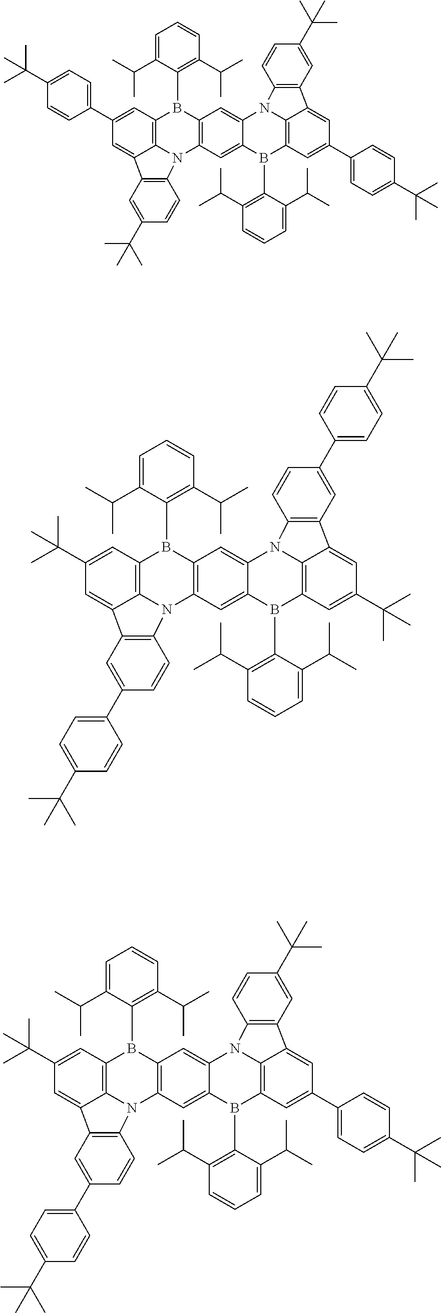

Hereinafter, another group of specific examples of the compound represented by the general formula (1a) will be given. Compounds of the general formula (1a) that can be used in the present invention are not construed as limiting to specific examples in the following group.

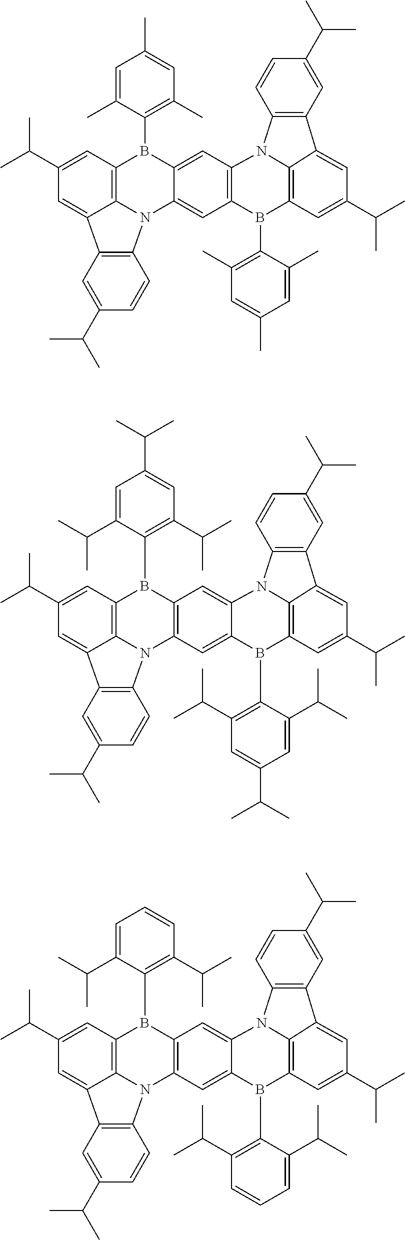

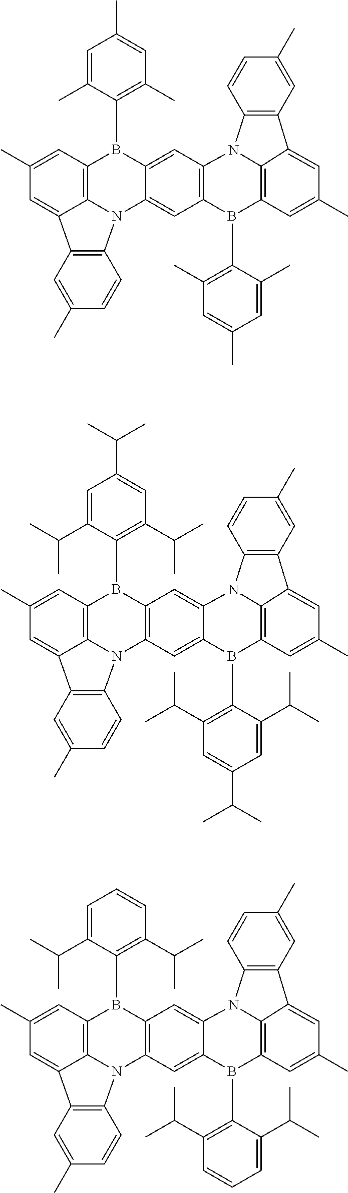

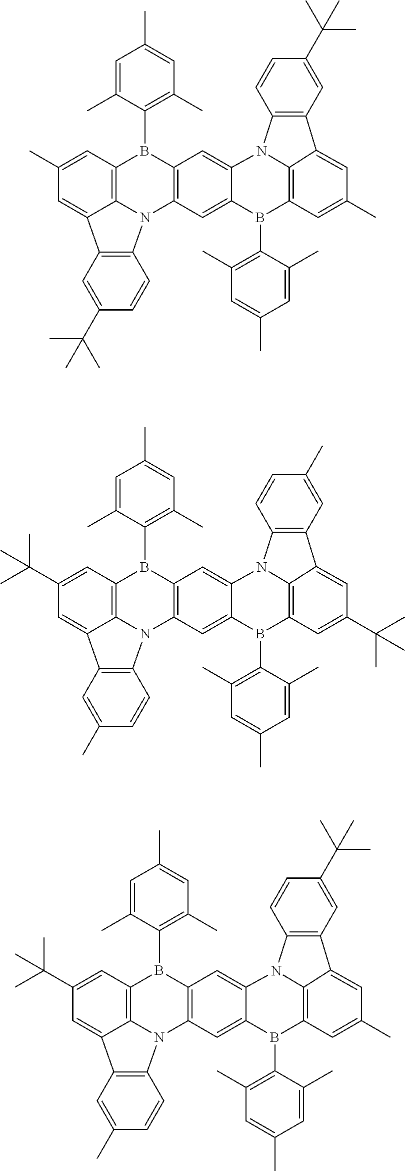

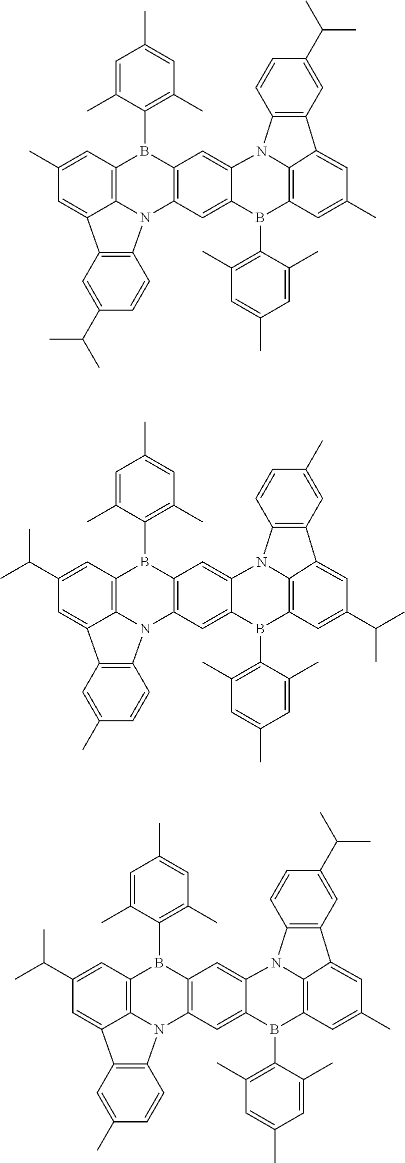

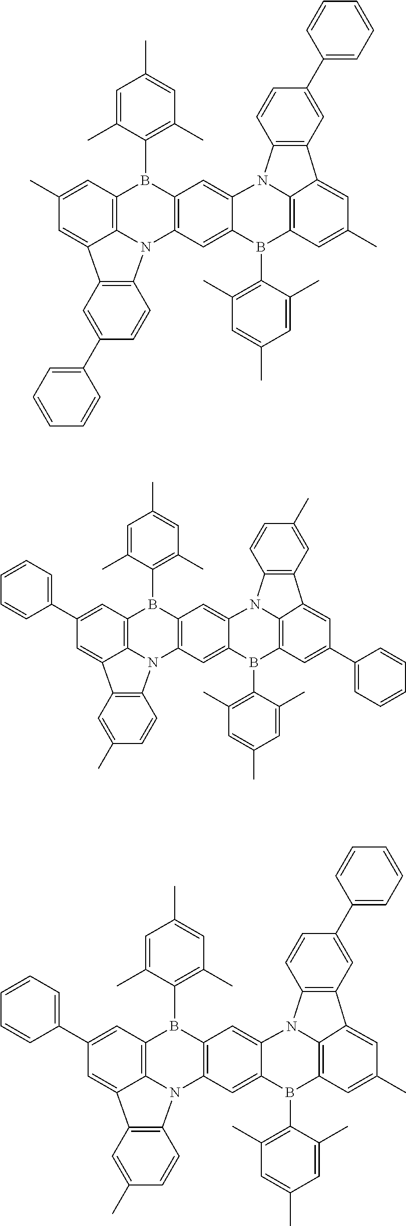

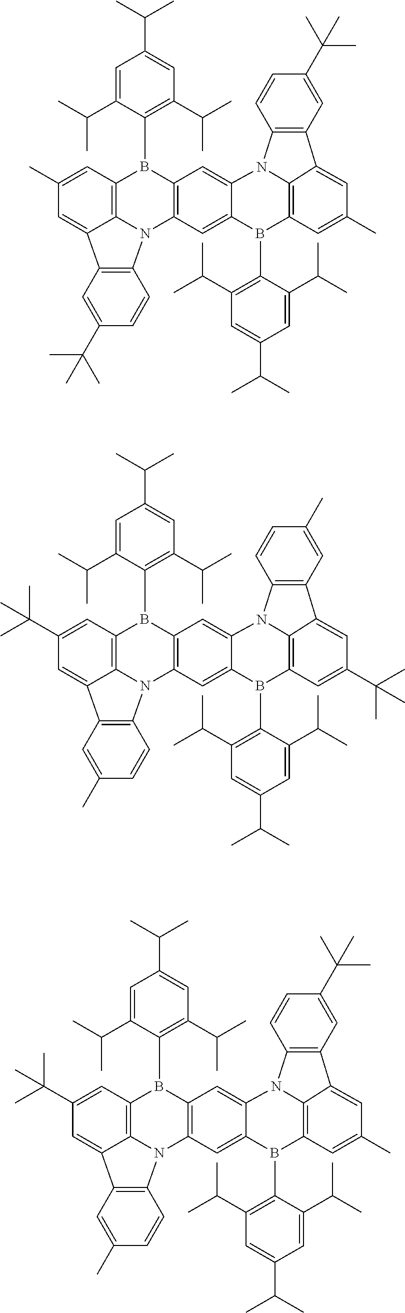

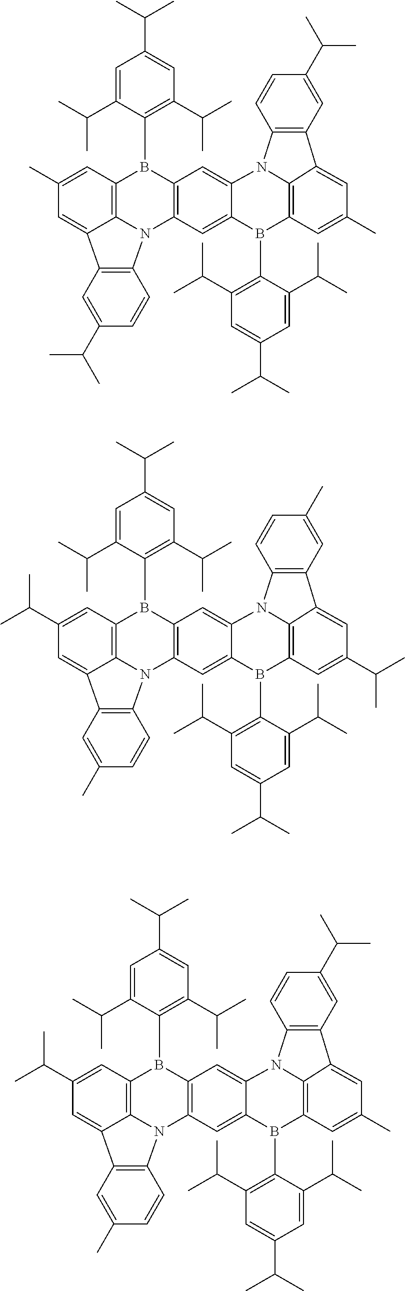

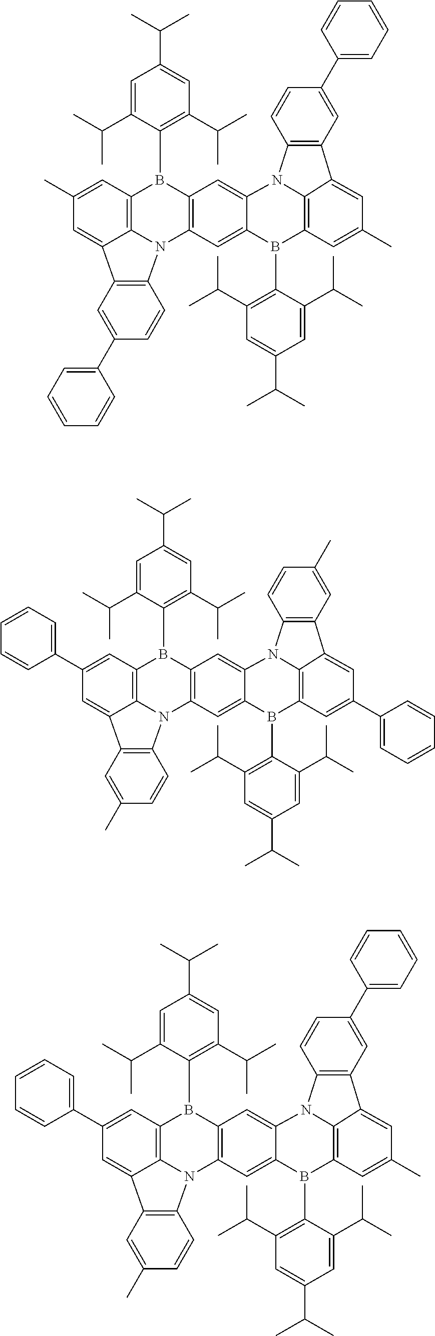

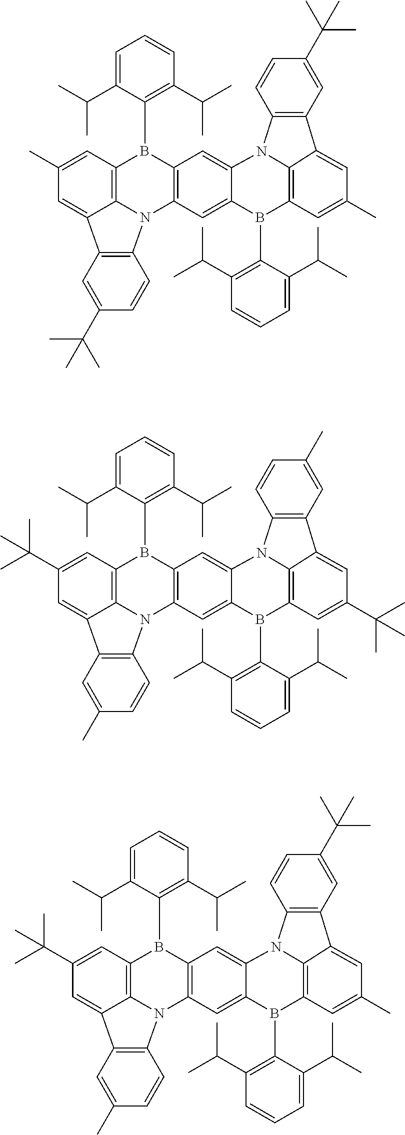

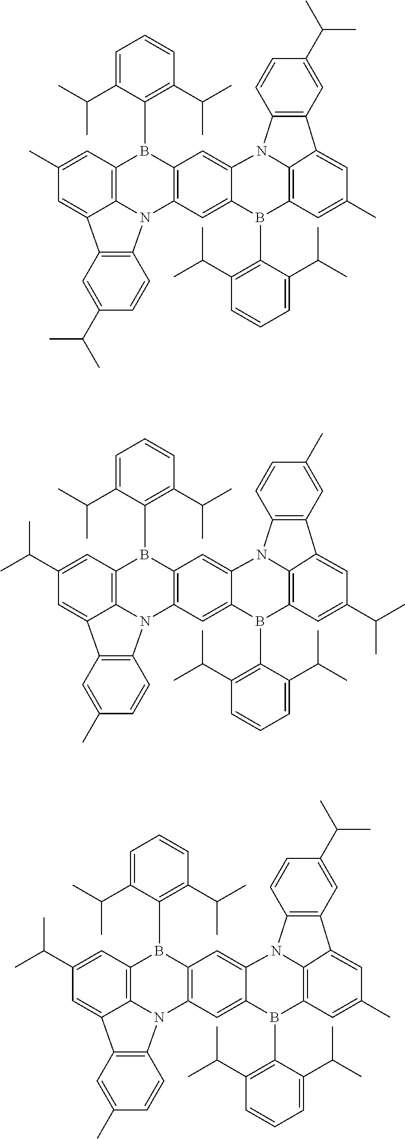

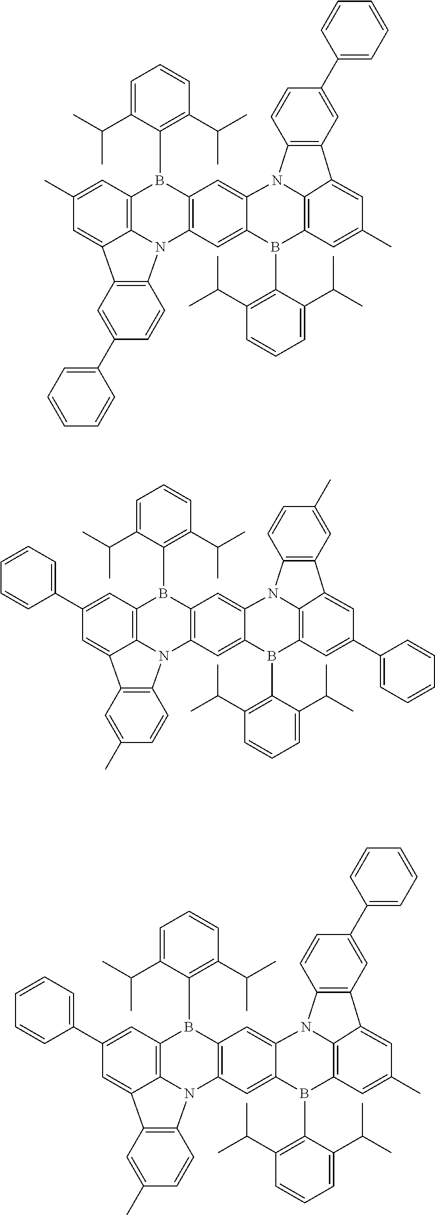

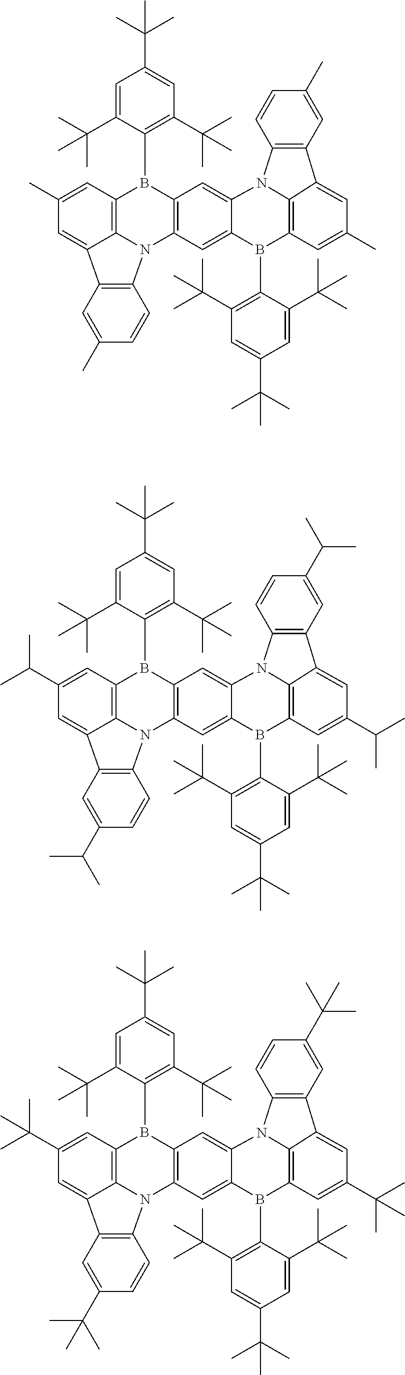

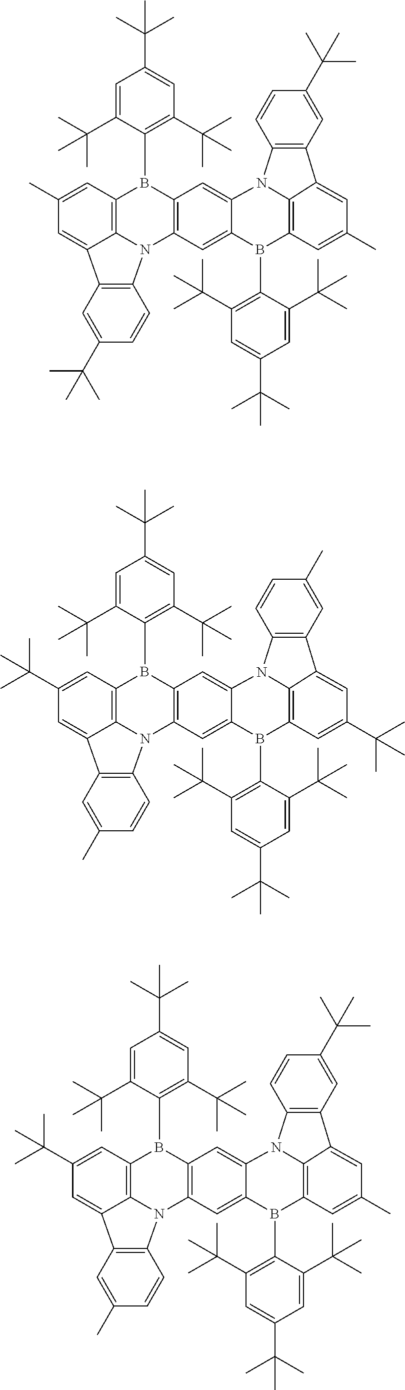

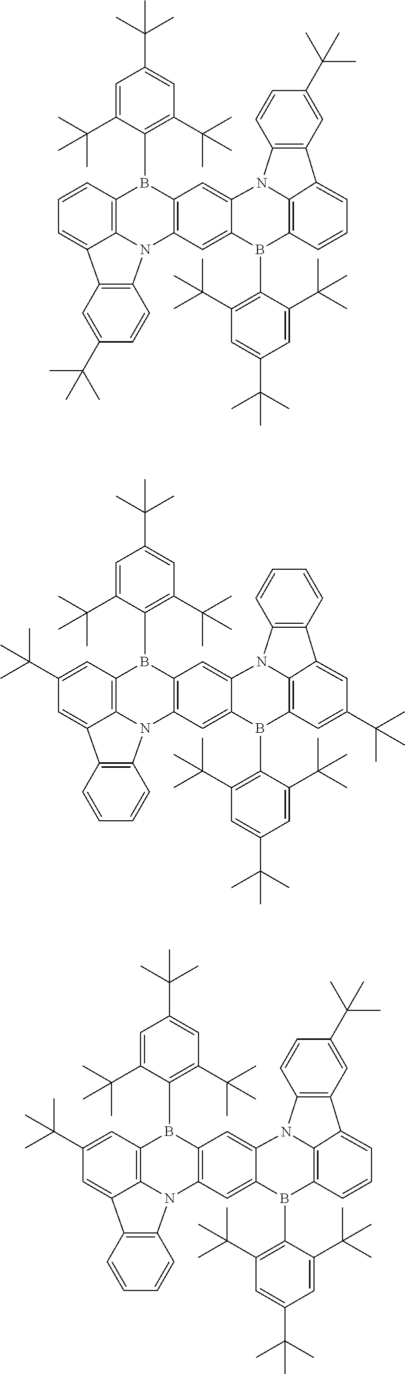

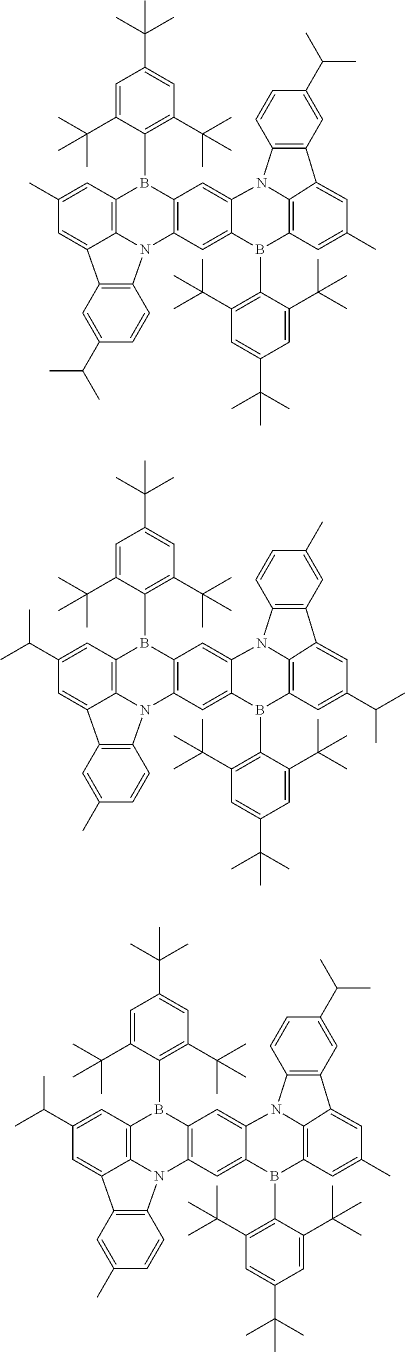

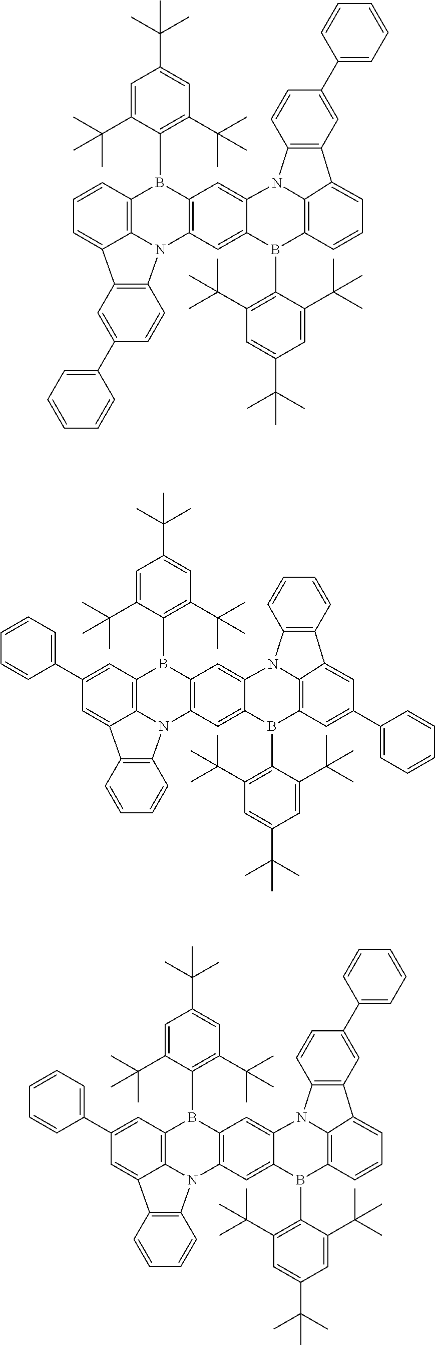

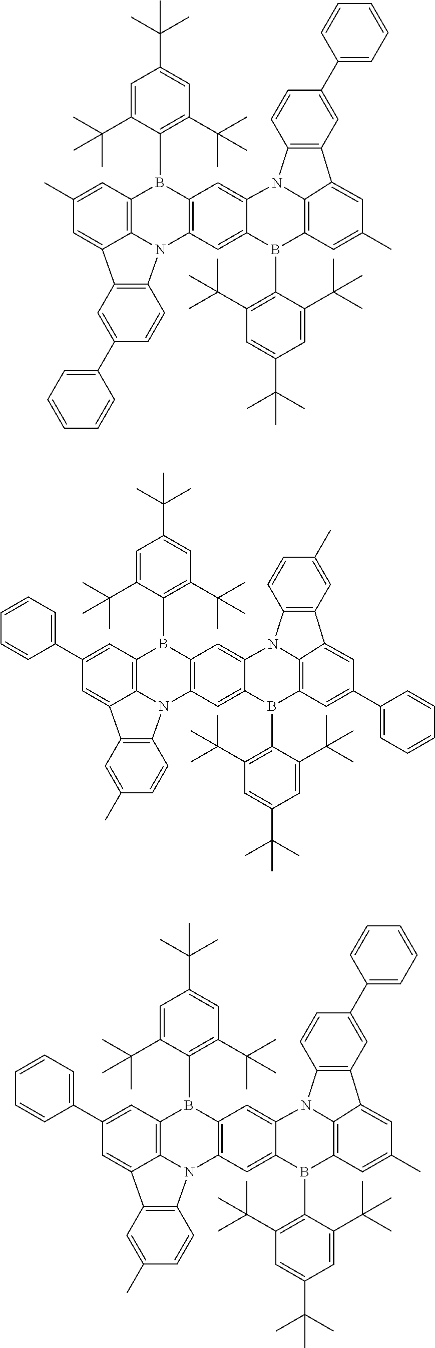

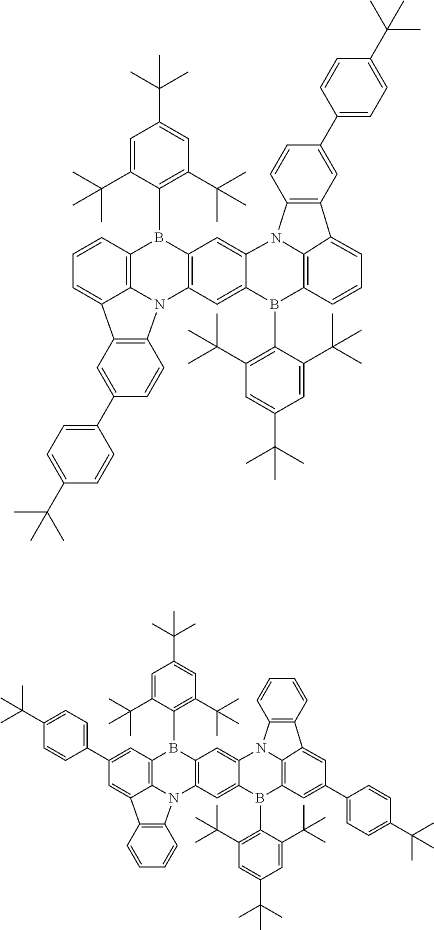

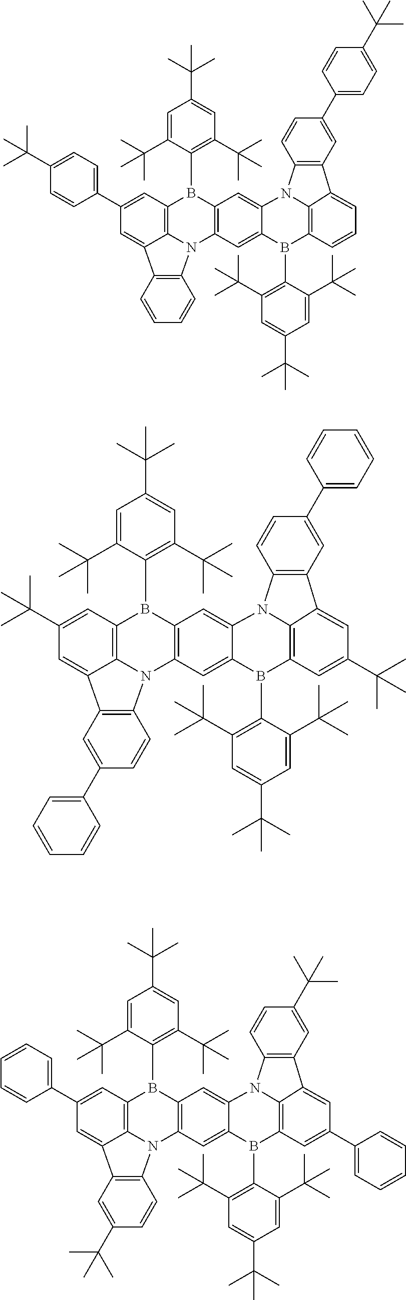

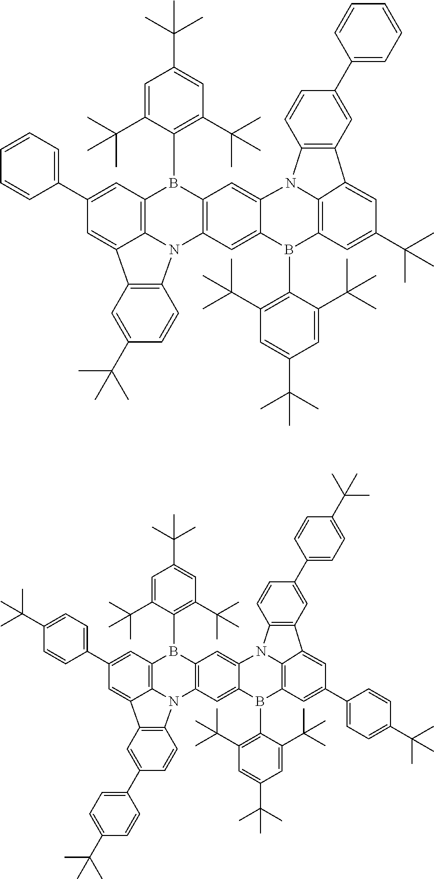

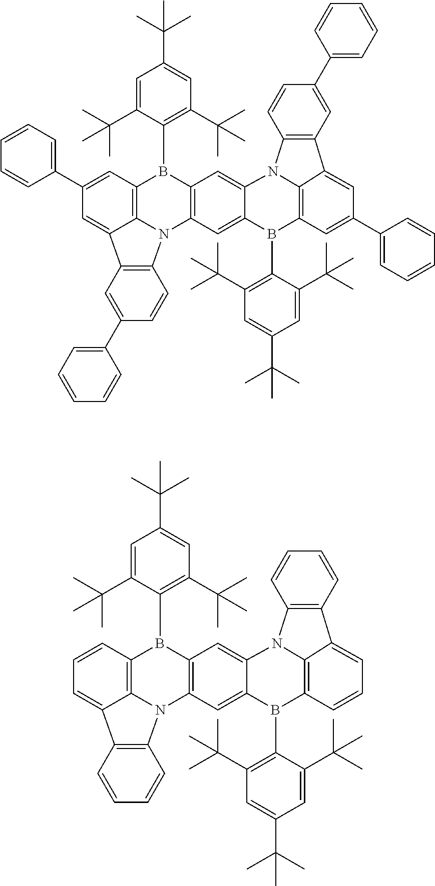

As one preferable group of compounds having the skeleton (1b), compounds represented by the following general formula (1b) can be exemplified.

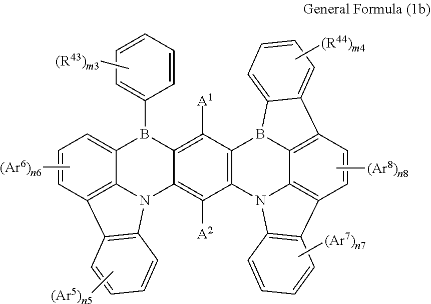

In the general formula (1b), each of Ar⁵ to Ar⁸ independently represents a substituted or unsubstituted aryl group, a substituted or unsubstituted heteroaryl group, or a substituted or unsubstituted alkyl group, and, for example, a substituted or unsubstituted aryl group can be preferably selected. Each of R⁴³ and R⁴⁴ independently represents a substituted or unsubstituted alkyl group. Each of m3 and m4 independently represents an integer of 0 to 5, each of n6 and n8 independently represents an integer of 0 to 3, and each of n5 and n7 independently represents an integer of 0 to 4. Each of A¹ and A² independently represents a hydrogen atom, a deuterium atom, or a substituent. In relation to details of Ar⁵ to Ar⁸, R⁴³ and R⁴⁴, m3 and m4, n5 to n8, A¹, and A², the descriptions on Ar¹ to Ar⁴, R⁴¹ and R⁴², m1 and m2, n1 to n4, A¹, and A² in the general formula (1a) can be referred to. It is preferable that at least one of n5 to n8 is 1 or more, and each of m3 and m4 is independently any integer of 1 to 5.

Hereinafter, specific examples of the compound represented by the general formula (1b) will be given. Compounds of the general formula (1b) that can be used in the present invention are not construed as limiting to the following specific examples.

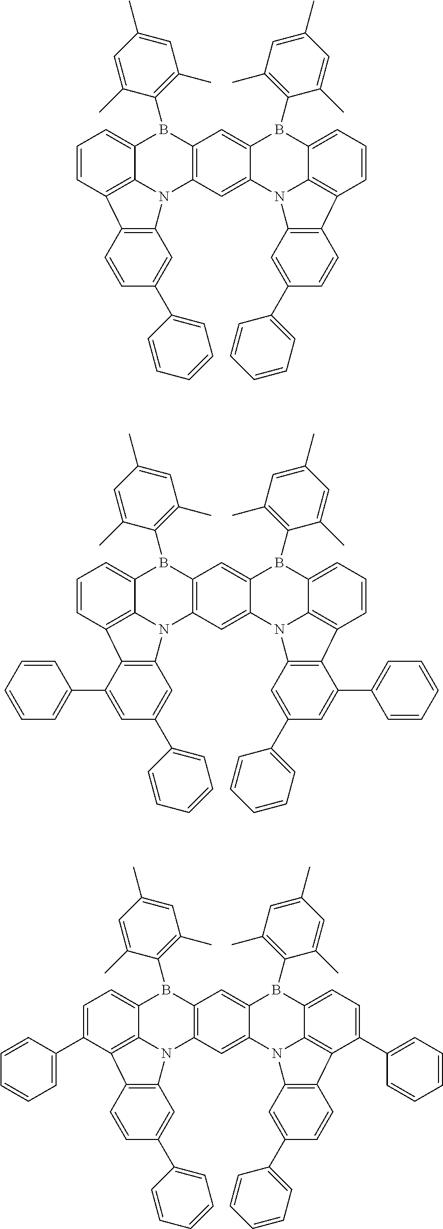

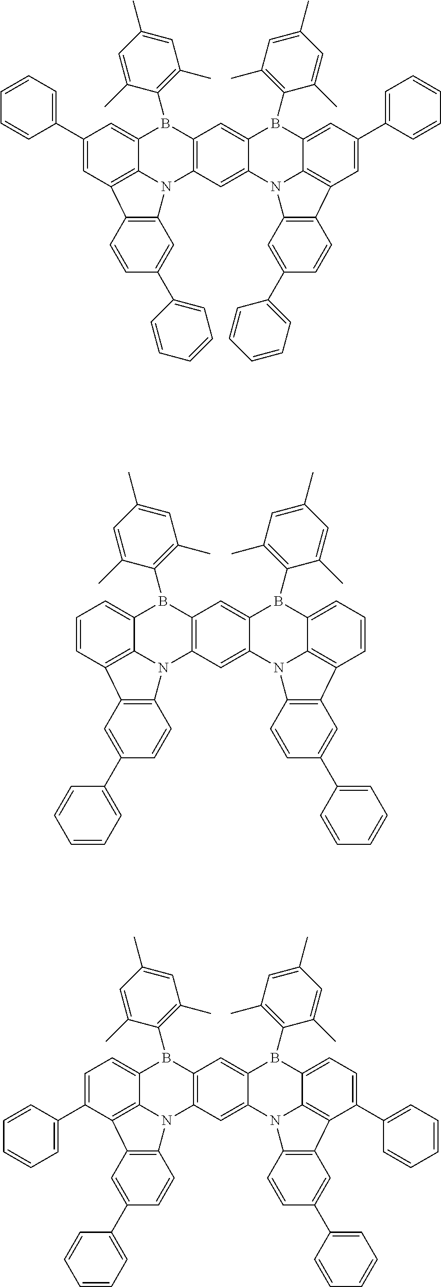

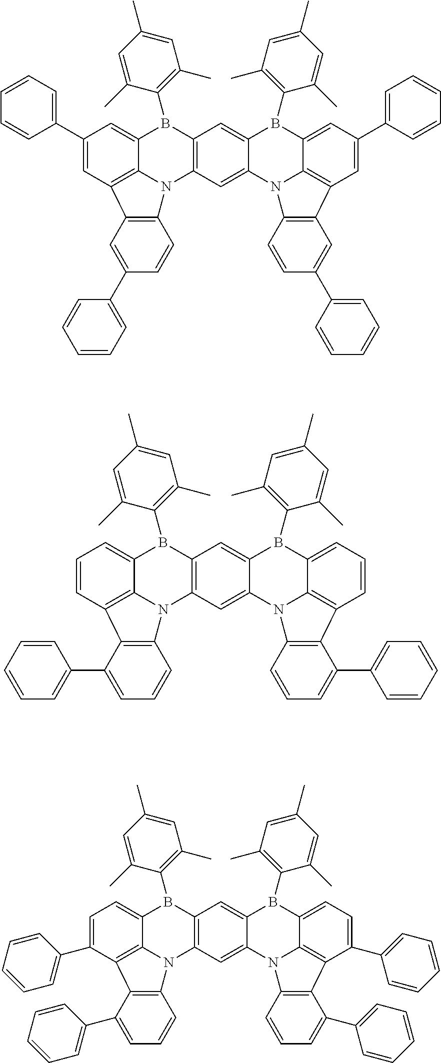

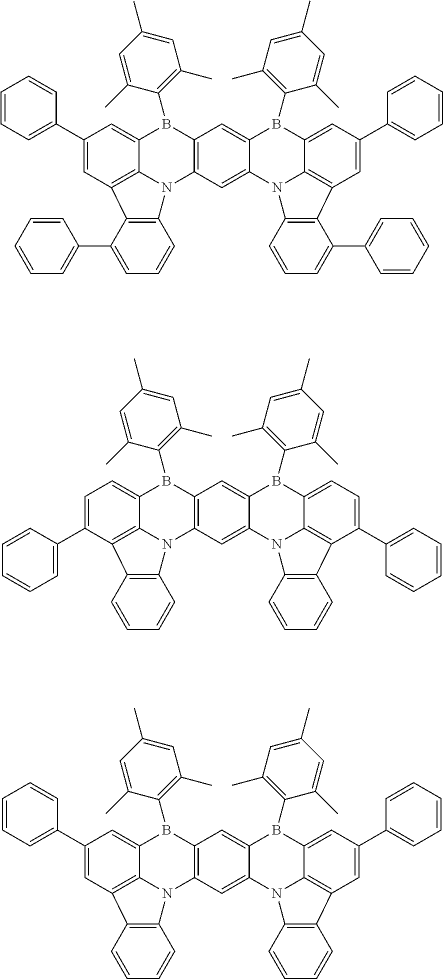

When R⁷ and R⁸ in the general formula (G) bond to each other to form N-Ph, the compound of the present invention has, for example, the following skeleton (2a) where X¹ is a nitrogen atom, and, has for example, the following skeleton (2b) where X² is a nitrogen atom. Ph is a phenyl group.

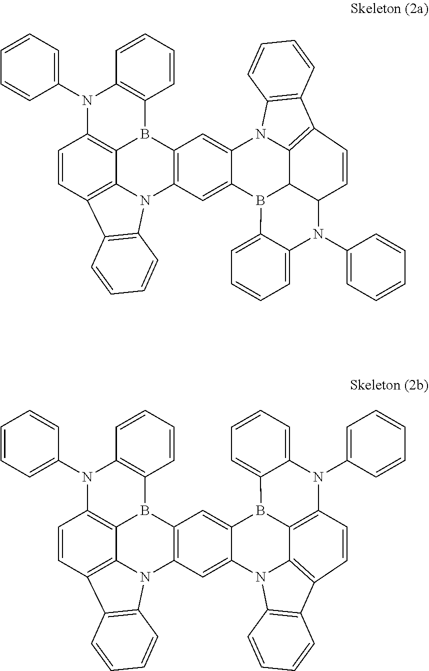

In the skeletons (2a) and (2b), each hydrogen atom can be substituted with a deuterium atom or a substituent. Further, it can be substituted with a linking group together with an adjacent hydrogen atom to form a cyclic structure. For details, corresponding descriptions on R¹ to R²⁶, A¹, and A² in the general formula (G) can be referred to. At least one hydrogen atom of a benzene ring forming a carbazole partial structure included in the skeleton (2a) is substituted with a substituted or unsubstituted aryl group. In one aspect of the present invention, each hydrogen atom in the skeletons (2a) and (2b) is not substituted with a linking group together with an adjacent hydrogen atom to form a cyclic structure.

As one preferable group of compounds having the skeleton (2a), compounds represented by the following general formula (2a) can be exemplified.

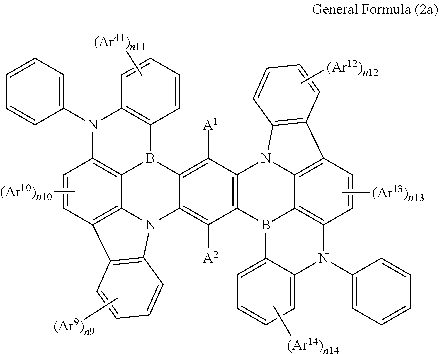

In the general formula (2a), each of Ar⁹ to Ar¹⁴ independently represents a substituted or unsubstituted aryl group, a substituted or unsubstituted heteroaryl group, or a substituted or unsubstituted alkyl group, and for example, a substituted or unsubstituted aryl group can be preferably selected. Each of n9, n11, n12, and n14 independently represents an integer of 0 to 4, and each of n10 and n13 independently represents an integer of 0 to 2. Meanwhile, at least one of n9, n10, n12, and n13 is 1 or more. Each of A¹ and A² independently represents a hydrogen atom, a deuterium atom, or a substituent.

In one aspect of the present invention, n9 to n14 each independently represent an integer of 0 to 2. In one aspect of the present invention, at least one of n9 to n14 is 1 or more, and for example, n9 and n12 can be 1 or more or n10 and n13 can be 1 or more. In one preferred aspect of the present invention, at least one of n9, n10, n12, and n13 is 1 or more. In one aspect of the present invention, each of n9 and n12 is independently 1 or 2, and n10, n11, n13, and n14 are 0. In one aspect of the present invention, each of n10 and n13 is independently 1 or 2, and n9, n11, n12, and n14 are 0. In one aspect of the present invention, each of n9 and n12 is independently 1 or 2, each of n10 and n13 is independently 1 or 2, and n1 and n14 are 0. In one aspect of the present invention, n9 to n14 are all 1. The bonding sites of A⁹ to Ar¹⁴ can be 3 and 6 positions of a carbazole ring, or can be other positions. In one preferred aspect of the present invention, Ar⁹ to Ar¹⁴ are all the same group. For preferable groups for Ar⁹ to Ar¹⁴, corresponding descriptions on Ar¹ to A⁴ can be referred to. For descriptions and preferable ranges of A¹ and A², corresponding descriptions on the general formula (G) can be referred to.

Hereinafter, specific examples of the compound represented by the general formula (2a) will be given. Compounds of the general formula (2a) that can be used in the present invention are not construed as limiting to the following specific examples.

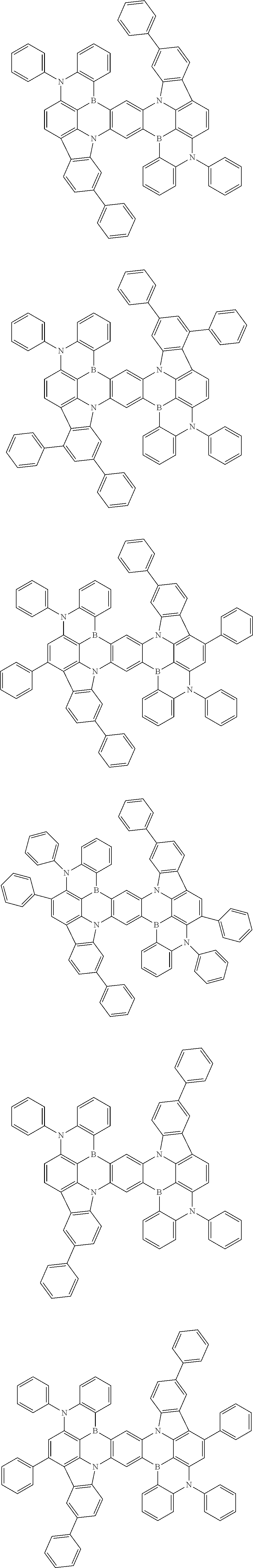

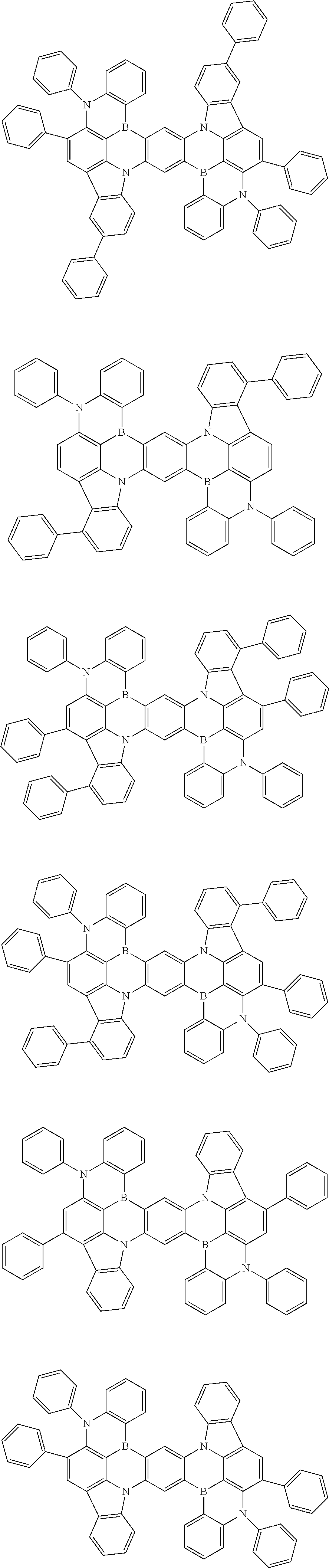

As one preferable group of compounds having the skeleton (2b), compounds represented by the following general formula (2b) can be exemplified.

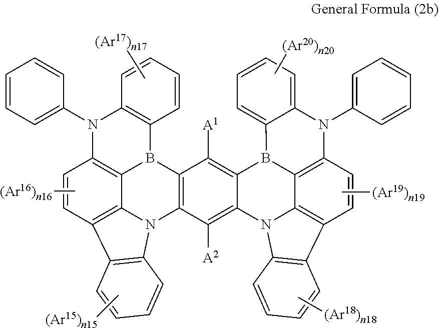

In the general formula (2b), each of Ar¹⁵ to Ar²⁰ independently represents a substituted or unsubstituted aryl group, a substituted or unsubstituted heteroaryl group, or a substituted or unsubstituted alkyl group, and, for example, a substituted or unsubstituted aryl group can be preferably selected. Each of n15, n17, n18, and n20 independently represents an integer of 0 to 4, and each of n16 and n19 independently represents an integer of 0 to 2. Each of A¹ and A² independently represents a hydrogen atom, a deuterium atom, or a substituent. For details of Ar¹⁵ to Ar²⁰, n15 to n20, A¹, and A², descriptions on Ar⁹ to Ar¹⁴, n9 to n14, A¹, and A² in the general formula (2a) can be referred to in this order.

Hereinafter, specific examples of the compound represented by the general formula (2b) will be given. Compounds of the general formula (2b) that can be used in the present invention are not construed as limiting to the following specific examples.

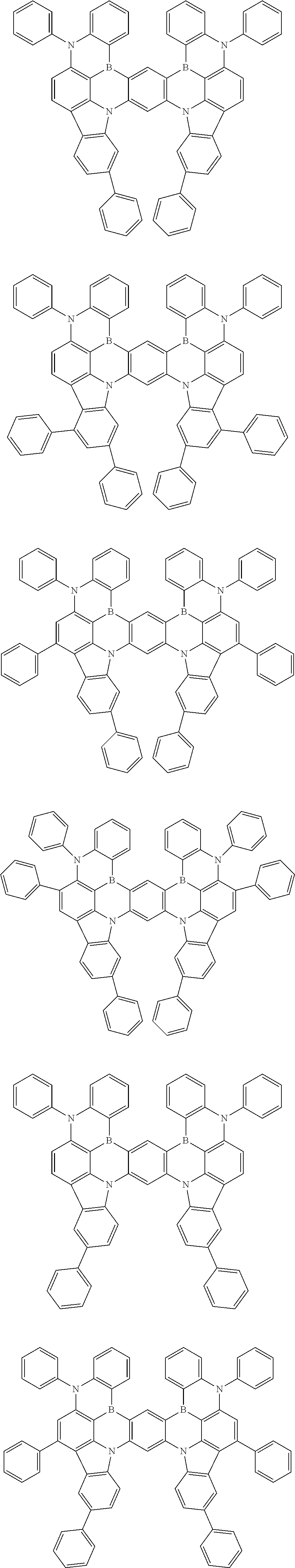

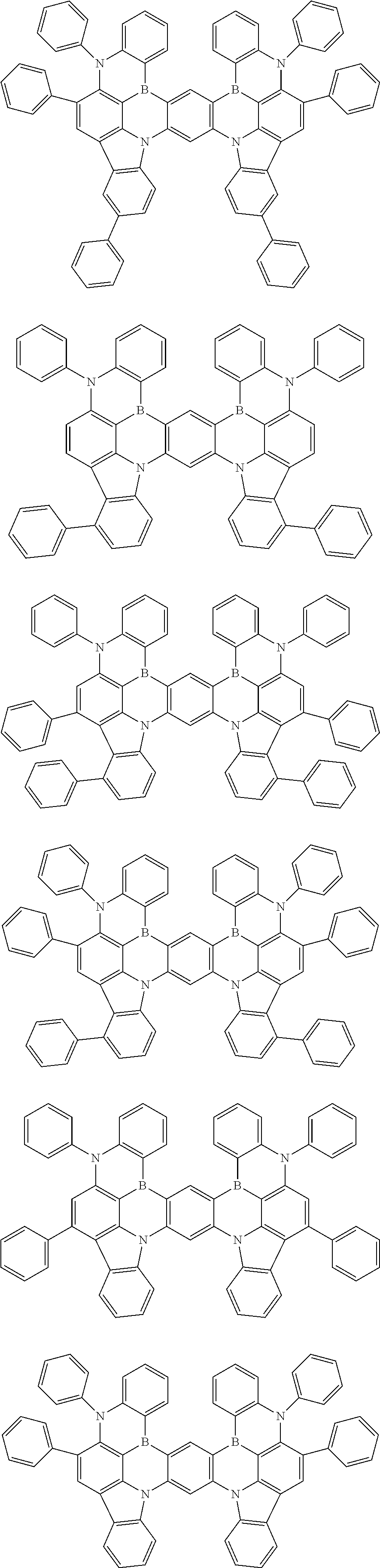

When R⁷ and R⁸ in the general formula (G) bond to each other to form a single bond, the compound of the present invention has, for example, the following skeleton (3a) if X¹ is a nitrogen atom, and has, for example, the following skeleton (3b) if X² is a nitrogen atom.

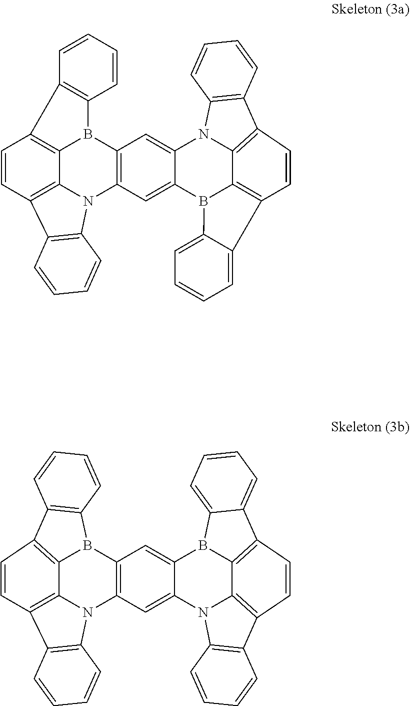

In the skeletons (3a) and (3b), each hydrogen atom can be substituted with a deuterium atom or a substituent. Further, it can be substituted with a linking group together with an adjacent hydrogen atom to form a cyclic structure. For details, corresponding descriptions on R¹ to R²⁶, A¹, and A² in the general formula (G) can be referred to. In one aspect of the present invention, each hydrogen atom in the skeletons (3a) and (3b) is not substituted with a linking group together with an adjacent hydrogen atom to form a cyclic structure.

As one preferable group of compounds having the skeleton (3a), compounds represented by the following general formula (3a) can be exemplified.

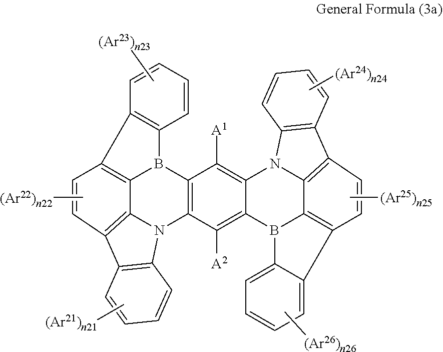

In the general formula (3a), each of Ar²¹ to Ar²⁶ independently represents a substituted or unsubstituted aryl group, a substituted or unsubstituted heteroaryl group, or a substituted or unsubstituted alkyl group, and for example, a substituted or unsubstituted aryl group can be preferably selected. Each of n21, n23, n24, and n26 independently represents an integer of 0 to 4, and each of n22 and n25 independently represents an integer of 0 to 2. Each of A¹ and A² independently represents a hydrogen atom, a deuterium atom, or a substituent. For details of Ar²¹ to Ar²⁵, and n21 to n25, descriptions on Ar⁹ to Ar¹⁴, n9 to n14, A¹, and A² in the general formula (2a) can be referred to.

Hereinafter, specific examples of the compound represented by the general formula (3a) will be given. Compounds of the general formula (3a) that can be used in the present invention are not construed as limiting to the following specific examples.

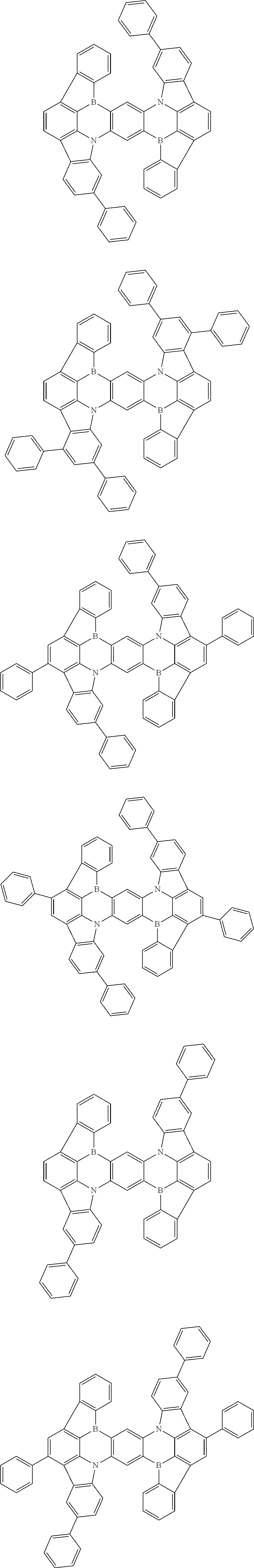

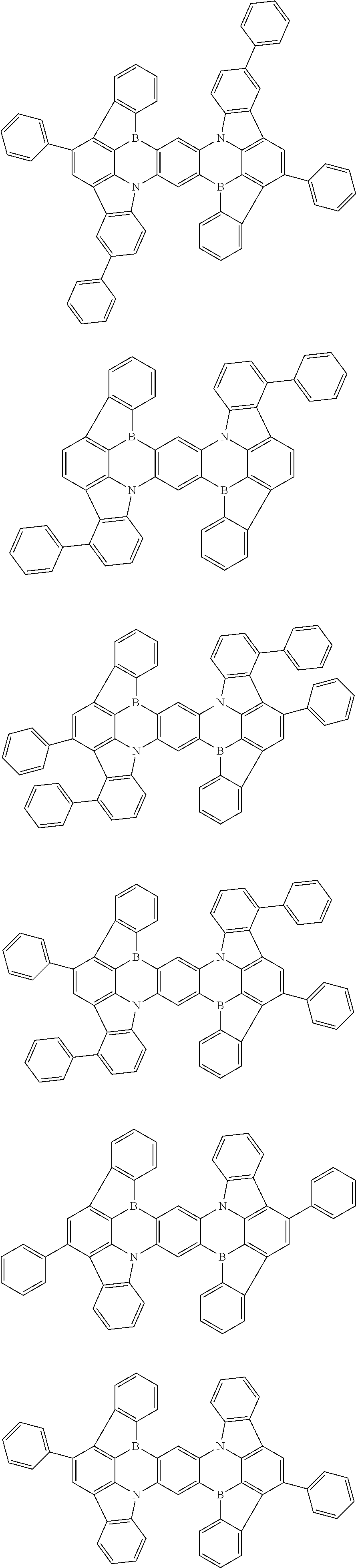

As one preferable group of compounds having the skeleton (3b), compounds represented by the following general formula (3b) can be exemplified.

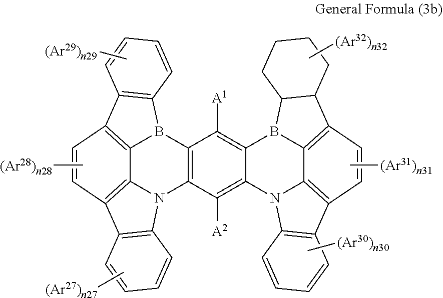

In the general formula (3b), each of Ar²⁷ to Ar³² independently represents a substituted or unsubstituted aryl group, a substituted or unsubstituted heteroaryl group, or a substituted or unsubstituted alkyl group, and for example, a substituted or unsubstituted aryl group can be preferably selected. Each of n27, n29, n30, and n32 independently represents an integer of 0 to 4, and each of n28 and n31 independently represents an integer of 0 to 2. Each of A¹ and A² independently represents a hydrogen atom, a deuterium atom, or a substituent. For details of Ar²⁷ to Ar³², n27 to n32, A¹, and A², descriptions on Ar¹⁵ to Ar²⁰, n15 to n20, A¹, and A² in the general formula (2b) can be referred to in this order.

Hereinafter, specific examples of the compound represented by the general formula (3b) will be given. Compounds of the general formula (3b) that can be used in the present invention are not construed as limiting to the following specific examples.

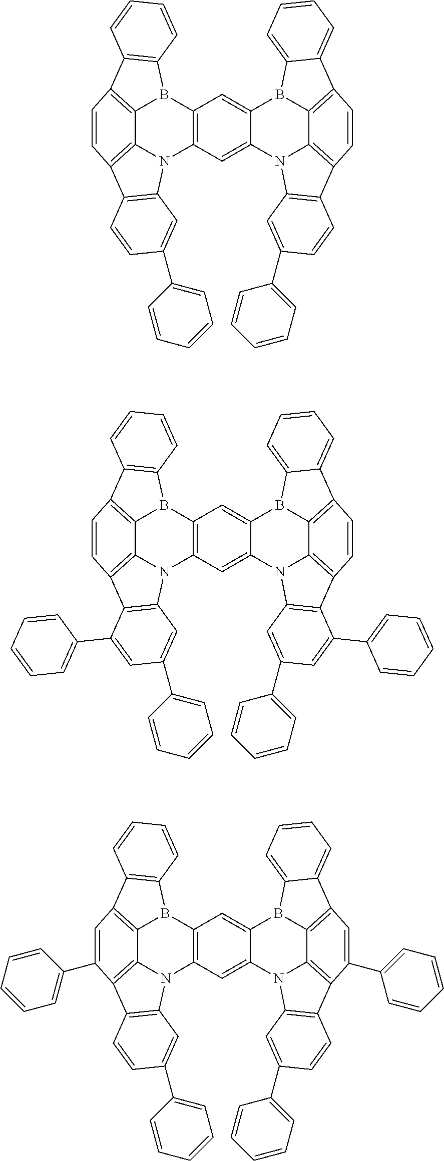

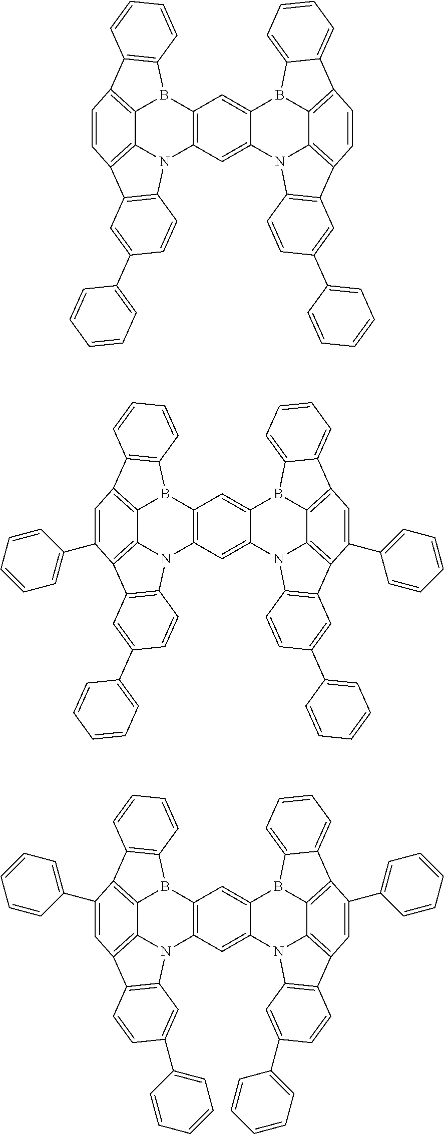

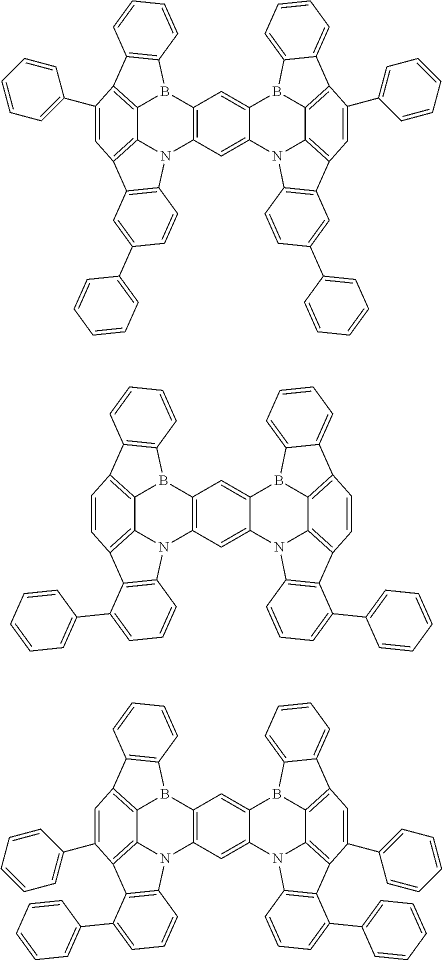

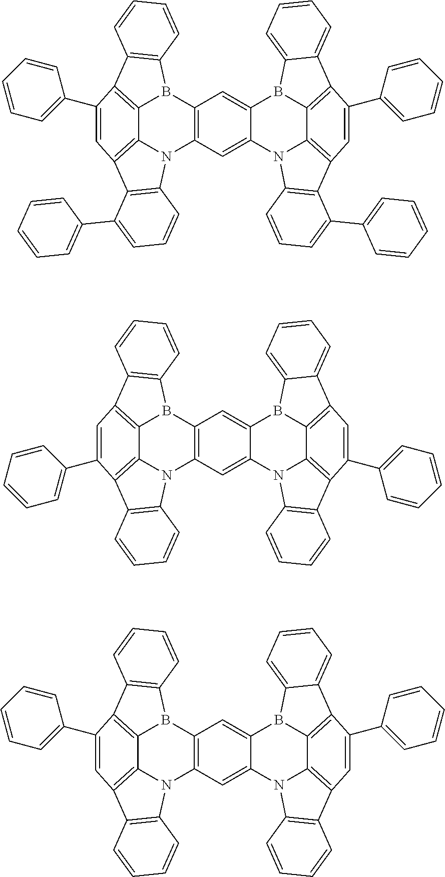

In one preferred aspect of the present invention, compounds in which another ring is fused with two benzene rings forming a carbazole partial structure existing in the general formula (G) are selected. Among them, a compound in which a benzofuran ring is fused, a compound in which a benzothiophene ring is fused, and a compound in which a benzene ring is fused can be particularly preferably selected. Hereinafter, compounds in which these rings are fused will be described with reference to specific examples.

A compound in which a benzofuran ring or a benzothiophene ring is fused with a benzene ring to which a boron atom does not directly bond, between two benzene rings forming a carbazole partial structure existing in the general formula (G), can be preferably mentioned. Examples of such a compound include a compound having the following skeleton (4a), and a compound having the following skeleton (4b).

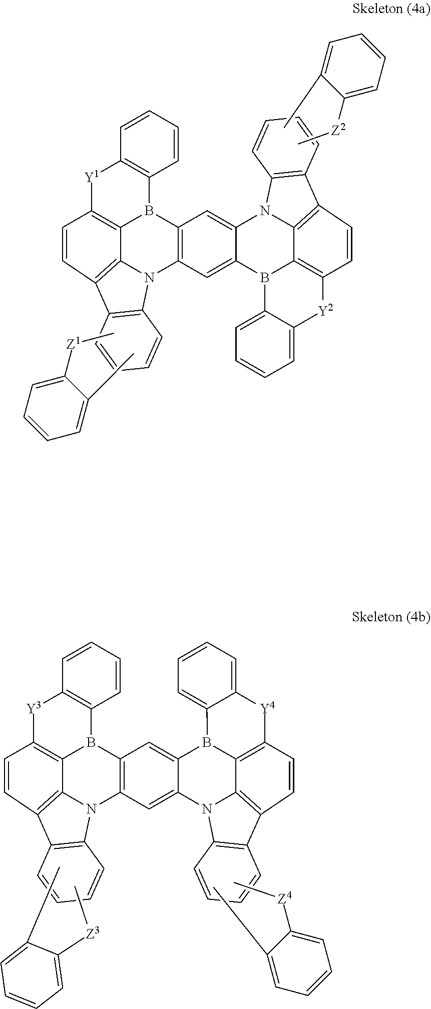

In the skeletons (4a) and (4b), each of Y¹ to Y⁴ independently represents two hydrogen atoms, a single bond or N(R²⁷). Two hydrogen atoms mentioned herein indicate a state where two benzene rings bonding to a boron atom are not linked to each other. It is preferable that Y¹ and Y² are the same, and Y³ and Y⁴ are the same, but they can be different from each other. In one aspect of the present invention, Y¹ to Y⁴ are single bonds. In one aspect of the present invention, Y¹ to Y⁴ are N(R²⁷). R²⁷ represents a hydrogen atom, a deuterium atom, or a substituent.

Each of Z¹ to Z⁴ independently represents an oxygen atom or a sulfur atom. It is preferable that Z¹ and Z² are the same, and Z³ and Z⁴ are the same, but they can be different from each other. In one aspect of the present invention, Z¹ to Z⁴ are oxygen atoms. Here, a furan ring of benzofuran is fused with the benzene ring constituting the carbazole partial structure in (4a) and (4b). The orientation of the fused furan ring is not limited. In one aspect of the present invention, Z¹ to Z⁴ are sulfur atoms. Here, a thiophene ring of benzothiophene is fused with the benzene ring constituting the carbazole partial structure in (4a) and (4b). The orientation of the fused thiophene ring is not limited.

Each hydrogen atom in the skeletons (4a) and (4b) can be substituted with a deuterium atom or a substituent. Further, it can be substituted with a linking group together with an adjacent hydrogen atom to form a cyclic structure. For details, corresponding descriptions on R¹ to R²⁶, A¹, and A² in the general formula (G) can be referred to. In one aspect of the present invention, each hydrogen atom in the skeletons (4a) and (4b) is not substituted with a linking group together with an adjacent hydrogen atom to form a cyclic structure.

As one preferable group of compounds having the skeleton (4a), compounds represented by the following general formula (4a) can be exemplified. It is assumed that X in specific examples is an oxygen atom or a sulfur atom, and a compound in which X is an oxygen atom and a compound in which X is a sulfur atom are disclosed, respectively. Further, in specific examples of compounds represented by other subsequent general formulas, X has the same meaning.

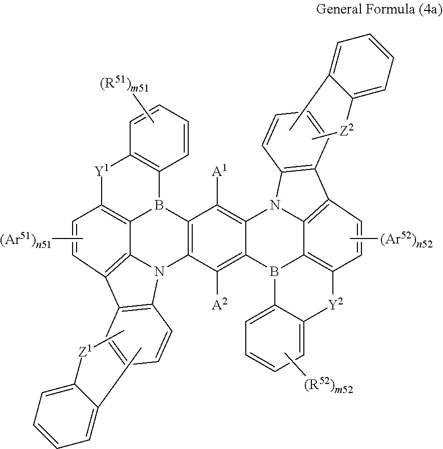

In the general formula (4a), each of Ar⁵¹ and Ar⁵² independently represents a substituted or unsubstituted aryl group, a substituted or unsubstituted heteroaryl group, or a substituted or unsubstituted alkyl group, and for example, a substituted or unsubstituted aryl group can be preferably selected. Each of R⁵¹ and R⁵² independently represents a substituted or unsubstituted alkyl group. Each of m51 and m52 independently represents an integer of 0 to 4. Each of n51 and n52 independently represents an integer of 0 to 2. Each of Y¹ to Y⁴ independently represents two hydrogen atoms, a single bond or N(R²⁷). R²⁷ represents a hydrogen atom, a deuterium atom, or a substituent. Each of Z¹ to Z⁴ independently represents an oxygen atom or a sulfur atom. Each of A¹ and A² independently represents a hydrogen atom, a deuterium atom, or a substituent.

In one aspect of the present invention, n51 and n52 are the same number. For example, n51 and n52 can be 0, and n5l and n52 can be 1. In one aspect of the present invention, m5l and m52 are the same number. In one aspect of the present invention, m5l and m52 are integers of 0 to 3. For example, m51 and m52 can be 0, m51 and m52 can be 1, m51 and m52 can be 2, and m51 and m52 can be 3. In relation to preferable groups for Ar⁵¹, Ar⁵², R⁵¹, R⁵², A¹, and A², corresponding descriptions on Ar¹ to Ar⁴, R⁴¹ to R⁴², A¹, and A² in the general formula (1a) can be referred to.

Hereinafter, specific examples of the compound represented by the general formula (4a) will be given. Compounds of the general formula (4a) that can be used in the present invention are not construed as limiting to specific examples in the following one group. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

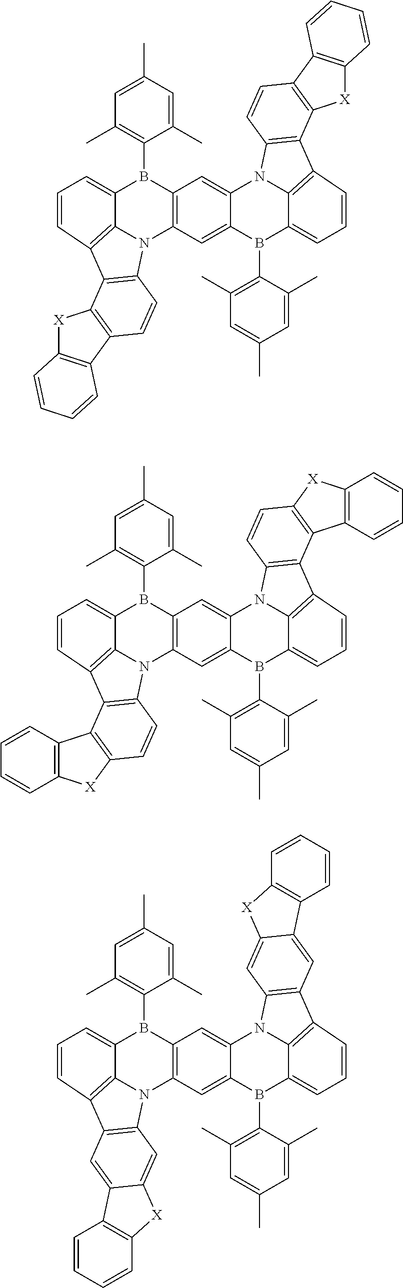

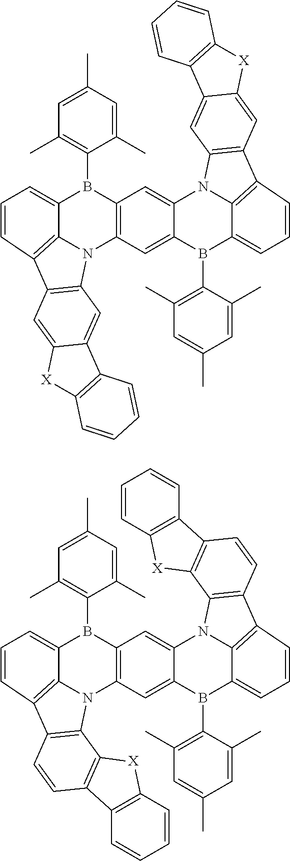

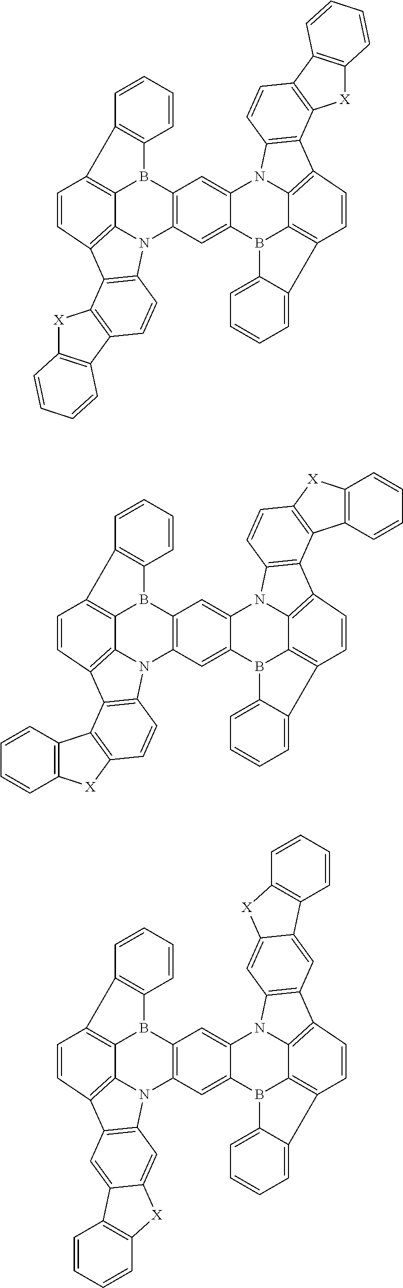

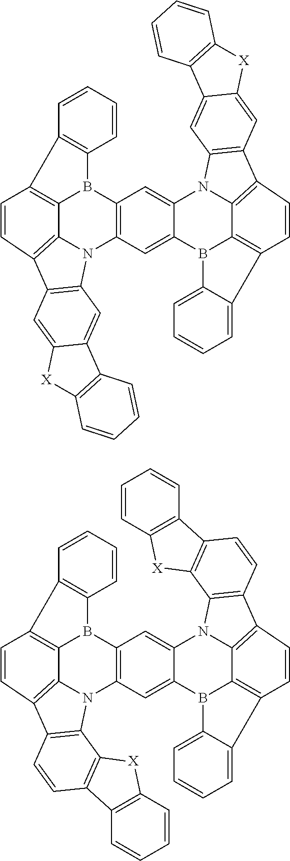

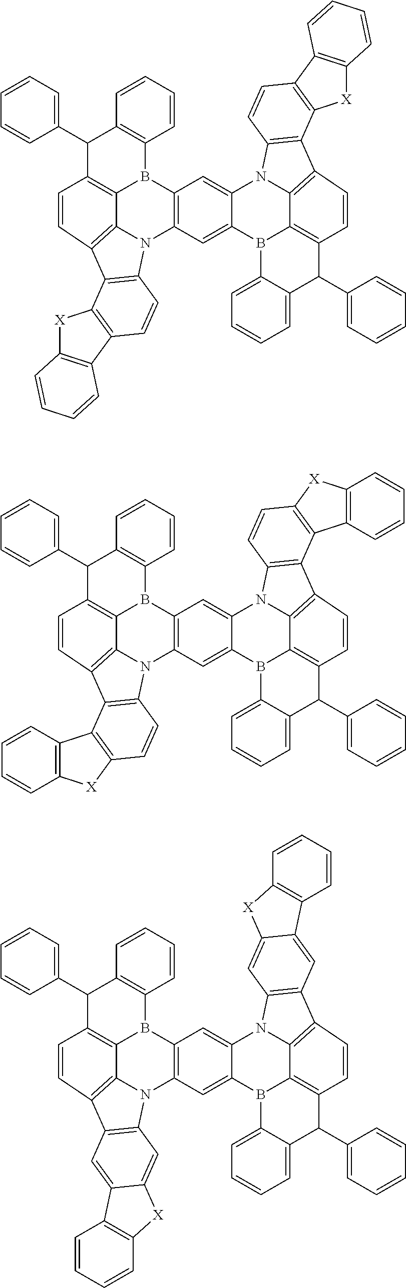

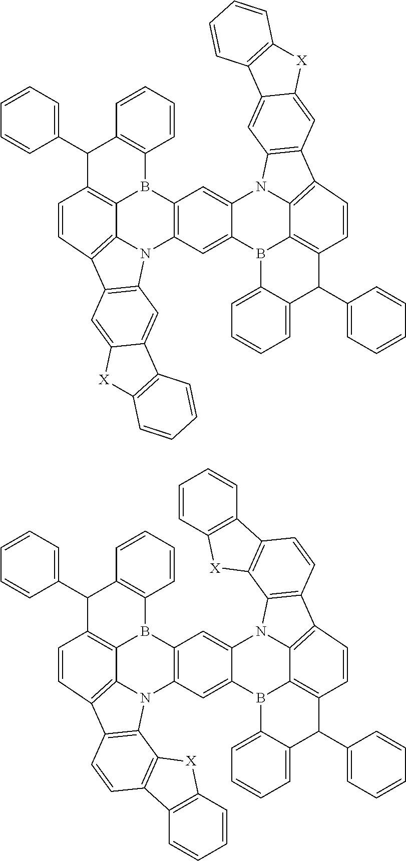

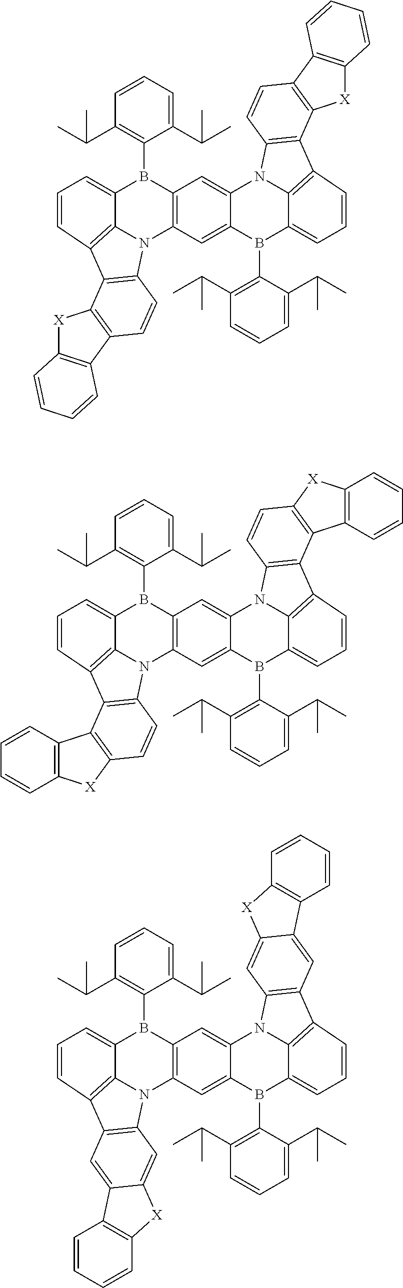

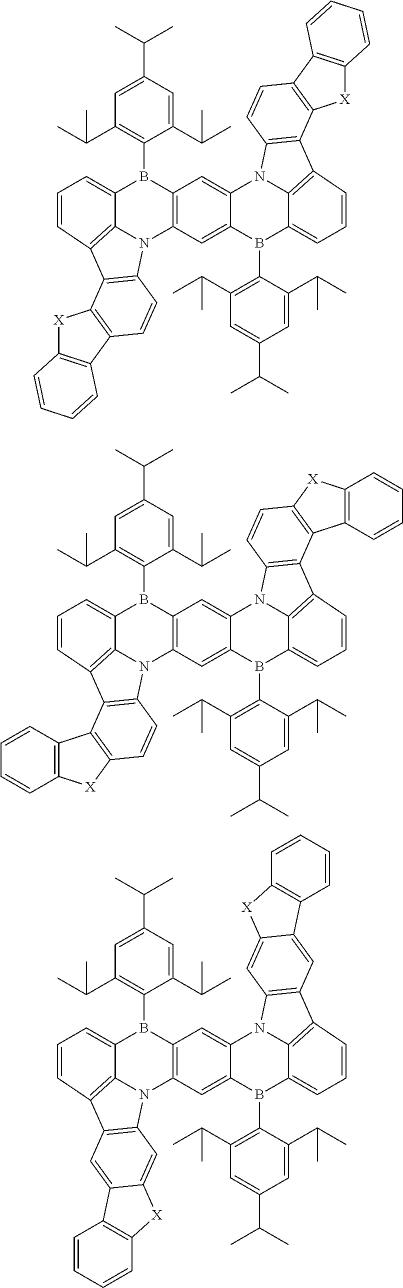

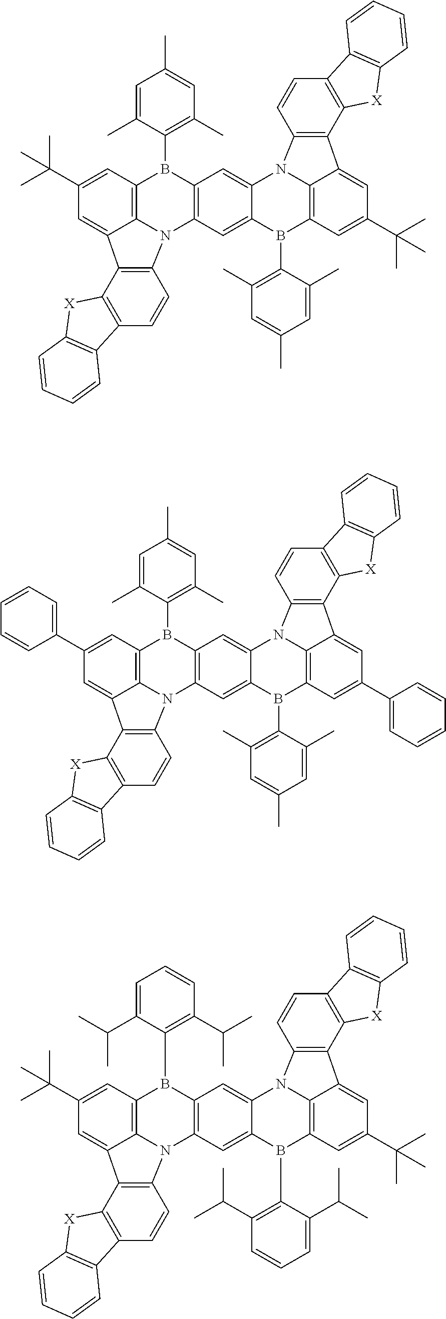

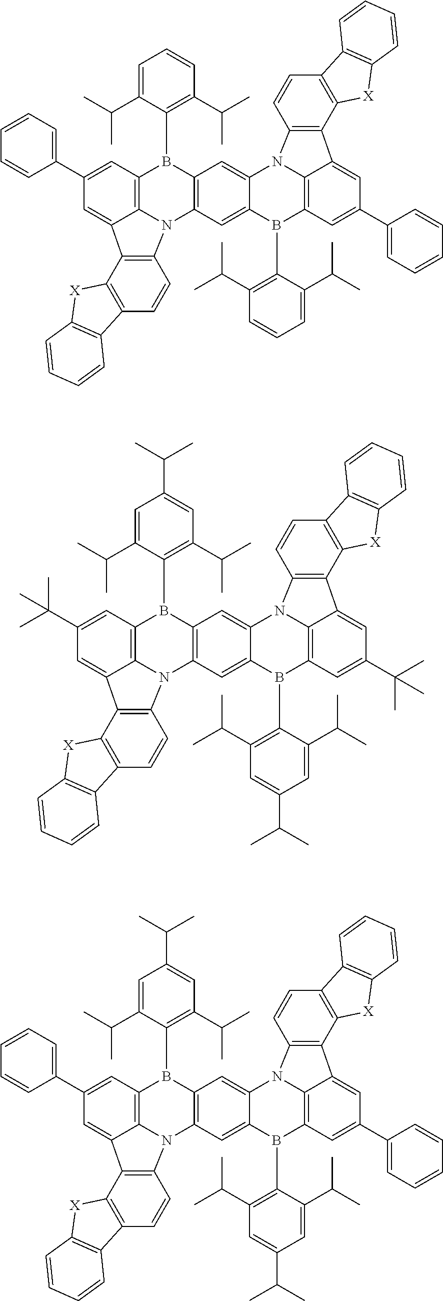

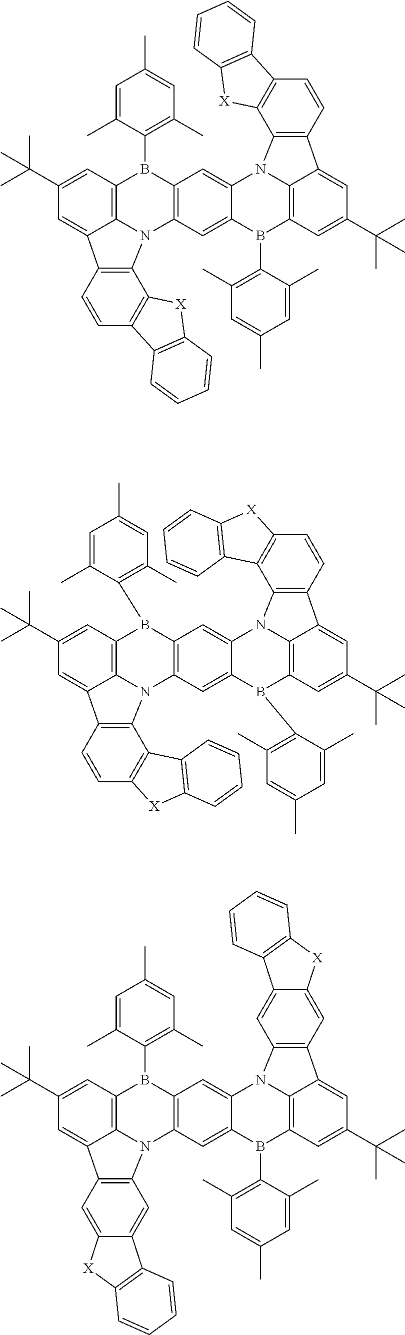

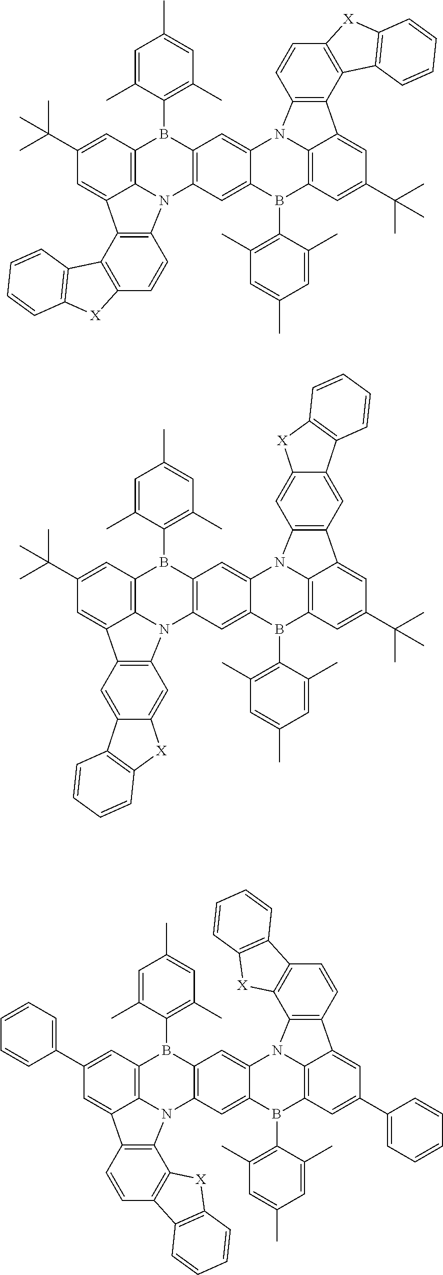

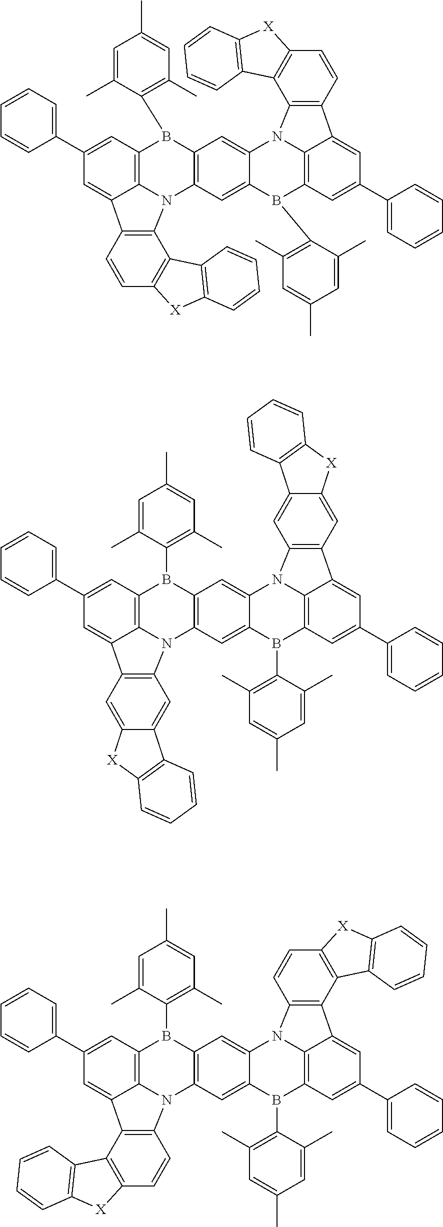

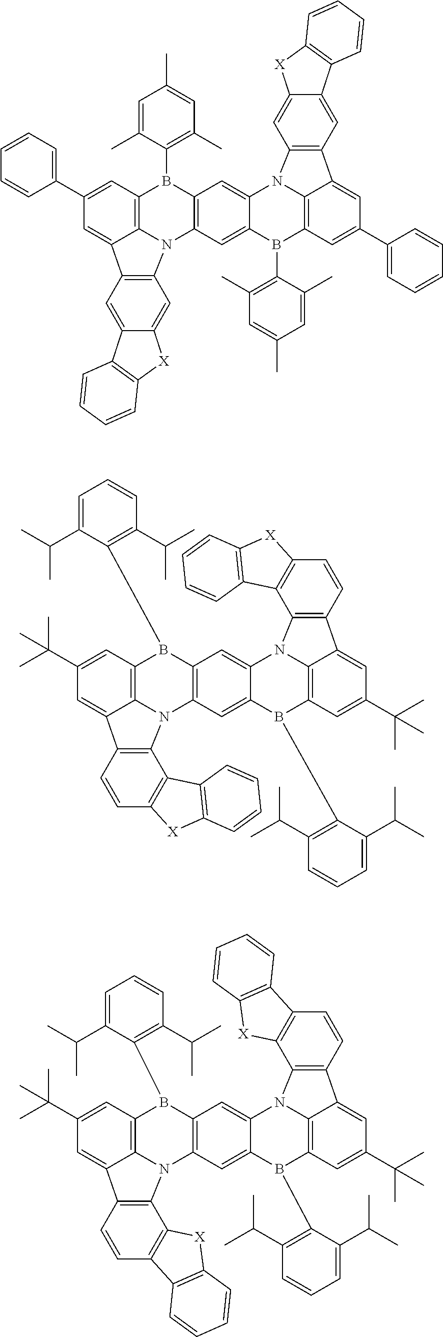

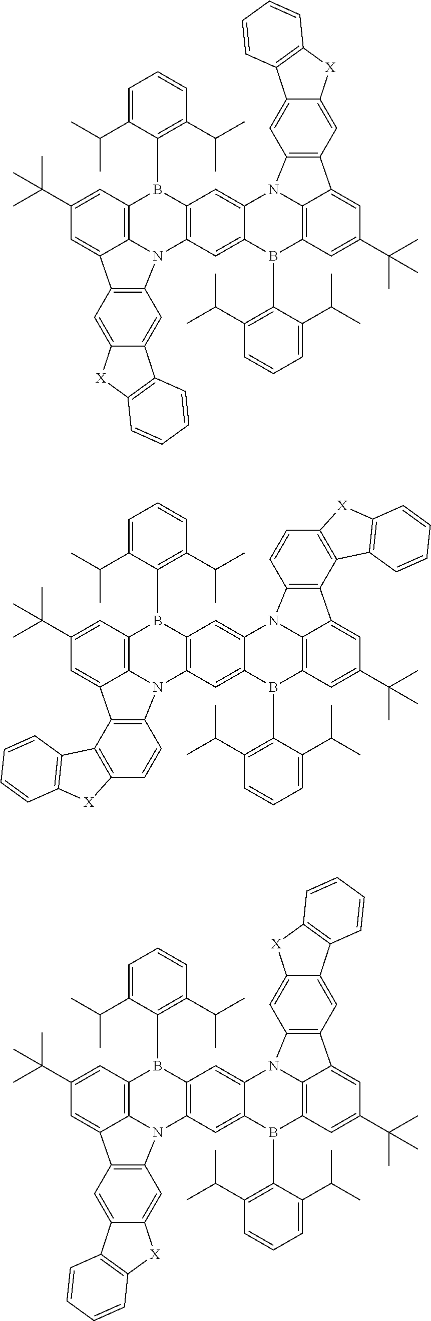

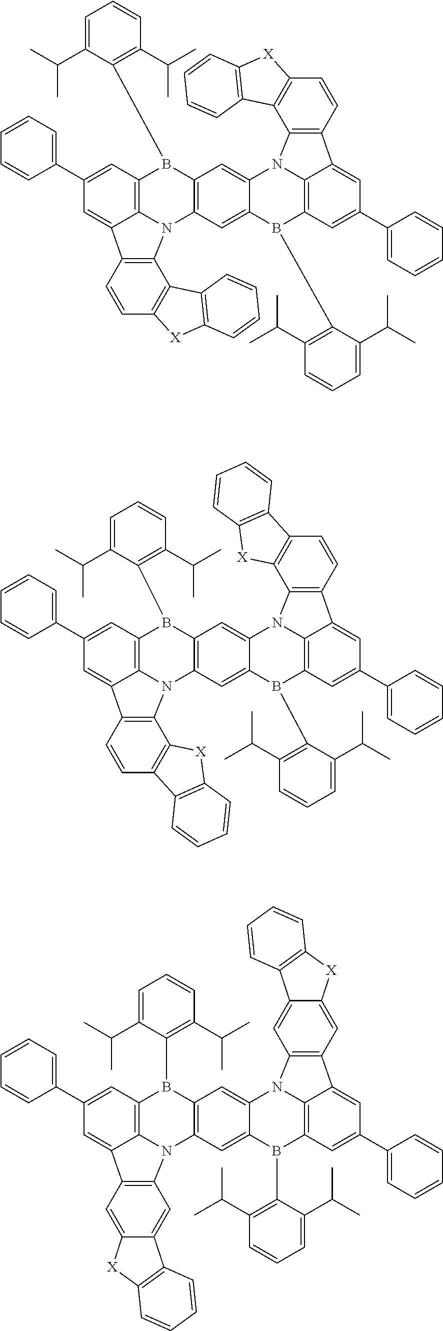

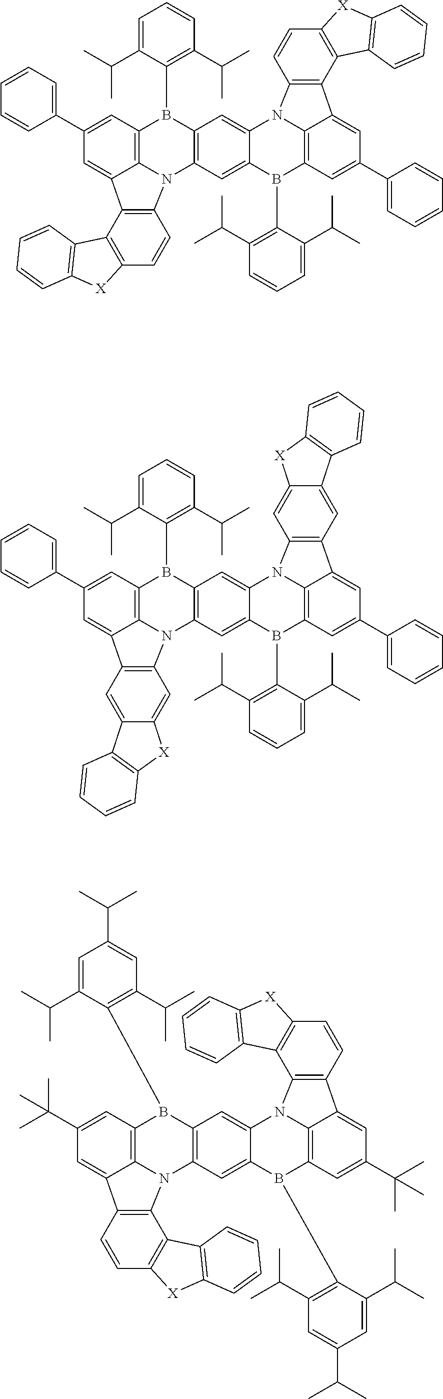

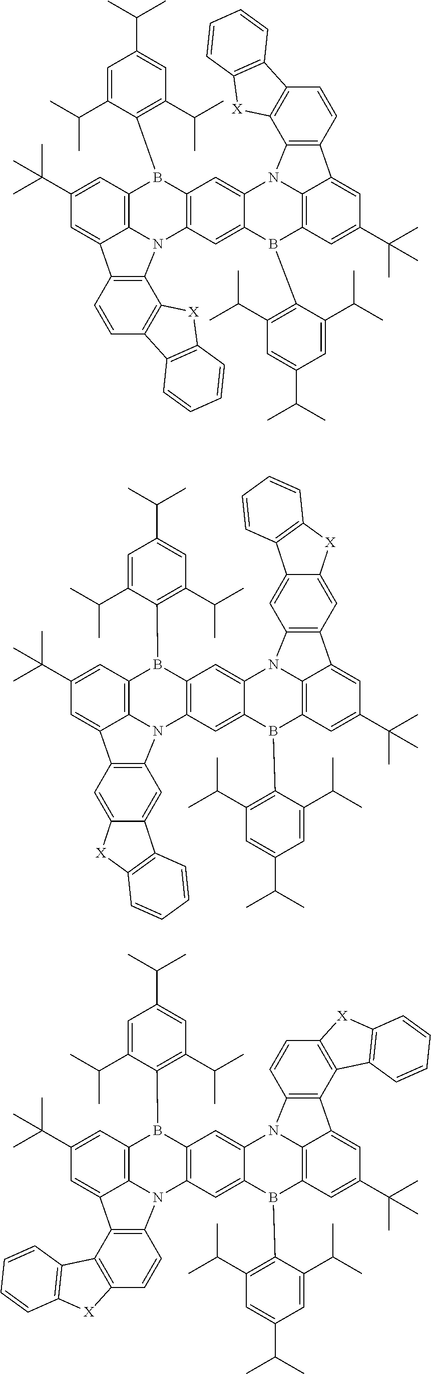

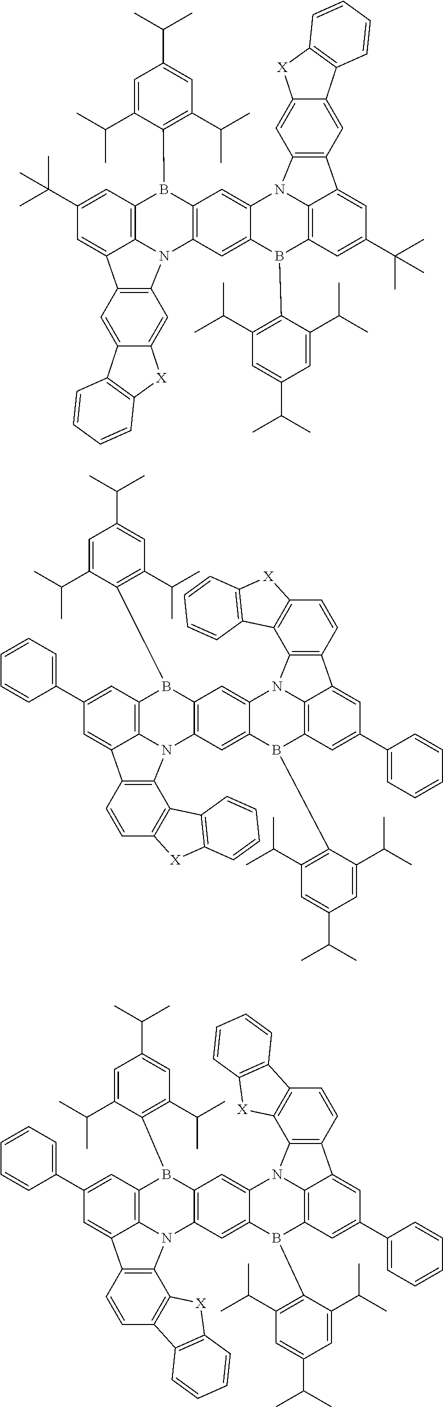

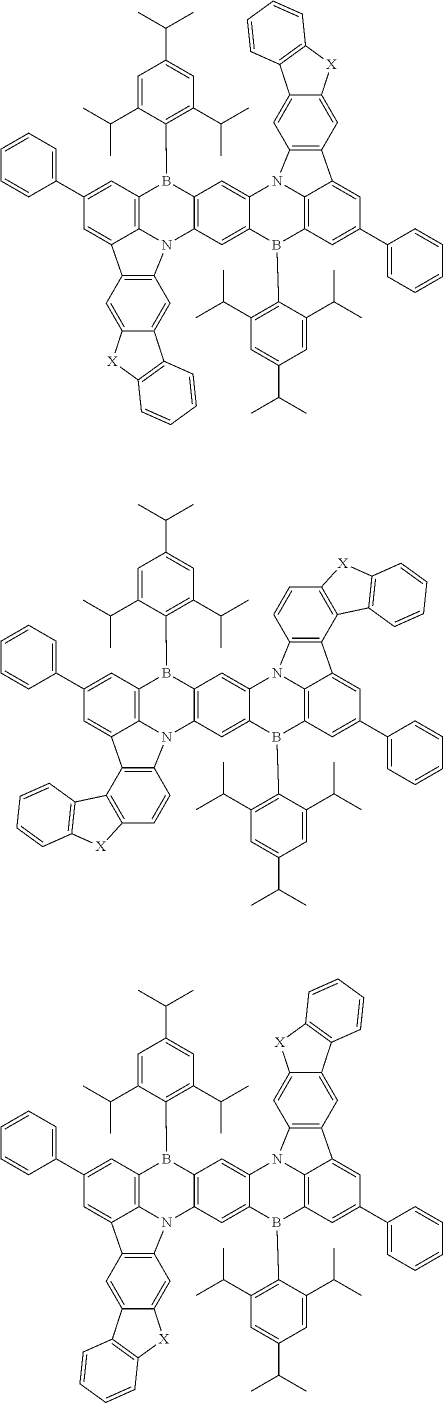

Hereinafter, another group of specific examples of the compound represented by the general formula (4a) will be given. Compounds of the general formula (4a) that can be used in the present invention are not construed as limiting to specific examples in the following one group.

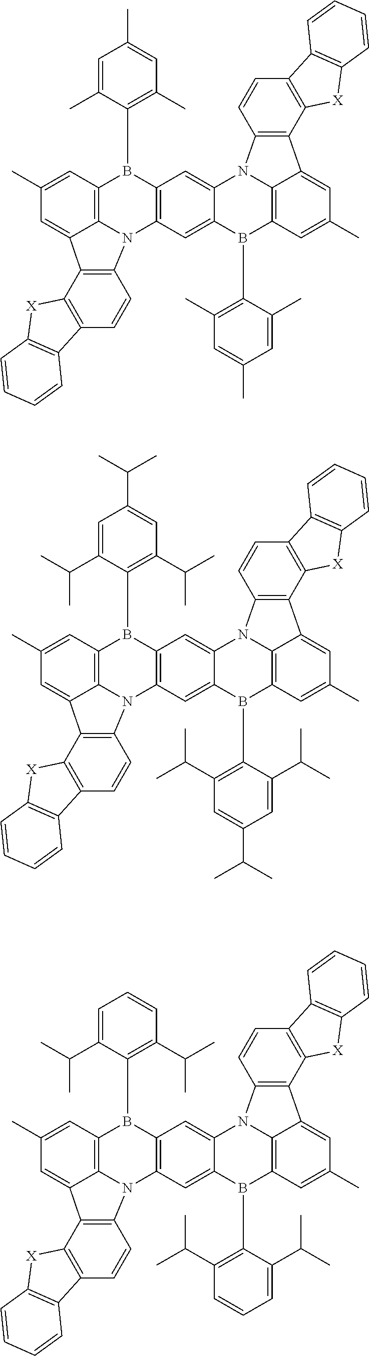

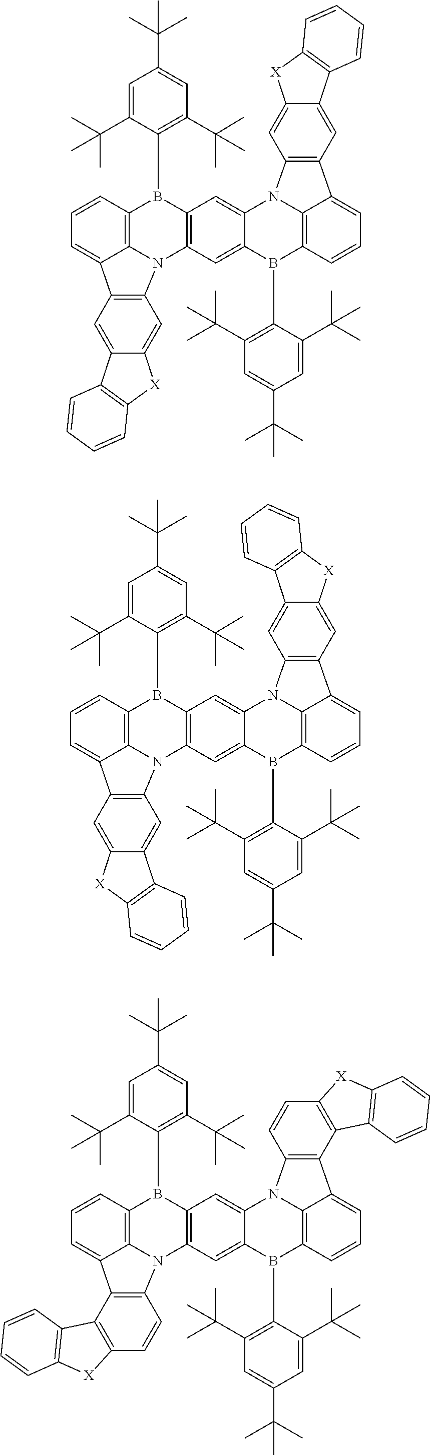

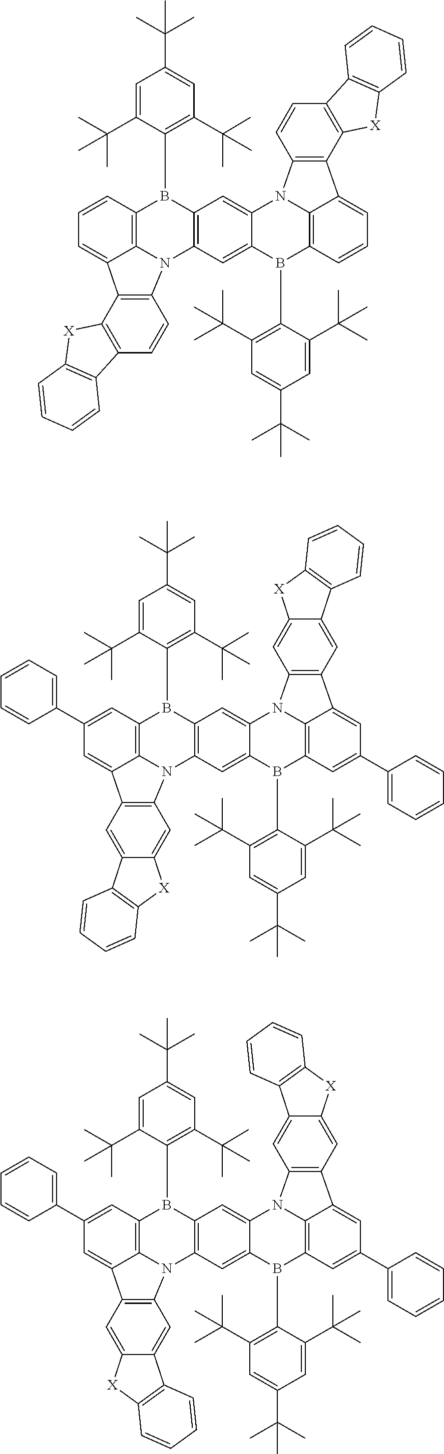

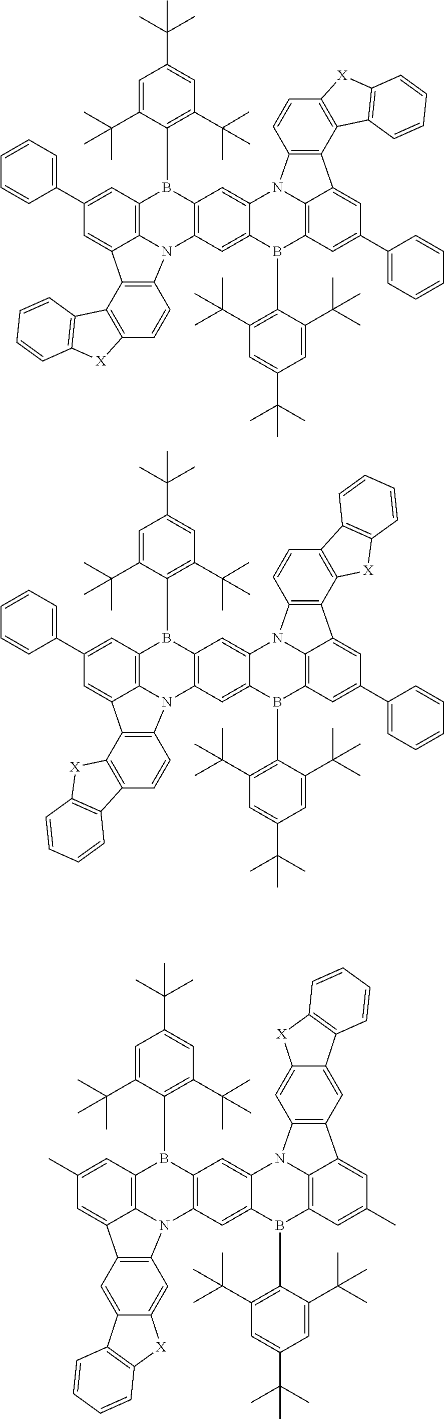

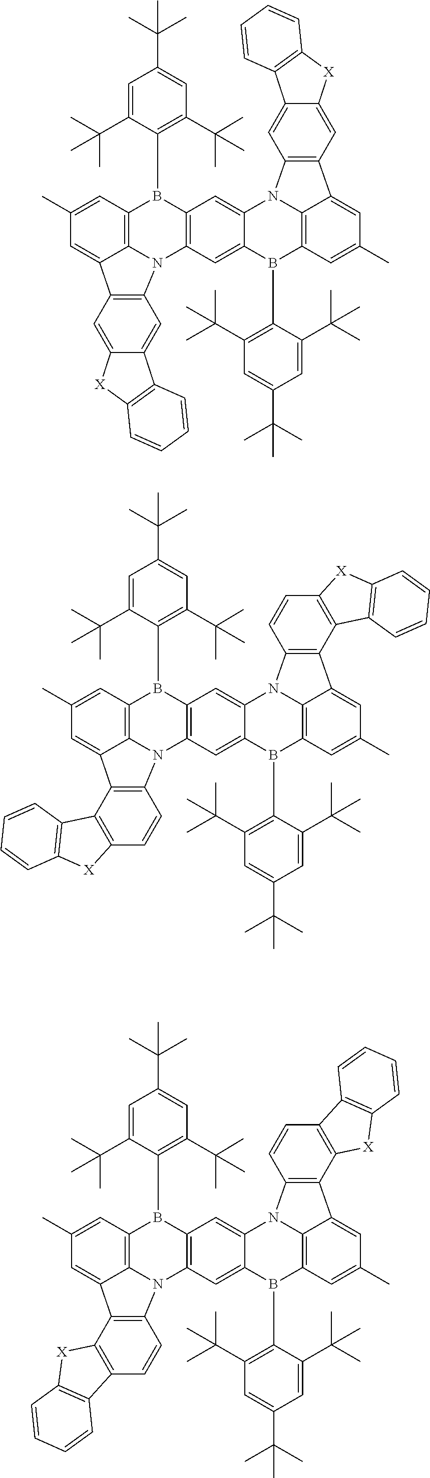

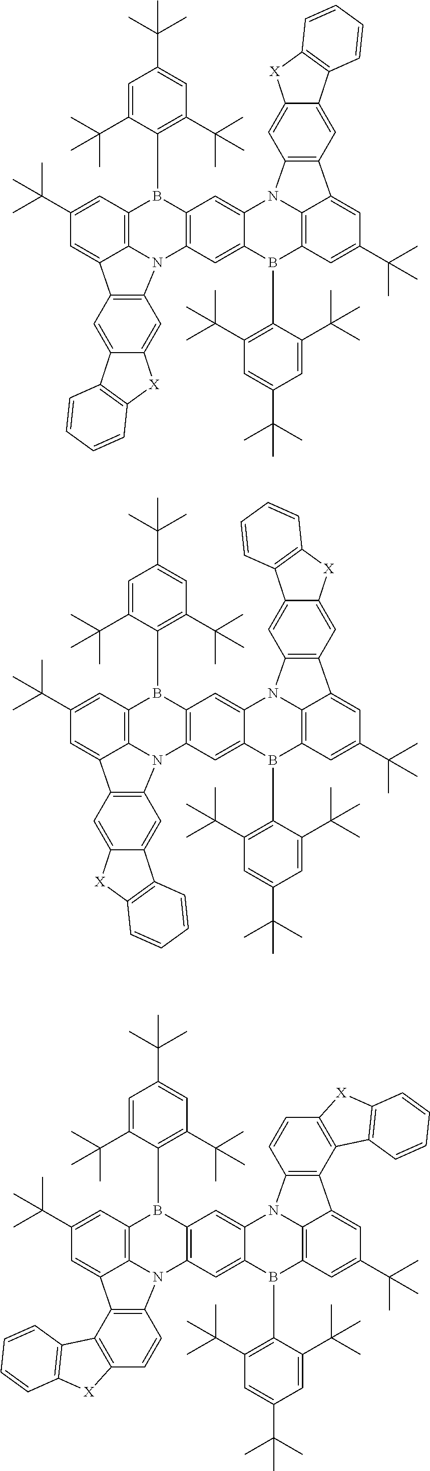

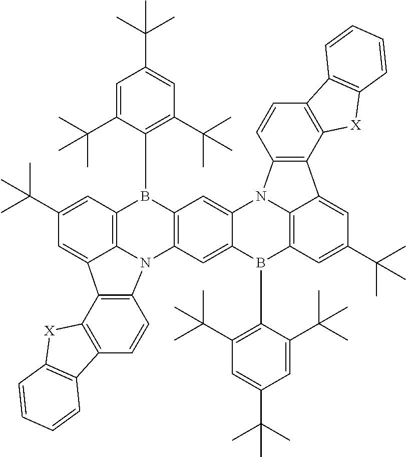

As one preferable group of compounds having the skeleton (4b), compounds represented by the following general formula (4b) can be exemplified.

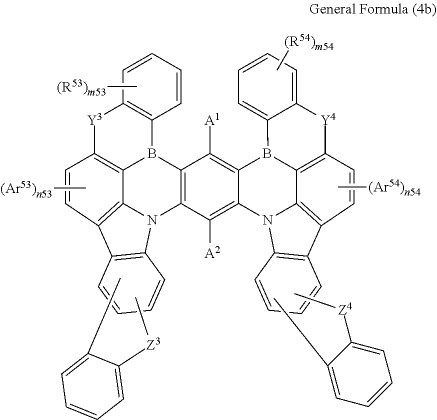

In the general formula (4b), each of Ar⁵³ and Ar⁵⁴ independently represents a substituted or unsubstituted aryl group, a substituted or unsubstituted heteroaryl group, or a substituted or unsubstituted alkyl group, and for example, a substituted or unsubstituted aryl group can be preferably selected. Each of R⁵³ and R⁵⁴ independently represents a substituted or unsubstituted alkyl group. Each of m53 and m54 independently represents an integer of 0 to 4. Each of n53 and n54 independently represents an integer of 0 to 2. Each of Y³ and Y⁴ independently represents two hydrogen atoms, a single bond or N(R²⁷). R²⁷ represents a hydrogen atom, a deuterium atom, or a substituent. Each of Z³ and Z⁴ independently represents an oxygen atom or a sulfur atom. Each of A¹ and A² independently represents a hydrogen atom, a deuterium atom, or a substituent. In relation to details of Ar⁵³, Ar⁵⁴, R⁵³, R⁵⁴, m53, m54, n53, n54, A¹, and A², the descriptions on Ar⁵¹, Ar⁵², R⁵¹, R⁵², m51, m52, n51, n52, A¹, and A² in the general formula (4a) can be referred to.

Hereinafter, specific examples of the compound represented by the general formula (4b) will be given. Compounds of the general formula (4b) that can be used in the present invention are not construed as limiting to the following specific examples. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

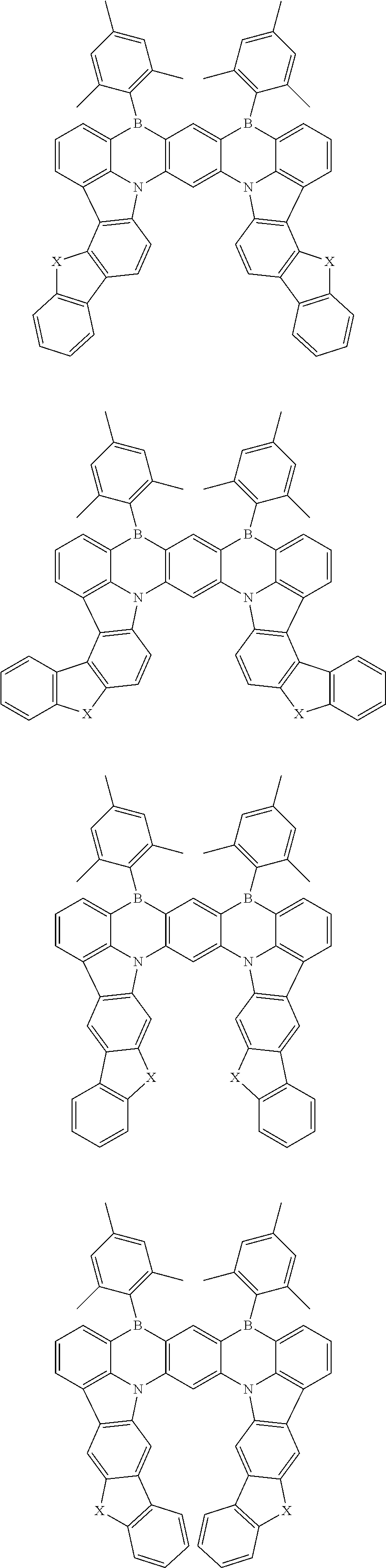

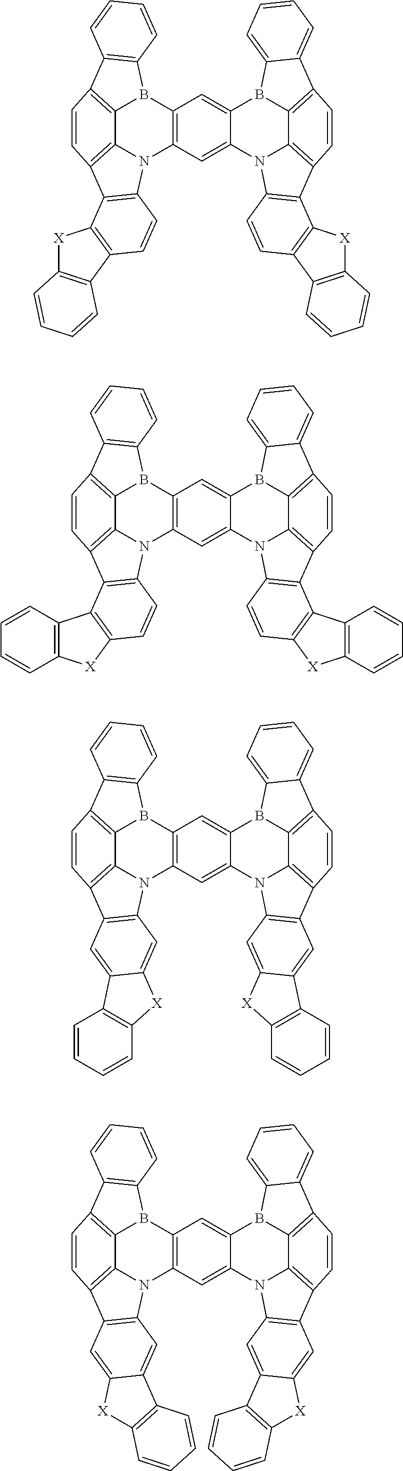

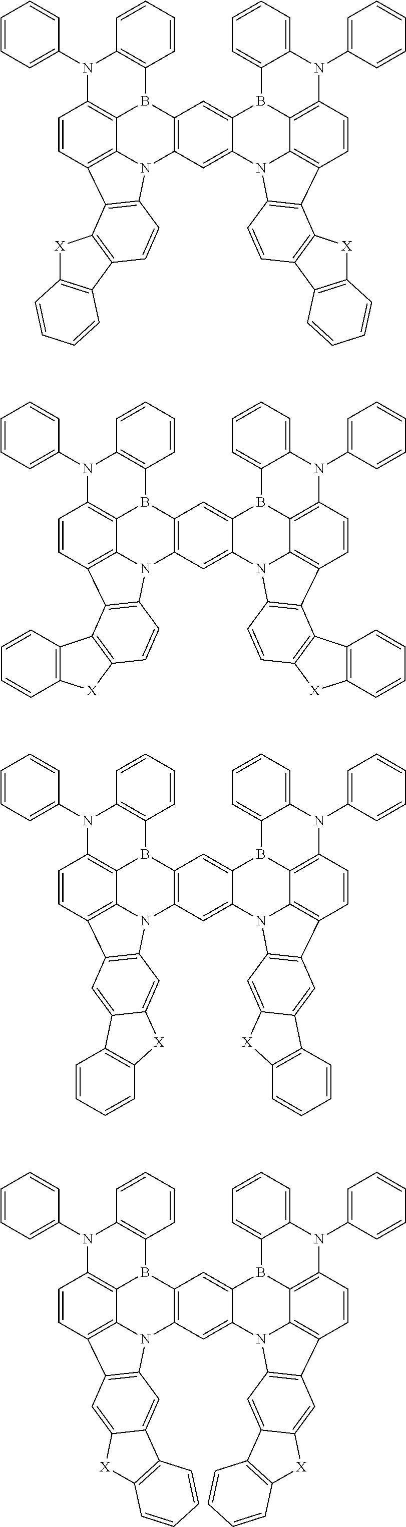

A compound in which a benzofuran ring or a benzothiophene ring is fused with a benzene ring to which a boron atom directly bonds, between two benzene rings forming a carbazole partial structure existing in the general formula (G), can be preferably mentioned. Examples of such a compound include a compound having the following skeleton (5a) and a compound having the following skeleton (5b).

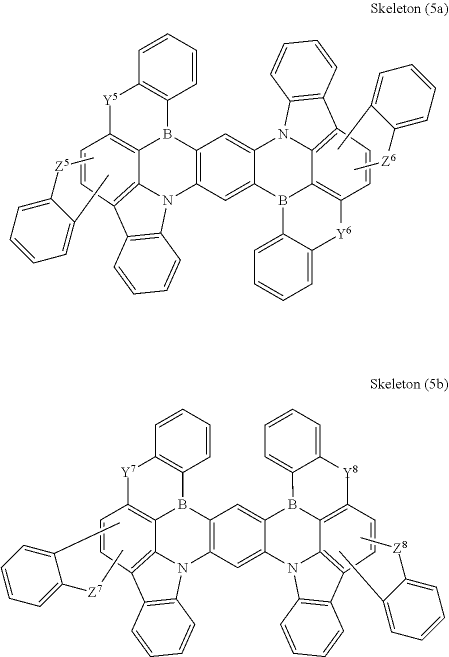

In the skeletons (5a) and (5b), each of Y⁵ to Y⁸ independently represents two hydrogen atoms, a single bond or N(R²⁷). Each of Z⁵ to Z⁸ independently represents an oxygen atom or a sulfur atom. In relation to details of Y⁵ to Y⁸, and Z⁵ to Z⁸, corresponding descriptions for the skeletons (4a) and (4b) can be referred to. In one aspect of the present invention, each hydrogen atom in the skeletons (5a) and (5b) is not substituted with a linking group together with an adjacent hydrogen atom to form a cyclic structure.

As one preferable group of compounds having the skeleton (5a), compounds represented by the following general formula (5a) can be exemplified.

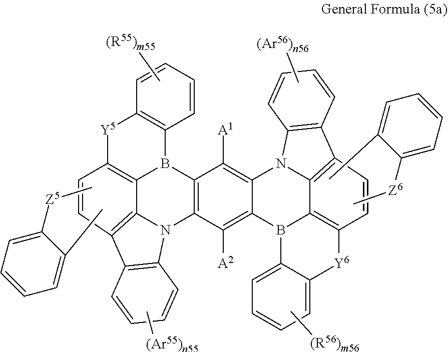

In the general formula (5a), each of Ar⁵⁵ and Ar⁵⁶ independently represents a substituted or unsubstituted aryl group, a substituted or unsubstituted heteroaryl group, or a substituted or unsubstituted alkyl group, and for example, a substituted or unsubstituted aryl group can be preferably selected. Each of R⁵⁵ and R⁵⁶ independently represents a substituted or unsubstituted alkyl group. Each of m55 and m56 independently represents an integer of 0 to 4. Each of n55 and n56 independently represents an integer of 0 to 4. Each of Y⁵ and Y⁶ independently represents two hydrogen atoms, a single bond or N(R²⁷). R²⁷ represents a hydrogen atom, a deuterium atom, or a substituent. Each of Z⁵ and Z⁶ independently represents an oxygen atom or a sulfur atom. Each of A¹ and A² independently represents a hydrogen atom, a deuterium atom, or a substituent.

In one aspect of the present invention, n55 and n56 are integers of 0 to 2. For example, n55 and n56 can be 0, and n55 and n56 can be 1. In one aspect of the present invention, m51 and m52 are the same number. In relation to details of m55 and m56, descriptions on m51 and m52 in the general formula (4a) can be referred to. In relation to preferable groups for Ar⁵⁵, Ar⁵⁶, R⁵⁵, R⁵⁶, A¹, and A², corresponding descriptions on Ar¹, Ar³, R⁴¹, R⁴², A¹, and A² in the general formula (1a) can be referred to.

Hereinafter, specific examples of the compound represented by the general formula (5a) will be given. Compounds of the general formula (5a) that can be used in the present invention are not construed as limiting to specific examples in the following one group. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

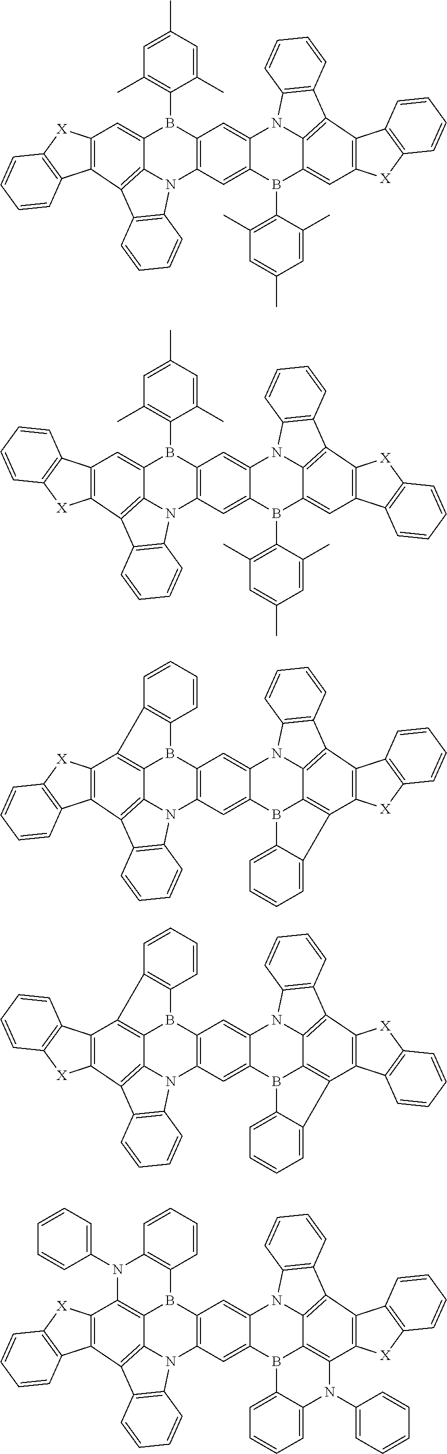

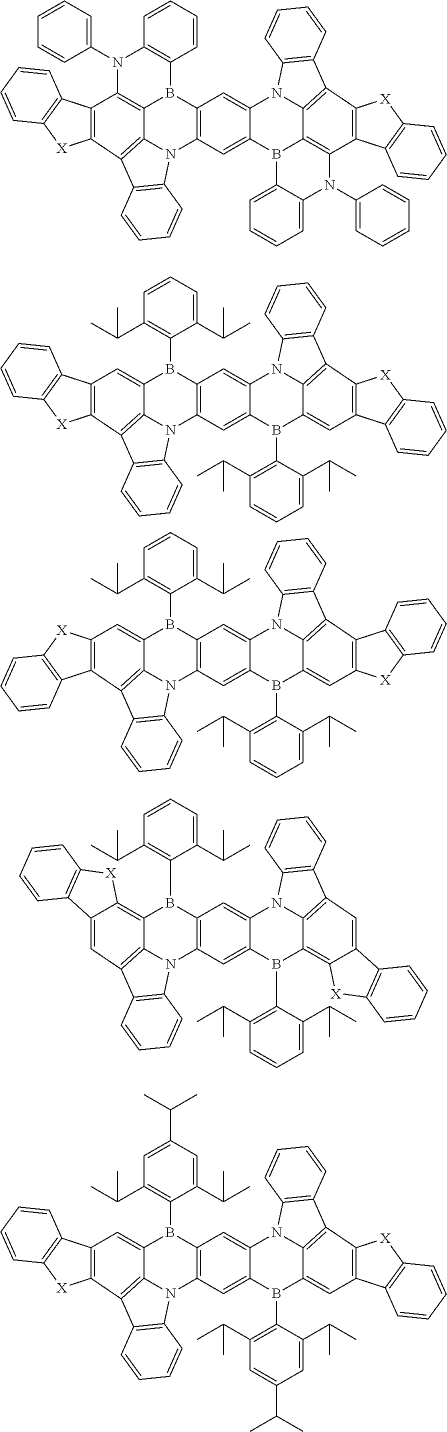

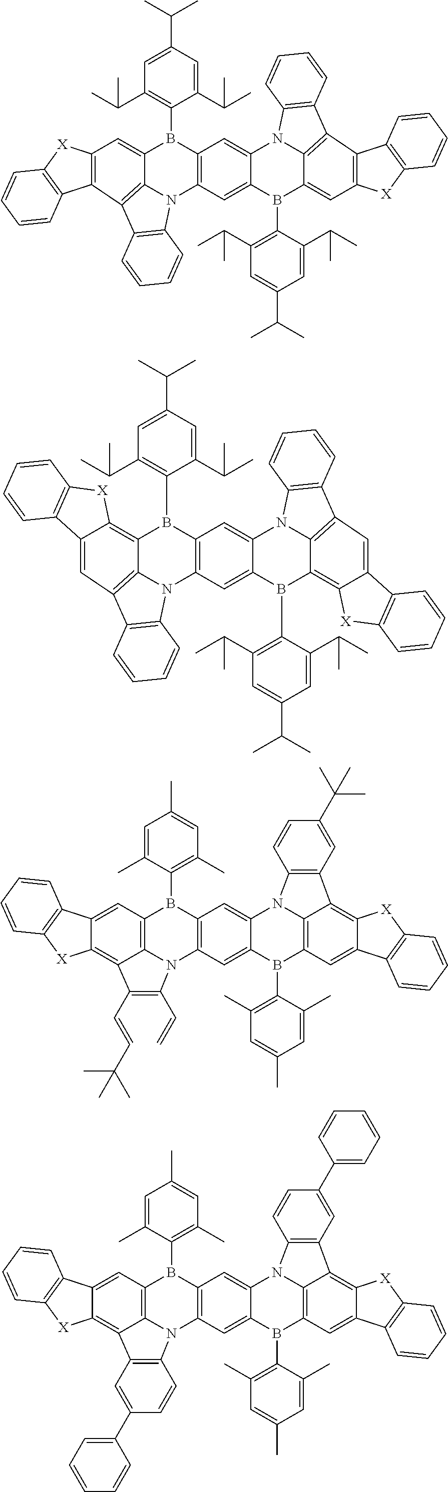

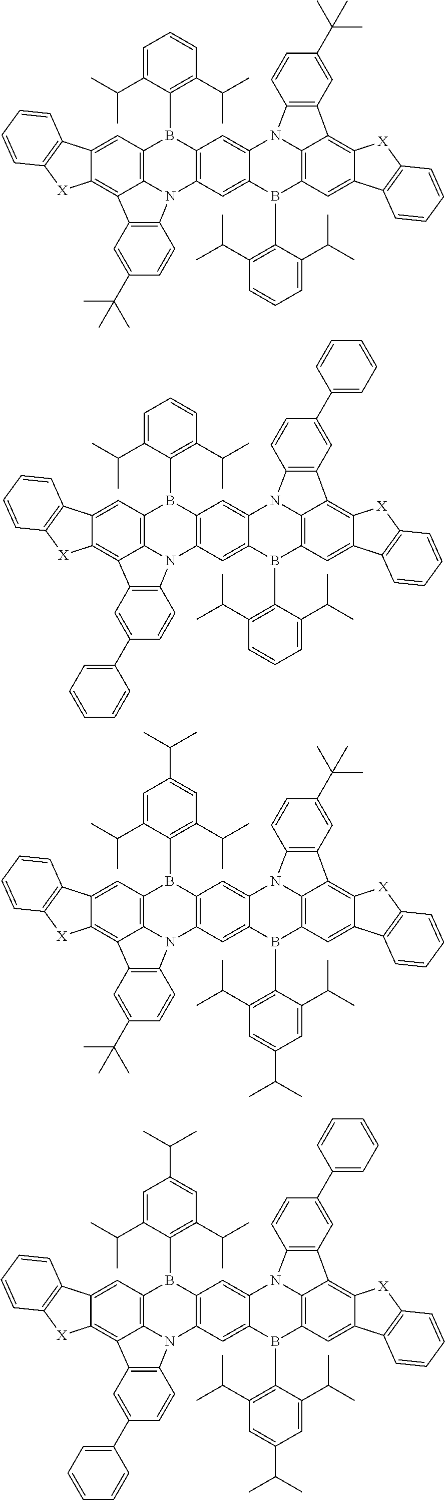

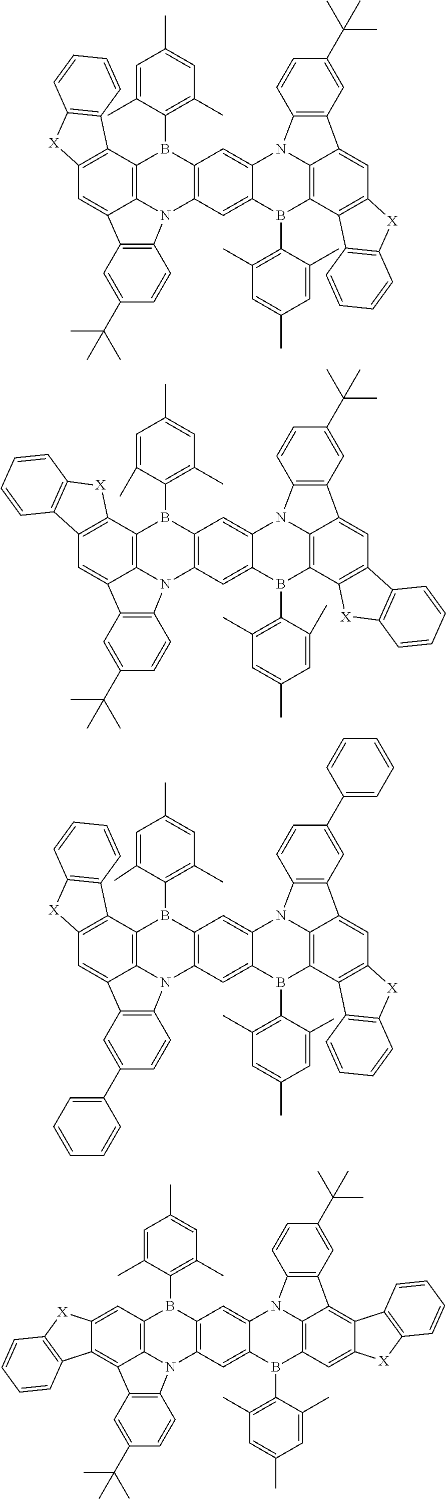

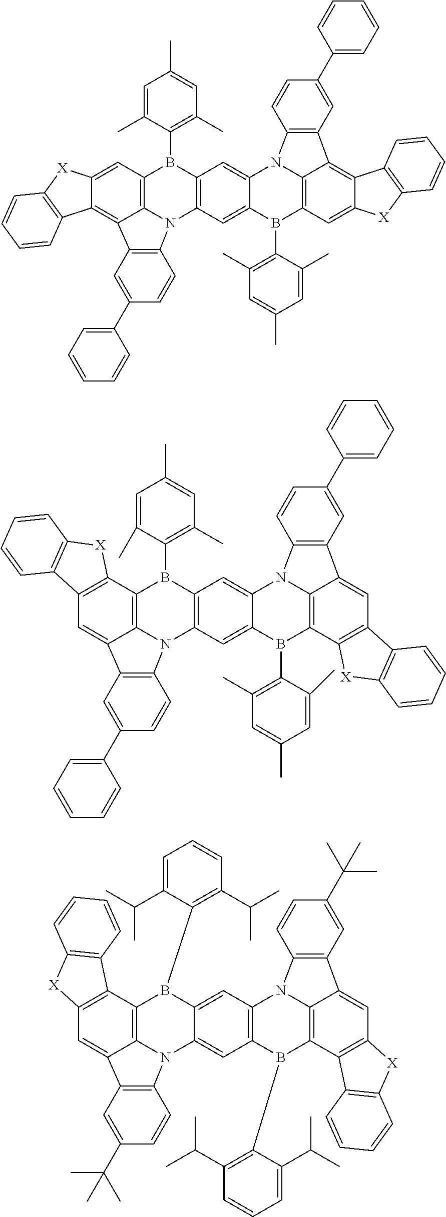

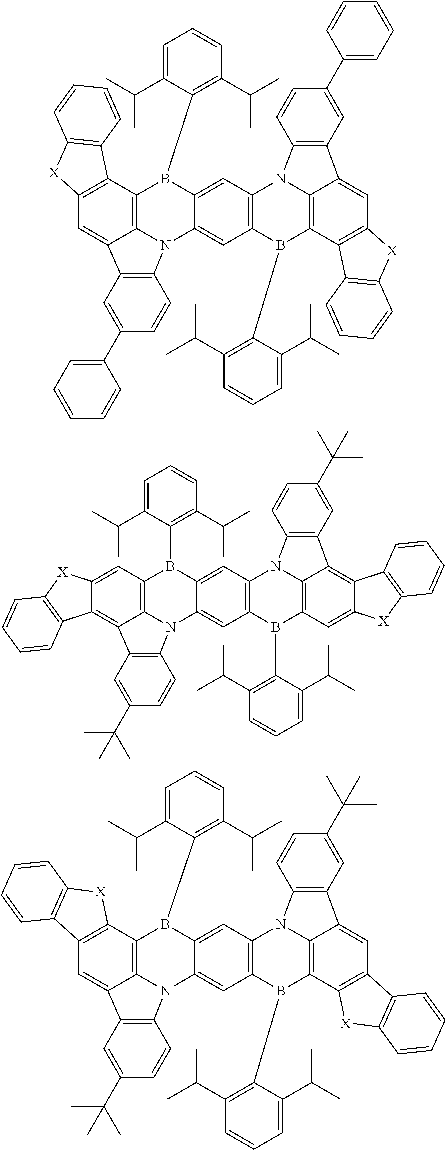

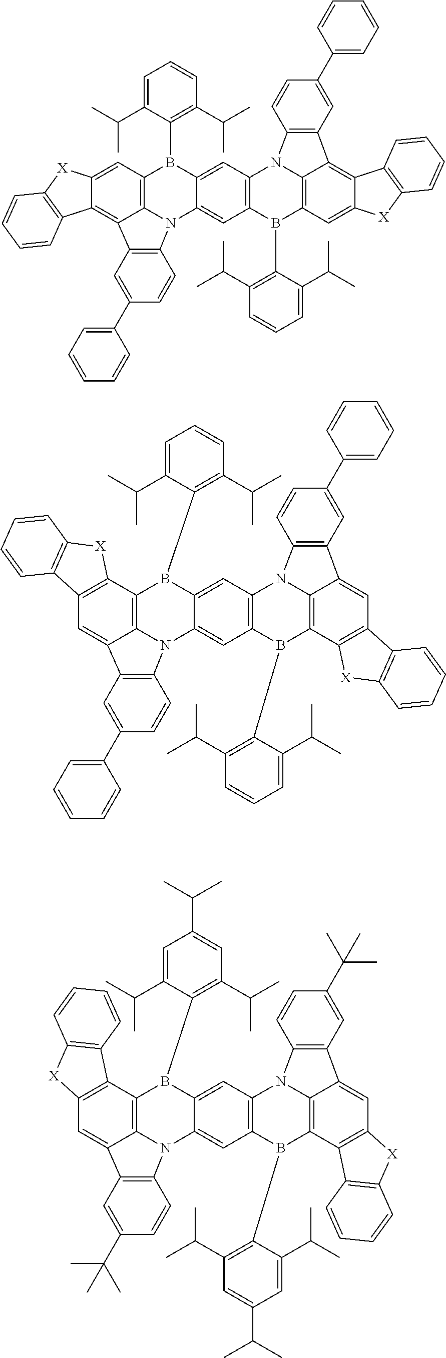

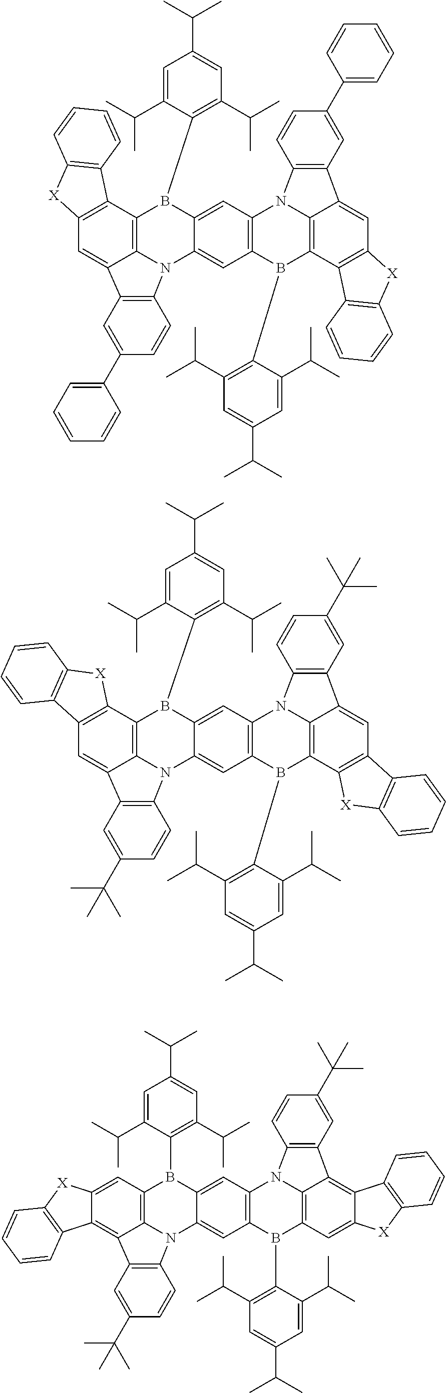

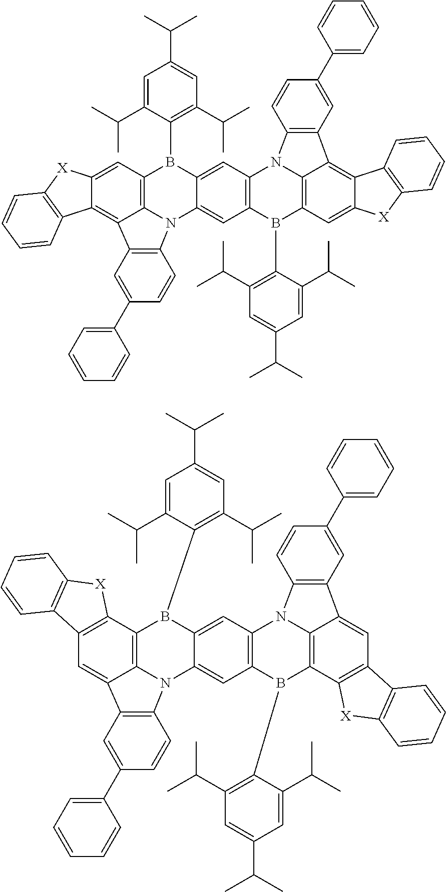

Hereinafter, another group of specific examples of the compound represented by the general formula (5a) will be given. Compounds of the general formula (5a) that can be used in the present invention are not construed as limiting to specific examples in the following one group.

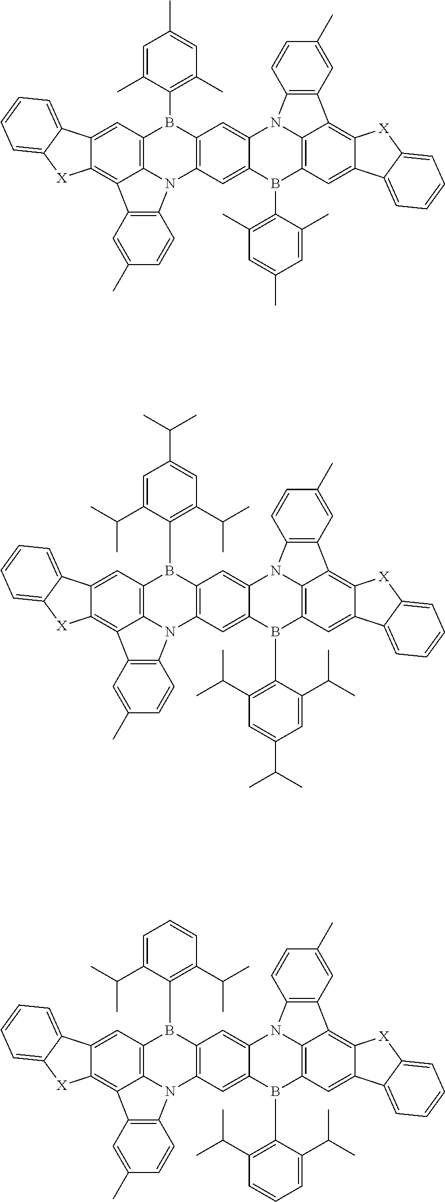

As one preferable group of compounds having the skeleton (5b), compounds represented by the following general formula (5b) can be exemplified.

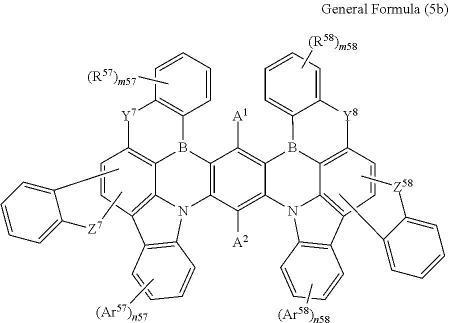

In the general formula (5b), each of Ar⁵⁷ and Ar⁵⁸ independently represents a substituted or unsubstituted aryl group, a substituted or unsubstituted heteroaryl group, or a substituted or unsubstituted alkyl group, and for example, a substituted or unsubstituted aryl group can be preferably selected. Each of R⁵⁷ and R⁵⁸ independently represents a substituted or unsubstituted alkyl group. Each of m57 and m58 independently represents an integer of 0 to 4. Each of n57 and n58 independently represents an integer of 0 to 4. Each of Y⁷ and Y⁸ independently represents two hydrogen atoms, a single bond or N(R²⁷). R²⁷ represents a hydrogen atom, a deuterium atom, or a substituent. Each of Z⁷ and Z⁸ independently represents an oxygen atom or a sulfur atom. Each of A¹ and A² independently represents a hydrogen atom, a deuterium atom, or a substituent. In relation to details of Ar⁵⁷, Ar⁵⁸, R⁵⁷, R⁵⁸, m57, m58, n57, n58, A¹, and A², descriptions on Ar⁵⁵, Ar⁵⁶, R⁵⁵, R⁵⁶, m55, m56, n55, n56, A¹, and A² in the general formula (5a) can be referred to.

Hereinafter, specific examples of the compound represented by the general formula (5b) will be given. Compounds of the general formula (5b) that can be used in the present invention are not construed as limiting to specific examples in the following one group. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

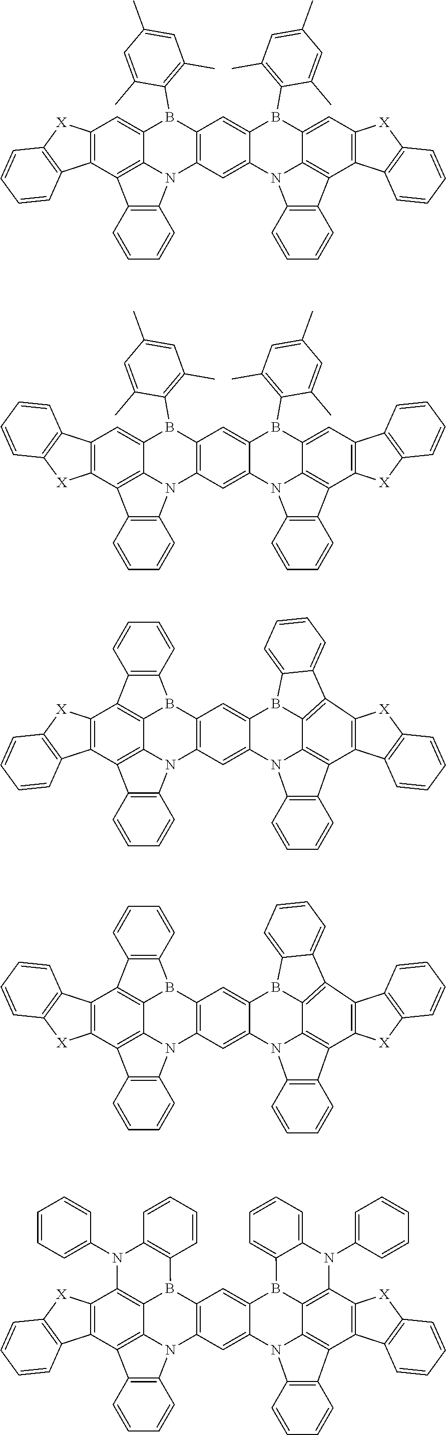

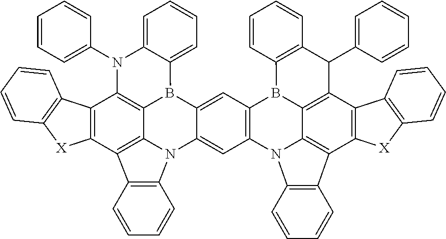

Hereinafter, another group of specific examples of the compound represented by the general formula (5b) will be given. Compounds of the general formula (5b) that can be used in the present invention are not construed as limiting to specific examples in the following one group.

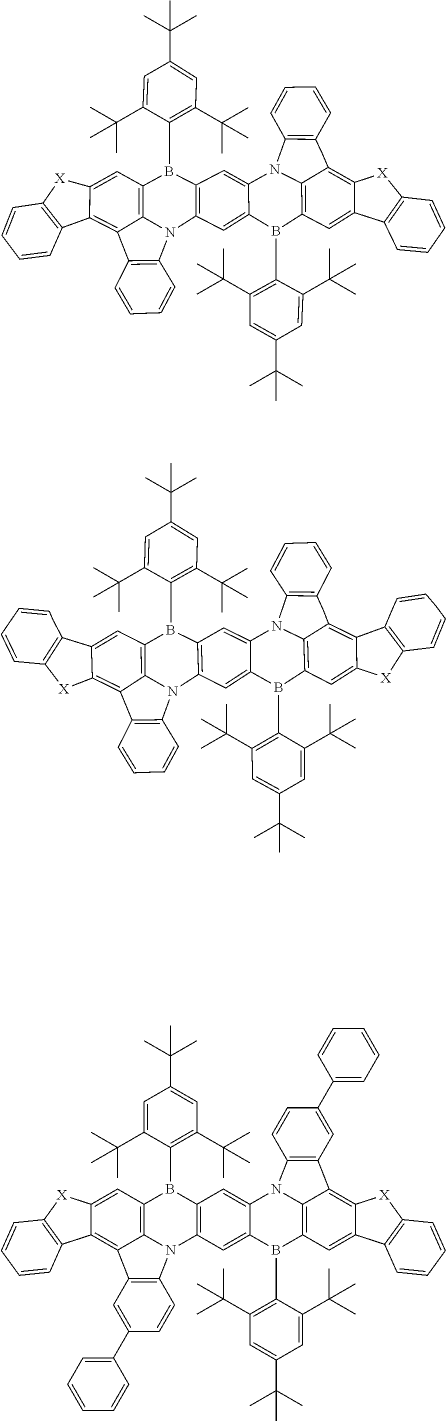

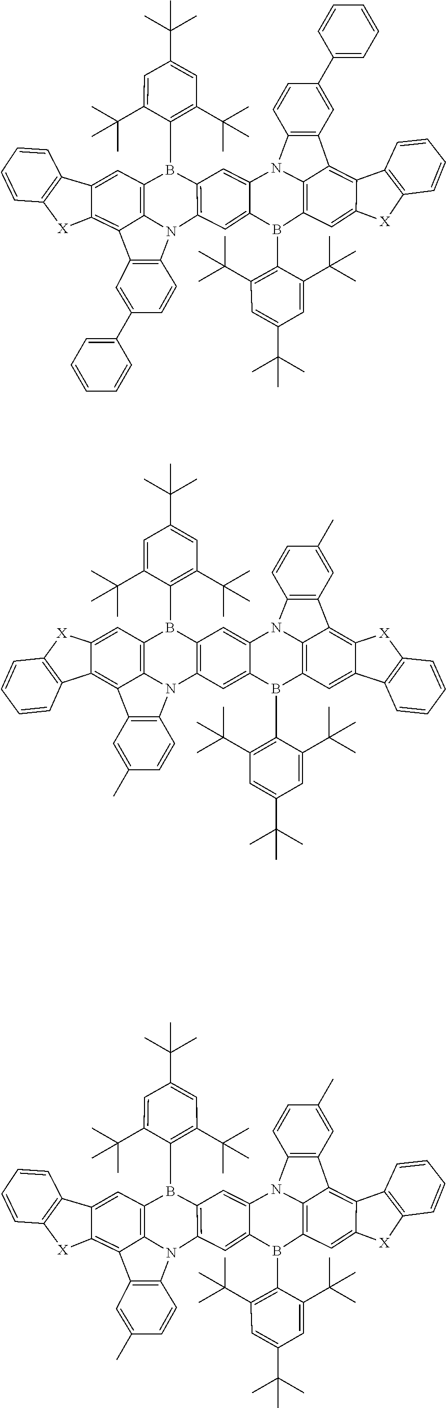

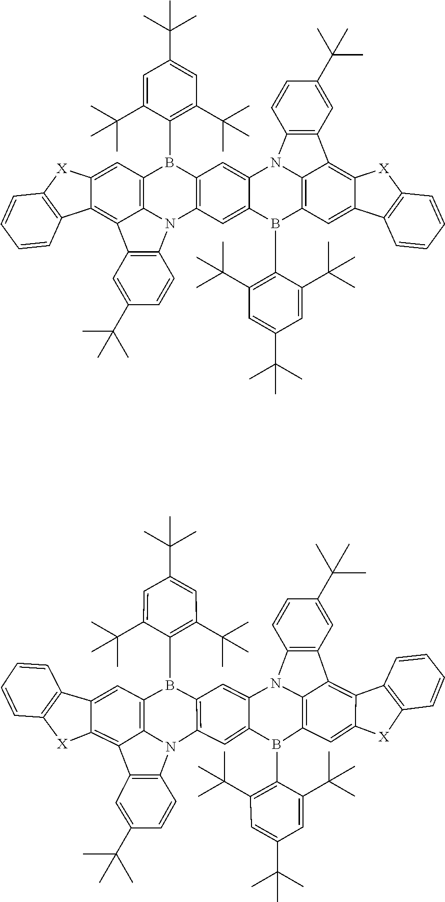

A compound in which benzofuran rings or benzothiophene rings are fused with both of two benzene rings forming a carbazole partial structure existing in the general formula (G) can be preferably mentioned. Examples of such a compound include a compound having the following skeleton (6a), and a compound having the following skeleton (6b).

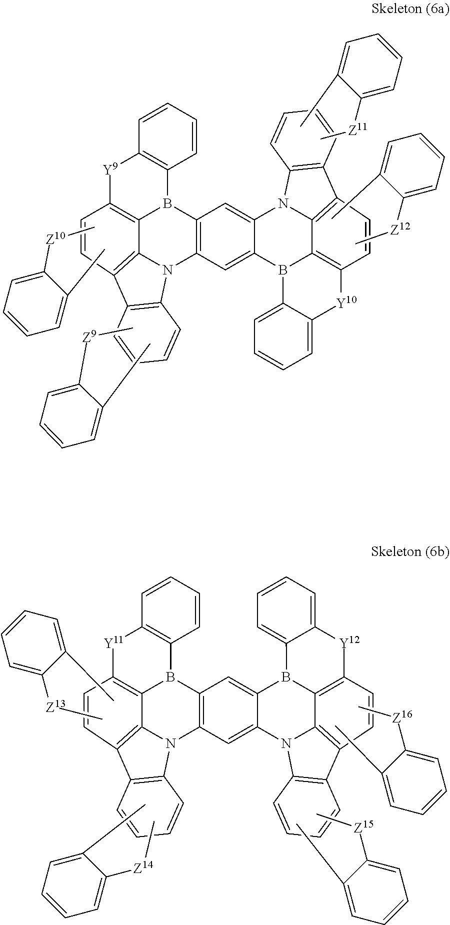

In the skeletons (6a) and (6b), each of Y⁹ to Y¹² independently represents two hydrogen atoms, a single bond or N(R²⁷). Each of Z⁹ to Z¹⁶ independently represents an oxygen atom or a sulfur atom. It is preferable that Z⁹ to Z¹⁶ are the same, but they can be different. In one aspect of the present invention, Z⁹ to Z¹⁶ are oxygen atoms. In one aspect of the present invention, Z⁹ to Z¹⁶ are sulfur atoms. In relation to details of Y⁹ to Y¹², corresponding descriptions for the skeletons (4a) and (4b) can be referred to. In one aspect of the present invention, each hydrogen atom in the skeletons (6a) and (6b) is not substituted with a linking group together with an adjacent hydrogen atom to form a cyclic structure.

As one preferable group of compounds having the skeleton (6a), compounds represented by the following general formula (6a) can be exemplified.

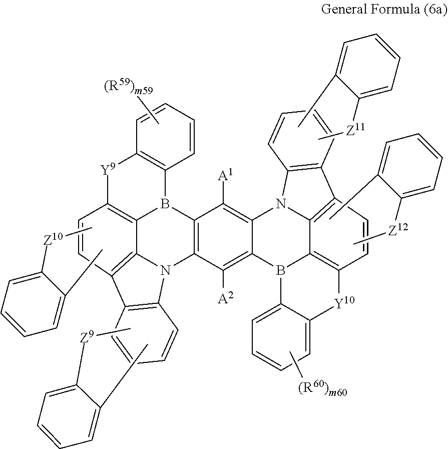

In the general formula (6a), each of R⁵⁹ and R⁶⁰ independently represents a substituted or unsubstituted alkyl group. Each of m59 and m60 independently represents an integer of 0 to 4. Each of Y⁹ and Y¹⁰ independently represents two hydrogen atoms, a single bond or N(R²⁷). R²⁷ represents a hydrogen atom, a deuterium atom, or a substituent. Each of Z⁹ to Z¹² independently represents an oxygen atom or a sulfur atom. Each of A¹ and A² independently represents a hydrogen atom, a deuterium atom, or a substituent. In relation to details of R⁵⁹, R⁶⁰, m59, m60, Z⁹ to Z¹², A¹, and A², descriptions on R⁵⁵, R⁵⁶, m55, m56, A¹, and A² in the general formula (5a) and Z⁹ to Z¹² in the skeleton (6a) can be referred to.

Hereinafter, specific examples of the compound represented by the general formula (6a) will be given. Compounds of the general formula (6a) that can be used in the present invention are not construed as limiting to the following specific examples. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

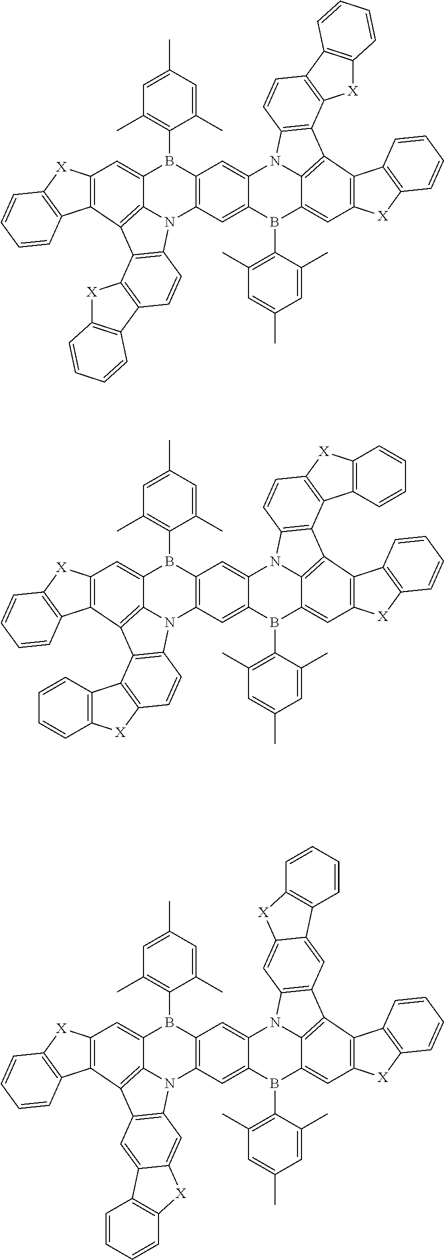

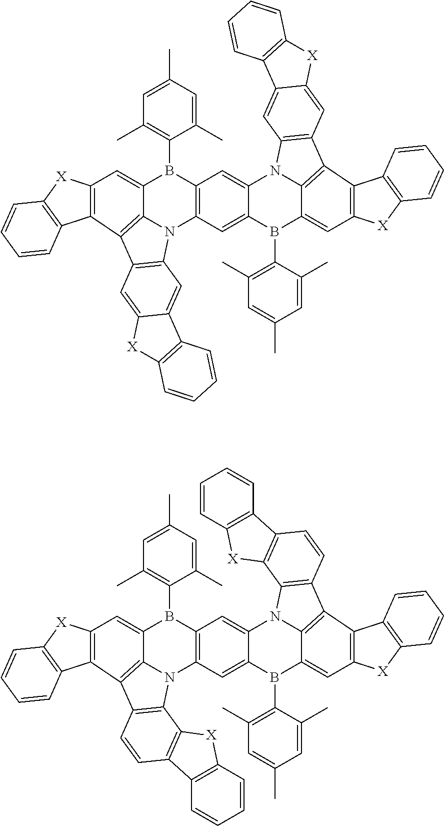

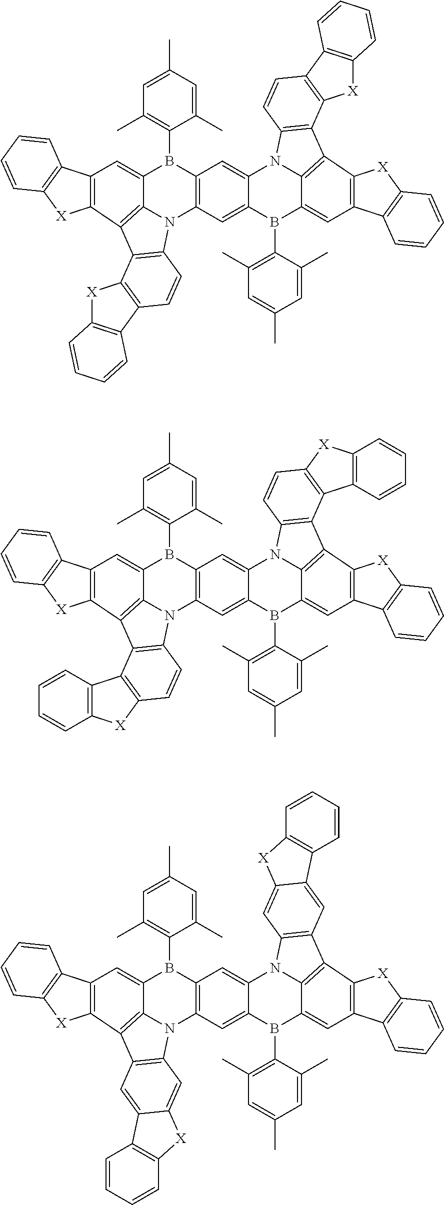

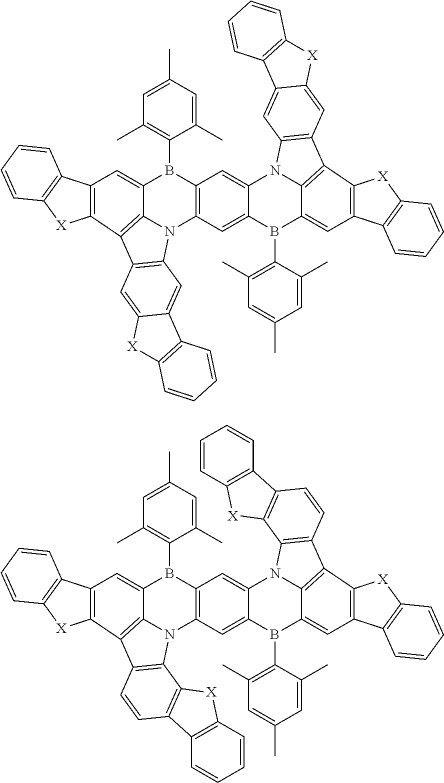

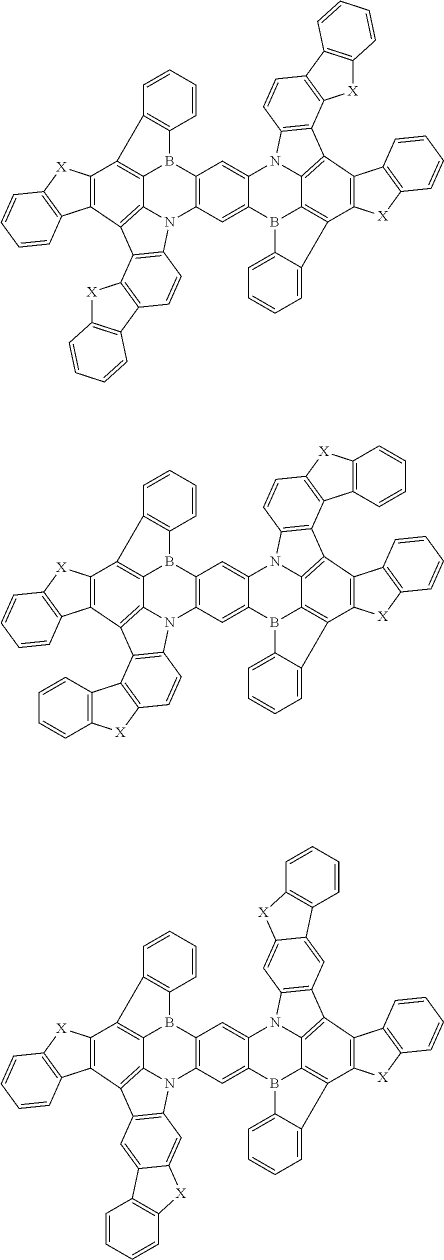

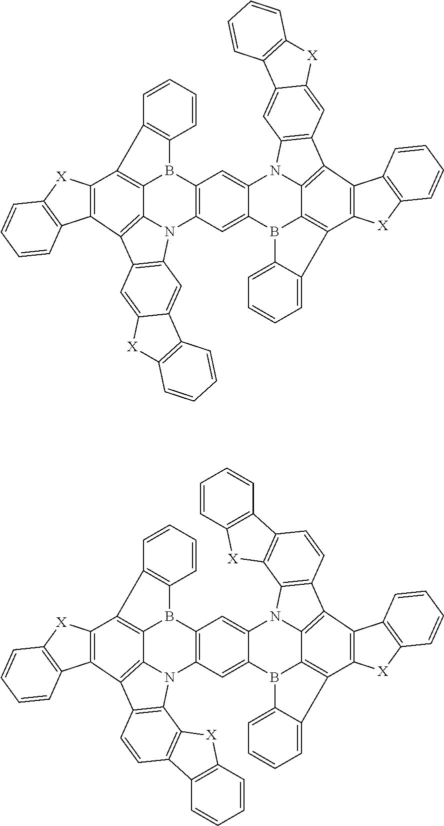

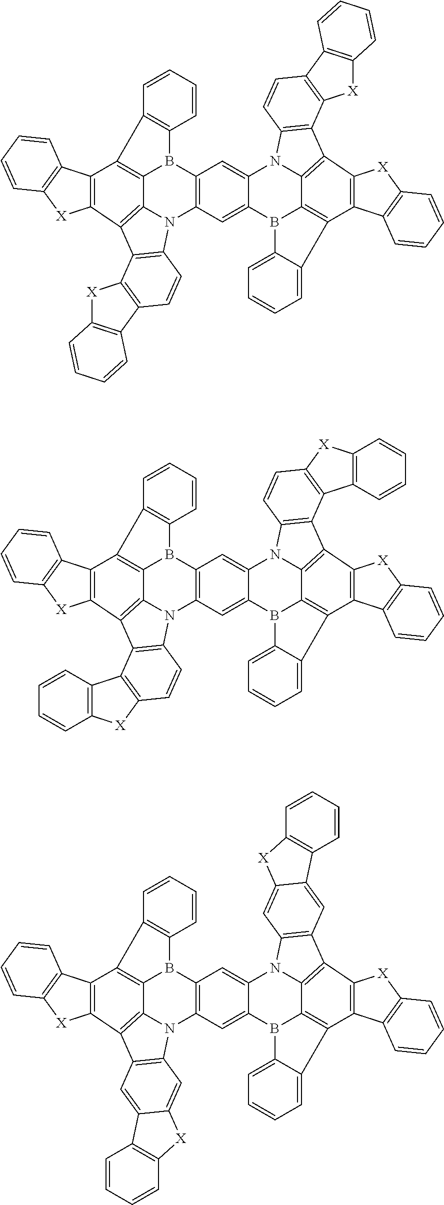

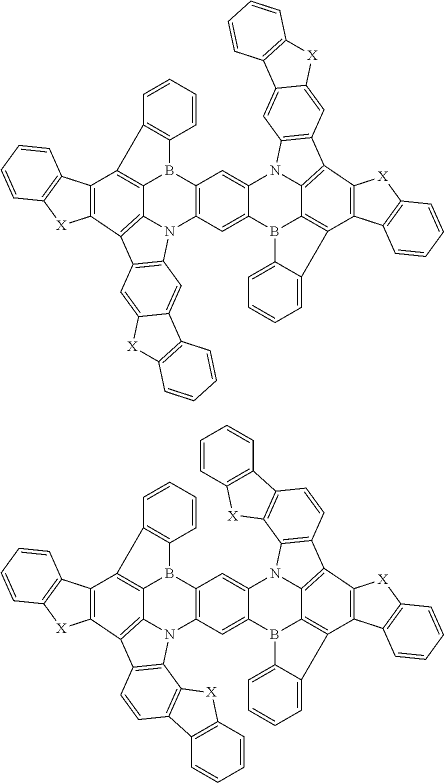

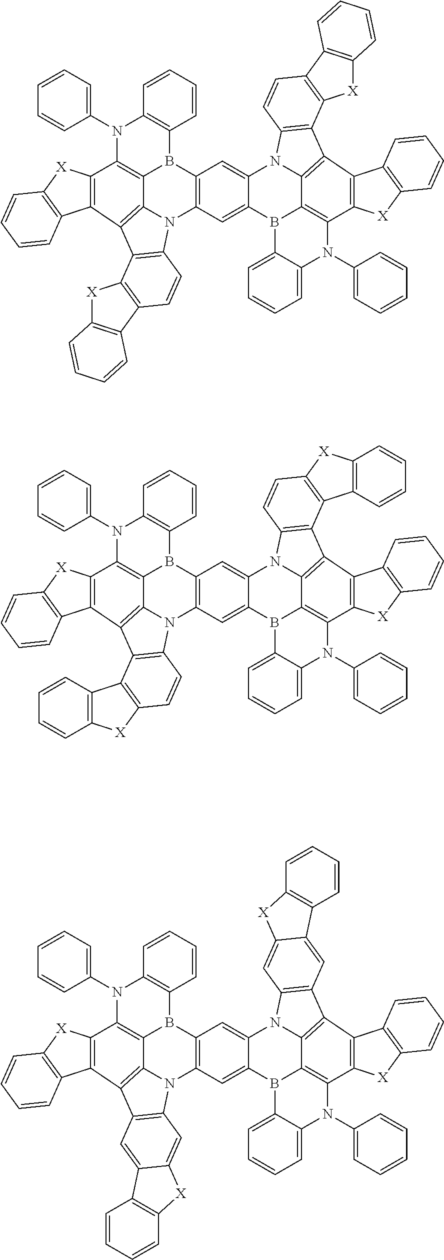

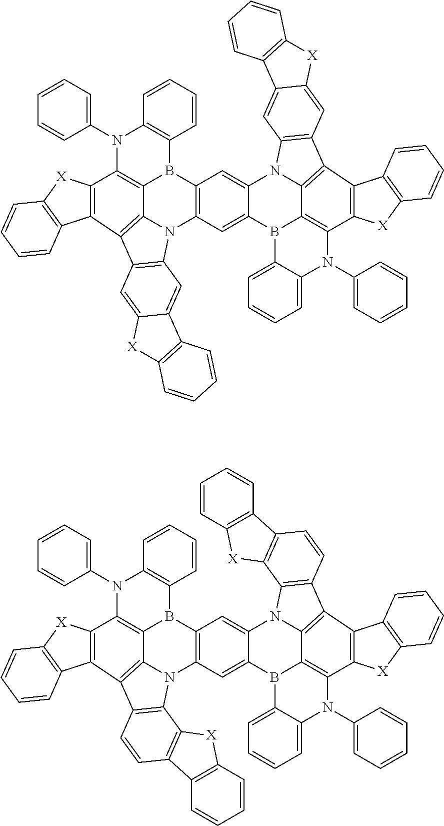

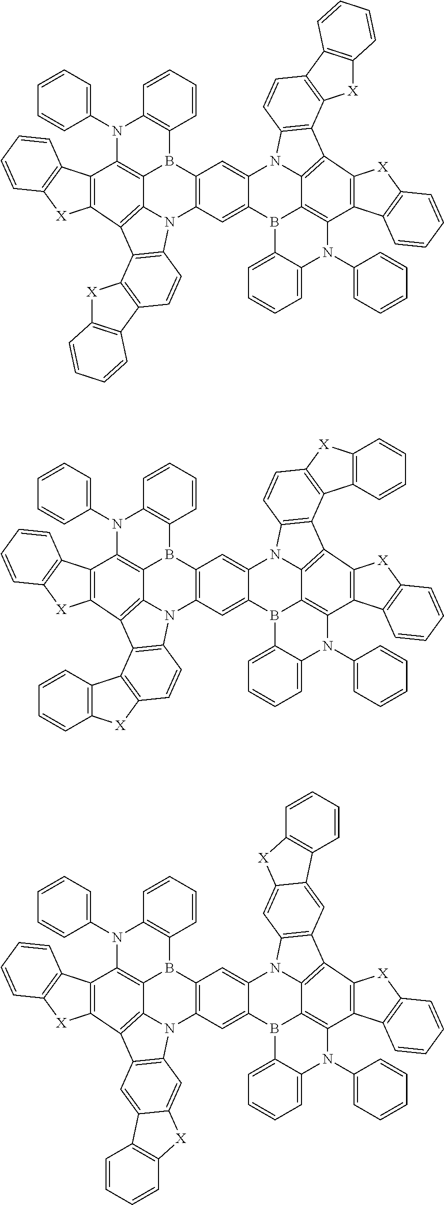

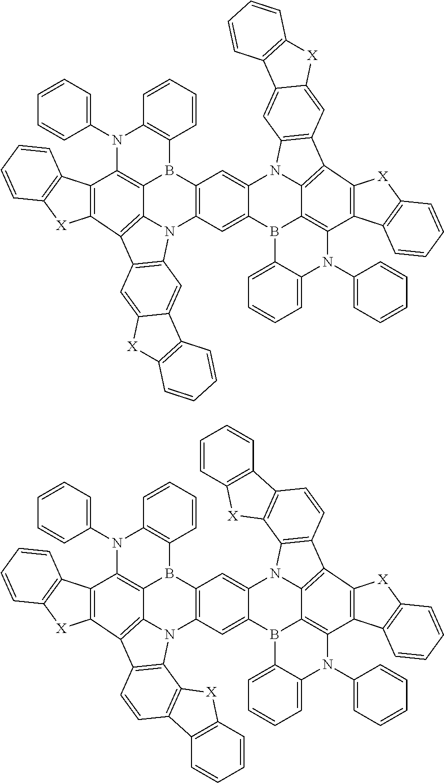

As one preferable group of compounds having the skeleton (6b), compounds represented by the following general formula (6b) can be exemplified.

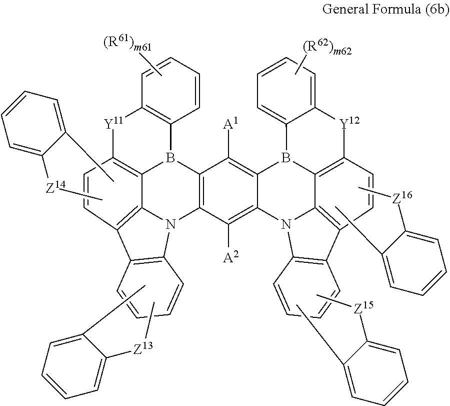

In the general formula (6b), each of R⁶¹ and R⁶² independently represents a substituted or unsubstituted alkyl group. Each of m61 and m60 independently represents an integer of 0 to 4. Each of Y¹¹ and Y¹² independently represents two hydrogen atoms, a single bond or N(R²⁷). R²⁷ represents a hydrogen atom, a deuterium atom, or a substituent. Each of Z¹³ to Z¹⁶ independently represents an oxygen atom or a sulfur atom. Each of A¹ and A² independently represents a hydrogen atom, a deuterium atom, or a substituent. In relation to details of R⁶¹, R⁶², m61, m62, Z¹³ to Z¹⁶, A¹, and A², descriptions on R⁵⁹, R⁶⁰, m59, m60, A¹, and A² in the general formula (6a), and Z¹³ to Z¹⁶ in the skeleton (6b) can be referred to.

Hereinafter, specific examples of the compound represented by the general formula (6b) will be given. Compounds of the general formula (6b) that can be used in the present invention are not construed as limiting to the following specific examples. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

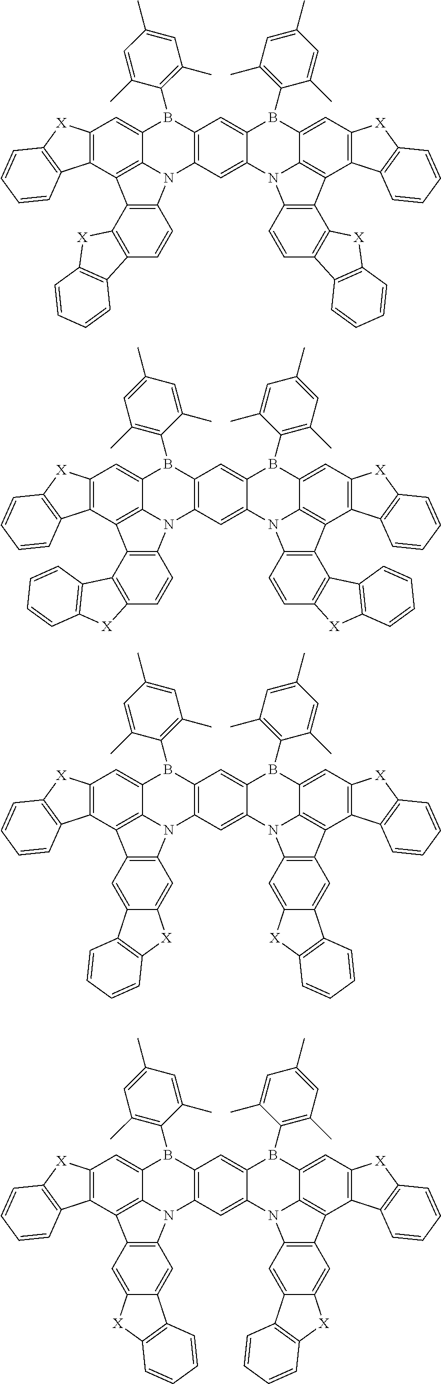

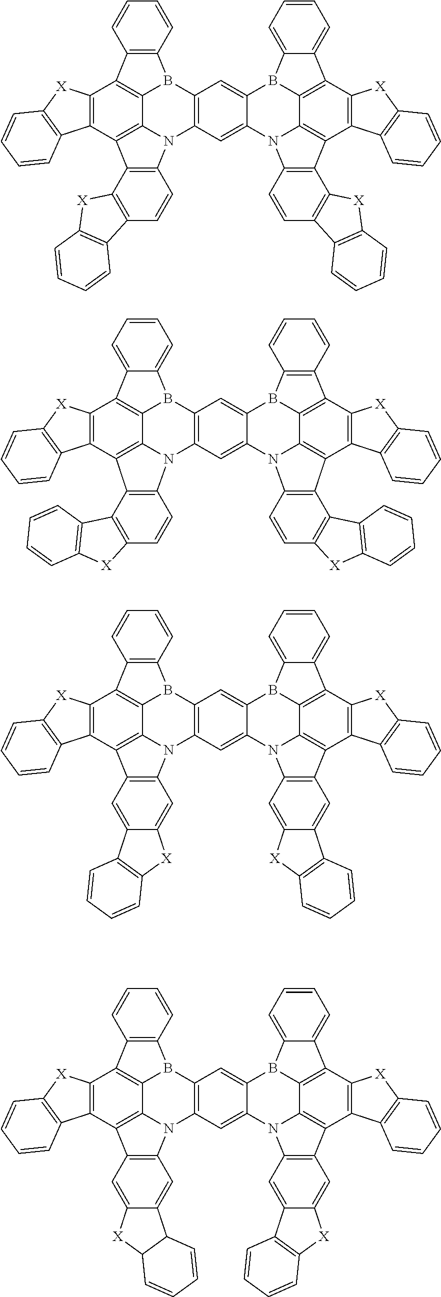

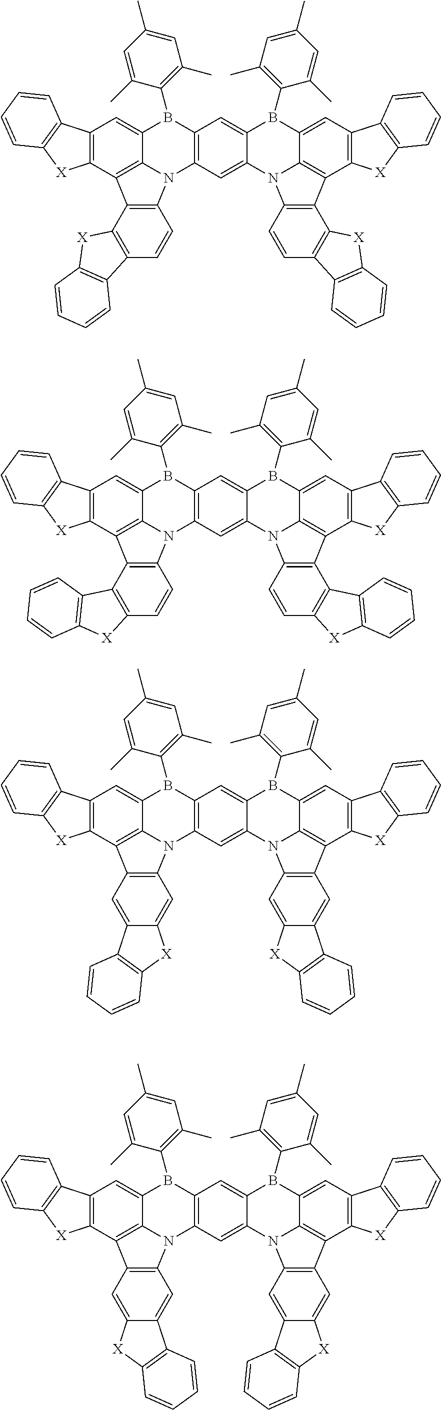

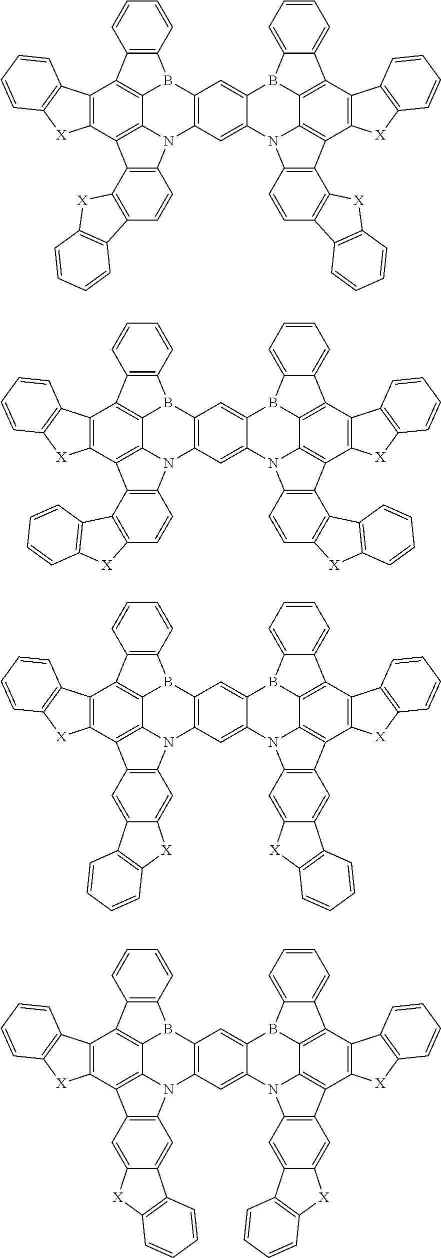

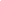

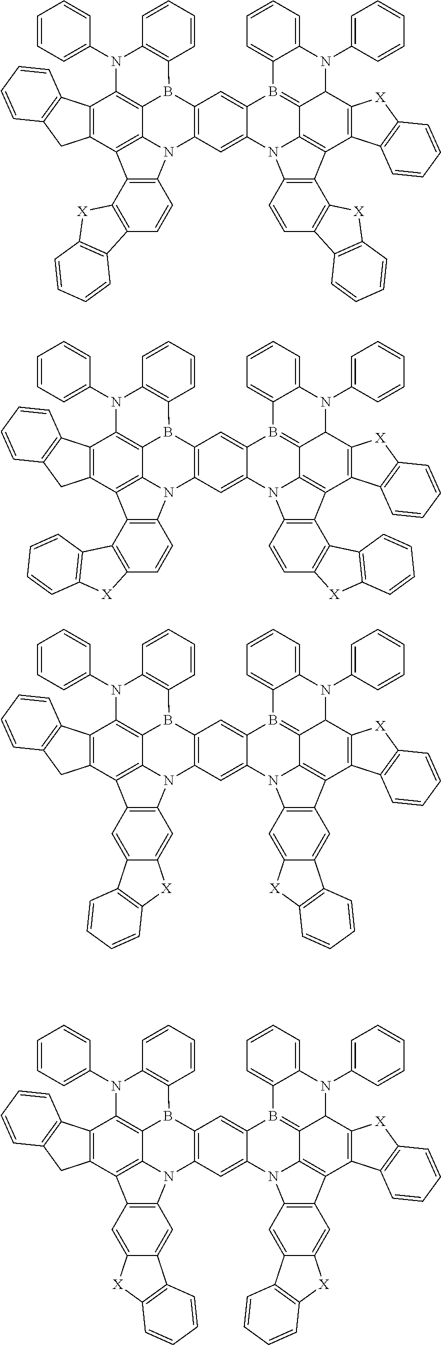

A compound in which a benzene ring is fused with a benzene ring to which a boron atom does not directly bond, between two benzene rings forming a carbazole partial structure existing in the general formula (G), can be preferably mentioned. Examples of such a compound include a compound having the following skeleton (7a), and a compound having the following skeleton (7b).

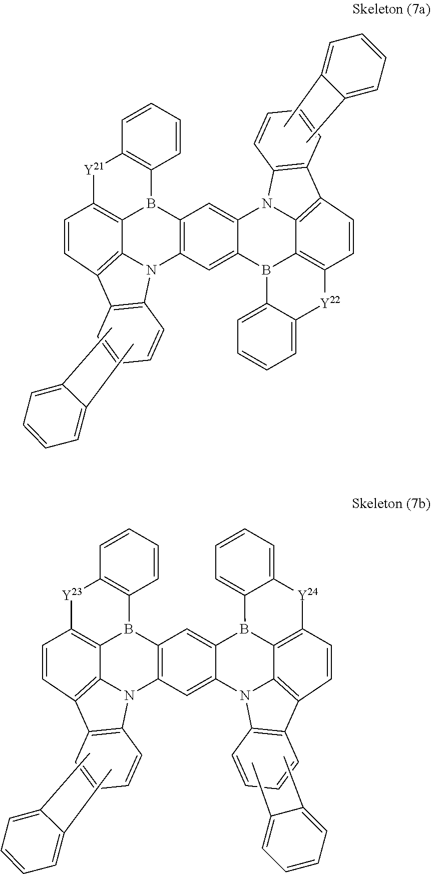

In the skeletons (7a) and (7b), each of Y²¹ to Y²⁴ independently represents two hydrogen atoms, a single bond or N(R²⁷). In relation to details of Y²¹ to Y²⁴, descriptions on Y¹ to Y⁴ in the skeletons (4a) and (4b) can be referred to. In one aspect of the present invention, each hydrogen atom in the skeletons (7a) and (7b) is not substituted with a linking group together with an adjacent hydrogen atom to form a cyclic structure.

As one preferable group of compounds having the skeleton (7a), compounds represented by the following general formula (7a) can be exemplified.

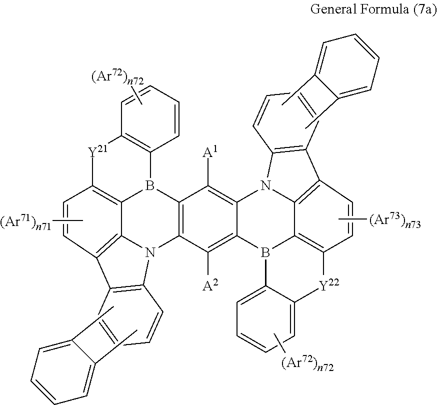

In the general formula (7a), each of Ar⁷¹ to Ar⁷⁴ independently represents a substituted or unsubstituted aryl group, a substituted or unsubstituted heteroaryl group, or a substituted or unsubstituted alkyl group, and, for example, a substituted or unsubstituted aryl group can be preferably selected. Each of n71 and n73 independently represents an integer of 0 to 2. Each of n72 and n74 independently represents an integer of 0 to 4. Each of Y²¹ and Y²² independently represents two hydrogen atoms, a single bond or N(R²⁷). R²⁷ represents a hydrogen atom, a deuterium atom, or a substituent. Each of A¹ and A² independently represents a hydrogen atom, a deuterium atom, or a substituent.

In one aspect of the present invention, n71 to n74 are integers of 0 to 2. In one aspect of the present invention, n71 and n73 are the same number, and n72 and n74 are the same number. n71 to n74 can be the same number. For example, n71 to n74 can be 0. n71 to n74 can be all 1. Further, for example, n71 and n73 can be 0, and n72 and n74 can be 1. In relation to preferable groups for Ar⁷¹ to Ar⁷⁴, A¹, and A², corresponding descriptions on Ar¹ to Ar⁴, A¹, and A² in the general formula (I a) can be referred to.

Hereinafter, specific examples of the compound represented by the general formula (7a) will be given. Compounds of the general formula (7a) that can be used in the present invention are not construed as limiting to the following specific examples.

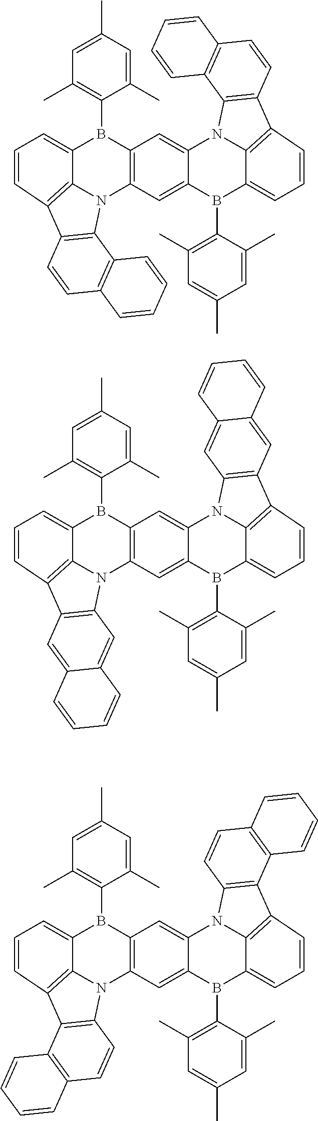

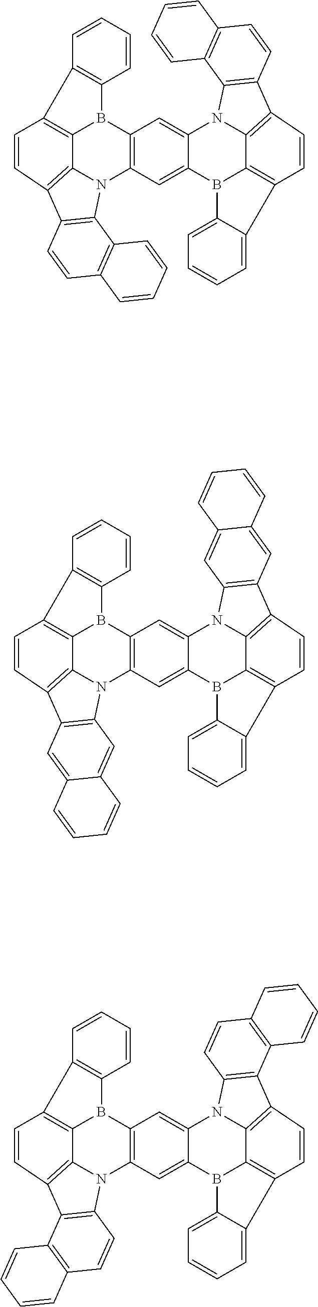

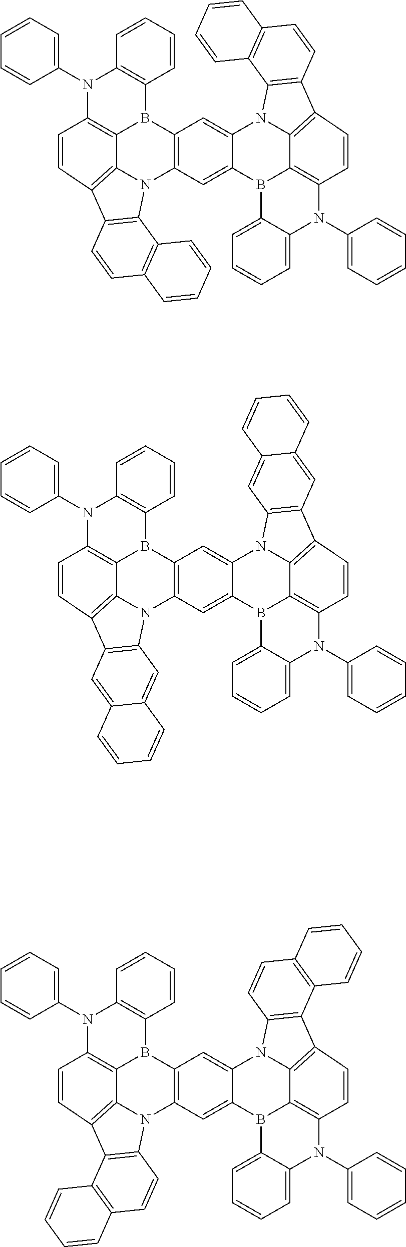

As one preferable group of compounds having the skeleton (7b), compounds represented by the following general formula (7b) can be exemplified.

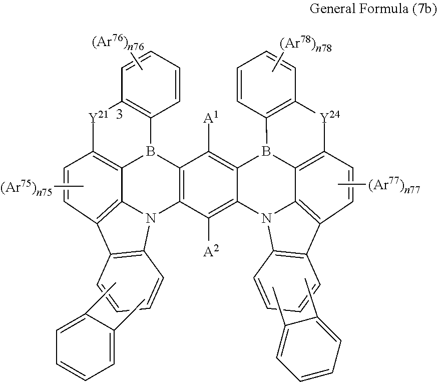

In the general formula (7b), each of Ar⁷⁵ to Ar⁷⁸ independently represents a substituted or unsubstituted aryl group, a substituted or unsubstituted heteroaryl group, or a substituted or unsubstituted alkyl group, and, for example, a substituted or unsubstituted aryl group can be preferably selected. Each of n75 and n77 independently represents an integer of 0 to 2. Each of n76 and n78 independently represents an integer of 0 to 4. Each of Y²³ and Y²⁴ independently represents two hydrogen atoms, a single bond or N(R²⁷). R²⁷ represents a hydrogen atom, a deuterium atom, or a substituent. For detailed descriptions of n75 to n78, descriptions on n71 to n74 in the general formula (7a) can be referred to in this order. In relation to preferable groups for Ar⁷⁵ to Ar⁷⁸, corresponding descriptions on Ar¹ to Ar⁴ in the general formula (1a) can be referred to.

Hereinafter, specific examples of the compound represented by the general formula (7b) will be given. Compounds of the general formula (7b) that can be used in the present invention are not construed as limiting to the following specific examples.

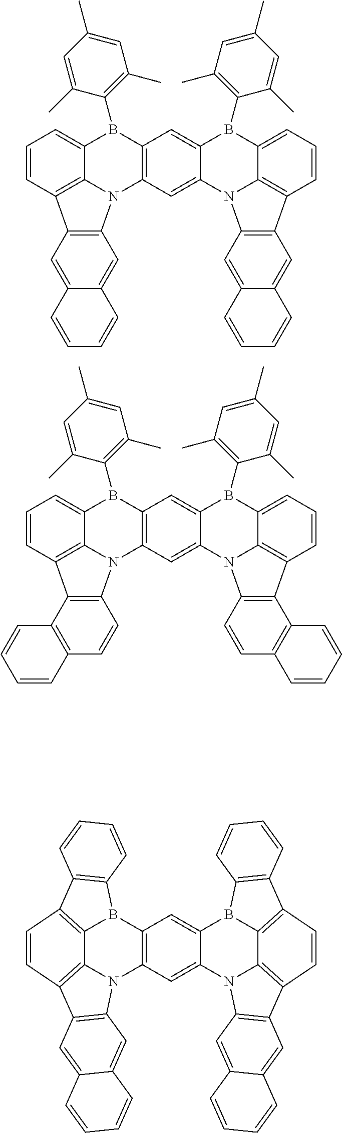

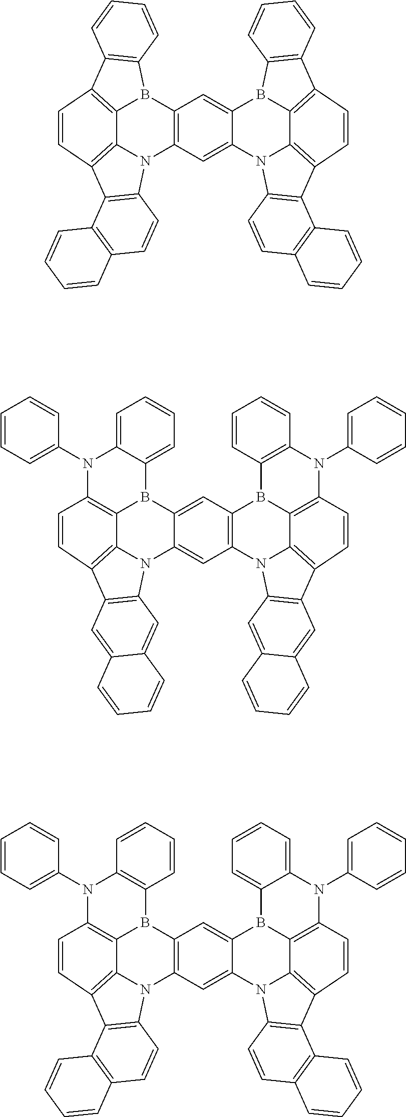

A compound in which a benzene ring is fused with a benzene ring to which a boron atom directly bonds, between two benzene rings forming a carbazole partial structure existing in the general formula (G), can be preferably mentioned. Examples of such a compound include a compound having the following skeleton (8a), and a compound having the following skeleton (8b).

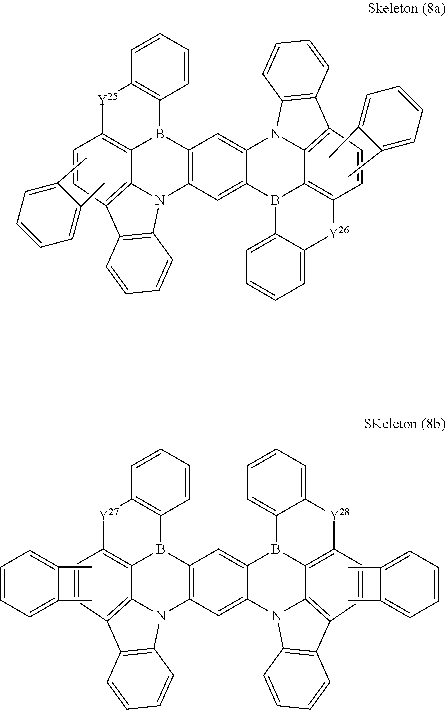

In the skeletons (8a) and (8b), each of Y²⁵ to Y²⁸ independently represents two hydrogen atoms, a single bond or N(R²⁷). In relation to details of Y²⁵ to Y²⁸, corresponding descriptions for the skeletons (4a) and (4b) can be referred to. In one aspect of the present invention, each hydrogen atom in the skeletons (8a) and (8b) is not substituted with a linking group together with an adjacent hydrogen atom to form a cyclic structure.

As one preferable group of compounds having the skeleton (8a), compounds represented by the following general formula (8a) can be exemplified.

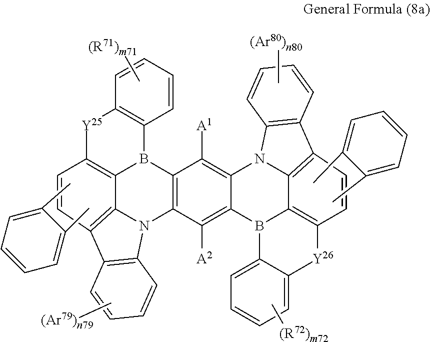

In the general formula (8a), each of Ar⁷⁹ and Ar⁸⁰ independently represents a substituted or unsubstituted aryl group, a substituted or unsubstituted heteroaryl group, or a substituted or unsubstituted alkyl group, and, for example, a substituted or unsubstituted aryl group can be preferably selected. Each of R⁷¹ and R⁷² independently represents a substituted or unsubstituted alkyl group. Each of m71 and m72 independently represents an integer of 0 to 4. Each of n79 and n80 independently represents an integer of 0 to 4. Each of Y²⁵ and Y²⁶ independently represents two hydrogen atoms, a single bond or N(R²⁷). R²⁷ represents a hydrogen atom, a deuterium atom, or a substituent. Each of A¹ and A² independently represents a hydrogen atom, a deuterium atom, or a substituent.

In one aspect of the present invention, n79 and n80 are integers of 0 to 2. In one aspect of the present invention, n79 and n80 are the same number, and for example, can be all 0, or can be all 1. In one aspect of the present invention, m71 and m72 are integers of 0 to 2. In one aspect of the present invention, m71 and m72 are the same number, and for example, can be all 0, or can be all 1. In relation to preferable groups for Ar⁷⁹, Ar⁸⁰, R⁷¹, R⁷², A¹, and A², corresponding descriptions on Ar¹, Ar³, R⁴¹, R⁴², A¹, and A² in the general formula (1a) can be referred to.

Hereinafter, specific examples of the compound represented by the general formula (8a) will be given. Compounds of the general formula (8a) that can be used in the present invention are not construed as limiting to the following specific examples.

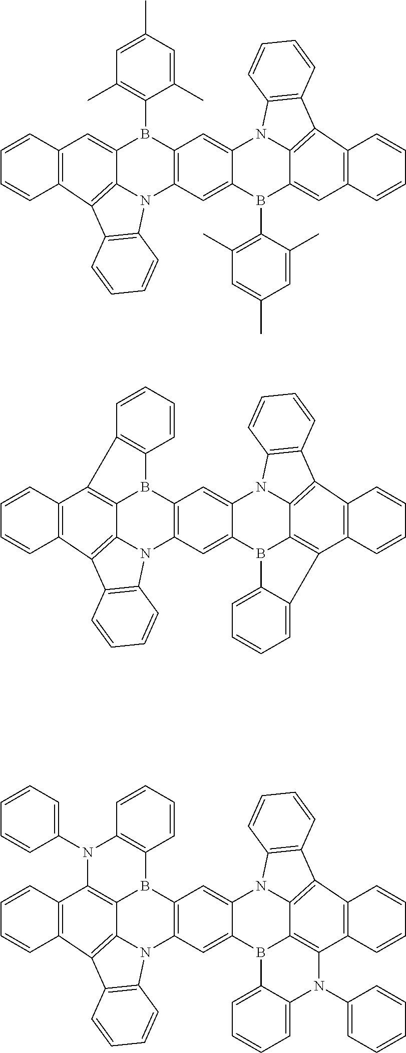

As one preferable group of compounds having the skeleton (8b), compounds represented by the following general formula (8b) can be exemplified.

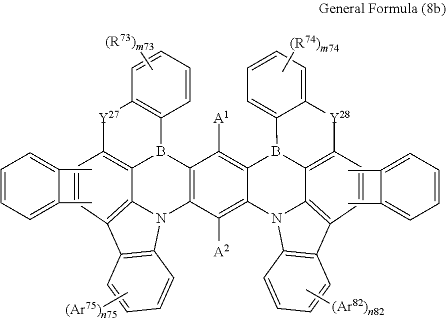

In the general formula (8b), each of Ar⁸¹ and Ar⁸² independently represents a substituted or unsubstituted aryl group, a substituted or unsubstituted heteroaryl group, or a substituted or unsubstituted alkyl group, and, for example, a substituted or unsubstituted aryl group can be preferably selected. Each of R⁷³ and R⁷⁴ independently represents a substituted or unsubstituted alkyl group. Each of m73 and m74 independently represents an integer of 0 to 4. Each of n81 and n82 independently represents an integer of 0 to 4. Each of Y²⁷ and Y²⁸ independently represents two hydrogen atoms, a single bond or N(R²⁷). R²⁷ represents a hydrogen atom, a deuterium atom, or a substituent. Each of A¹ and A² independently represents a hydrogen atom, a deuterium atom, or a substituent.

In relation to detailed descriptions of m73, m74, n81, and n82, descriptions on m71, m72, n79, and n80 in the general formula (8a) can be referred to. In relation to preferable groups for Ar⁸¹, Ar⁸², R⁷³, R⁷⁴, A¹, and A², corresponding descriptions on Ar¹, Ar³, R⁴¹, R⁴², A¹, and A² in the general formula (1a) can be referred to.

Hereinafter, specific examples of the compound represented by the general formula (8b) will be given. Compounds of the general formula (8b) that can be used in the present invention are not construed as limiting to the following specific examples.

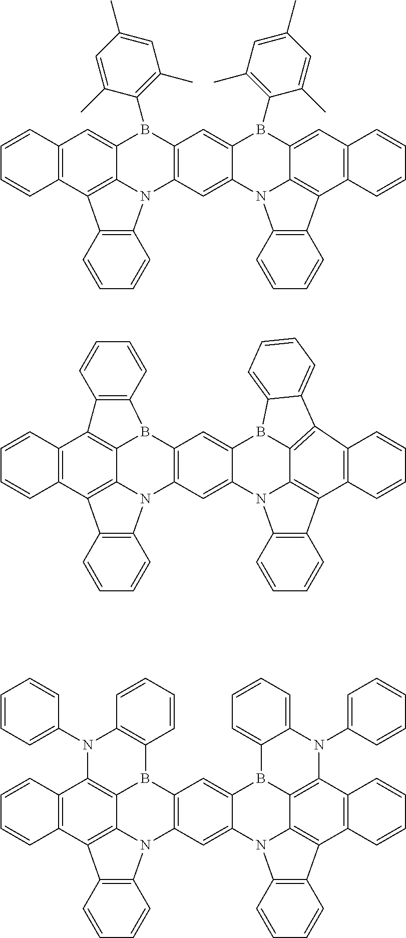

A compound in which benzene rings are fused with both of two benzene rings forming a carbazole partial structure existing in the general formula (G) can be preferably mentioned. Examples of such a compound include a compound having the following skeleton (9a), and a compound having the following skeleton (9b).

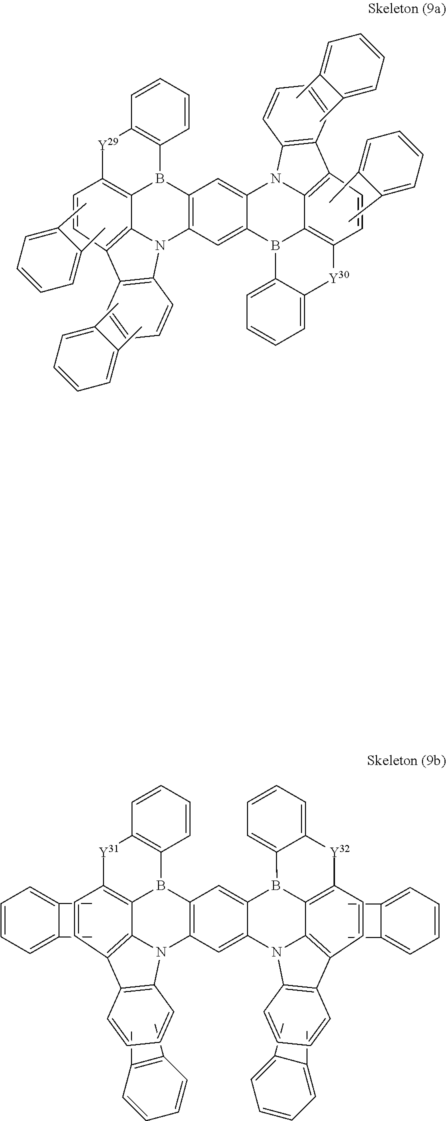

In the skeletons (9a) and (9b), each of Y²⁹ to Y³² independently represents two hydrogen atoms, a single bond or N(R²⁷). In relation to details of Y²⁹ to Y³², corresponding descriptions for the skeletons (4a) and (4b) can be referred to. In one aspect of the present invention, each hydrogen atom in the skeletons (9a) and (9b) is not substituted with a linking group together with an adjacent hydrogen atom to form a cyclic structure.

As one preferable group of compounds having the skeleton (9a), compounds represented by the following general formula (9a) can be exemplified.

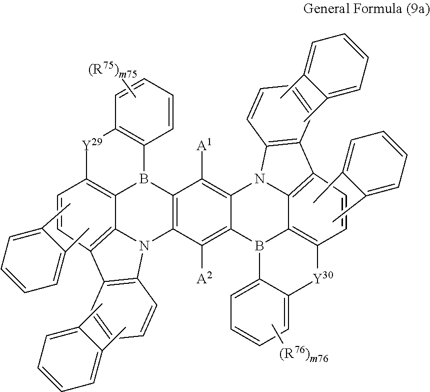

In the general formula (9a), each of R⁷⁵ and R⁷⁶ independently represents a substituted or unsubstituted alkyl group. Each of m75 and m76 independently represents an integer of 0 to 4. Each of Y²⁹ and Y³⁰ independently represents two hydrogen atoms, a single bond or N(R²⁷). R²⁷ represents a hydrogen atom, a deuterium atom, or a substituent. Each of A¹ and A² independently represents a hydrogen atom, a deuterium atom, or a substituent. In relation to details of R⁷⁵, R⁷⁶, m75, m76, A¹, and A², descriptions on R⁷¹, R⁷², m71, m72, A¹, and A² in the general formula (8a) can be referred to.

Hereinafter, specific examples of the compound represented by the general formula (9a) will be given. Compounds of the general formula (9a) that can be used in the present invention are not construed as limiting to the following specific examples.

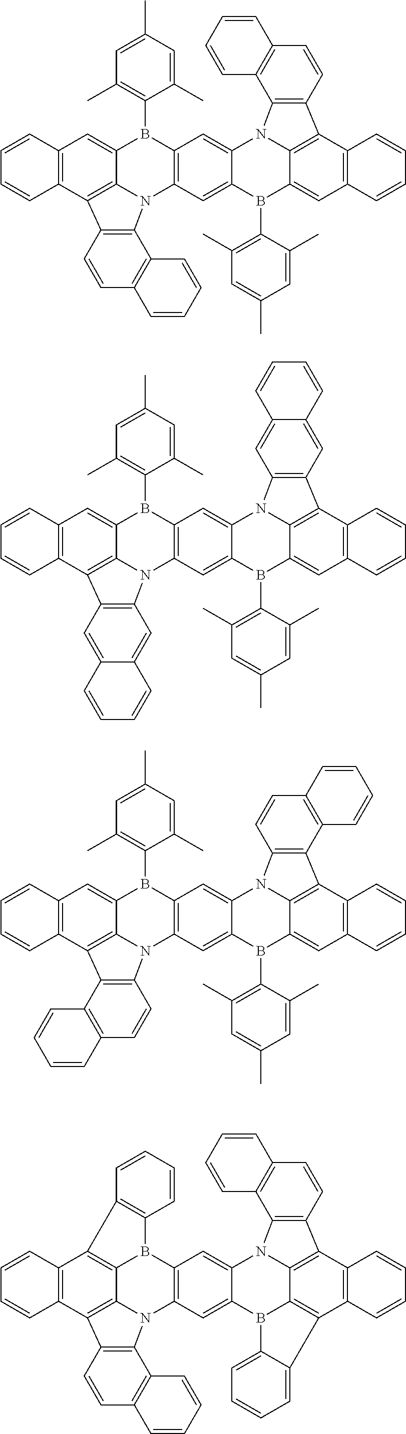

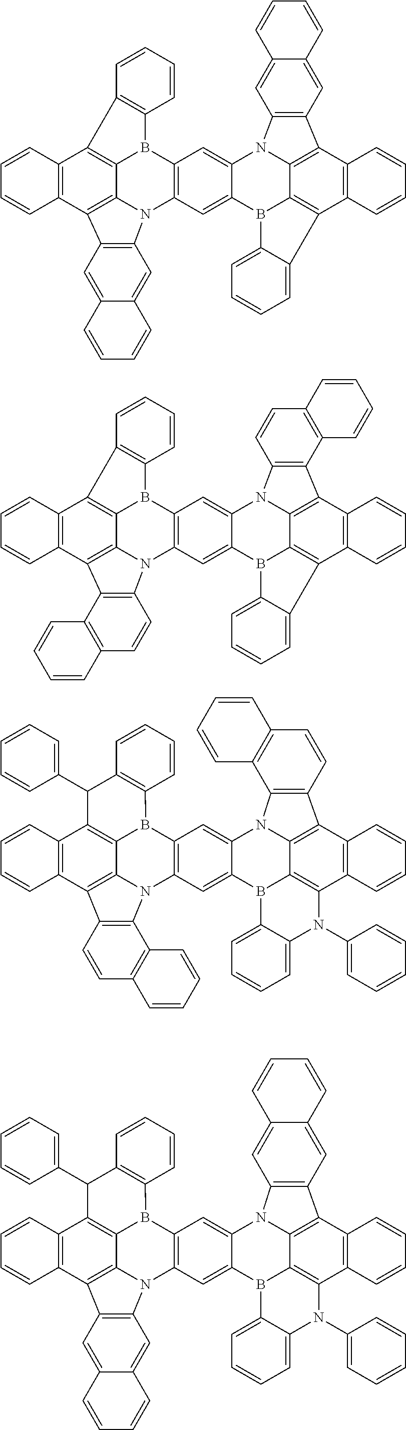

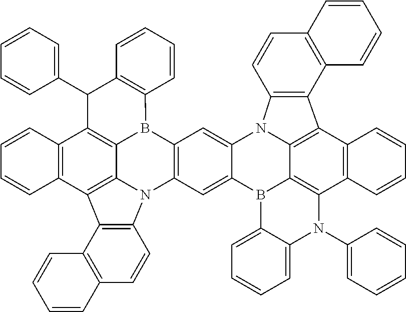

As one preferable group of compounds having the skeleton (9b), compounds represented by the following general formula (9b) can be exemplified.

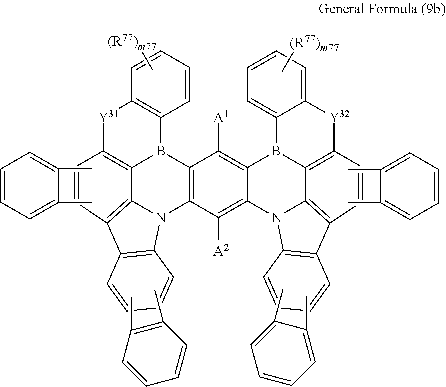

In the general formula (9b), each of R⁷⁷ and R⁷⁸ independently represents a substituted or unsubstituted alkyl group. Each of m77 and m78 independently represents an integer of 0 to 4. Each of Y³¹ and Y³² independently represents two hydrogen atoms, a single bond or N(R²⁷). R²⁷ represents a hydrogen atom, a deuterium atom, or a substituent. Each of A¹ and A² independently represents a hydrogen atom, a deuterium atom, or a substituent. In relation to details of R⁷⁷, R⁷⁸, m77, m78, A¹, and A², descriptions on R⁷¹, R⁷², m71, m72, A¹, and A² in the general formula (8a) can be referred to.

Hereinafter, specific examples of the compound represented by the general formula (9b) will be given. Compounds of the general formula (9b) that can be used in the present invention are not construed as limiting to the following specific examples.

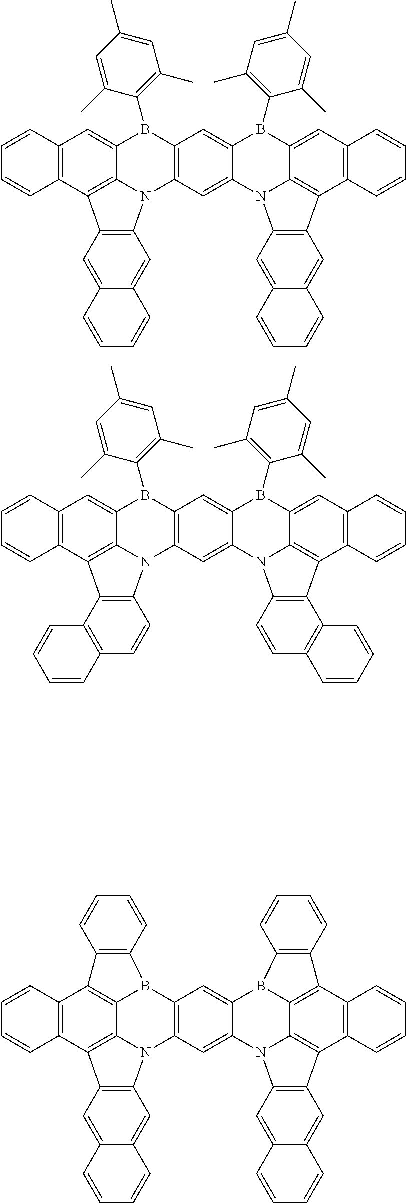

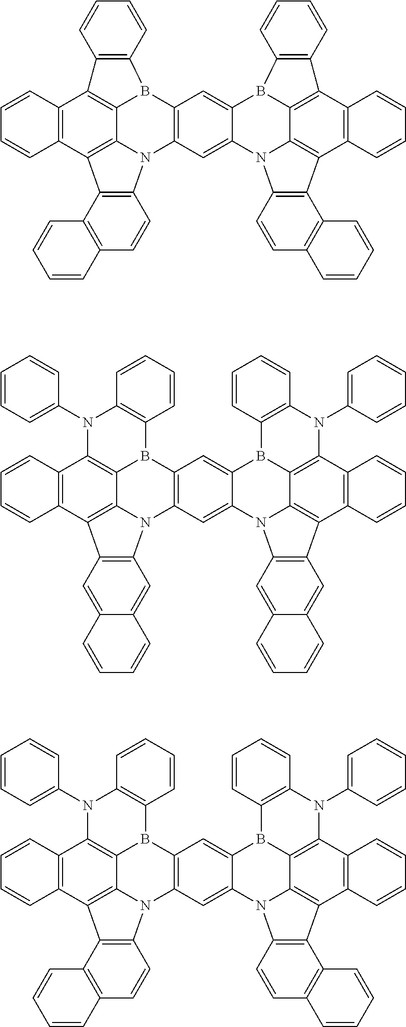

As the compound represented by the general formula (G), a compound in which four or more carbazole partial structures are included in the molecule is also preferable. As an example of such a compound, a compound having the following skeleton (10) can be exemplified.

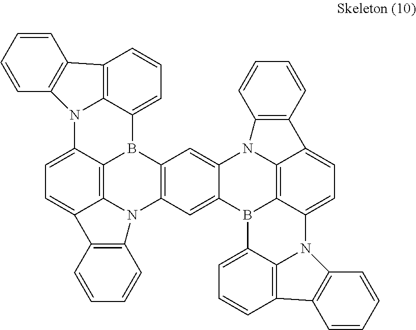

Each hydrogen atom in the skeleton (10) can be substituted with a deuterium atom or a substituent. Further, it can be substituted with a linking group together with an adjacent hydrogen atom to form a cyclic structure. For details, corresponding descriptions on R¹ to R²⁶, A¹, and A² in the general formula (G) can be referred to. At least one hydrogen atom of a benzene ring forming a carbazole partial structure included in the skeleton (10) is substituted with a substituted or unsubstituted aryl group. In one aspect of the present invention, each hydrogen atom in the skeleton (10) is not substituted with a linking group together with an adjacent hydrogen atom to form a cyclic structure.

As one preferable group of compounds having the skeleton (10), compounds represented by the following general formula (10) can be exemplified.

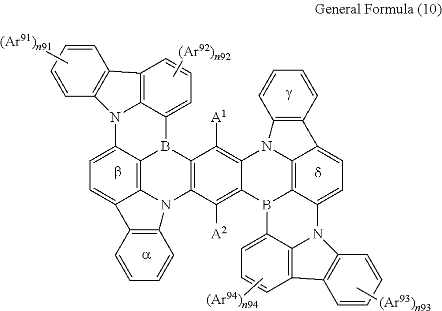

In the general formula (10), each of Ar⁹¹ to Ar⁹⁴ independently represents a substituted or unsubstituted aryl group, a substituted or unsubstituted heteroaryl group, or a substituted or unsubstituted alkyl group, and, for example, a substituted or unsubstituted aryl group can be preferably selected. Each of n91 and n93 independently represents an integer of 0 to 4, and each of n92 and n94 independently represents an integer of 0 to 3. An α ring, a β ring, a γ ring, and a δ ring can be substituted, and at least one ring is substituted with a substituted or unsubstituted aryl group, is fused with a benzene ring that can be substituted, or is fused with a substituted or unsubstituted furan ring of benzofuran or a substituted or unsubstituted thiophene ring of thiophene. Each of A¹ and A² independently represents a hydrogen atom, a deuterium atom, or a substituent.

In one aspect of the present invention, n91 to n94 are integers of 0 to 2. In one aspect of the present invention, n91 and n93 are the same number, and n92 and n94 are the same number. n91 to n94 can be all the same number, and for example can be all 0, or can be all 1. In relation to preferable groups for Ar⁹¹ to Ar⁹⁴, corresponding descriptions on Ar¹ to Ar⁴ in the general formula (1a) can be referred to. In one aspect of the present invention, the α ring and the γ ring have the same substituents or have the same fused structures, and the β ring and the δ ring have the same substituents or have the same fused structures. In one aspect of the present invention, both the β ring and the δ ring are substituted with substituted or unsubstituted aryl groups, are fused with benzene rings that can be substituted, or are fused with substituted or unsubstituted furan rings of benzofuran or substituted or unsubstituted thiophene rings of thiophene. In one aspect of the present invention, both the α ring and the γ ring are substituted with substituted or unsubstituted aryl groups, are fused with benzene rings that can be substituted, or are fused with substituted or unsubstituted furan rings of benzofuran or substituted or unsubstituted thiophene rings of thiophene. In one aspect of the present invention, all of the α ring, the β ring, the γ ring, and the δ ring are substituted with substituted or unsubstituted aryl groups, are fused with benzene rings that can be substituted, or are fused with substituted or unsubstituted furan rings of benzofuran or substituted or unsubstituted thiophene rings of thiophene. For descriptions and preferable ranges of A¹ and A², corresponding descriptions on the general formula (G) can be referred to.

Hereinafter, specific examples of the compound represented by the general formula (10) will be given. Compounds of the general formula (10) that can be used in the present invention are not construed as limiting to the following specific examples.

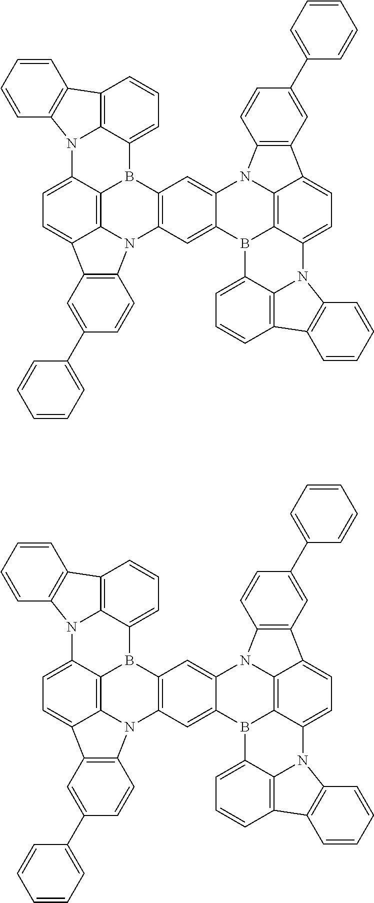

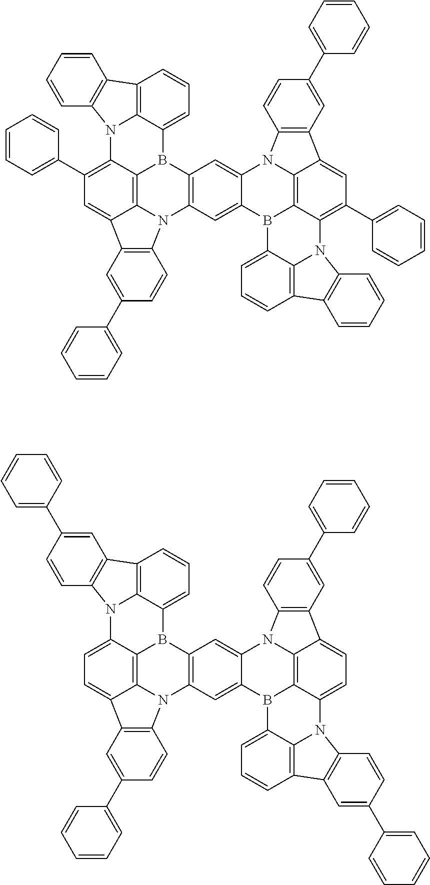

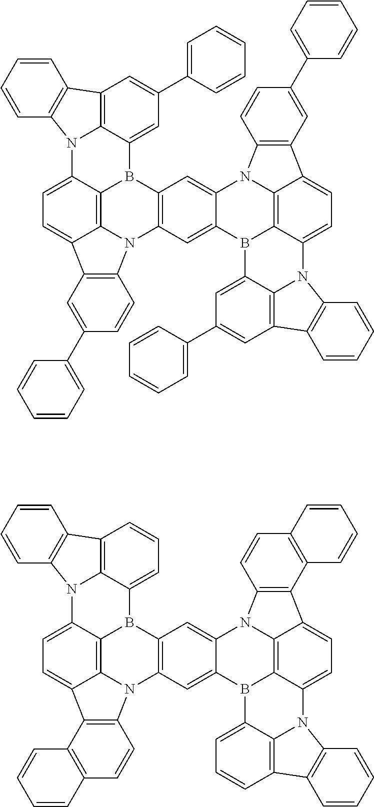

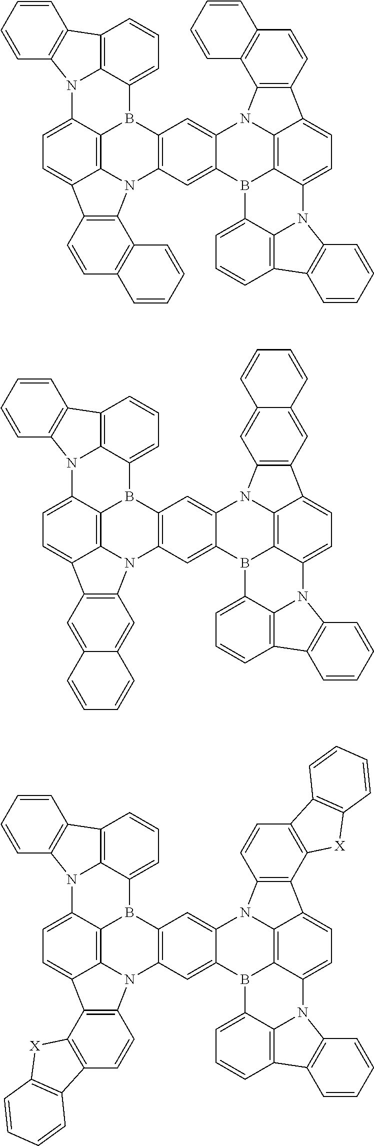

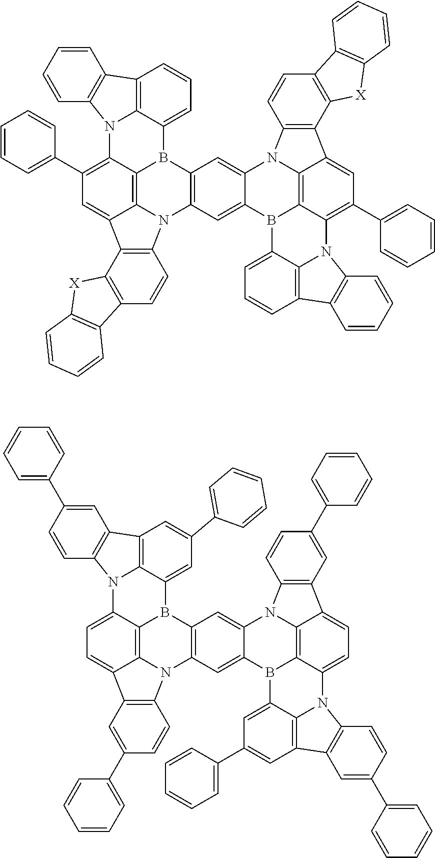

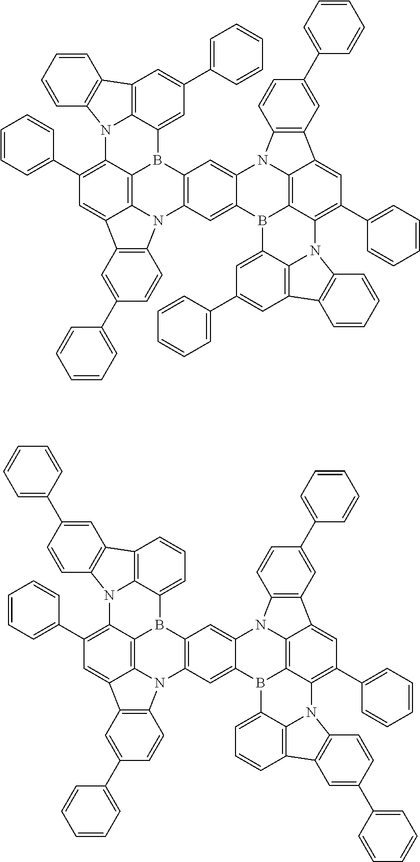

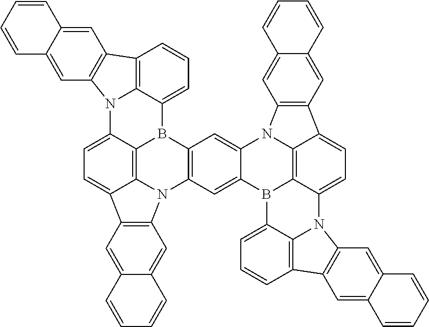

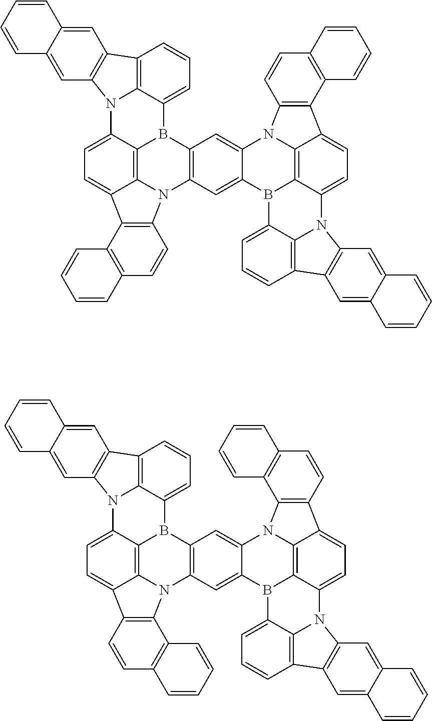

The compound represented by the general formula (G) can have a skeleton having no symmetry. For example, it can be a compound having an asymmetric skeleton such as the following skeleton (11a) or the following skeleton (11b).

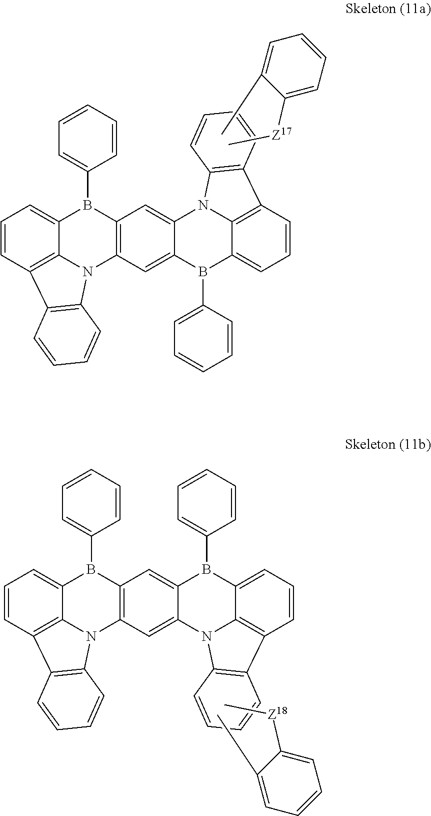

In the skeletons (11a) and (11b), each of Z¹⁷ and Z¹⁸ independently represents an oxygen atom or a sulfur atom. In one aspect of the present invention, each hydrogen atom in the skeletons (11a) and (11b) is not substituted with a linking group together with an adjacent hydrogen atom to form a cyclic structure.

As one preferable group of compounds having the skeleton (11a), compounds represented by the following general formula (11a) can be exemplified.

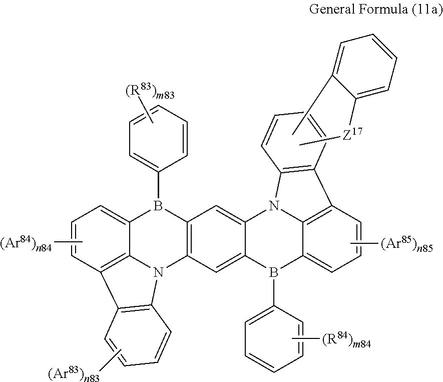

In the general formula (11a), each of Ar⁸³ to Ar⁸⁵ independently represents a substituted or unsubstituted aryl group, a substituted or unsubstituted heteroaryl group, or a substituted or unsubstituted alkyl group, and, for example, a substituted or unsubstituted aryl group can be preferably selected. Each of R⁸³ and R⁸⁴ independently represents a substituted or unsubstituted alkyl group. Z¹⁷ represents an oxygen atom or a sulfur atom. Each of m83 and m84 independently represents an integer of 0 to 5. n83 represents an integer of 0 to 4, and each of n84 and n85 independently represents an integer of 0 to 3.

For detailed descriptions and preferable ranges of Ar⁸³ to Ar⁸⁵, R⁸³, R⁸⁴, m83, m84, and n83 to n85, descriptions on Ar¹, Ar², Ar⁴, R⁴¹, R⁴², m1, m2, n1, n2, and n4 in the general formula (1a) can be referred to.

Hereinafter, specific examples of the compound represented by the general formula (11a) will be given. Compounds of the general formula (11a) that can be used in the present invention are not construed as limiting to the following specific examples. In relation to the following specific examples, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

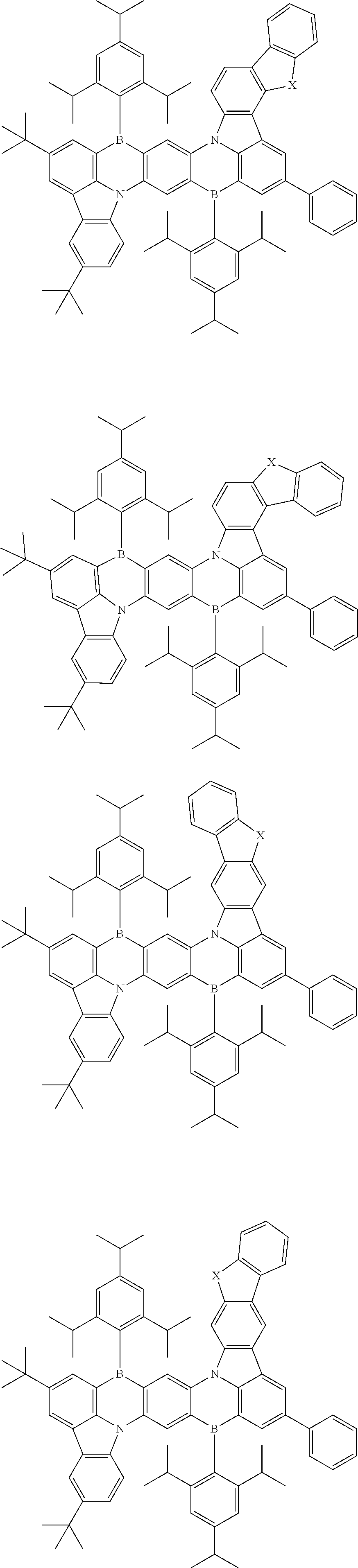

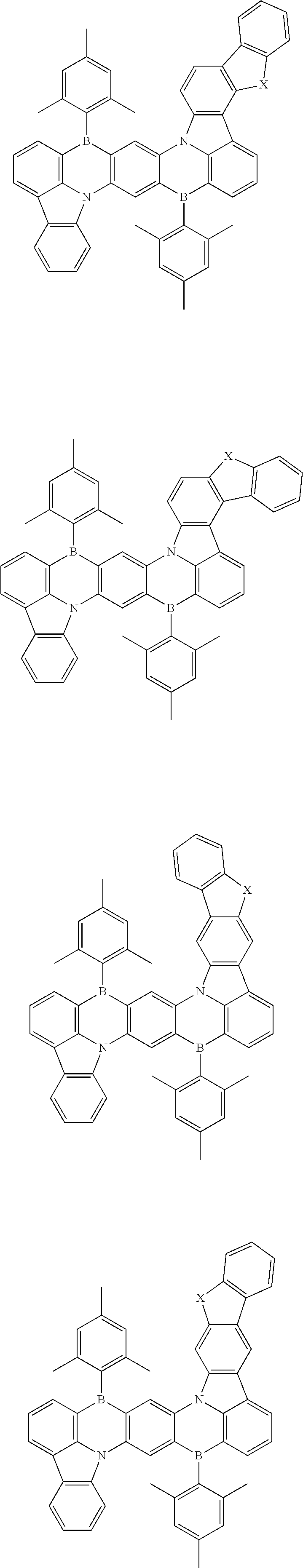

As one preferable group of compounds having the skeleton (11b), compounds represented by the following general formula (11b) can be exemplified.

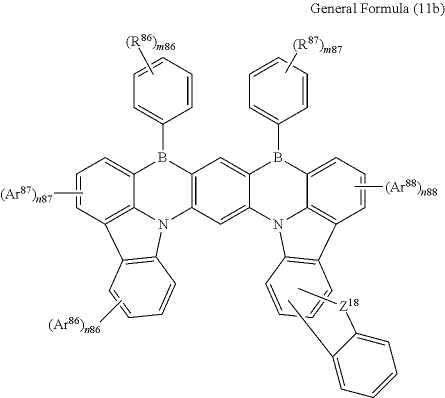

In the general formula (11b), each of Ar⁸⁶ to Ar⁸⁸ independently represents a substituted or unsubstituted aryl group, a substituted or unsubstituted heteroaryl group, or a substituted or unsubstituted alkyl group, and, for example, a substituted or unsubstituted aryl group can be preferably selected. Each of R⁸⁶ and R⁸⁷ independently represents a substituted or unsubstituted alkyl group. Z¹⁸ represents an oxygen atom or a sulfur atom. Each of m86 and m87 independently represents an integer of 0 to 5. n86 represents an integer of 0 to 4, and each of n87 and n88 independently represents an integer of 0 to 3.

For detailed descriptions and preferable ranges of Ar⁸⁶ to Ar⁸⁸, R⁸⁶, R⁸⁷, m86, m87, and n86 to n88, descriptions on Ar¹, Ar², Ar⁴, R⁴¹, R⁴², m1, m2, n1, n2, and n4 in the general formula (1a) can be referred to.

Hereinafter, specific examples of the compound represented by the general formula (11b) will be given. Compounds of the general formula (11b) that can be used in the present invention are not construed as limiting to the following specific examples. In relation to the following specific examples, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

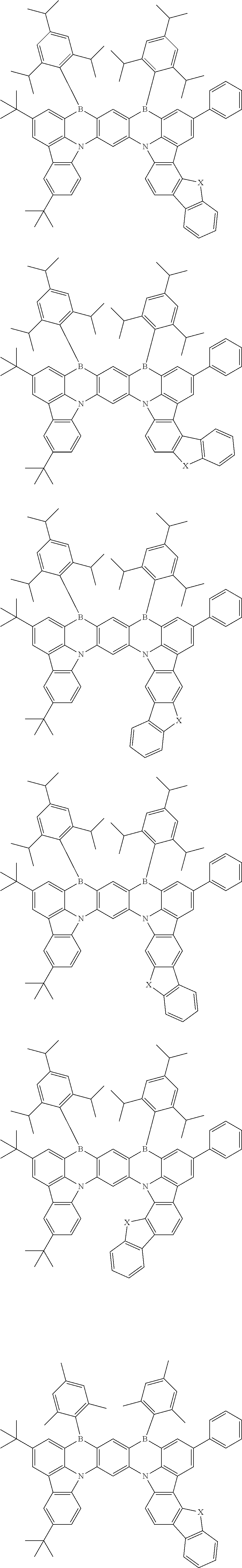

As the compound represented by the general formula (G), a compound in which R⁵ is a donor group can be preferably adopted. The compound in which R⁵ is a donor group has a high molar coefficient extinction, and thus tends to have a high luminous efficiency. For example, it exhibits excellent luminescence characteristics as compared to a compound in which R³ is a donor group. In one preferred aspect of the present invention, R³ is not a donor group. In one preferred aspect of the present invention, among R¹ to R⁷, only R⁵ is a donor group, or none of them is a donor group (in particular, a donor group having a σp value of −0.2 or less). The donor group is a group having a negative Hammett's σp value. The σp value of the donor group for R⁵ is preferably −0.2 or less, and can be, for example, −0.4 or less, or can be, for example, −0.6 or less. As a preferable donor group, a substituted amino group can be mentioned, and a substituted or unsubstituted diarylamino group is preferable. The aryl group can be a monocycle, or can be a fused ring in which two or more rings are fused. In the case of a fused ring, the number of rings after fusing is preferably two to six, and, for example, can be selected from two to four, or can be two. Two aryl groups constituting the diarylamino group can be the same or different. Further, the two aryl groups can be linked by a single bond or a linking group. As the substituted or unsubstituted diarylamino group, a substituted or unsubstituted diphenyl amino group is preferable. A substituted or unsubstituted carbazol-9-yl group in which two phenyl groups bond by a single bond can be adopted, or a substituted or unsubstituted diphenyl amino group in which two phenyl groups are not bonded by a single bond can be adopted. When any of R¹ to R⁷ in the general formula (G) is a substituted amino group, preferably at least R⁵ is a substituted amino group, more preferably only R⁵ is a substituted amino group. In one aspect of the present invention, R³ is not a substituted amino group.

When R⁵ is a donor group, and X¹ is a nitrogen atom, it is preferable that R¹⁶ or R¹⁹ is a donor group, and it is more preferable that R¹⁹ is a donor group. Here, all of the rest of R¹ to R²⁶ can be, for example, hydrogen atoms or deuterium atoms. For example, at least one of R³, R⁶, R¹⁵, and R²⁰ can be a substituent (preferably, a substituted or unsubstituted alkyl group, or a substituted or unsubstituted aryl group) and the others can be hydrogen atoms or deuterium atoms.

When R⁵ is a donor group, and X¹ is a boron atom, it is preferable that R²⁰ or R²³ is a donor group, and it is more preferable that R²⁰ is a donor group. Here, all of the rest of R¹ to R²⁶ can be, for example, hydrogen atoms or deuterium atoms. For example, at least one of R³, R⁶, R¹⁹, and R²⁴ can be a substituent (preferably, a substituted or unsubstituted alkyl group, or a substituted or unsubstituted aryl group) and the others can be hydrogen atoms or deuterium atom.

As one preferable group of compounds in which R⁵ is a donor group, a compound represented by the following general formula (12a) and a compound represented by the following general formula (12b) can be exemplified.

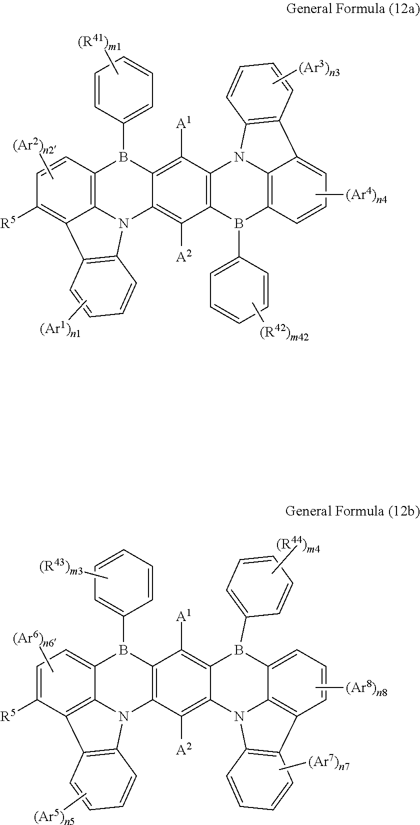

In the general formula (12a) and the general formula (12b), each of Ar¹ to Ar⁸ independently represents a substituted or unsubstituted aryl group, a substituted or unsubstituted heteroaryl group, or a substituted or unsubstituted alkyl group. For example, a substituted or unsubstituted alkyl group can be preferably selected, or a substituted or unsubstituted aryl group can be preferably selected. R⁵ represents a donor group. Each of R⁴¹ to R⁴⁴ independently represents a substituted or unsubstituted alkyl group. Each of m1 to m4 independently represents an integer of 0 to 5. Each of n1, n3, n5, and n7 independently represents an integer of 0 to 4, n4 and n8 represent integers of 0 to 3, and n2′ and n6′ represent integers of 0 to 2. Each of A¹ and A² independently represents a hydrogen atom, a deuterium atom, or a substituent. In relation to details of Ar¹ to Ar⁸, R⁴¹ to R⁴⁴, m1 to m4, n1, n3 to n5, n7, n8, A¹, and A², the corresponding descriptions for the general formula (1a) and the general formula (1b) can be referred to. Meanwhile, Ar¹'s bonded to adjacent carbon atoms, Ar³'s bonded to adjacent carbon atoms, Ar⁵'s bonded to adjacent carbon atoms, and Ar⁷'s bonded to adjacent carbon atoms can be bonded to each other to form cyclic structures. Preferably, benzofuran (fused as a furan ring) or benzothiophene (fused as a thiophene ring) can be formed.

Hereinafter, specific examples of the compounds represented by the general formula (12a) and the general formula (12b) will be given. Meanwhile, compounds of the general formula (12a) and the general formula (12b), which can be used in the present invention, are not construed as limiting to the following specific examples. In the following specific examples, R, Ar, and X in the formulas F1 to F56 are specified in the table so that the structure of each compound is defined. R is selected from A to D described below, Ar is selected from a to d described below, and X is selected from a to γ. For example, the No. 1 compound in the table is a compound of the formula F1, which has a structure in which R is A, and Ar is a.

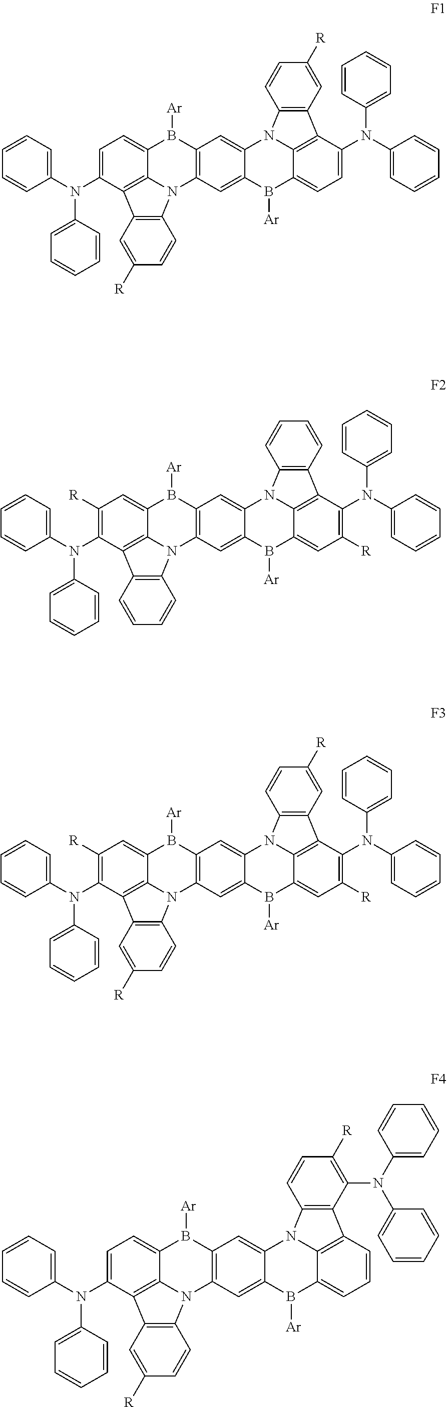

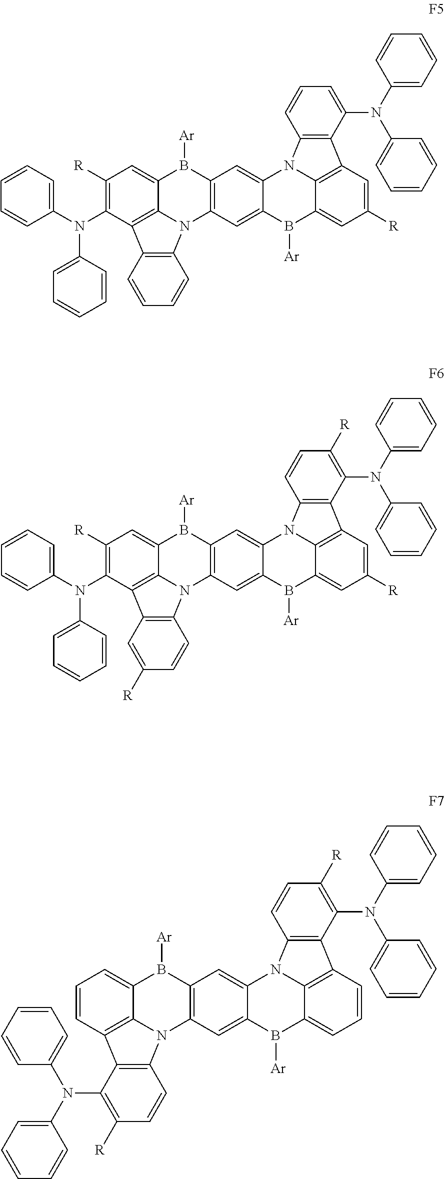

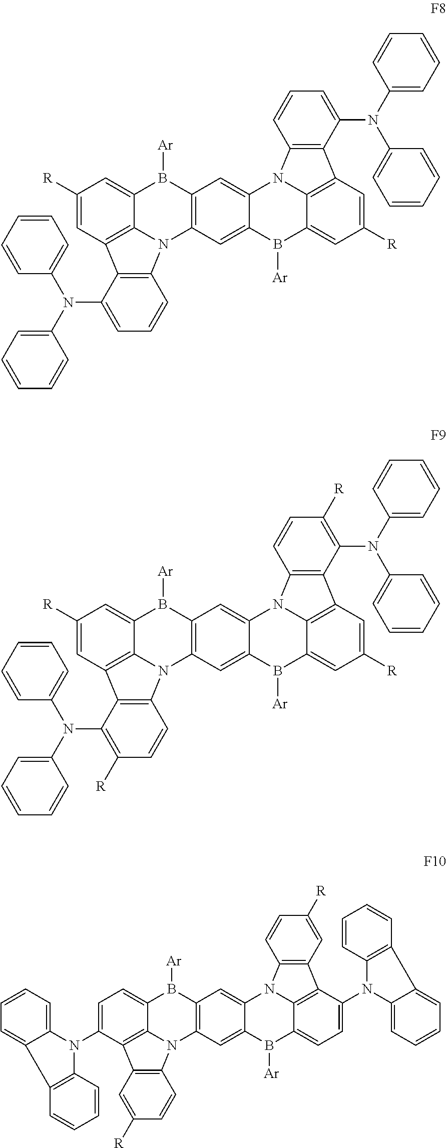

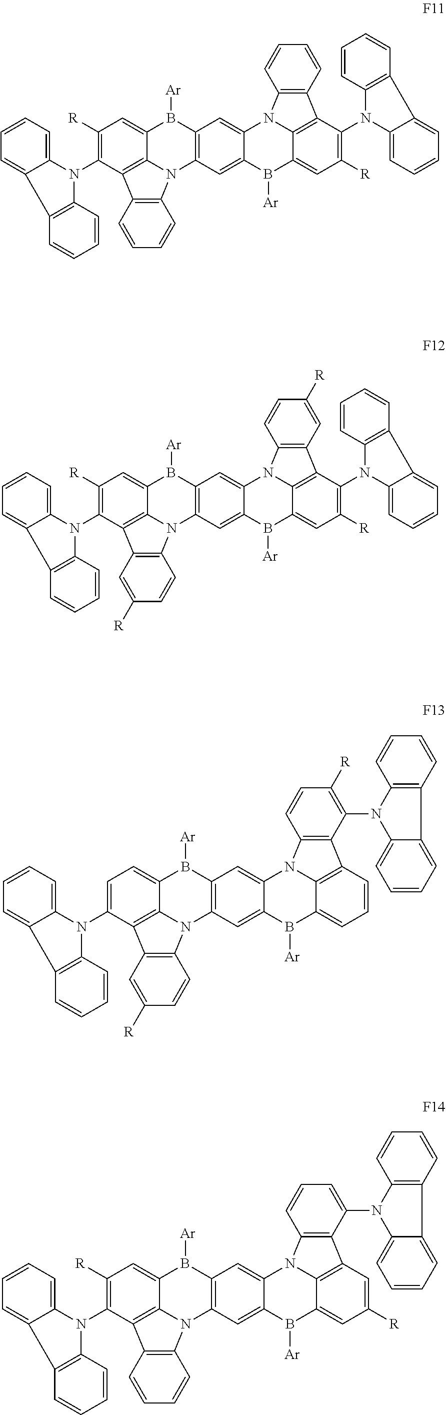

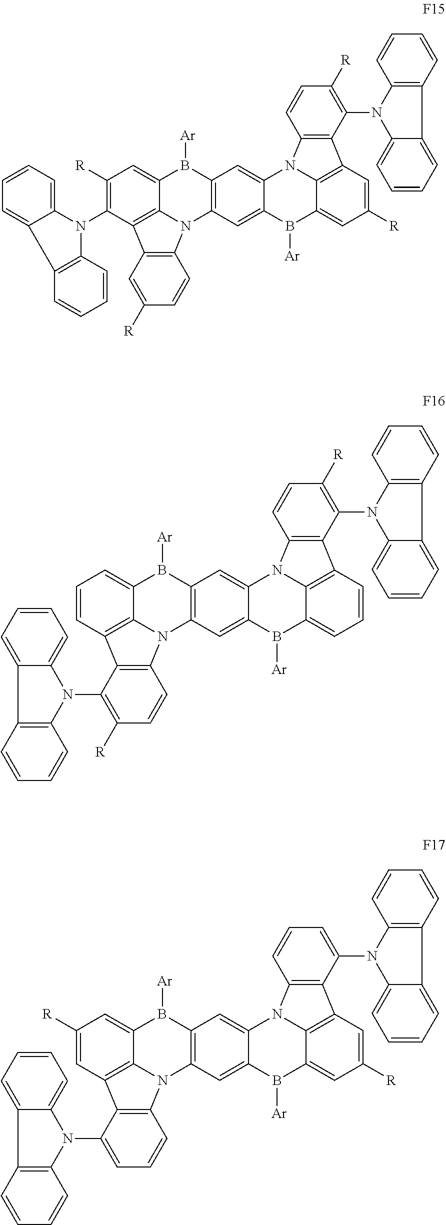

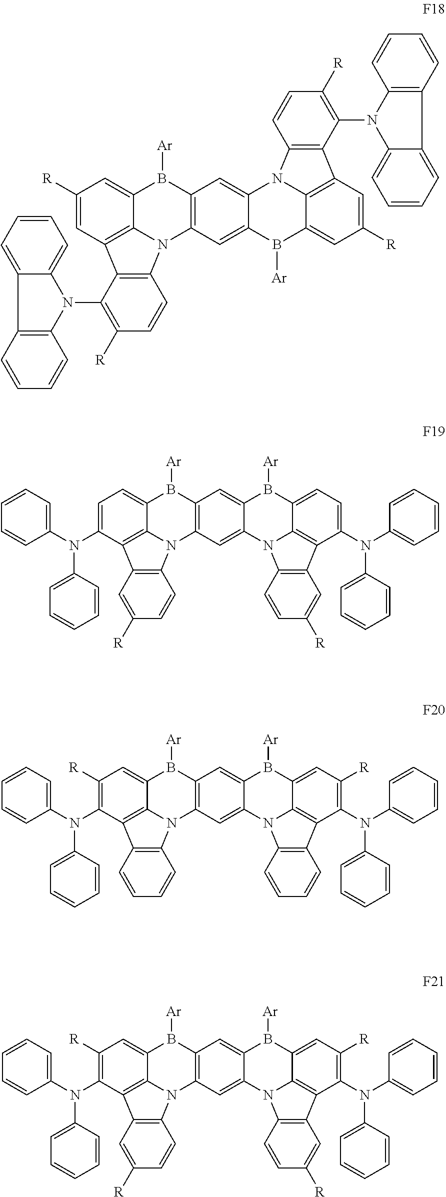

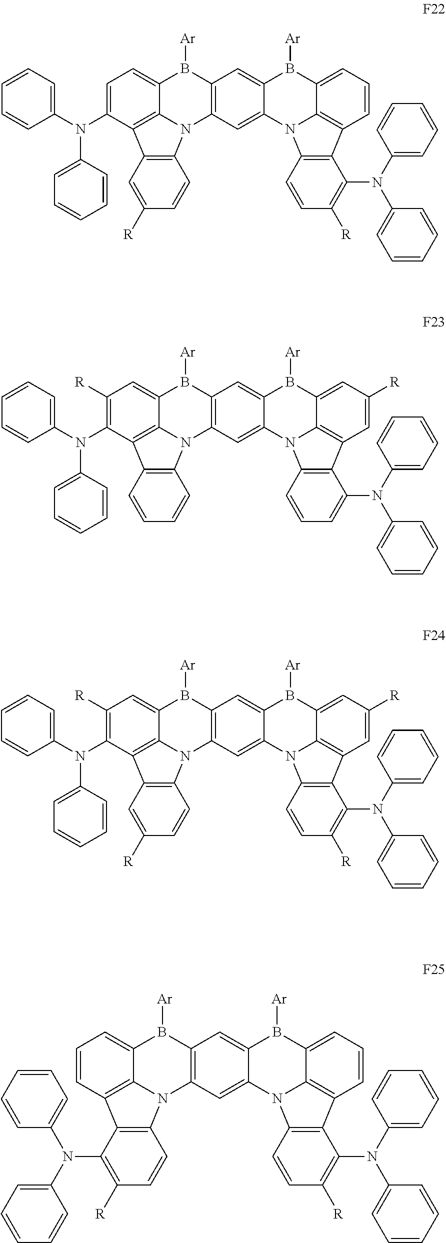

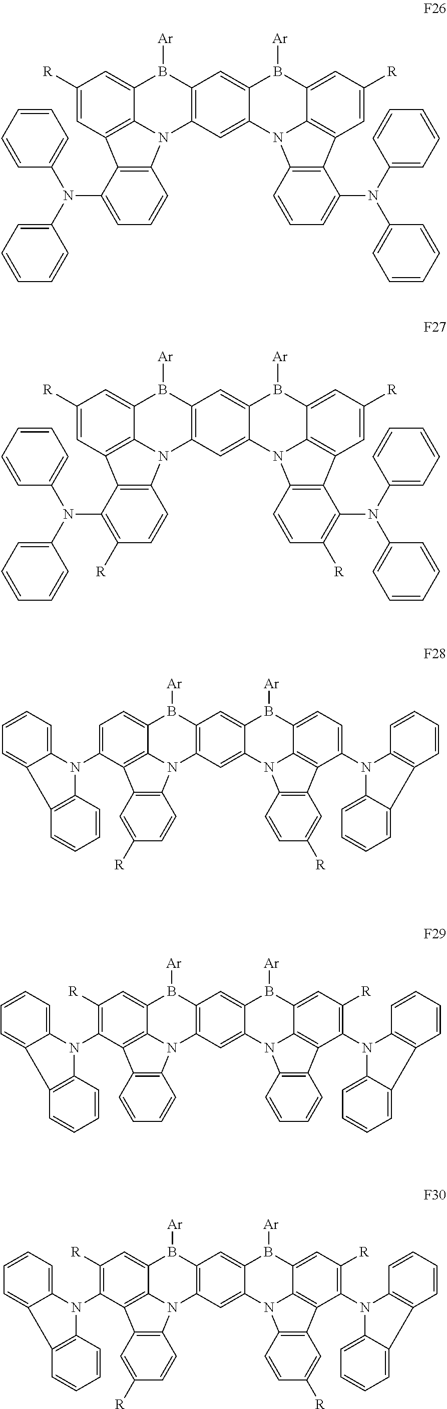

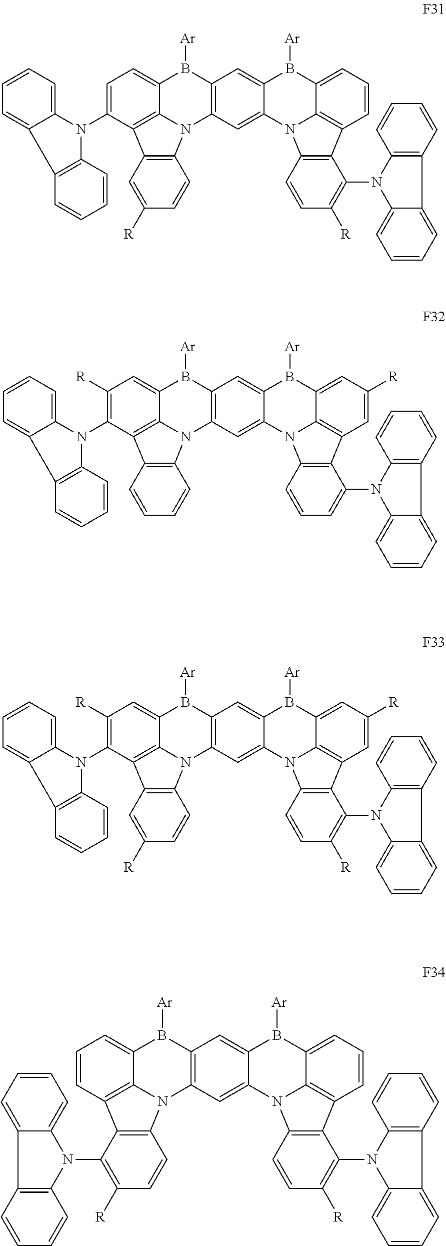

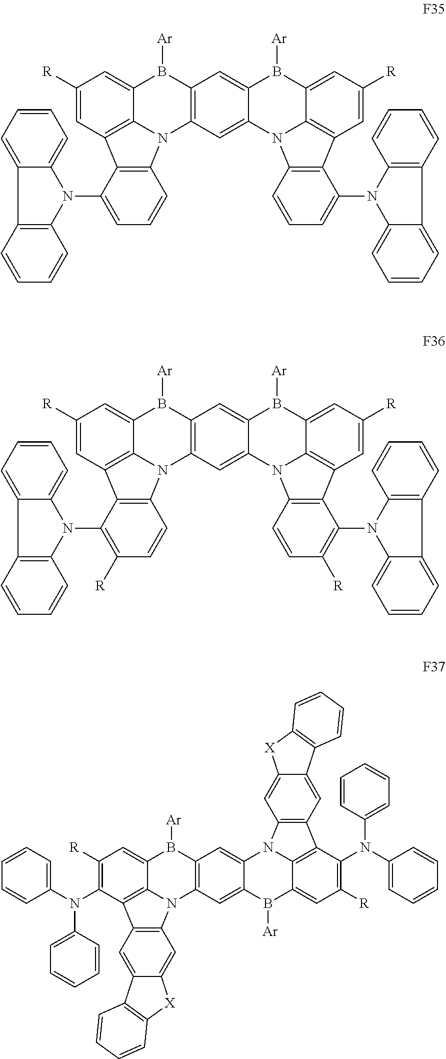

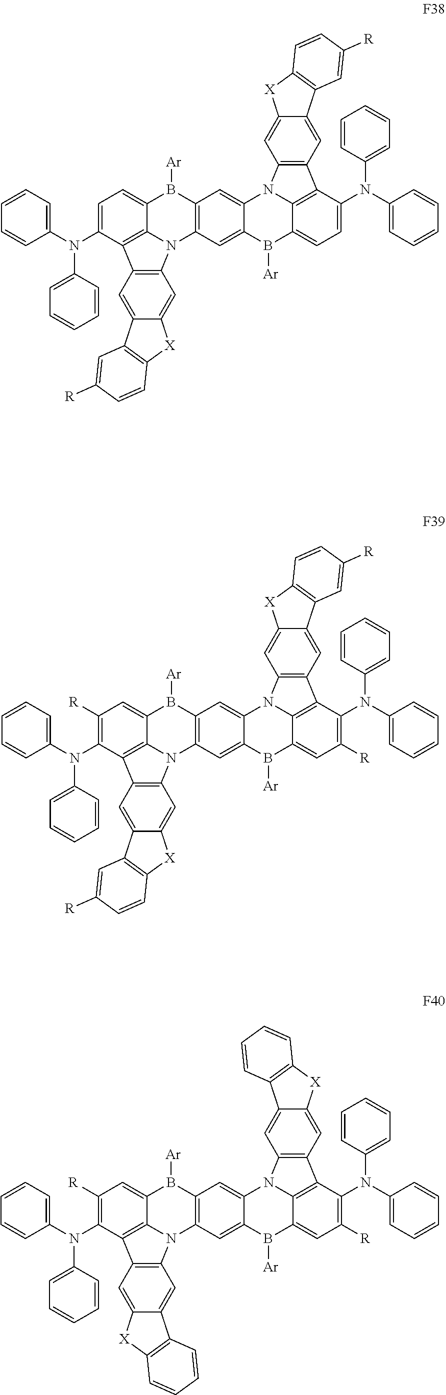

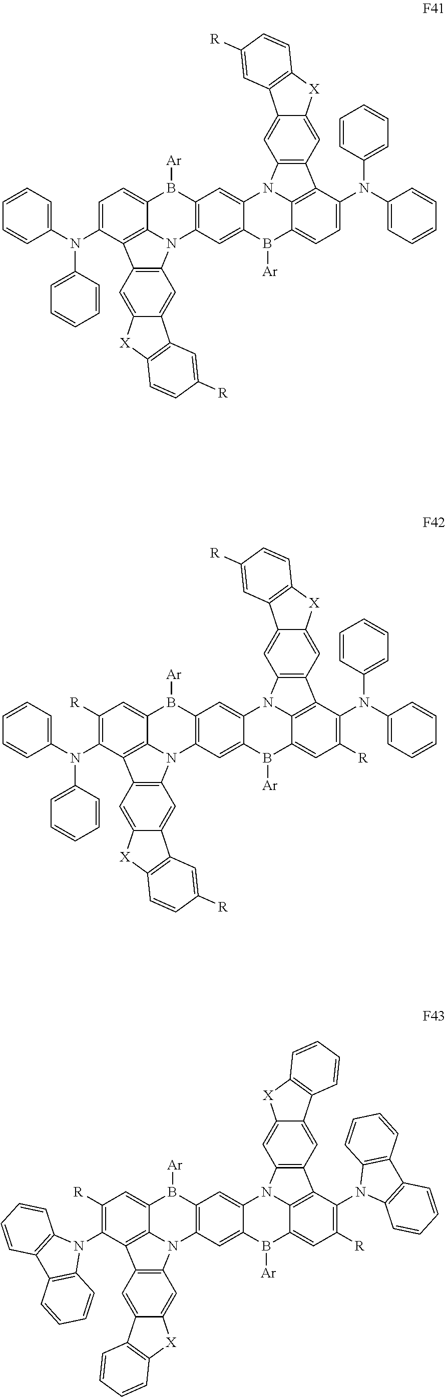

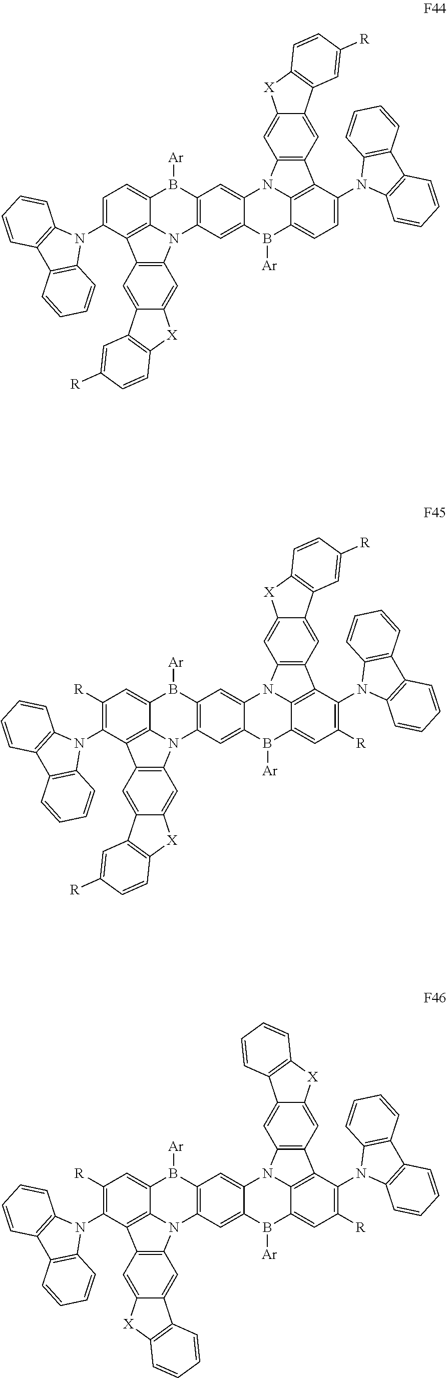

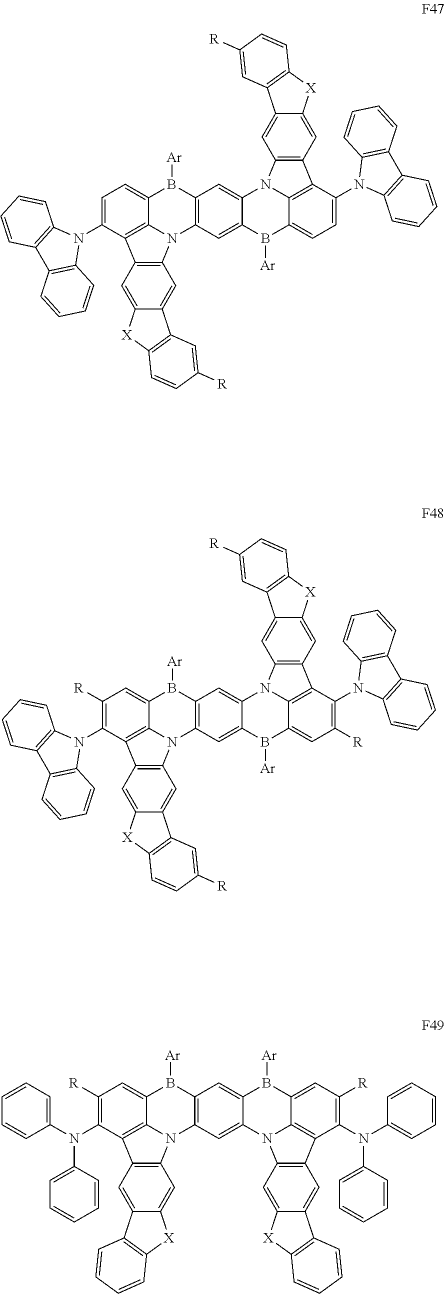

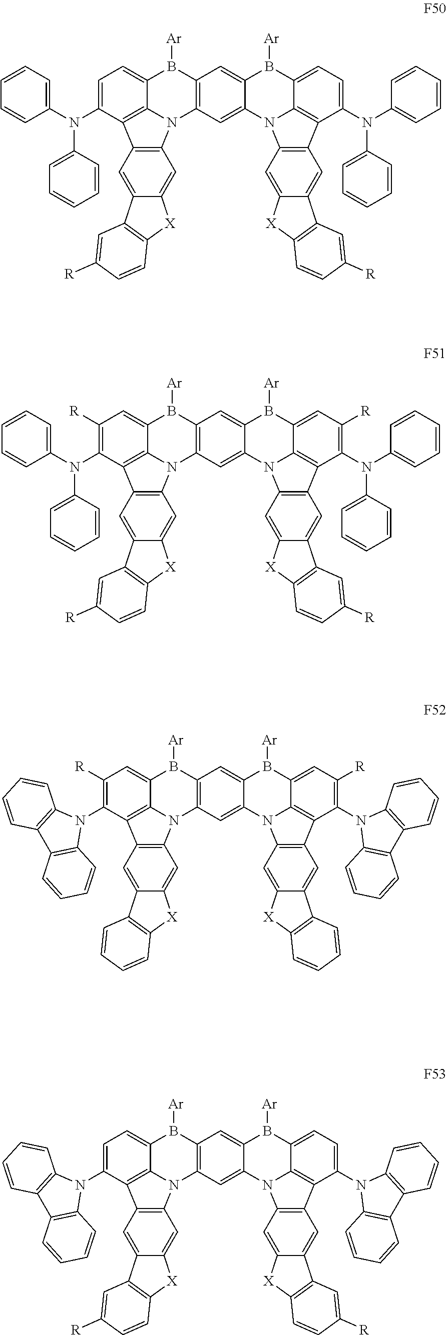

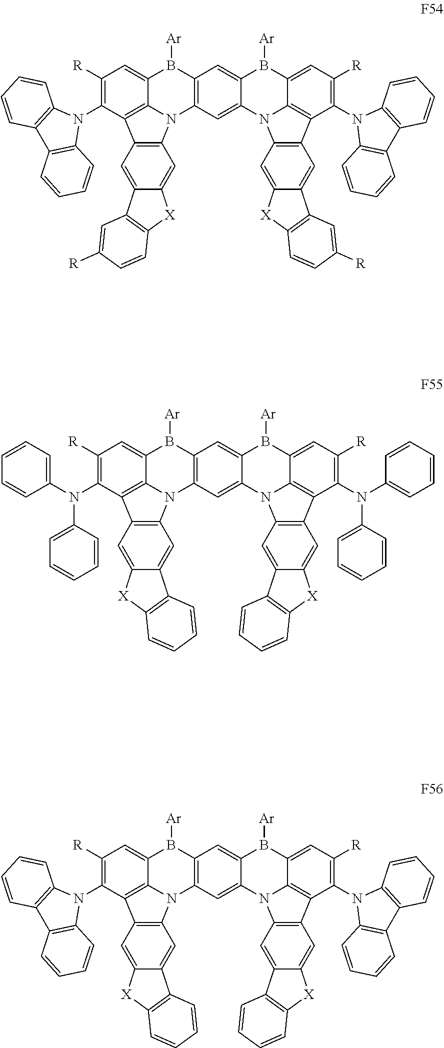

TABLE 2

| No. | F | R | Ar | |
|---|---|---|---|---|
| 1 | F1 | A | a | |
| 2 | F1 | A | b | |
| 3 | F1 | A | c | |
| 4 | F1 | A | d | |
| 5 | F1 | B | a | |
| 6 | F1 | B | b | |
| 7 | F1 | B | c | |
| 8 | F1 | B | d | |
| 9 | F1 | C | a | |
| 10 | F1 | C | b | |
| 11 | F1 | C | c | |
| 12 | F1 | C | d | |
| 13 | F1 | D | a | |
| 14 | F1 | D | b | |
| 15 | F1 | D | c | |
| 16 | F1 | D | d | |
| 17 | F2 | A | a | |
| 18 | F2 | A | b | |
| 19 | F2 | A | c | |
| 20 | F2 | A | d | |
| 21 | F2 | B | a | |
| 22 | F2 | B | b | |
| 23 | F2 | B | c | |
| 24 | F2 | B | d | |
| 25 | F2 | C | a | |
| 26 | F2 | C | b | |
| 27 | F2 | C | c | |
| 28 | F2 | C | d | |
| 29 | F3 | A | a | |
| 30 | F3 | A | b | |
| 31 | F3 | A | c | |
| 32 | F3 | A | d | |
| 33 | F3 | B | a | |
| 34 | F3 | B | b | |
| 35 | F3 | B | c | |
| 36 | F3 | B | d | |
| 37 | F3 | C | a | |
| 38 | F3 | C | b | |
| 39 | F3 | C | c | |
| 40 | F3 | C | d | |
| 41 | F4 | A | a | |
| 42 | F4 | A | b | |
| 43 | F4 | A | c | |
| 44 | F4 | A | d | |
| 45 | F4 | B | a | |
| 46 | F4 | B | b | |
| 47 | F4 | B | c | |
| 48 | F4 | B | d | |
| 49 | F4 | C | a | |
| 50 | F4 | C | b | |
| 51 | F4 | C | c | |
| 52 | F4 | C | d | |
| 53 | F4 | D | a | |
| 54 | F4 | D | b | |
| 55 | F4 | D | c | |
| 56 | F4 | D | d | |
| 57 | F5 | A | a | |
| 58 | F5 | A | b | |
| 59 | F5 | A | c | |
| 60 | F5 | A | d | |
| 61 | F5 | B | a | |
| 62 | F5 | B | b | |
| 63 | F5 | B | c | |
| 64 | F5 | B | d | |
| 65 | F5 | C | a | |
| 66 | F5 | C | b | |
| 67 | F5 | C | c | |
| 68 | F5 | C | d | |
| 69 | F6 | A | a | |
| 70 | F6 | A | b | |
| 71 | F6 | A | c | |
| 72 | F6 | A | d | |
| 73 | F6 | B | a | |
| 74 | F6 | B | b | |
| 75 | F6 | B | c | |
| 76 | F6 | B | d | |
| 77 | F6 | C | a | |
| 78 | F6 | C | b | |
| 79 | F6 | C | c | |
| 80 | F6 | C | d | |
| 81 | F7 | A | a | |
| 82 | F7 | A | b | |
| 83 | F7 | A | c | |
| 84 | F7 | A | d | |
| 85 | F7 | B | a | |
| 86 | F7 | B | b | |
| 87 | F7 | B | c | |
| 88 | F7 | B | d | |
| 89 | F7 | C | a | |
| 90 | F7 | C | b | |
| 91 | F7 | C | c | |
| 92 | F7 | C | d | |
| 93 | F7 | D | a | |
| 94 | F7 | D | b | |
| 95 | F7 | D | c | |
| 96 | F7 | D | d | |
| 97 | F8 | A | a | |
| 98 | F8 | A | b | |
| 99 | F8 | A | c | |
| 100 | F8 | A | d | |
| 101 | F8 | B | a | |
| 102 | F8 | B | b | |
| 103 | F8 | B | c | |
| 104 | F8 | B | d | |
| 105 | F8 | C | a | |
| 106 | F8 | C | b | |
| 107 | F8 | C | c | |
| 108 | F8 | C | d | |
| 109 | F9 | A | a | |
| 110 | F9 | A | b | |
| 111 | F9 | A | c | |
| 112 | F9 | A | d | |
| 113 | F9 | B | a | |
| 114 | F9 | B | b | |
| 115 | F9 | B | c | |
| 116 | F9 | B | d | |
| 117 | F9 | C | a | |
| 118 | F9 | C | b | |
| 119 | F9 | C | c | |
| 120 | F9 | C | d | |
| 121 | F10 | A | a | |
| 122 | F10 | A | b | |
| 123 | F10 | A | c | |
| 124 | F10 | A | d | |
| 125 | F10 | B | a | |
| 126 | F10 | B | b | |
| 127 | F10 | B | c | |
| 128 | F10 | B | d | |
| 129 | F10 | C | a | |
| 130 | F10 | C | b | |
| 131 | F10 | C | c | |
| 132 | F10 | C | d | |
| 133 | F10 | D | a | |
| 134 | F10 | D | b | |
| 135 | F10 | D | c | |
| 136 | F10 | D | d | |
| 137 | F11 | A | a | |
| 138 | F11 | A | b | |
| 139 | F11 | A | c | |
| 140 | F11 | A | d | |
| 141 | F11 | B | a | |
| 142 | F11 | B | b | |
| 143 | F11 | B | c | |
| 144 | F11 | B | d | |
| 145 | F11 | C | a | |
| 146 | F11 | C | b | |
| 147 | F11 | C | c | |
| 148 | F11 | C | d | |
| 149 | F12 | A | a | |
| 150 | F12 | A | b | |
| 151 | F12 | A | c | |
| 152 | F12 | A | d | |
| 153 | F12 | B | a | |
| 154 | F12 | B | b | |
| 155 | F12 | B | c | |
| 156 | F12 | B | d | |
| 157 | F12 | C | a | |
| 158 | F12 | C | b | |
| 159 | F12 | C | c | |
| 160 | F12 | C | d | |
| 161 | F13 | A | a | |
| 162 | F13 | A | b | |
| 163 | F13 | A | c | |
| 164 | F13 | A | d | |
| 165 | F13 | B | a | |
| 166 | F13 | B | b | |
| 167 | F13 | B | c | |
| 168 | F13 | B | d | |
| 169 | F13 | C | a | |
| 170 | F13 | C | b | |
| 171 | F13 | C | c | |
| 172 | F13 | C | d | |
| 173 | F13 | D | a | |
| 174 | F13 | D | b | |
| 175 | F13 | D | c | |
| 176 | F13 | D | d | |
| 177 | F14 | A | a | |
| 178 | F14 | A | b | |
| 179 | F14 | A | c | |
| 180 | F14 | A | d | |
| 181 | F14 | B | a | |
| 182 | F14 | B | b | |
| 183 | F14 | B | c | |
| 184 | F14 | B | d | |
| 185 | F14 | C | a | |
| 186 | F14 | C | b | |
| 187 | F14 | C | c | |
| 188 | F14 | C | d | |
| 189 | F15 | A | a | |
| 190 | F15 | A | b | |
| 191 | F15 | A | c | |
| 192 | F15 | A | d | |
| 193 | F15 | B | a | |
| 194 | F15 | B | b | |
| 195 | F15 | B | c | |
| 196 | F15 | B | d | |
| 197 | F15 | C | a | |
| 198 | F15 | C | b | |
| 199 | F15 | C | c | |
| 200 | F15 | C | d | |
| 201 | F16 | A | a | |
| 202 | F16 | A | b | |
| 203 | F16 | A | c | |
| 204 | F16 | A | d | |
| 205 | F16 | B | a | |
| 206 | F16 | B | b | |
| 207 | F16 | B | c | |
| 208 | F16 | B | d | |
| 209 | F16 | C | a | |
| 210 | F16 | C | b | |
| 211 | F16 | C | c | |
| 212 | F16 | C | d | |
| 213 | F16 | D | a | |
| 214 | F16 | D | b | |
| 215 | F16 | D | c | |
| 216 | F16 | D | d | |
| 217 | F17 | A | a | |
| 218 | F17 | A | b | |
| 219 | F17 | A | c | |
| 220 | F17 | A | d | |
| 221 | F17 | B | a | |
| 222 | F17 | B | b | |
| 223 | F17 | B | c | |
| 224 | F17 | B | d | |
| 225 | F17 | C | a | |
| 226 | F17 | C | b | |
| 227 | F17 | C | c | |
| 228 | F17 | C | d | |
| 229 | F18 | A | a | |
| 230 | F18 | A | b | |
| 231 | F18 | A | c | |
| 232 | F18 | A | d | |
| 233 | F18 | B | a | |
| 234 | F18 | B | b | |
| 235 | F18 | B | c | |
| 236 | F18 | B | d | |
| 237 | F18 | C | a | |
| 238 | F18 | C | b | |
| 239 | F18 | C | c | |
| 240 | F18 | C | d | |
| 241 | F19 | A | a | |
| 242 | F19 | A | b | |
| 243 | F19 | A | c | |
| 244 | F19 | A | d | |
| 245 | F19 | B | a | |
| 246 | F19 | B | b | |
| 247 | F19 | B | c | |
| 248 | F19 | B | d | |
| 249 | F19 | C | a | |
| 250 | F19 | C | b | |
| 251 | F19 | C | c | |
| 252 | F19 | C | d | |
| 253 | F19 | D | a | |
| 254 | F19 | D | b | |
| 255 | F19 | D | c | |
| 256 | F19 | D | d | |
| 257 | F20 | A | a | |
| 258 | F20 | A | b | |
| 259 | F20 | A | c | |
| 260 | F20 | A | d | |
| 261 | F20 | B | a | |
| 262 | F20 | B | b | |
| 263 | F20 | B | c | |
| 264 | F20 | B | d | |
| 265 | F20 | C | a | |
| 266 | F20 | C | b | |
| 267 | F20 | C | c | |
| 268 | F20 | C | d | |
| 269 | F21 | A | a | |
| 270 | F21 | A | b | |
| 271 | F21 | A | c | |
| 272 | F21 | A | d | |
| 273 | F21 | B | a | |
| 274 | F21 | B | b | |
| 275 | F21 | B | c | |
| 276 | F21 | B | d | |
| 277 | F21 | C | a | |
| 278 | F21 | C | b | |
| 279 | F21 | C | c | |
| 280 | F21 | C | d | |
| 281 | F22 | A | a | |
| 282 | F22 | A | b | |
| 283 | F22 | A | c | |
| 284 | F22 | A | d | |
| 285 | F22 | B | a | |
| 286 | F22 | B | b | |
| 287 | F22 | B | c | |
| 288 | F22 | B | d | |
| 289 | F22 | C | a | |
| 290 | F22 | C | b | |
| 291 | F22 | C | c | |
| 292 | F22 | C | d | |
| 293 | F22 | D | a | |
| 294 | F22 | D | b | |
| 295 | F22 | D | c | |
| 296 | F22 | D | d | |
| 297 | F23 | A | a | |
| 298 | F23 | A | b | |
| 299 | F23 | A | c | |
| 300 | F23 | A | d | |
| 301 | F23 | B | a | |
| 302 | F23 | B | b | |
| 303 | F23 | B | c | |
| 304 | F23 | B | d | |
| 305 | F23 | C | a | |
| 306 | F23 | C | b | |
| 307 | F23 | C | c | |
| 308 | F23 | C | d | |
| 309 | F24 | A | a | |
| 310 | F24 | A | b | |
| 311 | F24 | A | c | |
| 312 | F24 | A | d | |
| 313 | F24 | B | a | |
| 314 | F24 | B | b | |
| 315 | F24 | B | c | |
| 316 | F24 | B | d | |
| 317 | F24 | C | a | |
| 318 | F24 | C | b | |
| 319 | F24 | C | c | |
| 320 | F24 | C | d | |
| 321 | F25 | A | a | |
| 322 | F25 | A | b | |
| 323 | F25 | A | c | |
| 324 | F25 | A | d | |
| 325 | F25 | B | a | |
| 326 | F25 | B | b | |
| 327 | F25 | B | c | |
| 328 | F25 | B | d | |
| 329 | F25 | C | a | |
| 330 | F25 | C | b | |
| 331 | F25 | C | c | |
| 332 | F25 | C | d | |
| 333 | F25 | D | a | |
| 334 | F25 | D | b | |
| 335 | F25 | D | c | |
| 336 | F25 | D | d | |
| 337 | F26 | A | a | |
| 338 | F26 | A | b | |
| 339 | F26 | A | c | |
| 340 | F26 | A | d | |
| 341 | F26 | B | a | |
| 342 | F26 | B | b | |
| 343 | F26 | B | c | |
| 344 | F26 | B | d | |
| 345 | F26 | C | a | |
| 346 | F26 | C | b | |
| 347 | F26 | C | c | |
| 348 | F26 | C | d | |
| 349 | F27 | A | a | |
| 350 | F27 | A | b | |
| 351 | F27 | A | c | |
| 352 | F27 | A | d | |
| 353 | F27 | B | a | |
| 354 | F27 | B | b | |
| 355 | F27 | B | c | |
| 356 | F27 | B | d | |
| 357 | F27 | C | a | |
| 358 | F27 | C | b | |
| 359 | F27 | C | c | |
| 360 | F27 | C | d | |
| 361 | F28 | A | a | |
| 362 | F28 | A | b | |
| 363 | F28 | A | c | |
| 364 | F28 | A | d | |
| 365 | F28 | B | a | |
| 366 | F28 | B | b | |
| 367 | F28 | B | c | |
| 368 | F28 | B | d | |
| 369 | F28 | C | a | |
| 370 | F28 | C | b | |
| 371 | F28 | C | c | |
| 372 | F28 | C | d | |
| 373 | F28 | D | a | |
| 374 | F28 | D | b | |
| 375 | F28 | D | c | |
| 376 | F28 | D | d | |
| 377 | F29 | A | a | |
| 378 | F29 | A | b | |
| 379 | F29 | A | c | |
| 380 | F29 | A | d | |
| 381 | F29 | B | a | |
| 382 | F29 | B | b | |
| 383 | F29 | B | c | |
| 384 | F29 | B | d | |
| 385 | F29 | C | a | |
| 386 | F29 | C | b | |
| 387 | F29 | C | c | |
| 388 | F29 | C | d | |
| 389 | F30 | A | a | |
| 390 | F30 | A | b | |
| 391 | F30 | A | c | |
| 392 | F30 | A | d | |
| 393 | F30 | B | a | |
| 394 | F30 | B | b | |
| 395 | F30 | B | c | |
| 396 | F30 | B | d | |
| 397 | F30 | C | a | |
| 398 | F30 | C | b | |
| 399 | F30 | C | c | |
| 400 | F30 | C | d | |
| 401 | F31 | A | a | |
| 402 | F31 | A | b | |
| 403 | F31 | A | c | |
| 404 | F31 | A | d | |
| 405 | F31 | B | a | |
| 406 | F31 | B | b | |
| 407 | F31 | B | c | |
| 408 | F31 | B | d | |
| 409 | F31 | C | a | |
| 410 | F31 | C | b | |
| 411 | F31 | C | c | |
| 412 | F31 | C | d | |
| 413 | F31 | D | a | |
| 414 | F31 | D | b | |
| 415 | F31 | D | c | |
| 416 | F31 | D | d | |
| 417 | F32 | A | a | |
| 418 | F32 | A | b | |
| 419 | F32 | A | c | |
| 420 | F32 | A | d | |
| 421 | F32 | B | a | |
| 422 | F32 | B | b | |
| 423 | F32 | B | c | |
| 424 | F32 | B | d | |
| 425 | F32 | C | a | |
| 426 | F32 | C | b | |
| 427 | F32 | C | c | |
| 428 | F32 | C | d | |
| 429 | F33 | A | a | |
| 430 | F33 | A | b | |
| 431 | F33 | A | c | |
| 432 | F33 | A | d | |
| 433 | F33 | B | a | |
| 434 | F33 | B | b | |
| 435 | F33 | B | c | |
| 436 | F33 | B | d | |
| 437 | F33 | C | a | |
| 438 | F33 | C | b | |
| 439 | F33 | C | c | |
| 440 | F33 | C | d | |
| 441 | F34 | A | a | |
| 442 | F34 | A | b | |
| 443 | F34 | A | c | |
| 444 | F34 | A | d | |
| 445 | F34 | B | a | |
| 446 | F34 | B | b | |
| 447 | F34 | B | c | |
| 448 | F34 | B | d | |
| 449 | F34 | C | a | |
| 450 | F34 | C | b | |
| 451 | F34 | C | c | |
| 452 | F34 | C | d | |
| 453 | F34 | D | a | |
| 454 | F34 | D | b | |
| 455 | F34 | D | c | |
| 456 | F34 | D | d | |
| 457 | F35 | A | a | |
| 458 | F35 | A | b | |
| 459 | F35 | A | c | |
| 460 | F35 | A | d | |
| 461 | F35 | B | a | |
| 462 | F35 | B | b | |
| 463 | F35 | B | c | |
| 464 | F35 | B | d | |
| 465 | F35 | C | a | |
| 466 | F35 | C | b | |
| 467 | F35 | C | c | |
| 468 | F35 | C | d | |
| 469 | F36 | A | a | |
| 470 | F36 | A | b | |
| 471 | F36 | A | c | |
| 472 | F36 | A | d | |
| 473 | F36 | B | a | |
| 474 | F36 | B | b | |
| 475 | F36 | B | c | |
| 476 | F36 | B | d | |
| 477 | F36 | C | a | |
| 478 | F36 | C | b | |
| 479 | F36 | C | c | |
| 480 | F36 | C | d | |
| No. | F | R | Ar | X |
| 481 | F37 | A | a | α |
| 482 | F37 | A | a | β |
| 483 | F37 | A | a | γ |
| 484 | F37 | A | b | α |
| 485 | F37 | A | b | β |
| 486 | F37 | A | b | γ |
| 487 | F37 | A | c | α |
| 488 | F37 | A | c | β |
| 489 | F37 | A | c | γ |
| 490 | F37 | A | d | α |
| 491 | F37 | A | d | β |
| 492 | F37 | A | d | γ |
| 493 | F37 | B | a | α |
| 494 | F37 | B | a | β |
| 495 | F37 | B | a | γ |
| 496 | F37 | B | b | α |
| 497 | F37 | B | b | β |
| 498 | F37 | B | b | γ |
| 499 | F37 | B | c | α |
| 500 | F37 | B | c | β |
| 501 | F37 | B | c | γ |
| 502 | F37 | B | d | α |
| 503 | F37 | B | d | β |
| 504 | F37 | B | d | γ |
| 505 | F37 | C | a | α |
| 506 | F37 | C | a | β |
| 507 | F37 | C | a | γ |
| 508 | F37 | C | b | α |
| 509 | F37 | C | b | β |
| 510 | F37 | C | b | γ |
| 511 | F37 | C | c | α |
| 512 | F37 | C | c | β |
| 513 | F37 | C | c | γ |
| 514 | F37 | C | d | α |
| 515 | F37 | C | d | β |
| 516 | F37 | C | d | γ |
| 517 | F37 | D | a | α |
| 518 | F37 | D | a | β |
| 519 | F37 | D | a | γ |
| 520 | F37 | D | b | α |
| 521 | F37 | D | b | β |
| 522 | F37 | D | b | γ |
| 523 | F37 | D | c | α |
| 524 | F37 | D | c | β |
| 525 | F37 | D | c | γ |
| 526 | F37 | D | d | α |
| 527 | F37 | D | d | β |
| 528 | F37 | D | d | γ |
| 529 | F38 | A | a | α |
| 530 | F38 | A | a | β |
| 531 | F38 | A | a | γ |
| 532 | F38 | A | b | α |
| 533 | F38 | A | b | β |
| 534 | F38 | A | b | γ |
| 535 | F38 | A | c | α |
| 536 | F38 | A | c | β |
| 537 | F38 | A | c | γ |
| 538 | F38 | A | d | α |
| 539 | F38 | A | d | β |
| 540 | F38 | A | d | γ |
| 541 | F38 | B | a | α |
| 542 | F38 | B | a | β |
| 543 | F38 | B | a | γ |
| 544 | F38 | B | b | α |
| 545 | F38 | B | b | β |
| 546 | F38 | B | b | γ |
| 547 | F38 | B | c | α |
| 548 | F38 | B | c | β |
| 549 | F38 | B | c | γ |
| 550 | F38 | B | d | α |
| 551 | F38 | B | d | β |
| 552 | F38 | B | d | γ |
| 553 | F38 | C | a | α |
| 554 | F38 | C | a | β |
| 555 | F38 | C | a | γ |
| 556 | F38 | C | b | α |
| 557 | F38 | C | b | β |
| 558 | F38 | C | b | γ |
| 559 | F38 | C | c | α |
| 560 | F38 | C | c | β |
| 561 | F38 | C | c | γ |
| 562 | F38 | C | d | α |
| 563 | F38 | C | d | β |
| 564 | F38 | C | d | γ |
| 565 | F39 | A | a | α |
| 566 | F39 | A | a | β |
| 567 | F39 | A | a | γ |
| 568 | F39 | A | b | α |
| 569 | F39 | A | b | β |
| 570 | F39 | A | b | γ |
| 571 | F39 | A | c | α |
| 572 | F39 | A | c | β |
| 573 | F39 | A | c | γ |
| 574 | F39 | A | d | α |
| 575 | F39 | A | d | β |
| 576 | F39 | A | d | γ |
| 577 | F39 | B | a | α |
| 578 | F39 | B | a | β |
| 579 | F39 | B | a | γ |
| 580 | F39 | B | b | α |
| 581 | F39 | B | b | β |
| 582 | F39 | B | b | γ |
| 583 | F39 | B | c | α |
| 584 | F39 | B | c | β |
| 585 | F39 | B | c | γ |
| 586 | F39 | B | d | α |
| 587 | F39 | B | d | β |
| 588 | F39 | B | d | γ |
| 589 | F39 | C | a | α |
| 590 | F39 | C | a | β |
| 591 | F39 | C | a | γ |
| 592 | F39 | C | b | α |
| 593 | F39 | C | b | β |
| 594 | F39 | C | b | γ |
| 595 | F39 | C | c | α |
| 596 | F39 | C | c | β |
| 597 | F39 | C | c | γ |
| 598 | F39 | C | d | α |
| 599 | F39 | C | d | β |
| 600 | F39 | C | d | γ |
| 601 | F40 | A | a | α |
| 602 | F40 | A | a | β |
| 603 | F40 | A | a | γ |
| 604 | F40 | A | b | α |
| 605 | F40 | A | b | β |
| 606 | F40 | A | b | γ |
| 607 | F40 | A | c | α |
| 608 | F40 | A | c | β |
| 609 | F40 | A | c | γ |
| 610 | F40 | A | d | α |
| 611 | F40 | A | d | β |
| 612 | F40 | A | d | γ |
| 613 | F40 | B | a | α |
| 614 | F40 | B | a | β |
| 615 | F40 | B | a | γ |
| 616 | F40 | B | b | α |
| 617 | F40 | B | b | β |
| 618 | F40 | B | b | γ |
| 619 | F40 | B | c | α |
| 620 | F40 | B | c | β |
| 621 | F40 | B | c | γ |
| 622 | F40 | B | d | α |
| 623 | F40 | B | d | β |
| 624 | F40 | B | d | γ |
| 625 | F40 | C | a | α |
| 626 | F40 | C | a | β |
| 627 | F40 | C | a | γ |
| 628 | F40 | C | b | α |
| 629 | F40 | C | b | β |
| 630 | F40 | C | b | γ |
| 631 | F40 | C | c | α |
| 632 | F40 | C | c | β |
| 633 | F40 | C | c | γ |
| 634 | F40 | C | d | α |
| 635 | F40 | C | d | β |
| 636 | F40 | C | d | γ |
| 637 | F40 | D | a | α |
| 638 | F40 | D | a | β |
| 639 | F40 | D | a | γ |
| 640 | F40 | D | b | α |
| 641 | F40 | D | b | β |
| 642 | F40 | D | b | γ |
| 643 | F40 | D | c | α |
| 644 | F40 | D | c | β |
| 645 | F40 | D | c | γ |
| 646 | F40 | D | d | α |
| 647 | F4 | D | d | β |
| 648 | F40 | D | d | γ |
| 649 | F41 | A | a | α |
| 650 | F41 | A | a | β |
| 651 | F41 | A | a | γ |
| 652 | F41 | A | b | α |
| 653 | F41 | A | b | β |
| 654 | F41 | A | b | γ |
| 655 | F41 | A | c | α |
| 656 | F41 | A | c | β |
| 657 | F41 | A | c | γ |
| 658 | F41 | A | d | α |
| 659 | F41 | A | d | β |
| 660 | F41 | A | d | γ |
| 661 | F41 | B | a | α |
| 662 | F41 | B | a | β |
| 663 | F41 | B | a | γ |
| 664 | F41 | B | b | α |
| 665 | F41 | B | b | β |
| 666 | F41 | B | b | γ |
| 667 | F41 | B | c | α |
| 668 | F41 | B | c | β |
| 669 | F41 | B | c | γ |
| 670 | F41 | B | d | α |
| 671 | F41 | B | d | β |
| 672 | F41 | B | d | γ |
| 673 | F41 | C | a | α |
| 674 | F41 | C | a | β |
| 675 | F41 | C | a | γ |
| 676 | F41 | C | b | α |
| 677 | F41 | C | b | β |
| 678 | F41 | C | b | γ |
| 679 | F41 | C | c | α |
| 680 | F41 | C | c | β |
| 681 | F41 | C | c | γ |
| 682 | F41 | C | d | α |
| 683 | F41 | C | d | β |
| 684 | F41 | C | d | γ |
| 685 | F42 | A | a | α |
| 686 | F42 | A | a | β |
| 687 | F42 | A | a | γ |
| 688 | F42 | A | b | α |
| 689 | F42 | A | b | β |
| 690 | F42 | A | b | γ |
| 691 | F42 | A | c | α |
| 692 | F42 | A | c | β |
| 693 | F42 | A | c | γ |
| 694 | F42 | A | d | α |
| 695 | F42 | A | d | β |
| 696 | F42 | A | d | γ |
| 697 | F42 | B | a | α |
| 698 | F42 | B | a | β |
| 699 | F42 | B | a | γ |
| 700 | F42 | B | b | α |
| 701 | F42 | B | b | β |
| 702 | F42 | B | b | γ |
| 703 | F42 | B | c | α |
| 704 | F42 | B | c | β |
| 705 | F42 | B | c | γ |
| 706 | F42 | B | d | α |
| 707 | F42 | B | d | β |
| 708 | F42 | B | d | γ |
| 709 | F42 | C | a | α |
| 710 | F42 | C | a | β |
| 711 | F42 | C | a | γ |
| 712 | F42 | C | b | α |
| 713 | F42 | C | b | β |
| 714 | F42 | C | b | γ |
| 715 | F42 | C | c | α |
| 716 | F42 | C | c | β |
| 717 | F42 | C | c | γ |
| 718 | F42 | C | d | α |
| 719 | F42 | C | d | β |
| 720 | F42 | C | d | γ |
| 721 | F43 | A | a | α |
| 722 | F43 | A | a | β |
| 723 | F43 | A | a | γ |
| 724 | F43 | A | b | α |
| 725 | F43 | A | b | β |
| 726 | F43 | A | b | γ |
| 727 | F43 | A | c | α |
| 728 | F43 | A | c | β |
| 729 | F43 | A | c | γ |
| 730 | F43 | A | d | α |
| 731 | F43 | A | a | β |
| 732 | F43 | A | a | γ |
| 733 | F43 | B | a | α |
| 734 | F43 | B | a | β |
| 735 | F43 | B | a | γ |
| 736 | F43 | B | b | α |
| 737 | F43 | B | b | β |
| 738 | F43 | B | b | γ |
| 739 | F43 | B | c | α |
| 740 | F43 | B | c | β |
| 741 | F43 | B | c | γ |
| 742 | F43 | B | d | α |
| 743 | F43 | B | d | β |
| 744 | F43 | B | d | γ |
| 745 | F43 | C | a | α |
| 746 | F43 | C | a | β |
| 747 | F43 | C | a | γ |
| 748 | F43 | C | b | α |
| 749 | F43 | C | b | β |
| 750 | F43 | C | b | γ |
| 751 | F43 | C | c | α |
| 752 | F43 | C | c | β |
| 753 | F43 | C | c | γ |
| 754 | F43 | C | d | α |
| 755 | F43 | C | d | β |
| 756 | F43 | C | d | γ |
| 757 | F43 | D | a | α |
| 758 | F43 | D | a | β |
| 759 | F43 | D | a | γ |
| 760 | F43 | D | b | α |
| 761 | F43 | D | b | β |
| 762 | F43 | D | b | γ |
| 763 | F43 | D | c | α |
| 764 | F43 | D | c | β |
| 765 | F43 | D | c | γ |
| 766 | F43 | D | d | α |
| 767 | F43 | D | d | β |
| 768 | F43 | D | d | γ |
| 769 | F44 | A | a | α |
| 770 | F44 | A | a | β |
| 771 | F44 | A | a | γ |
| 772 | F44 | A | b | α |
| 773 | F44 | A | b | β |
| 774 | F44 | A | b | γ |
| 775 | F44 | A | c | α |
| 776 | F44 | A | c | β |
| 777 | F44 | A | c | γ |
| 778 | F44 | A | d | α |
| 779 | F44 | A | d | β |
| 780 | F44 | A | d | γ |
| 781 | F44 | B | a | α |
| 782 | F44 | B | a | β |
| 783 | F44 | B | a | γ |
| 784 | F44 | B | b | α |
| 785 | F44 | B | b | β |
| 786 | F44 | B | b | γ |
| 787 | F44 | B | c | α |
| 788 | F44 | B | c | β |
| 789 | F44 | B | c | γ |
| 790 | F44 | B | d | α |
| 791 | F44 | B | d | β |
| 792 | F44 | B | d | γ |
| 793 | F44 | C | a | α |
| 794 | F44 | C | a | β |
| 795 | F44 | C | a | γ |
| 796 | F44 | C | b | α |
| 797 | F44 | C | b | β |
| 798 | F44 | C | b | γ |
| 799 | F44 | C | c | α |
| 800 | F44 | C | c | β |
| 801 | F44 | C | c | γ |
| 802 | F44 | C | d | α |
| 803 | F44 | C | d | β |
| 804 | F44 | C | d | γ |
| 805 | F45 | A | a | α |
| 806 | F45 | A | a | β |
| 807 | F45 | A | a | γ |
| 808 | F45 | A | b | α |
| 809 | F45 | A | b | β |
| 810 | F45 | A | b | γ |
| 811 | F45 | A | c | α |
| 812 | F45 | A | c | β |
| 813 | F45 | A | c | γ |
| 814 | F45 | A | d | α |
| 815 | F45 | A | d | β |
| 816 | F45 | A | d | γ |
| 817 | F45 | B | a | α |
| 818 | F45 | B | a | β |
| 819 | F45 | B | a | γ |
| 820 | F45 | B | b | α |
| 821 | F45 | B | b | β |
| 822 | F45 | B | b | γ |
| 823 | F45 | B | c | α |
| 824 | F45 | B | c | β |
| 825 | F45 | B | c | γ |
| 826 | F45 | B | d | α |
| 827 | F45 | B | d | β |
| 828 | F45 | B | d | γ |
| 829 | F45 | C | a | α |
| 830 | F45 | C | a | β |
| 831 | F45 | C | a | γ |
| 832 | F45 | C | b | α |
| 833 | F45 | C | b | β |
| 834 | F45 | C | b | γ |
| 835 | F45 | C | c | α |
| 836 | F45 | C | c | β |
| 837 | F45 | C | c | γ |
| 838 | F45 | C | d | α |
| 839 | F45 | C | d | β |
| 840 | F45 | C | d | γ |
| 841 | F46 | A | a | α |
| 842 | F46 | A | a | β |
| 843 | F46 | A | a | γ |
| 844 | F46 | A | b | α |
| 845 | F46 | A | b | β |
| 846 | F46 | A | b | γ |
| 847 | F46 | A | c | α |
| 848 | F46 | A | c | β |
| 849 | F46 | A | c | γ |
| 850 | F46 | A | d | α |
| 851 | F46 | A | d | β |
| 852 | F46 | A | d | γ |
| 853 | F46 | B | a | α |
| 854 | F46 | B | a | β |
| 855 | F46 | B | a | γ |
| 856 | F46 | B | b | α |
| 857 | F46 | B | b | β |
| 858 | F46 | B | b | γ |
| 859 | F46 | B | c | α |
| 860 | F46 | B | c | β |
| 861 | F46 | B | c | γ |
| 862 | F46 | B | d | α |
| 863 | F46 | B | d | β |
| 864 | F46 | B | d | γ |
| 865 | F46 | C | a | α |
| 866 | F46 | C | a | β |
| 867 | F46 | C | a | γ |
| 868 | F46 | C | b | α |
| 869 | F46 | C | b | β |
| 870 | F46 | C | b | γ |
| 871 | F46 | C | c | α |
| 872 | F46 | C | c | β |
| 873 | F46 | C | c | γ |
| 874 | F46 | C | d | α |
| 875 | F46 | C | d | β |
| 876 | F46 | C | d | γ |
| 877 | F46 | D | a | α |
| 878 | F46 | D | a | β |
| 879 | F46 | D | a | γ |
| 880 | F46 | D | b | α |
| 881 | F46 | D | b | β |
| 882 | F46 | D | b | γ |
| 883 | F46 | D | c | α |
| 884 | F46 | D | c | β |
| 885 | F46 | D | c | γ |
| 886 | F46 | D | d | α |
| 887 | F46 | D | d | β |
| 888 | F46 | D | d | γ |
| 889 | F47 | A | a | α |
| 890 | F47 | A | a | β |
| 891 | F47 | A | a | γ |
| 892 | F47 | A | b | α |
| 893 | F47 | A | b | β |
| 894 | F47 | A | b | γ |
| 895 | F47 | A | c | α |
| 896 | F47 | A | c | β |
| 897 | F47 | A | c | γ |
| 898 | F47 | A | d | α |
| 899 | F47 | A | d | β |
| 900 | F47 | A | d | γ |
| 901 | F47 | B | d | α |
| 902 | F47 | B | a | β |
| 903 | F47 | B | a | γ |
| 904 | F47 | B | b | α |
| 905 | F47 | B | b | β |
| 906 | F47 | B | b | γ |
| 907 | F47 | B | c | α |
| 908 | F47 | B | c | β |
| 909 | F47 | B | c | γ |
| 910 | F47 | B | d | α |
| 911 | F47 | B | d | β |
| 912 | F47 | B | d | γ |
| 913 | F47 | C | a | α |
| 914 | F47 | C | a | β |
| 915 | F47 | C | a | γ |
| 916 | F47 | C | b | α |
| 917 | F47 | C | b | β |
| 918 | F47 | C | b | γ |
| 919 | F47 | C | c | α |
| 920 | F47 | C | c | β |
| 921 | F47 | C | c | γ |
| 922 | F47 | C | d | α |
| 923 | F47 | C | d | β |
| 924 | F47 | C | d | γ |
| 925 | F48 | A | a | α |
| 926 | F48 | A | a | β |
| 927 | F48 | A | a | γ |
| 928 | F48 | A | b | α |
| 929 | F48 | A | b | β |
| 930 | F48 | A | b | γ |
| 931 | F48 | A | c | α |
| 932 | F48 | A | c | β |
| 933 | F48 | A | c | γ |
| 934 | F48 | A | d | α |
| 935 | F48 | A | d | β |
| 936 | F48 | A | d | γ |
| 937 | F48 | B | a | α |
| 938 | F48 | B | a | β |
| 939 | F48 | B | a | γ |
| 940 | F48 | B | b | α |
| 941 | F48 | B | b | β |
| 942 | F48 | B | b | γ |
| 943 | F48 | B | c | α |
| 944 | F48 | B | c | β |
| 945 | F48 | B | c | γ |
| 946 | F48 | B | d | α |
| 947 | F48 | B | d | β |
| 948 | F48 | B | d | γ |
| 949 | F48 | C | a | α |
| 950 | F48 | C | a | β |
| 951 | F48 | C | a | γ |
| 952 | F48 | C | b | α |
| 953 | F48 | C | b | β |
| 954 | F48 | C | b | γ |
| 955 | F48 | C | c | α |
| 956 | F48 | C | c | β |
| 957 | F48 | C | c | γ |
| 958 | F48 | C | d | α |
| 959 | F48 | C | d | β |
| 960 | F48 | C | d | γ |
| 961 | F49 | A | a | α |
| 962 | F49 | A | a | β |
| 963 | F49 | A | a | γ |
| 964 | F49 | A | b | α |
| 965 | F49 | A | b | β |
| 966 | F49 | A | b | γ |
| 967 | F49 | A | c | α |
| 968 | F49 | A | c | β |
| 969 | F49 | A | c | γ |
| 970 | F49 | A | d | α |
| 971 | F49 | A | d | β |
| 972 | F49 | A | d | γ |
| 973 | F49 | B | a | α |
| 974 | F49 | B | a | β |
| 975 | F49 | B | a | γ |
| 976 | F49 | B | b | α |
| 977 | F49 | B | b | β |
| 978 | F49 | B | b | γ |
| 979 | F49 | B | c | α |
| 980 | F49 | B | c | β |
| 981 | F49 | B | c | γ |
| 982 | F49 | B | d | α |
| 983 | F49 | B | d | β |
| 984 | F49 | B | d | γ |
| 985 | F49 | C | a | α |
| 986 | F49 | C | a | β |
| 987 | F49 | C | a | γ |
| 988 | F49 | C | b | α |
| 989 | F49 | C | b | β |
| 990 | F49 | C | b | γ |
| 991 | F49 | C | c | α |
| 992 | F49 | C | c | β |
| 993 | F49 | C | c | γ |
| 994 | F49 | C | d | α |
| 995 | F49 | C | d | β |
| 996 | F49 | C | d | γ |
| 997 | F49 | D | a | α |
| 998 | F49 | D | a | β |
| 999 | F49 | D | a | γ |
| 1000 | F49 | D | b | α |
| 1001 | F49 | D | b | β |
| 1002 | F49 | D | b | γ |
| 1003 | F49 | D | c | α |
| 1004 | F49 | D | c | β |
| 1005 | F49 | D | c | γ |
| 1006 | F49 | D | d | α |
| 1007 | F49 | D | d | β |
| 1008 | F49 | D | d | γ |
| 1009 | F50 | A | a | α |
| 1010 | F50 | A | a | β |
| 1011 | F50 | A | a | γ |
| 1012 | F50 | A | b | α |
| 1013 | F50 | A | b | β |
| 1014 | F50 | A | b | γ |
| 1015 | F50 | A | c | α |
| 1016 | F50 | A | c | β |
| 1017 | F50 | A | c | γ |
| 1018 | F50 | A | d | α |
| 1019 | F50 | A | d | β |
| 1020 | F50 | A | d | γ |
| 1021 | F50 | B | a | α |
| 1022 | F50 | B | a | β |
| 1023 | F50 | B | a | γ |
| 1024 | F50 | B | b | α |
| 1025 | F50 | B | b | β |
| 1026 | F50 | B | b | γ |
| 1027 | F50 | B | c | α |
| 1028 | F50 | B | c | β |
| 1029 | F50 | B | c | γ |
| 1030 | F50 | B | d | α |
| 1031 | F50 | B | d | β |
| 1032 | F50 | B | d | γ |
| 1033 | F50 | C | a | α |
| 1034 | F50 | C | a | β |
| 1035 | F50 | C | a | γ |
| 1036 | F50 | C | b | α |
| 1037 | F50 | C | b | β |
| 1038 | F50 | C | b | γ |
| 1039 | F50 | C | c | α |
| 1040 | F50 | C | c | β |
| 1041 | F50 | C | c | γ |
| 1042 | F50 | C | d | α |
| 1043 | F50 | C | d | β |
| 1044 | F50 | C | d | γ |
| 1045 | F51 | A | a | α |
| 1046 | F51 | A | a | β |
| 1047 | F51 | A | a | γ |
| 1048 | F51 | A | b | α |
| 1049 | F51 | A | b | β |
| 1050 | F51 | A | b | γ |
| 1051 | F51 | A | c | α |
| 1052 | F51 | A | c | β |
| 1053 | F51 | A | c | γ |
| 1054 | F51 | A | d | α |
| 1055 | F51 | A | d | β |
| 1056 | F51 | A | d | γ |
| 1057 | F51 | B | a | α |
| 1058 | F51 | B | a | β |
| 1059 | F51 | B | a | γ |
| 1060 | F51 | B | b | α |
| 1061 | F51 | B | b | β |
| 1062 | F51 | B | b | γ |
| 1063 | F51 | B | c | α |
| 1064 | F51 | B | c | β |
| 1065 | F51 | B | c | γ |
| 1066 | F51 | B | d | α |
| 1067 | F51 | B | d | β |
| 1068 | F51 | B | d | γ |
| 1069 | F51 | C | a | α |
| 1070 | F51 | C | a | β |
| 1071 | F51 | C | a | γ |
| 1072 | F51 | C | b | α |
| 1073 | F51 | C | b | β |
| 1074 | F51 | C | b | γ |
| 1075 | F51 | C | c | α |
| 1076 | F51 | C | c | β |
| 1077 | F51 | C | c | γ |
| 1078 | F51 | C | d | α |
| 1079 | F51 | C | d | β |
| 1080 | F51 | C | d | γ |
| 1081 | F52 | A | a | α |
| 1082 | F52 | A | a | β |
| 1083 | F52 | A | a | γ |
| 1084 | F52 | A | b | α |
| 1085 | F52 | A | b | β |
| 1086 | F52 | A | b | γ |
| 1087 | F52 | A | c | α |
| 1088 | F52 | A | c | β |
| 1089 | F52 | A | c | γ |
| 1090 | F52 | A | d | α |
| 1091 | F52 | A | d | β |
| 1092 | F52 | A | d | γ |
| 1093 | F52 | B | a | α |
| 1094 | F52 | B | a | β |
| 1095 | F52 | B | a | γ |
| 1096 | F52 | B | b | α |
| 1097 | F52 | B | b | β |
| 1098 | F52 | B | b | γ |
| 1099 | F52 | B | c | α |
| 1100 | F52 | B | c | β |
| 1101 | F52 | B | c | γ |
| 1102 | F52 | B | d | α |
| 1103 | F52 | B | d | β |
| 1104 | F52 | B | d | γ |
| 1105 | F52 | C | a | α |
| 1106 | F52 | C | a | β |
| 1107 | F52 | C | a | γ |
| 1108 | F52 | C | b | α |
| 1109 | F52 | C | b | β |
| 1110 | F52 | C | b | γ |
| 1111 | F52 | C | c | α |
| 1112 | F52 | C | c | β |
| 1113 | F52 | C | c | γ |
| 1114 | F52 | C | d | α |
| 1115 | F52 | C | d | β |
| 1116 | F52 | C | d | γ |
| 1117 | F52 | D | a | α |
| 1118 | F52 | D | a | β |
| 1119 | F52 | D | a | γ |
| 1120 | F52 | D | b | α |
| 1121 | F52 | D | b | β |
| 1122 | F52 | D | b | γ |
| 1123 | F52 | D | c | α |
| 1124 | F52 | D | c | β |
| 1125 | F52 | D | c | γ |
| 1126 | F52 | D | d | α |
| 1127 | F52 | D | d | β |
| 1128 | F52 | D | d | γ |
| 1129 | F53 | A | a | α |
| 1130 | F53 | A | a | β |
| 1131 | F53 | A | a | γ |
| 1132 | F53 | A | b | α |
| 1133 | F53 | A | b | β |
| 1134 | F53 | A | b | γ |
| 1135 | F53 | A | c | α |
| 1136 | F53 | A | c | β |
| 1137 | F53 | A | c | γ |
| 1138 | F53 | A | a | α |
| 1139 | F53 | A | d | β |
| 1140 | F53 | A | d | γ |
| 1141 | F53 | B | a | α |
| 1142 | F53 | B | a | β |
| 1143 | F53 | B | a | γ |
| 1144 | F53 | B | b | α |
| 1145 | F53 | B | b | β |
| 1146 | F53 | B | b | γ |
| 1147 | F53 | B | c | α |
| 1148 | F53 | B | c | β |
| 1149 | F53 | B | c | γ |
| 1150 | F53 | B | d | α |
| 1151 | F53 | B | d | β |
| 1152 | F53 | B | d | γ |
| 1153 | F53 | C | a | α |
| 1154 | F53 | C | a | β |
| 1155 | F53 | C | a | γ |
| 1156 | F53 | C | b | α |
| 1157 | F53 | C | b | β |
| 1158 | F53 | C | b | γ |
| 1159 | F53 | C | c | α |
| 1160 | F53 | C | c | β |
| 1161 | F53 | C | c | γ |
| 1162 | F53 | C | d | α |
| 1163 | F53 | C | d | β |
| 1164 | F53 | C | d | γ |
| 1165 | F54 | A | a | α |
| 1166 | F54 | A | a | β |
| 1167 | F54 | A | a | γ |
| 1168 | F54 | A | b | α |
| 1169 | F54 | A | b | β |
| 1170 | F54 | A | b | γ |
| 1171 | F54 | A | c | α |
| 1172 | F54 | A | c | β |
| 1173 | F54 | A | c | γ |
| 1174 | F54 | A | d | α |
| 1175 | F54 | A | d | β |
| 1176 | F54 | A | d | γ |
| 1177 | F54 | B | a | α |
| 1178 | F54 | B | a | β |
| 1179 | F54 | B | a | γ |
| 1180 | F54 | B | b | α |
| 1181 | F54 | B | b | β |
| 1182 | F54 | B | b | γ |
| 1183 | F54 | B | c | α |
| 1184 | F54 | B | c | β |
| 1185 | F54 | B | c | γ |
| 1186 | F54 | B | d | α |
| 1187 | F54 | B | d | β |
| 1188 | F54 | B | d | γ |
| 1189 | F54 | C | a | α |
| 1190 | F54 | C | a | β |
| 1191 | F54 | C | a | γ |
| 1192 | F54 | C | b | α |
| 1193 | F54 | C | b | β |
| 1194 | F54 | C | b | γ |
| 1195 | F54 | C | c | α |
| 1196 | F54 | C | c | β |
| 1197 | F54 | C | c | γ |
| 1198 | F54 | C | d | α |
| 1199 | F54 | C | d | β |
| 1200 | F54 | C | d | γ |
| 1201 | F55 | A | a | α |
| 1202 | F55 | A | a | β |
| 1203 | F55 | A | a | γ |
| 1204 | F55 | A | b | α |
| 1205 | F55 | A | b | β |
| 1206 | F55 | A | b | γ |
| 1207 | F55 | A | c | α |
| 1208 | F55 | A | c | β |
| 1209 | F55 | A | c | γ |
| 1210 | F55 | A | d | α |
| 1211 | F55 | A | d | β |
| 1212 | F55 | A | d | γ |
| 1213 | F55 | B | a | α |
| 1214 | F55 | B | a | β |
| 1215 | F55 | B | a | γ |
| 1216 | F55 | B | b | α |
| 1217 | F55 | B | b | β |
| 1218 | F55 | B | b | γ |
| 1219 | F55 | B | c | α |
| 1220 | F55 | B | c | β |
| 1221 | F55 | B | c | γ |
| 1222 | F55 | B | d | α |
| 1223 | F55 | B | d | β |
| 1224 | F55 | B | d | γ |
| 1225 | F55 | C | a | α |
| 1226 | F55 | C | a | β |
| 1227 | F55 | C | a | γ |
| 1228 | F55 | C | b | α |
| 1229 | F55 | C | b | β |
| 1230 | F55 | C | b | γ |
| 1231 | F55 | C | c | α |
| 1232 | F55 | C | c | β |
| 1233 | F55 | C | c | γ |
| 1234 | F55 | C | d | α |
| 1235 | F55 | C | d | β |
| 1236 | F55 | C | d | γ |
| 1237 | F55 | D | a | α |
| 1238 | F55 | D | a | β |
| 1239 | F55 | D | a | γ |
| 1240 | F55 | D | b | α |
| 1241 | F55 | D | b | β |
| 1242 | F55 | D | b | γ |
| 1243 | F55 | D | c | α |
| 1244 | F55 | D | c | β |
| 1245 | F55 | D | c | γ |
| 1246 | F55 | D | d | α |
| 1247 | F55 | D | d | β |
| 1248 | F55 | D | d | γ |
| 1249 | F56 | A | a | α |
| 1250 | F56 | A | a | β |
| 1251 | F56 | A | a | γ |
| 1252 | F56 | A | b | α |
| 1253 | F56 | A | b | β |
| 1254 | F56 | A | b | γ |
| 1255 | F56 | A | c | α |
| 1256 | F56 | A | c | β |
| 1257 | F56 | A | c | γ |
| 1258 | F56 | A | d | α |
| 1259 | F56 | A | d | β |
| 1260 | F56 | A | d | γ |
| 1261 | F56 | B | a | α |
| 1262 | F56 | B | a | β |
| 1263 | F56 | B | a | γ |
| 1264 | F56 | B | b | α |
| 1265 | F56 | B | b | β |
| 1266 | F56 | B | b | γ |
| 1267 | F56 | B | c | α |
| 1268 | F56 | B | c | β |
| 1269 | F56 | B | c | γ |
| 1270 | F56 | B | d | α |
| 1271 | F56 | B | d | β |
| 1272 | F56 | B | d | γ |
| 1273 | F56 | C | a | α |
| 1274 | F56 | C | a | β |
| 1275 | F56 | C | a | γ |
| 1276 | F56 | C | b | α |
| 1277 | F56 | C | b | β |
| 1278 | F56 | C | b | γ |
| 1279 | F56 | C | c | α |
| 1280 | F56 | C | c | β |
| 1281 | F56 | C | c | γ |
| 1282 | F56 | C | d | α |
| 1283 | F56 | C | d | β |
| 1284 | F56 | C | d | γ |
| 1285 | F56 | D | a | α |
| 1286 | F56 | D | a | β |
| 1287 | F56 | D | a | γ |
| 1288 | F56 | D | b | α |
| 1289 | F56 | D | b | β |
| 1290 | F56 | D | b | γ |
| 1291 | F56 | D | c | α |
| 1292 | F56 | D | c | β |

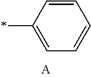 [SMILES: *c1ccccc1]

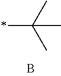 [SMILES: *C(C)(C)C]

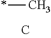 [SMILES: *C]

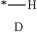 [SMILES: *[H]]

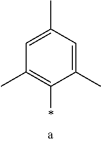

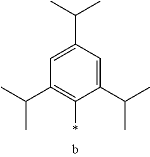 [SMILES: *c1c(C(C)C)cc(C(C)C)cc1C(C)C]

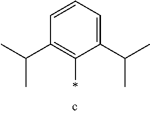 [SMILES: *c1c(C(C)C)cccc1C(C)C]

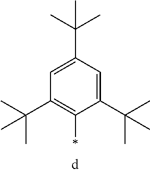 [SMILES: *c1c(C(C)(C)C)cc(C(C)(C)C)cc1C(C)(C)C]

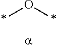 [SMILES: *O*]

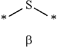 [SMILES: *S*]

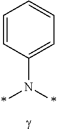 [SMILES: *N(*)c1ccccc1]

In one aspect of the present invention, the skeletons (1a) to (12b) are skeletons in which other rings are not further fused. In one aspect of the present invention, the skeletons (1a) to (12b) are skeletons in which other rings can be further fused. Regarding other rings mentioned herein, the above descriptions on the cyclic structures formed by bonding R¹ and R², R² and R³, R³ and R⁴, R⁴ and R⁵, R⁵ and R⁶, R⁶ and R⁷, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁸ and R¹⁹, R¹⁹ and R²⁰, R²⁰ and R²¹, R²² and R²³, R²³ and R²⁴, R²⁴ and R²⁵, and R²⁵ and R²⁶ to each other can be referred to.

In one aspect of the present invention, A¹ and A² in the general formula (G) are acceptor groups. For example, a compound having acceptor groups at positions of A¹ and A² and having any of the skeletons (1a) to (12b) can be mentioned. In relation to descriptions and specific examples of the acceptor group, descriptions, and specific examples of the acceptor group for A¹ and A² in the general formula (G) can be referred to.

Hereinafter, specific examples of a compound in which A¹ and A² are acceptor groups will be given. The compounds in which A¹ and A² are acceptor groups, which can be used in the present invention, are not construed as limiting to the following specific examples. The following specific examples have structures in which both A¹ and A² are “A”, and the structure of each compound is specified by individually specifying the “A”.

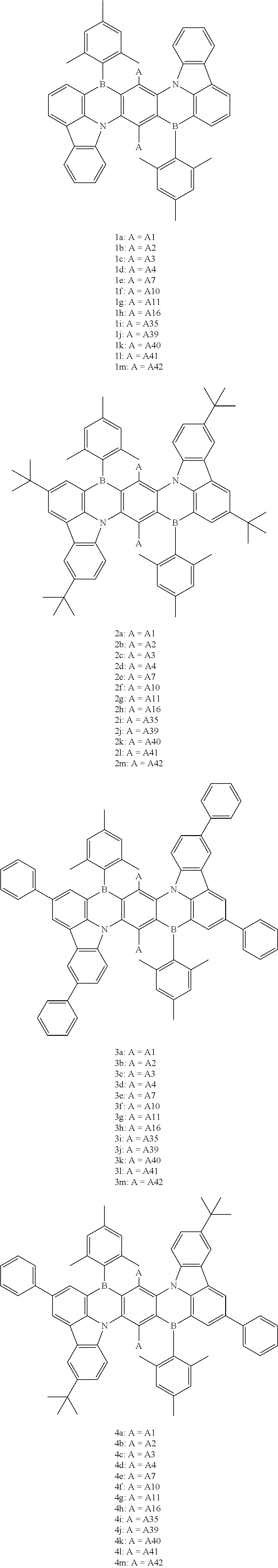

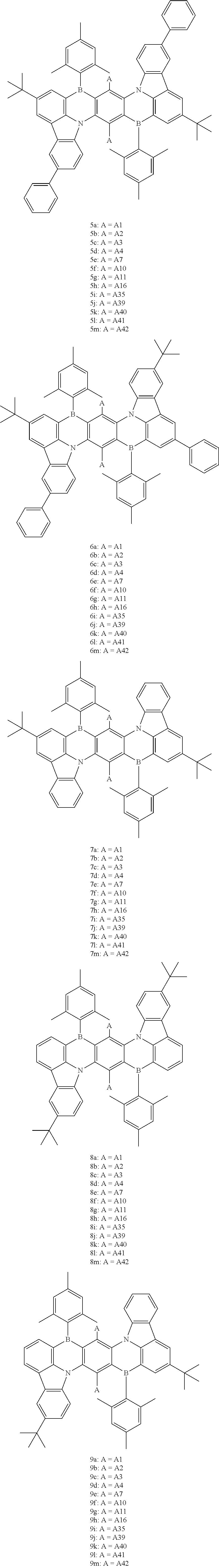

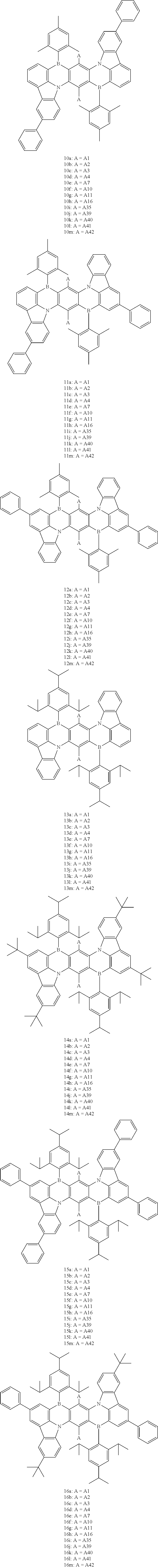

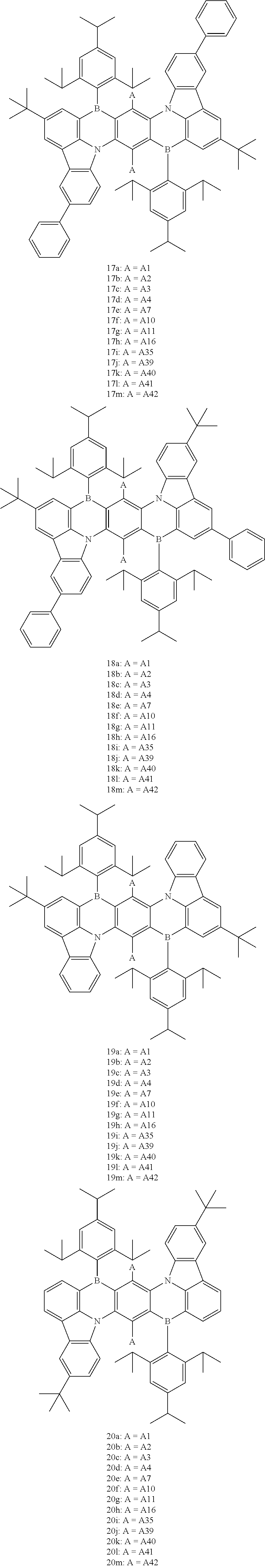

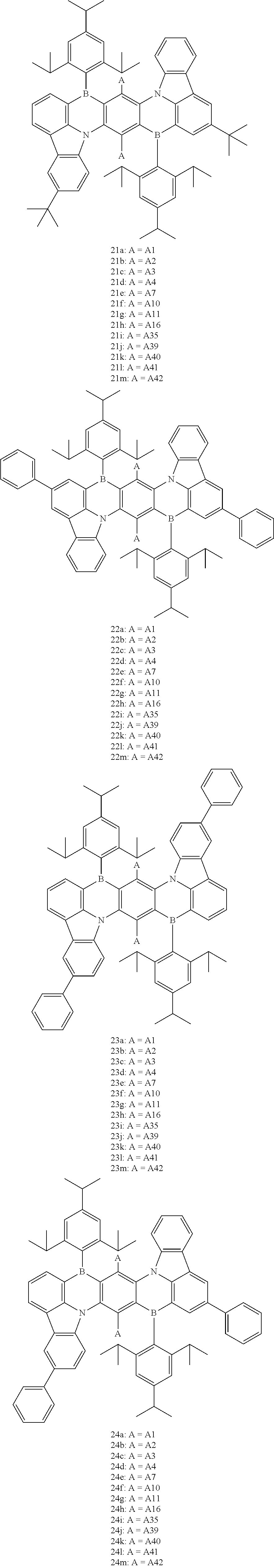

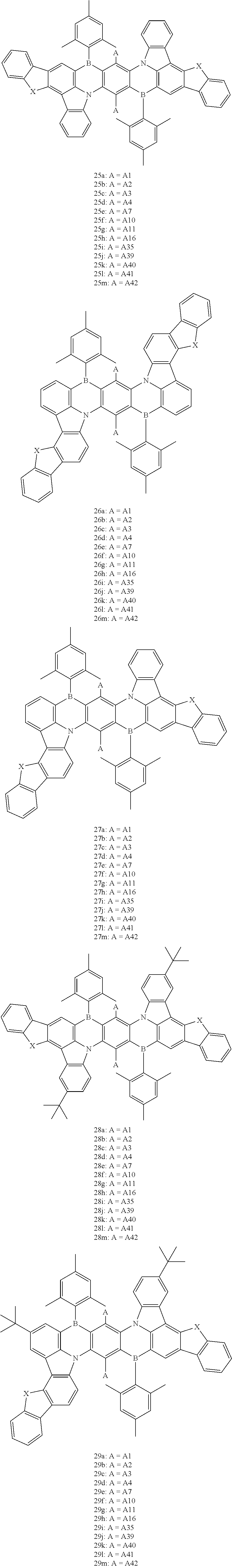

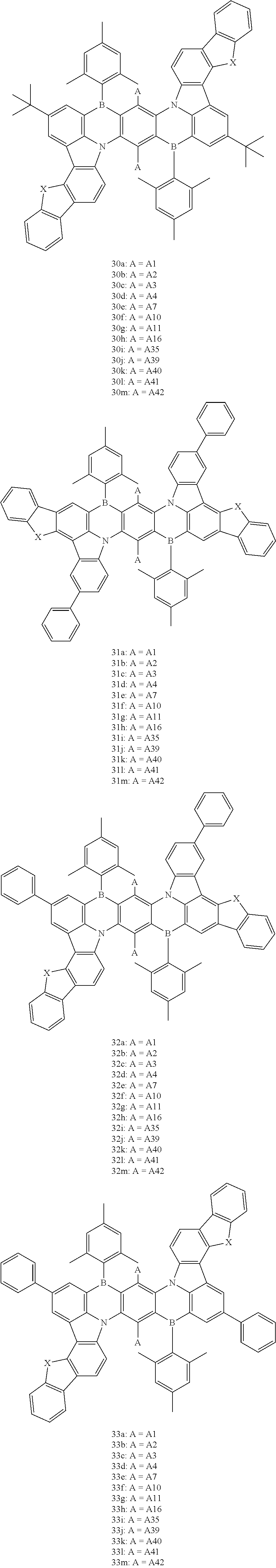

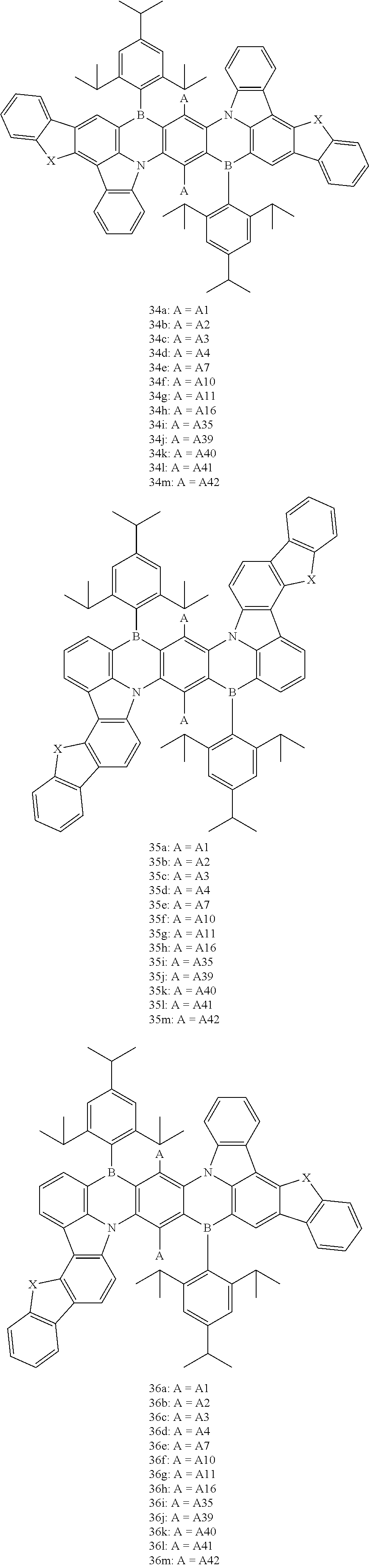

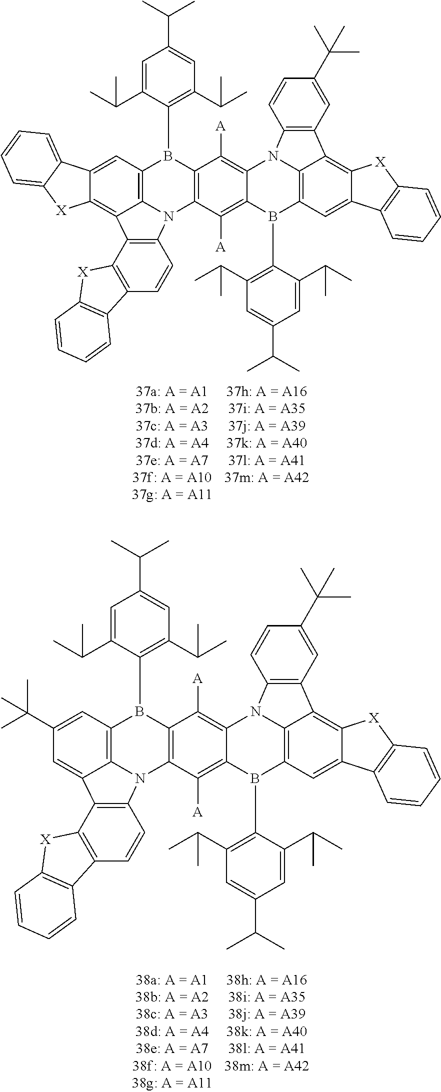

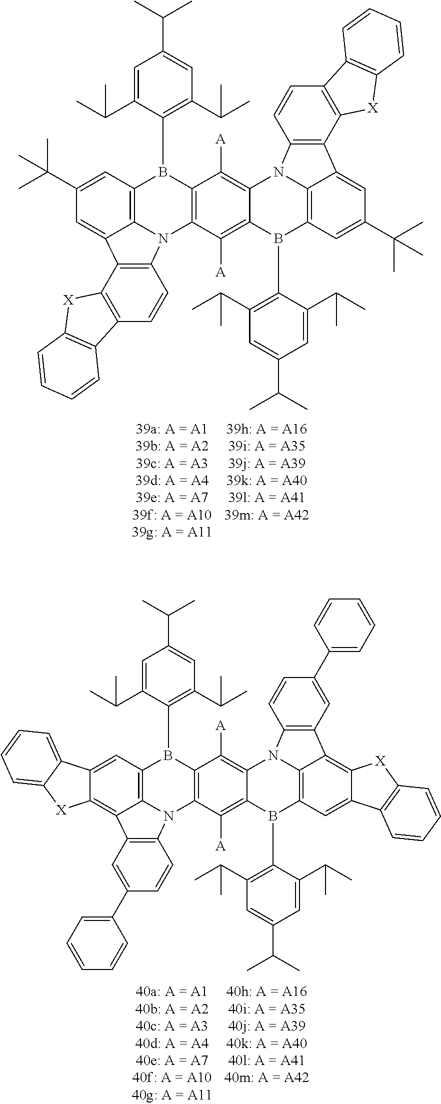

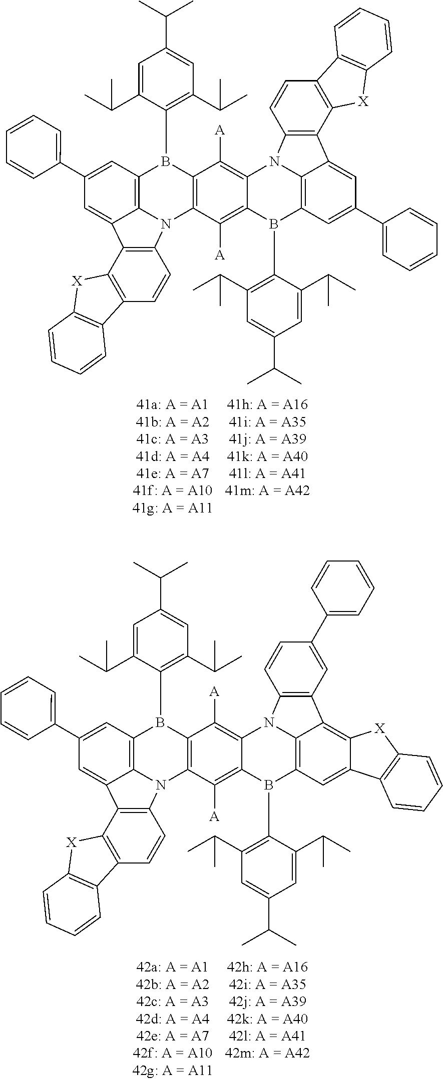

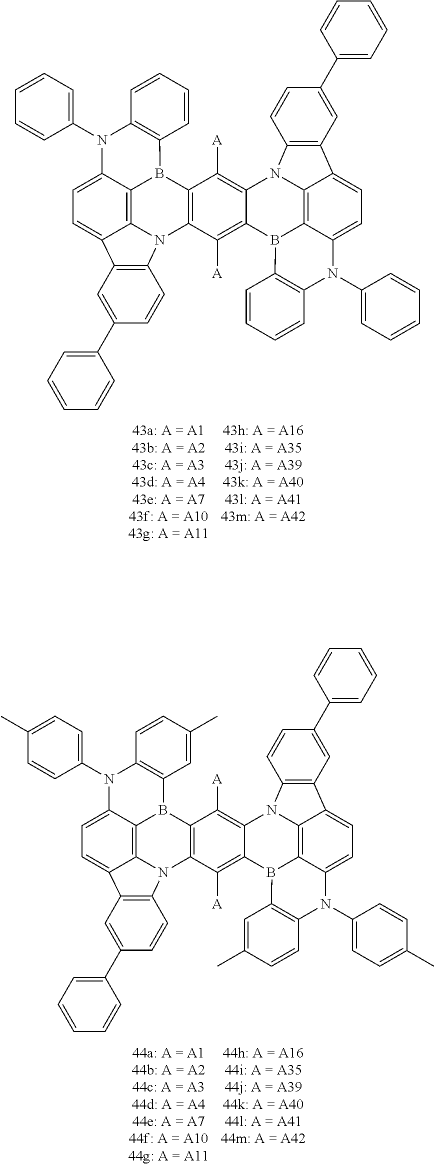

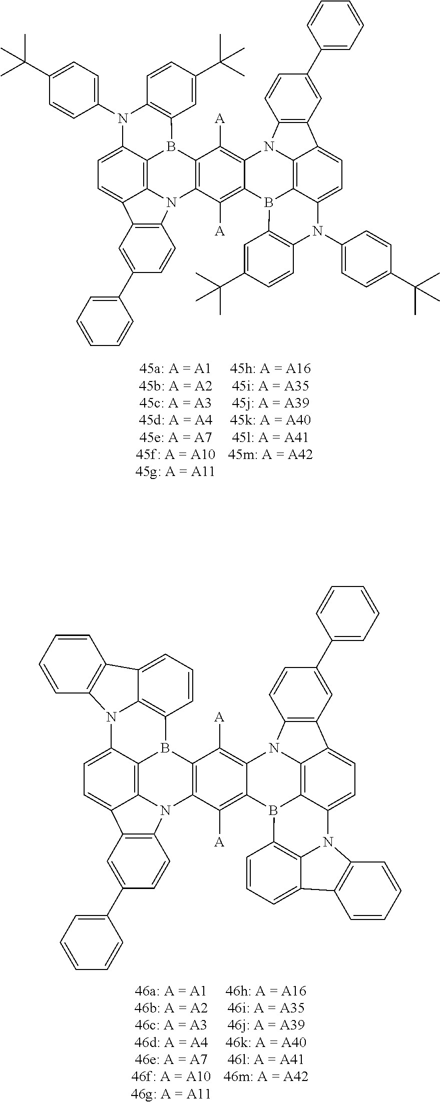

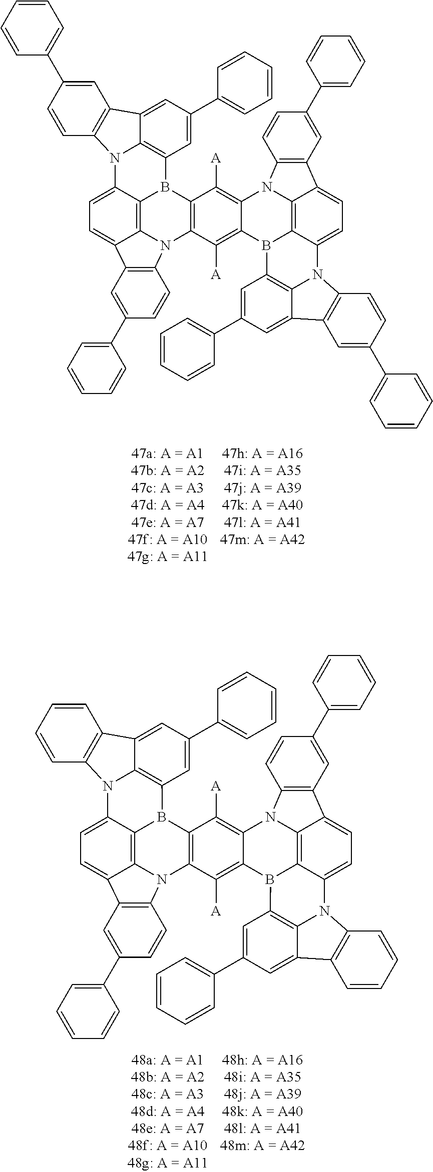

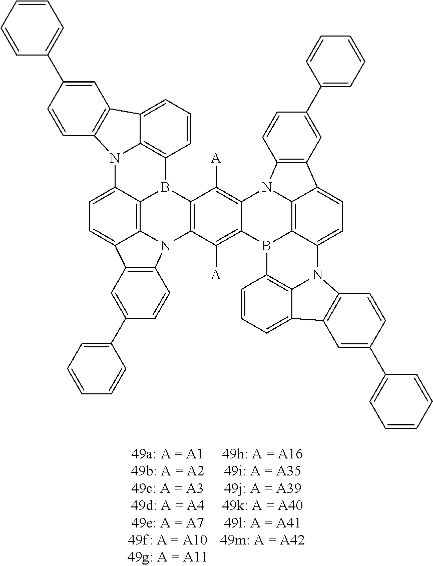

In one aspect of the present invention, as the compound represented by the general formula (G), a compound having a rotationally symmetric structure is selected. In one aspect of the present invention, as the compound represented by the general formula (G), a compound having an axisymmetric structure is selected. In one aspect of the present invention, as the compound represented by the general formula (G), a compound having an asymmetric structure is selected.

Specific examples of a compound having an asymmetric skeleton will be given below. The compounds having asymmetric skeletons or the compounds having asymmetric structures, which can be used in the present invention, are not construed as limiting to the following specific examples. In relation to specific examples including X, it is assumed that a compound in which all X's in the molecule are oxygen atoms, and a compound in which all X's in the molecule are sulfur atoms are disclosed, respectively. A compound in which some of X's in the molecule are oxygen atoms, and the rest are sulfur atoms may also be adopted.

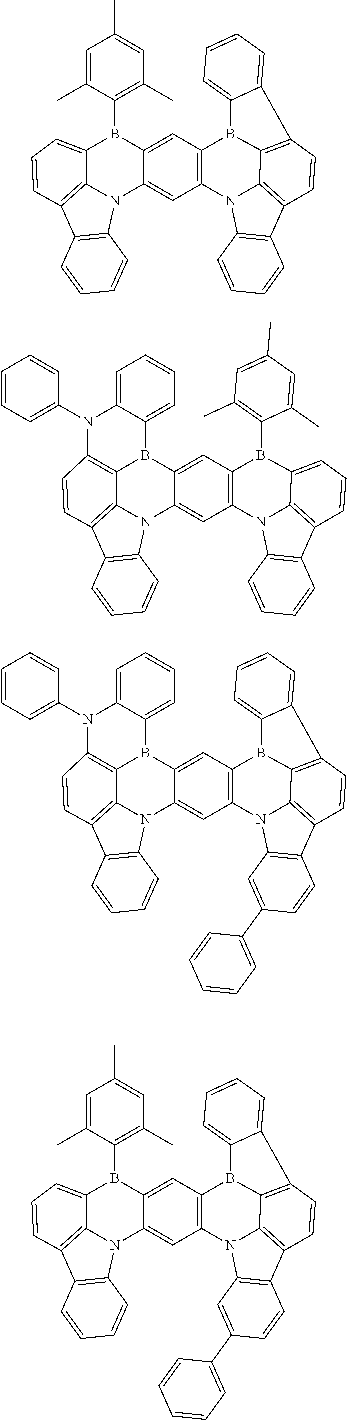

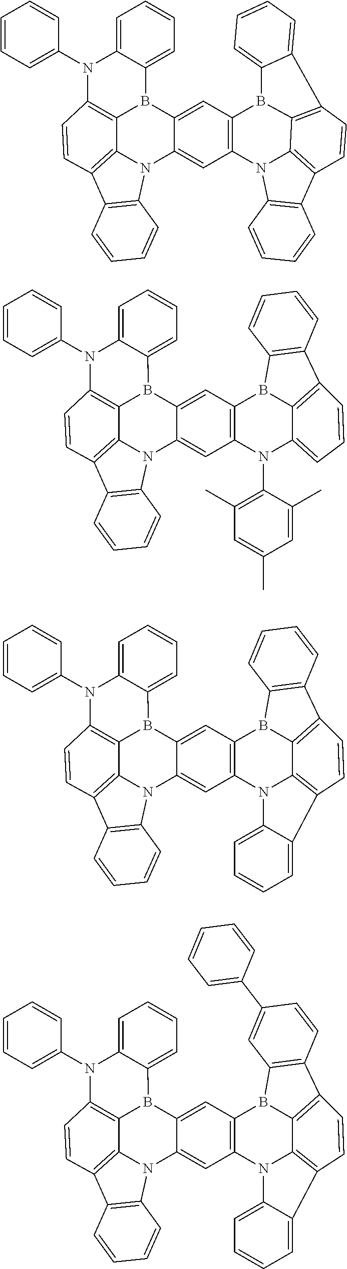

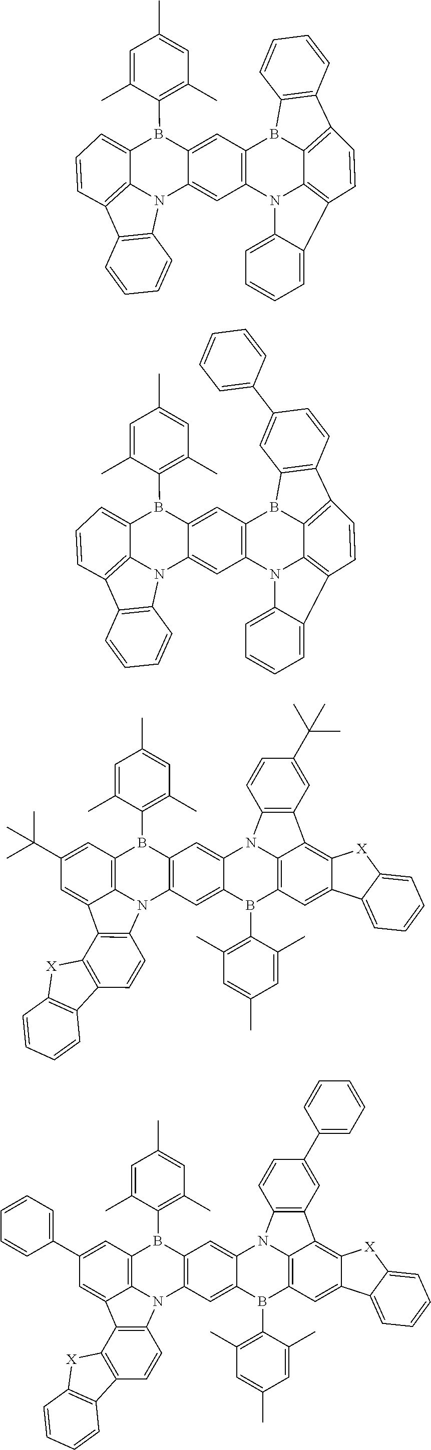

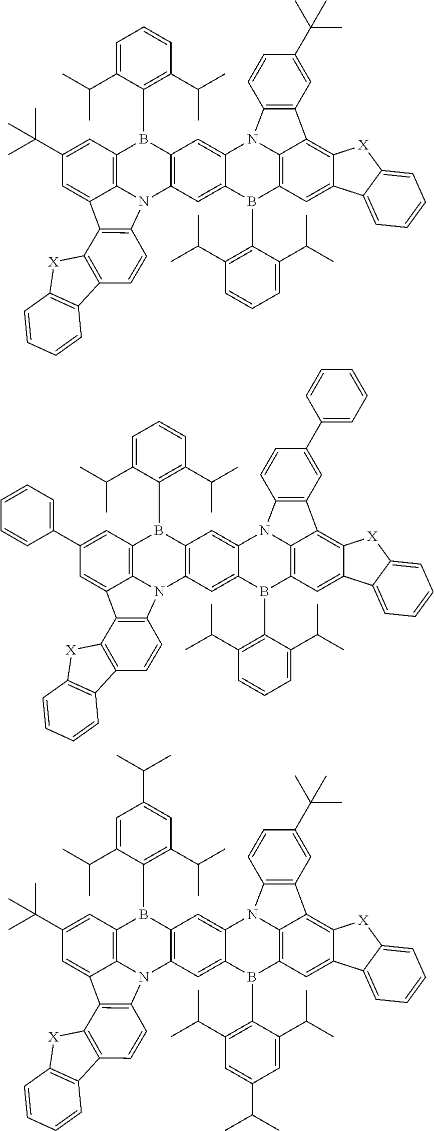

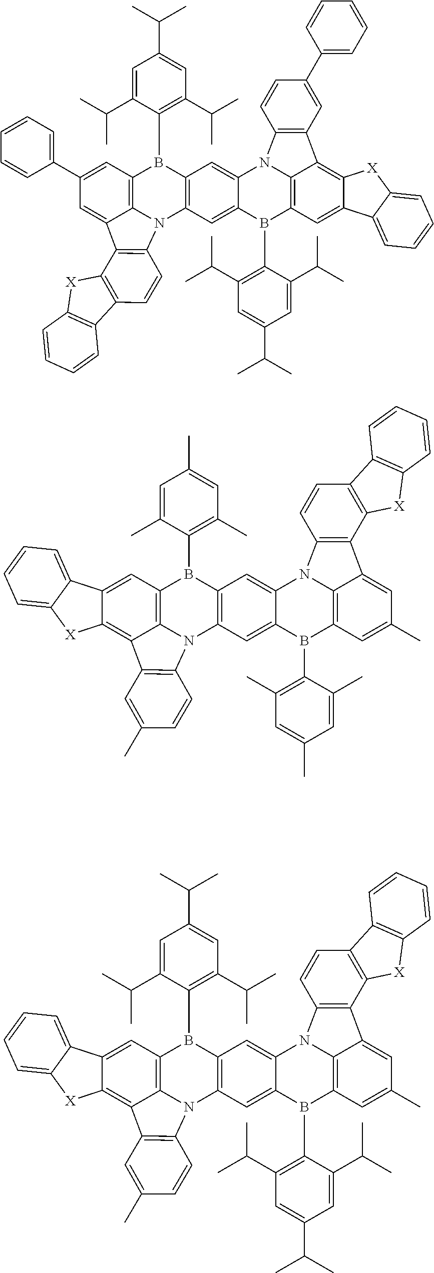

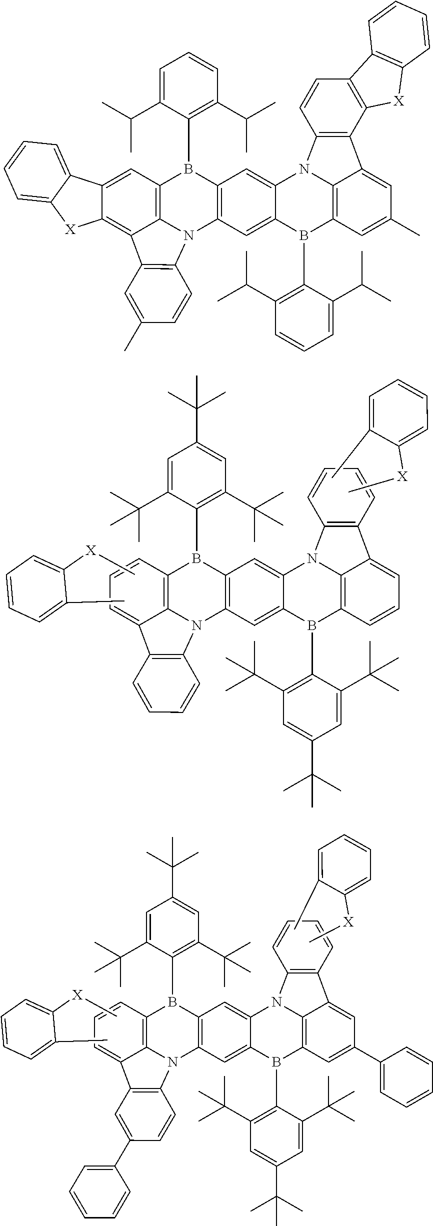

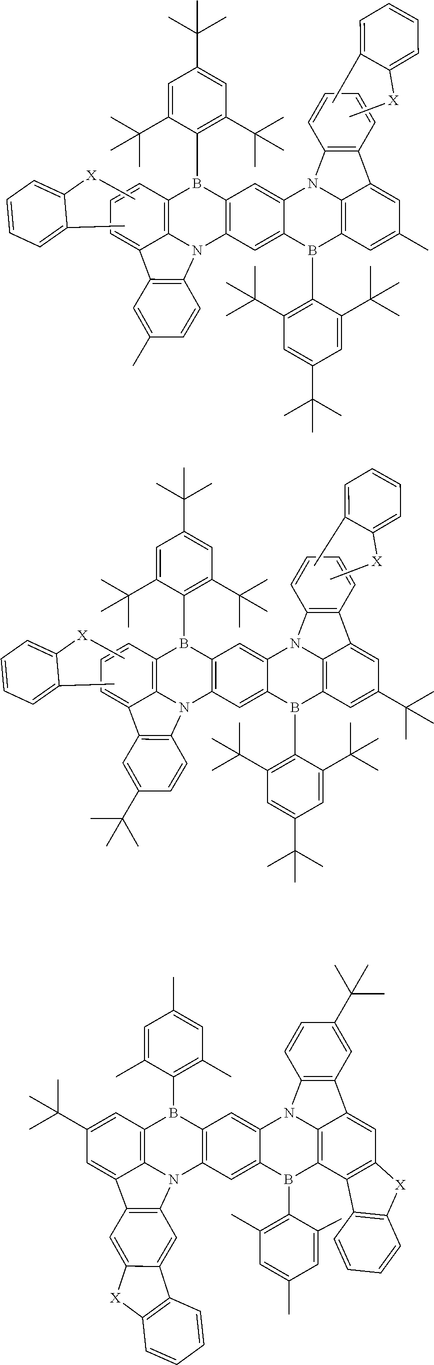

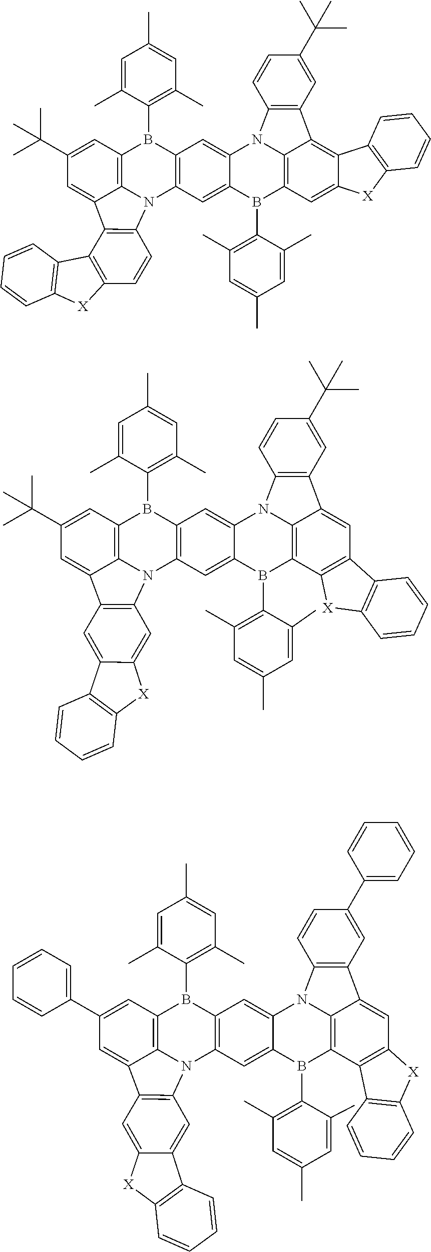

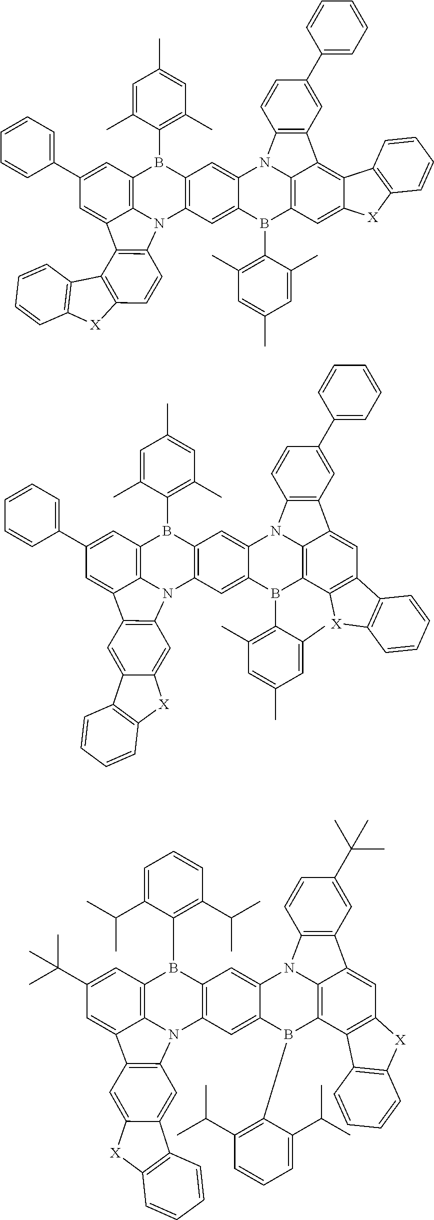

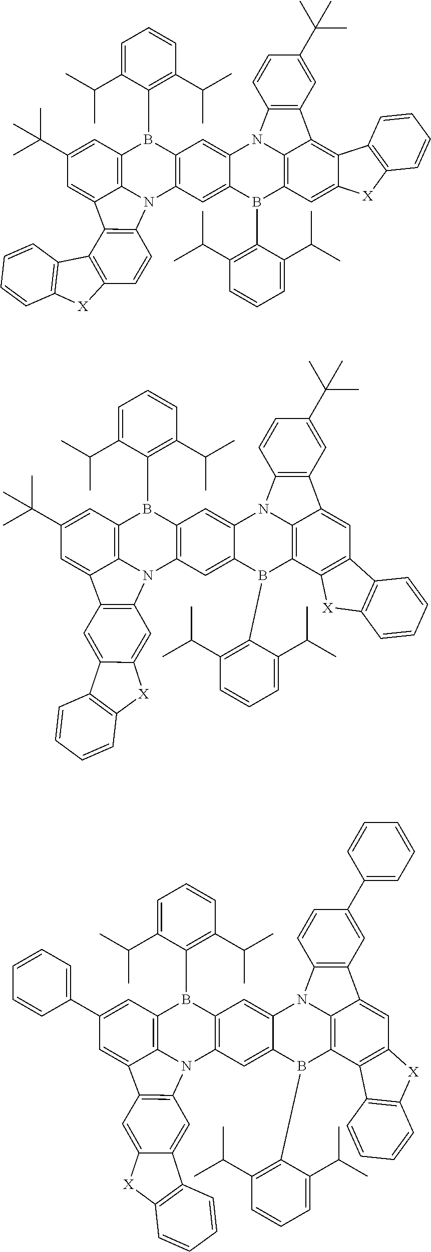

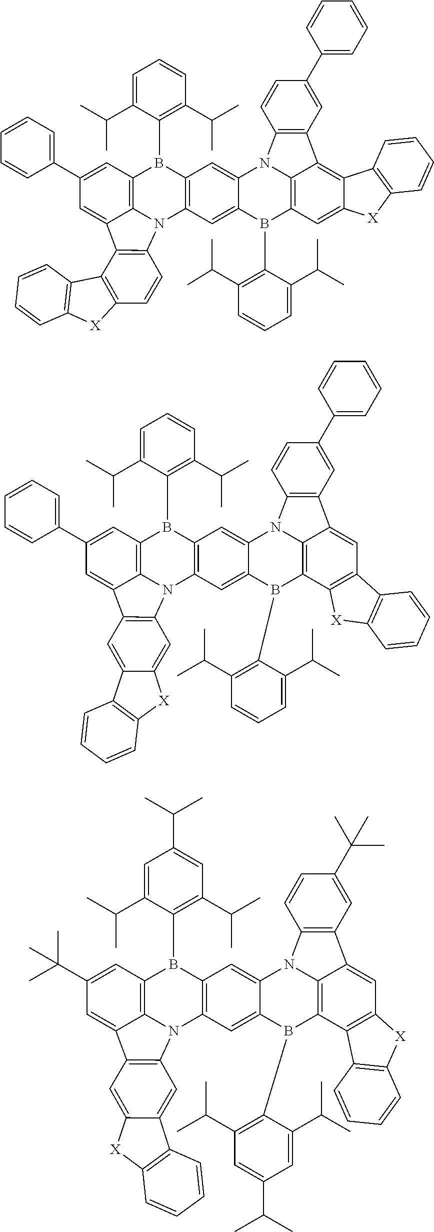

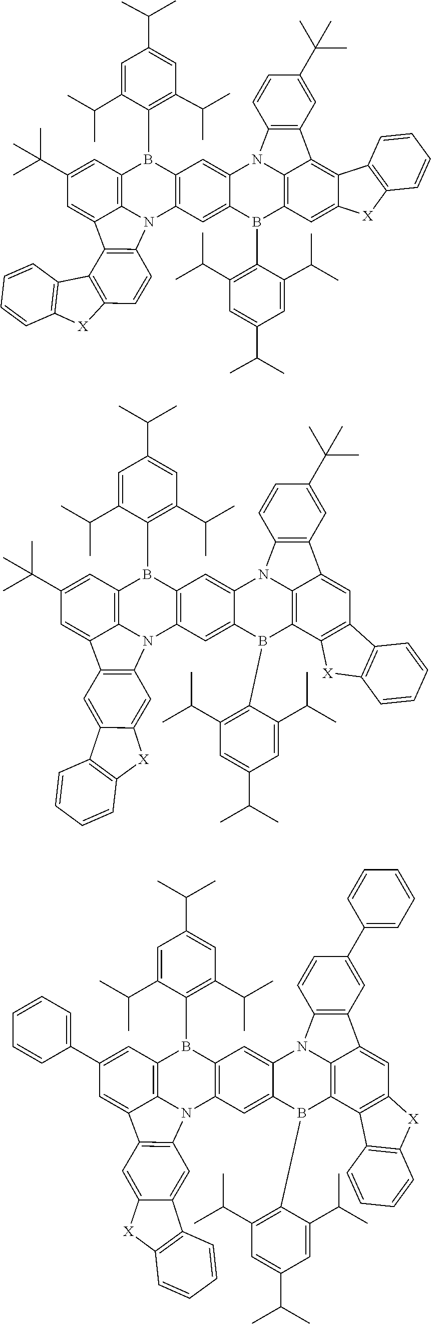

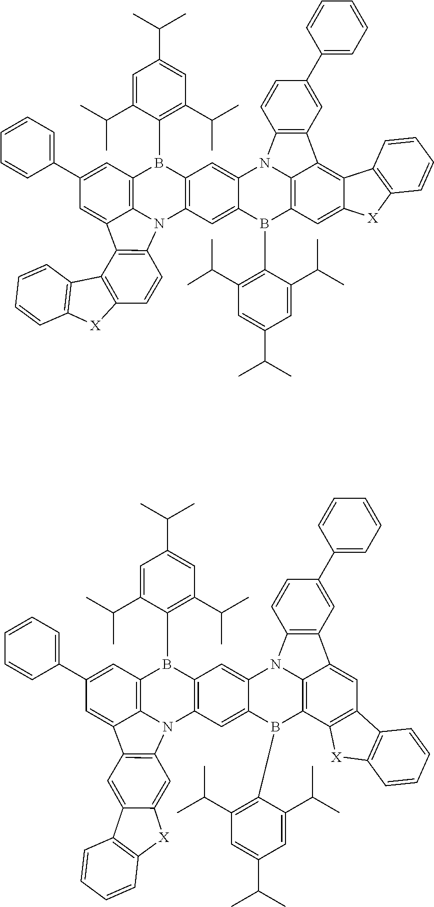

Hereinafter, specific examples of a compound that has a symmetric skeleton but has an asymmetric structure because a substituent is asymmetrically bonded will be given. The compounds having asymmetric structures, which can be used in the present invention, are not construed as limiting to the following specific examples.

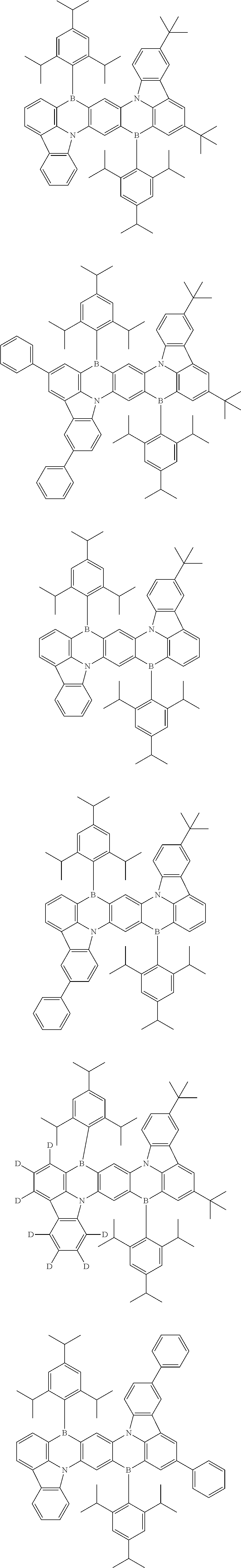

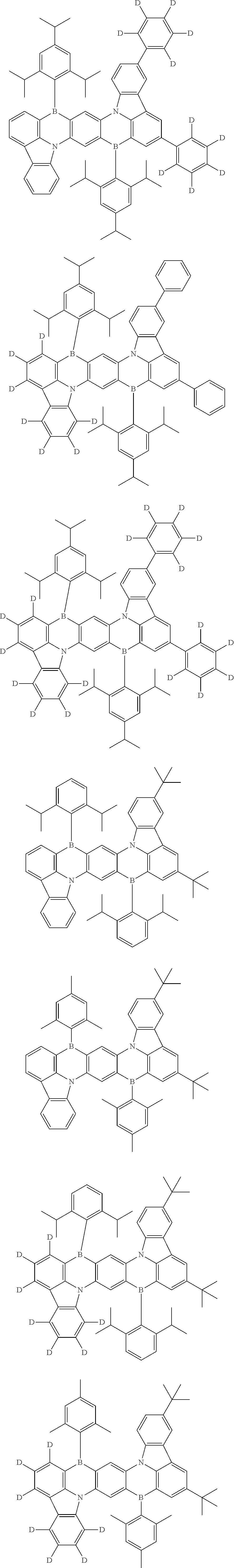

In one aspect of the present invention, R³ in the general formula (G) is not a diarylamino group (two aryl groups constituting the diarylamino group can be bonded to each other). In one preferred aspect of the present invention, R³ in the general formula (G) is a hydrogen atom, a deuterium atom, or an acceptor group (not a donor group).

In one aspect of the present invention, at least one of n1 to n4 in the general formula (1a) is 1 or more. In one preferred aspect of the present invention, at least one of m1 and m2 in the general formula (1a) is 1 or more. In a more preferable aspect of the present invention, at least one of n1 to n4 in the general formula (1a) is 1 or more, and moreover, at least one of m1 and m2 in the general formula (1a) is 1 or more.

In one aspect of the present invention, at least one of n5 to n8 in the general formula (1b) is 1 or more. In one preferred aspect of the present invention, at least one of m3 and m4 in the general formula (1b) is 1 or more. In a more preferable aspect of the present invention, at least one of n5 to n8 in the general formula (1b) is 1 or more, and moreover, at least one of m3 and m4 in the general formula (1b) is 1 or more.

When at least one of m1 and m2 is 1 or more, and at least one of m3 and m4 is 1 or more, it is preferable that at least one of R⁴¹ and R⁴² and at least one of R⁴³ and R⁴⁴ are alkyl groups which can be substituted with deuterium atoms, and for example, all of R⁴¹ to R⁴⁴ are alkyl groups which can be substituted with deuterium atoms. When at least one of n1 to n4 is 1 or more, and at least one of n5 to n8 is 1 or more, it is preferable that at least one of Ar¹ to A⁴ and at least one of Ar⁵ to Ar⁸ are aryl groups which can be substituted with deuterium atoms or alkyl groups, and for example, all of Ar¹ to Ar⁸ are aryl groups which can be substituted with deuterium atoms or alkyl groups.

In one aspect of the present invention, when X¹ in the general formula (G) is a boron atom, and R⁸, R¹⁰, R¹², R¹³, R¹⁵, and R¹⁷ are alkyl groups (or methyl groups), at least one of R¹ to R⁷, R¹⁸ to R²⁰, and R²³ to R²⁶ is a substituent, preferably a group of Substituent Group E, and is, for example, an aryl group that can be substituted with a deuterium atom or an alkyl group. In one aspect of the present invention, when X² in the general formula (G) is a boron atom, and R⁸, R¹⁰, R¹², R²², R²⁴, and R²⁶ are alkyl groups (or methyl groups), at least one of R¹ to R⁷, R¹³ to R¹⁶, and R¹⁹ to R²¹ is a substituent, preferably a group of Substituent Group E, and is, for example, an aryl group that can be substituted with a deuterium atom or an alkyl group.

In one aspect of the present invention, when X¹ in the general formula (G) is a boron atom, and any one combination of R⁸ and R⁹, and R⁹ and R¹⁰, and any one combination of R¹⁵ and R¹⁶, and R¹⁶ and R¹⁷ bond to each other to form an aromatic ring (or a benzene ring), at least one of R¹ to R⁷, R¹⁸ to R²⁰, and R²³ to R²⁶ is a substituent, preferably a group of Substituent Group E, and is, for example, an aryl group that can be substituted with a deuterium atom or an alkyl group. In one aspect of the present invention, when X² in the general formula (G) is a boron atom, and any one combination of R⁸ and R⁹, and R⁹ and R¹⁰, and any one combination of R²² and R²³, and R²³ and R²⁴ bond to each other to form an aromatic ring (or a benzene ring), at least one of R¹ to R⁷, R¹³ to R¹⁶, and R¹⁹ to R²¹ is a substituent, preferably a group of Substituent Group E, and is, for example, an aryl group that can be substituted with a deuterium atom or an alkyl group.

In one aspect of the present invention, R⁹ and R¹¹ in the general formula (G) are neither cyano groups nor alkyl groups. That is, R⁹ and R¹¹ are hydrogen atoms, deuterium atoms, or substituents other than cyano groups and alkyl groups. In one aspect of the present invention, R⁹ and R¹¹ in the general formula (G) are neither cyano groups nor tert-butyl groups.

In one preferred aspect of the present invention, at least one of R⁸ to R¹² in the general formula (G) is a substituent.

In one aspect of the present invention, R³ in the general formula (G) is not a substituted amino group or aryl group. In one aspect of the present invention, R³ in the general formula (G) is not a substituted amino group or phenyl group. In one aspect of the present invention, R³ in the general formula (G) is not a dimethyl amino group, a diphenyl amino group, or a phenyl group.

In one preferred aspect of the present invention, at least one of R¹ to R²⁶ in the general formula (G) is a substituent. More preferably, at least one of R¹ to R²⁶ is an alkyl group, and is, for example, an alkyl group having 1 to 4 carbon atoms.

### (Substrate)

In some embodiments, the organic electroluminescent device of the present invention is supported by a substrate, wherein the substrate is not particularly limited and can be any of those that have been commonly used in an organic electroluminescent device, for example those formed of glass, transparent plastics, quartz, and silicon.

### (Anode)

In some embodiments, the anode of the organic electroluminescent device is made of a metal, an alloy, a conductive compound, or a combination thereof. In some embodiments, the metal, alloy, or conductive compound has a large work function (4 eV or more). In some embodiments, the metal is Au. In some embodiments, the conductive transparent material is selected from CuI, indium tin oxide (ITO), SnO₂, and ZnO. In some embodiments, an amorphous material capable of forming a transparent conductive film, such as IDIXO (In₂O₃—ZnO), is used. In some embodiments, the anode is a thin film. In some embodiments, the thin film is made by vapor deposition or sputtering. In some embodiments, the film is patterned by a photolithography method. In some embodiments, when the pattern may not require high accuracy (for example, approximately 100 μm or more), the pattern can be formed with a mask having a desired shape on vapor deposition or sputtering of the electrode material. In some embodiments, when a material can be applied as a coating material, such as an organic conductive compound, a wet film forming method, such as a printing method and a coating method is used. In some embodiments, when the emitted light goes through the anode, the anode has a transmittance of more than 10%, and the anode has a sheet resistance of several hundred Ohm per square or less. In some embodiments, the thickness of the anode is from 10 to 1,000 nm. In some embodiments, the thickness of the anode is from 10 to 200 nm. In some embodiments, the thickness of the anode varies depending on the material used.

### (Cathode)

In some embodiments, the cathode is made of an electrode material such as a metal having a small work function (4 eV or less) (referred to as an electron injection metal), an alloy, a conductive compound, or a combination thereof. In some embodiments, the electrode material is selected from sodium, a sodium-potassium alloy, magnesium, lithium, a magnesium-copper mixture, a magnesium-silver mixture, a magnesium-aluminum mixture, a magnesium-indium mixture, an aluminum-aluminum oxide (Al₂O₃) mixture, indium, a lithium-aluminum mixture, and a rare earth element. In some embodiments, a mixture of an electron injection metal and a second metal that is a stable metal having a larger work function than the electron injection metal is used. In some embodiments, the mixture is selected from a magnesium-silver mixture, a magnesium-aluminum mixture, a magnesium-indium mixture, an aluminum-aluminum oxide (Al₂O₃) mixture, a lithium-aluminum mixture, and aluminum. In some embodiments, the mixture increases the electron injection property and the durability against oxidation. In some embodiments, the cathode is produced by forming the electrode material into a thin film by vapor deposition or sputtering. In some embodiments, the cathode has a sheet resistance of several hundred Ohm per square or less. In some embodiments, the thickness of the cathode is from 10 nm to 5 μm. In some embodiments, the thickness of the cathode is from 50 to 200 nm. In some embodiments, for transmitting the emitted light, any one of the anode and the cathode of the organic electroluminescent device is transparent or translucent. In some embodiments, the transparent or translucent electroluminescent devices enhance the light emission luminance.

In some embodiments, the cathode is formed with a conductive transparent material, as described for the anode, to form a transparent or translucent cathode. In some embodiments, a device comprises an anode and a cathode, both being transparent or translucent.

### (Injection Layer)

An injection layer is a layer between the electrode and the organic layer. In some embodiments, the injection layer decreases the drive voltage and enhances the light emission luminance. In some embodiments, the injection layer includes a hole injection layer and an electron injection layer. The injection layer can be positioned between the anode and the light emitting layer or the hole transport layer, and between the cathode and the light emitting layer or the electron transport layer. In some embodiments, an injection layer is present. In some embodiments, no injection layer is present.

Preferred compound examples for use as a hole injection material are shown below.

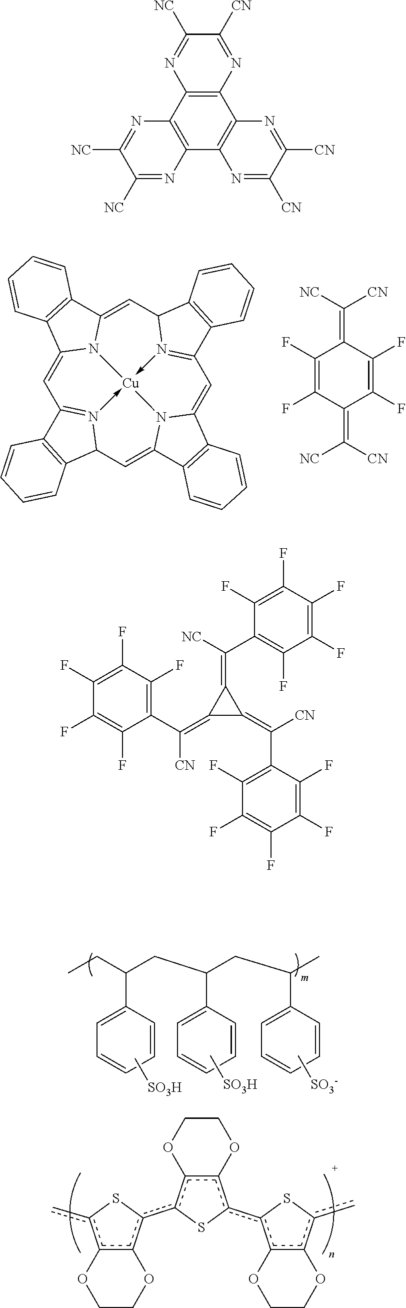

Next, preferred compound examples for use as an electron injection material are shown below.
LiF, CsF,

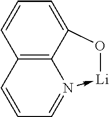

### (Barrier Layer)

A barrier layer is a layer capable of inhibiting charges (electrons or holes) and/or excitons present in the light emitting layer from being diffused outside the light emitting layer. In some embodiments, the electron barrier layer is between the light emitting layer and the hole transport layer, and inhibits electrons from passing through the light emitting layer toward the hole transport layer. In some embodiments, the hole barrier layer is between the light emitting layer and the electron transport layer, and inhibits holes from passing through the light emitting layer toward the electron transport layer. In some embodiments, the barrier layer inhibits excitons from being diffused outside the light emitting layer. In some embodiments, the electron barrier layer and the hole barrier layer form an exciton barrier layer. As used in the present description, the term “electron barrier layer” or “exciton barrier layer” includes a layer that has the functions of both electron barrier layer and of an exciton barrier layer.

### (Hole Barrier Layer)

A hole barrier layer acts as an electron transport layer. In some embodiments, the hole barrier layer inhibits holes from reaching the electron transport layer while transporting electrons. In some embodiments, the hole barrier layer enhances the recombination probability of electrons and holes in the light emitting layer. The material for the hole barrier layer can be the same materials as the ones described for the electron transport layer.

Preferred compound examples for use for the hole barrier layer are shown below.

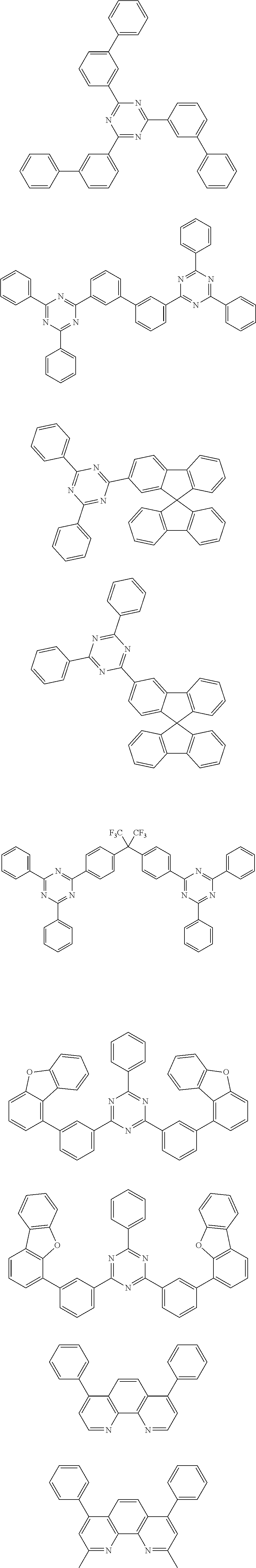

### (Exciton Barrier Layer)

An exciton barrier layer inhibits excitons generated through recombination of holes and electrons in the light emitting layer from being diffused to the charge transport layer. In some embodiments, the exciton barrier layer enables effective confinement of excitons in the light emitting layer. In some embodiments, the light emission efficiency of the device is enhanced. In some embodiments, the exciton barrier layer is adjacent to the light emitting layer on any of the side of the anode and the side of the cathode, or on both the sides. In some embodiments, when the exciton barrier layer is on the side of the anode, the layer can be between the hole transport layer and the light emitting layer and adjacent to the light emitting layer. In some embodiments, when the exciton barrier layer is on the side of the cathode, the layer can be between the light emitting layer and the cathode and adjacent to the light emitting layer. In some embodiments, a hole injection layer, an electron barrier layer, or a similar layer is between the anode and the exciton barrier layer that is adjacent to the light emitting layer on the side of the anode. In some embodiments, a hole injection layer, an electron barrier layer, a hole barrier layer, or a similar layer is between the cathode and the exciton barrier layer that is adjacent to the light emitting layer on the side of the cathode. In some embodiments, the exciton barrier layer comprises excited singlet energy and excited triplet energy, at least one of which is higher than the excited singlet energy and the excited triplet energy of the light emitting material, respectively.

### (Hole Transport Layer)

The hole transport layer comprises a hole transport material. In some embodiments, the hole transport layer is a single layer. In some embodiments, the hole transport layer comprises a plurality of layers.

In some embodiments, the hole transport material has one of injection or transport property of holes and barrier property of electrons. In some embodiments, the hole transport material is an organic material. In some embodiments, the hole transport material is an inorganic material. Examples of known hole transport materials that can be used in the present invention include but are not limited to a triazole derivative, an oxadiazole derivative, an imidazole derivative, a carbazole derivative, an indolocarbazole derivative, a polyarylalkane derivative, a pyrazoline derivative, a pyrazolone derivative, a phenylenediamine derivative, an allylamine derivative, an amino-substituted chalcone derivative, an oxazole derivative, a styrylanthracene derivative, a fluorenone derivative, a hydrazone derivative, a stilbene derivative, a silazane derivative, an aniline copolymer and a conductive polymer oligomer (particularly a thiophene oligomer), or a combination thereof. In some embodiments, the hole transport material is selected from a porphyrin compound, an aromatic tertiary amine compound, and a styrylamine compound. In some embodiments, the hole transport material is an aromatic tertiary amine compound. Preferred specific examples of a compound for use as the hole transport material are shown below.

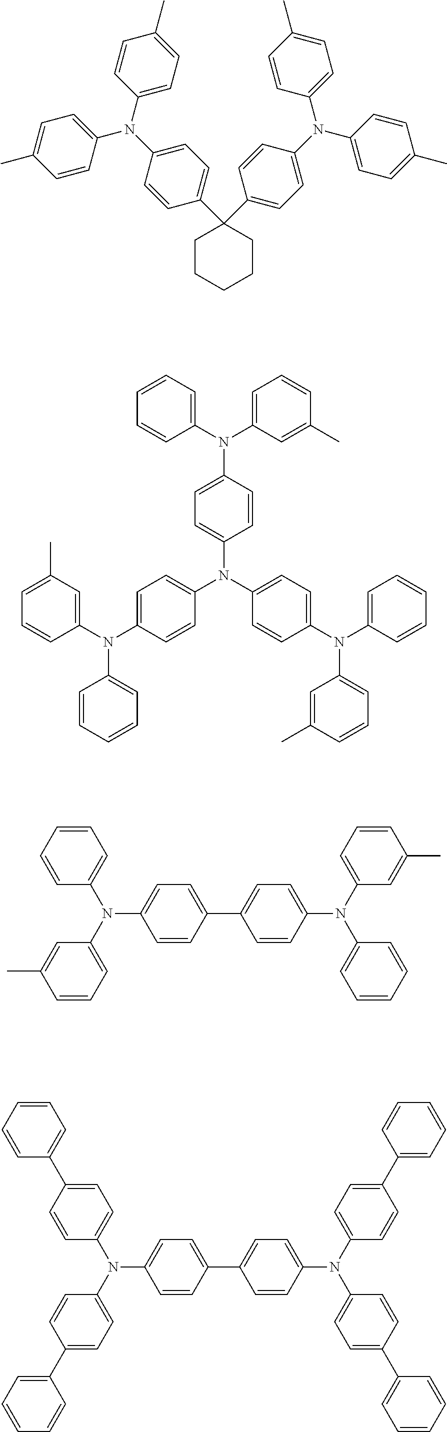

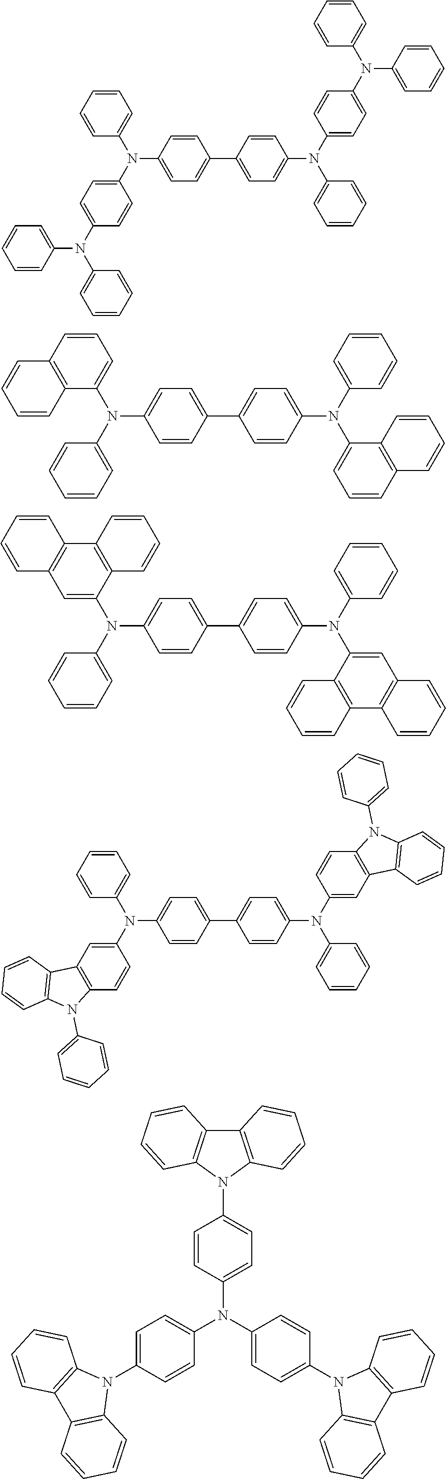

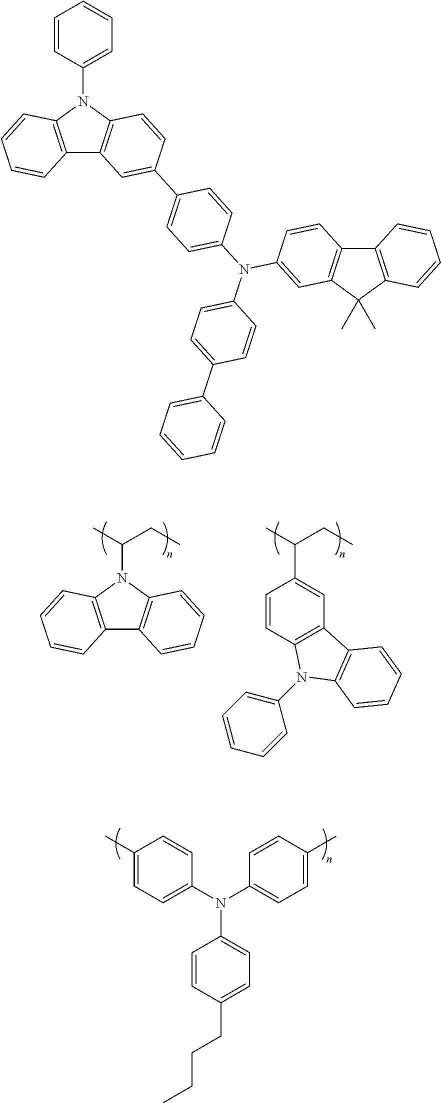

### Electron Transport Layer:

The electron transport layer comprises an electron transport material. In some embodiments, the electron transport layer is a single layer. In some embodiments, the electron transport layer comprises a plurality of layers.

In some embodiments, the electron transport material needs only to have a function of transporting electrons, which are injected from the cathode, to the light emitting layer. In some embodiments, the electron transport material also functions as a hole barrier material. Examples of the electron transport layer that can be used in the present invention include but are not limited to a nitro-substituted fluorene derivative, a diphenylquinone derivative, a thiopyran dioxide derivative, a carbodiimide, a fluorenylidenemethane derivative, an anthraquinodimethane, an anthrone derivative, an oxadiazole derivative, an azole derivative, an azine derivative, or a combination thereof, or a polymer thereof. In some embodiments, the electron transport material is a thiadiazole derivative, or a quinoxaline derivative. In some embodiments, the electron transport material is a polymer material. Preferred specific examples of a compound for use as the electron transport material are shown below.

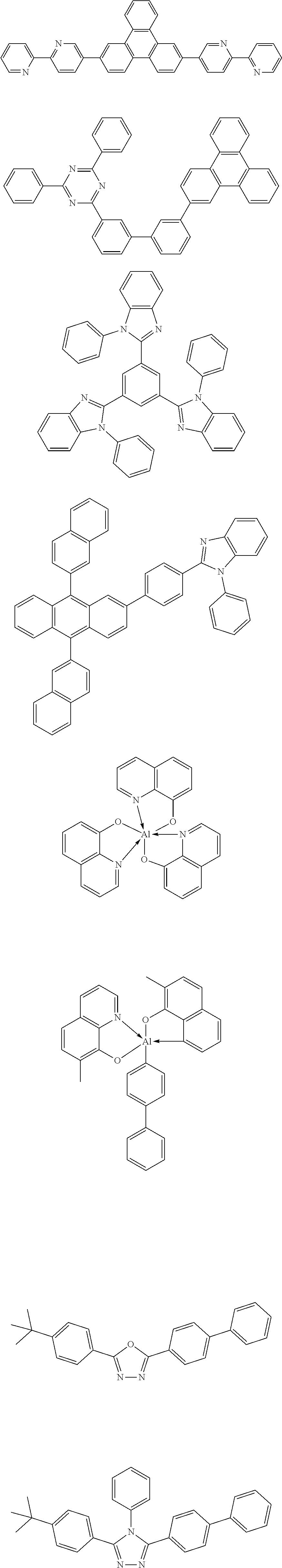

Preferred examples of compounds usable as materials that can be added to each organic layer are shown below. For example, the addition of a compound as a stabilizing material can be taken into consideration.

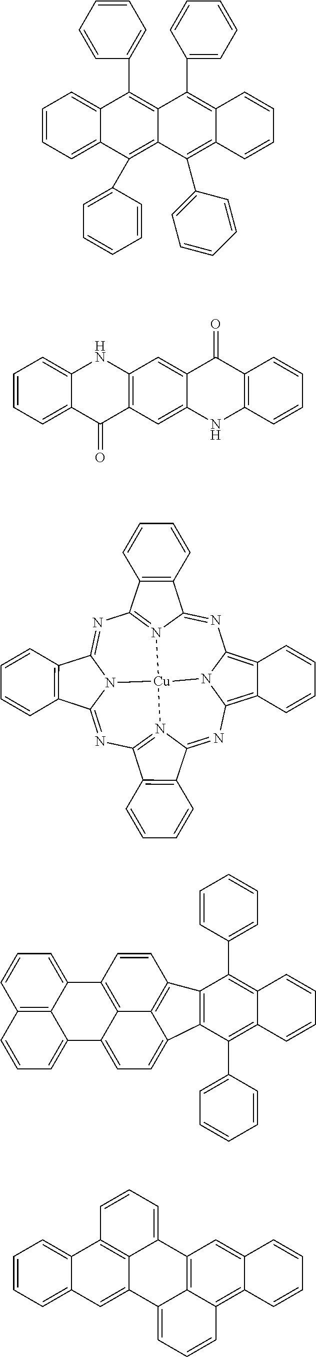

Hereinabove preferred materials for use in an organic electroluminescent device are specifically shown; however, the materials which can be used in the present invention are not construed as limiting to the exemplary compounds. Compounds that are exemplified as materials having a specific function can also be used as materials having any other function.

Each organic layer of the organic electroluminescent device can be formed in a wet process. In a wet process, a solution prepared by dissolving a composition containing the compound to constitute an organic layer is applied onto a surface, and then the solvent is removed to form a film. The wet process includes a spin coating method, a slit coating method, an ink jet method (a spraying method), a gravure printing method, an offset printing method and a flexographic printing method, which, however, are not limitative. In the wet process, an appropriate organic solvent capable of dissolving the compound to constitute an organic layer is selected and used. In some embodiments, a substituent (for example, an alkyl group) capable of increasing the solubility in an organic solvent can be introduced into the compound to constitute an organic layer.

In some embodiments, the organic layer can be formed in a dry process. In some embodiments, a vacuum evaporation method is employable as a dry process, which, however, is not limitative. In the case where a vacuum evaporation method is employed, compounds to constitute organic layers can be co-evaporated from individual evaporation sources, or can be co-evaporated from a single evaporation source formed by mixing the compounds. In the case where a single evaporation source is used, a mixed powder prepared by mixing compound powders can be used, or a compression molded body prepared by compressing the mixed powder can be used, or a mixture prepared by heating and melting the constituent compounds and cooling the resulting melt can be used. In some embodiments, by co-evaporation under the condition where the evaporation rate (weight reduction rate) of the plural compounds contained in a single evaporation source is the same or is nearly the same, an organic layer having a compositional ratio corresponding to the compositional ratio of the plural compounds contained in the evaporation source can be formed. When plural compounds are mixed in the same compositional ratio as the compositional ratio of the organic layer to be formed to prepare an evaporation source, an organic layer having a desired compositional ratio can be formed in a simplified manner. In some embodiments, the temperature at which the compounds to be co-evaporated has the same weight reduction ratio is specifically defined, and the temperature can be employed as the temperature of co-evaporation.

### [Devices]

In some embodiments, the light emitting layers are incorporated into a device. For example, the device includes, but is not limited to an OLED bulb, an OLED lamp, a television screen, a computer monitor, a mobile phone, and a tablet.

In some embodiments, an electronic device comprises an OLED comprising an anode, a cathode, and at least one organic layer comprising a light emitting layer between the anode and the cathode.

In some embodiments, compositions described in the present description can be incorporated into various light-sensitive or light-activated devices, such as OLEDs or photoelectronic devices. In some embodiments, the composition can be useful in facilitating charge transfer or energy transfer within a device and/or as a hole transport material. The device can be, for example, an organic light-emitting diode (OLED), an organic integrated circuit (OIC), an organic field-effect transistor (O-FET), an organic thin-film transistor (O-TFT), an organic light-emitting transistor (O-LET), an organic solar cell (O—SC), an organic optical detector, an organic photoreceptor, an organic field-quench device (O-FQD), a light-emitting electrochemical cell (LEC) or an organic laser diode (O-laser).

### [Bulbs or Lamps]

In some embodiments, an electronic device comprises an OLED comprising an anode, a cathode, and at least one organic layer comprising a light emitting layer between the anode and the cathode.

In some embodiments, a device comprises OLEDs that differ in color. In some embodiments, a device comprises an array comprising a combination of OLEDs. In some embodiments, the combination of OLEDs is a combination of three colors (for example, having RGB). In some embodiments, the combination of OLEDs is a combination of colors that are not red, green, or blue (for example, orange and yellow green). In some embodiments, the combination of OLEDs is a combination of two, four, or more colors.

In some embodiments, a device is an OLED light comprising,
a circuit board having a first side with a mounting surface and an opposing second side, and defining at least one opening;
at least one OLED on the mounting surface, the at least one OLED configured to emanate light, comprising an anode, a cathode, and at least one organic layer comprising a light emitting layer between the anode and the cathode;
a housing for the circuit board; and
at least one connector arranged at an end of the housing, the housing and the connector defining a package adapted for installation in a light fixture.

In some embodiments, the OLED light comprises a plurality of OLEDs mounted on a circuit board such that light emanates in a plurality of directions. In some embodiments, a portion of the light emanated in a first direction is deflected to emanate in a second direction. In some embodiments, a reflector is used to deflect the light emanated in a first direction.

### [Displays or Screens]

In some embodiments, the light emitting layer in the present invention can be used in a screen or a display. In some embodiments, the compounds in the present invention are deposited onto a substrate using a process including, but not limited to, vacuum evaporation, deposition, vapor deposition, or chemical vapor deposition (CVD). In some embodiments, the substrate is a photoplate structure useful in a two-sided etching that provides a unique aspect ratio pixel. The screen (which may also be referred to as a mask) is used in a process in the manufacturing of OLED displays. The corresponding artwork pattern design facilitates a very steep and narrow tie-bar between the pixels in the vertical direction and a large, sweeping bevel opening in the horizontal direction. This allows the close patterning of pixels needed for high resolution displays while optimizing the chemical vapor deposition onto a TFT backplane.

The internal patterning of the pixel allows the construction of a three-dimensional pixel opening with varying aspect ratios in the horizontal and vertical directions. Additionally, the use of imaged “stripes” or halftone circles within the pixel area inhibits etching in specific areas until these specific patterns are undercut and fall off the substrate. At that point, the entire pixel area is subjected to a similar etching rate but the depths are varying depending on the halftone pattern. Varying the size and spacing of the halftone pattern allows etching to be inhibited at different rates within the pixel allowing for a localized deeper etching needed to create steep vertical bevels.

A preferred material for the deposition mask is invar. Invar is a metal alloy that is cold rolled into long thin sheet in a steel mill. Invar cannot be electrodeposited onto a rotating mandrel as the nickel mask. An appropriate and more cost feasible method for forming the opening areas in the mask used for deposition is through a wet chemical etching.

In some embodiments, a screen or display pattern is a pixel matrix on a substrate. In some embodiments, a screen or display pattern is fabricated using lithography (for example, having photolithography and e-beam lithography). In some embodiments, a screen or display pattern is fabricated using a wet chemical etching. In further embodiments, a screen or display pattern is fabricated using plasma etching.

### [Methods of Manufacturing Devices]

An OLED display is generally manufactured by forming a large mother panel and then cutting the mother panel in units of cell panels. In general, each of the cell panels on the mother panel is formed by forming a thin film transistor (TFT) including an active layer and a source/drain electrode on a base substrate, applying a planarization film to the TFT, and sequentially forming a pixel electrode, a light emitting layer, a counter electrode, and an encapsulation layer, and then is cut from the mother panel.

In another aspect, provided herein is a method of manufacturing an organic light-emitting diode (OLED) display, the method comprising:
forming a barrier layer on a base substrate of a mother panel;
forming a plurality of display units in units of cell panels on the barrier layer;
forming an encapsulation layer on each of the display units of the cell panels; and
applying an organic film to an interface portion between the cell panels.

In some embodiments, the barrier layer is an inorganic film formed of, for example, SiNx, and an edge portion of the barrier layer is covered with an organic film formed of polyimide or acryl. In some embodiments, the organic film helps the mother panel to be softly cut in units of the cell panel.

In some embodiments, the thin film transistor (TFT) layer includes a light emitting layer, a gate electrode, and a source/drain electrode. Each of the plurality of display units may include a thin film transistor (TFT) layer, a planarization film formed on the TFT layer, and a light-emitting unit formed on the planarization film, wherein the organic film applied to the interface portion is formed of a same material as a material of the planarization film and is formed at a same time as the planarization film is formed. In some embodiments, the light-emitting unit is connected to the TFT layer with a passivation layer and a planarization film therebetween and an encapsulation layer that covers and protects the light-emitting unit. In some embodiments of the method of manufacturing, the organic film is connected to neither the display units nor the encapsulation layer.

Each of the organic film and the planarization film may include any one of polyimide and acryl. In some embodiments, the barrier layer can be an inorganic film. In some embodiments, the base substrate can be formed of polyimide. The method may further include, before the forming of the barrier layer on one surface of the base substrate formed of polyimide, attaching a carrier substrate formed of a glass material to another surface of the base substrate, and before the cutting along the interface portion, separating the carrier substrate from the base substrate. In some embodiments, the OLED display is a flexible display.

In some embodiments, the passivation layer is an organic film disposed on the TFT layer to cover the TFT layer. In some embodiments, the planarization film is an organic film formed on the passivation layer. In some embodiments, the planarization film is formed of polyimide or acryl, like the organic film formed on the edge portion of the barrier layer. In some embodiments, the planarization film and the organic film are simultaneously formed when the OLED display is manufactured. In some embodiments, the organic film can be formed on the edge portion of the barrier layer such that a portion of the organic film directly contacts the base substrate and a remaining portion of the organic film contacts the barrier layer while surrounding the edge portion of the barrier layer.

In some embodiments, the light emitting layer includes a pixel electrode, a counter electrode, and an organic light emitting layer disposed between the pixel electrode and the counter electrode. In some embodiments, the pixel electrode is connected to the source/drain electrode of the TFT layer.

In some embodiments, when a voltage is applied to the pixel electrode through the TFT layer, an appropriate voltage is formed between the pixel electrode and the counter electrode, and thus the organic light emitting layer emits light, thereby forming an image. Hereinafter, an image forming unit including the TFT layer and the light-emitting unit is referred to as a display unit.

In some embodiments, the encapsulation layer that covers the display unit and prevents penetration of external moisture can be formed to have a thin film encapsulation structure in which an organic film and an inorganic film are alternately stacked. In some embodiments, the encapsulation layer has a thin film encapsulation structure in which a plurality of thin films are stacked. In some embodiments, the organic film applied to the interface portion is spaced apart from each of the plurality of display units. In some embodiments, the organic film is formed such that a portion of the organic film directly contacts the base substrate and a remaining portion of the organic film contacts the barrier layer while surrounding the edge portion of the barrier layer.

In some embodiments, the OLED display is flexible and uses the soft base substrate formed of polyimide. In some embodiments, the base substrate is formed on a carrier substrate formed of a glass material, and then the carrier substrate is separated.

In some embodiments, the barrier layer is formed on a surface of the base substrate opposite to the carrier substrate. In some embodiments, the barrier layer is patterned according to a size of each of the cell panels. For example, while the base substrate is formed over the entire surface of a mother panel, the barrier layer is formed according to a size of each of the cell panels, and thus a groove is formed at an interface portion between the barrier layers of the cell panels. Each of the cell panels can be cut along the groove.

In some embodiments, the method of manufacture further comprises cutting along the interface portion, wherein a groove is formed in the barrier layer, wherein at least a portion of the organic film is formed in the groove, and wherein the groove does not penetrate into the base substrate. In some embodiments, the TFT layer of each of the cell panels is formed, and the passivation layer which is an inorganic film and the planarization film which is an organic film are disposed on the TFT layer to cover the TFT layer. At the same time as the planarization film formed of, for example, polyimide or acryl is formed, the groove at the interface portion is covered with the organic film formed of, for example, polyimide or acryl. This is to prevent cracks from occurring by allowing the organic film to absorb an impact generated when each of the cell panels is cut along the groove at the interface portion. That is, if the entire barrier layer is entirely exposed without the organic film, an impact generated when each of the cell panels is cut along the groove at the interface portion is transferred to the barrier layer, thereby increasing the risk of cracks. However, in one embodiment, since the groove at the interface portion between the barrier layers is covered with the organic film and the organic film absorbs an impact that would otherwise be transferred to the barrier layer, each of the cell panels can be softly cut and cracks can be prevented from occurring in the barrier layer. In one embodiment, the organic film covering the groove at the interface portion and the planarization film are spaced apart from each other. For example, if the organic film and the planarization film are connected to each other as one layer, since external moisture may penetrate into the display unit through portions where the planarization film and the organic film remain, the organic film and the planarization film are spaced apart from each other such that the organic film is spaced apart from the display unit.

In some embodiments, the display unit is formed by forming the light-emitting unit, and the encapsulation layer is disposed on the display unit to cover the display unit. As such, once the mother panel is completely manufactured, the carrier substrate that supports the base substrate is separated from the base substrate. In some embodiments, when a laser beam is emitted toward the carrier substrate, the carrier substrate is separated from the base substrate due to a difference in a thermal expansion coefficient between the carrier substrate and the base substrate.

In some embodiments, the mother panel is cut in units of the cell panels. In some embodiments, the mother panel is cut along an interface portion between the cell panels by using a cutter. In some embodiments, since the groove at the interface portion along which the mother panel is cut is covered with the organic film, the organic film absorbs an impact during the cutting. In some embodiments, cracks can be prevented from occurring in the barrier layer during the cutting.

In some embodiments, the methods reduce a defect rate of a product and stabilize its quality.

Another aspect is an OLED display including: a barrier layer that is formed on a base substrate; a display unit that is formed on the barrier layer; an encapsulation layer that is formed on the display unit; and an organic film that is applied to an edge portion of the barrier layer.

### EXAMPLES

The characteristics of the present invention will be explained in more detail with reference to Examples below. The materials, processes, procedures and the like shown below can be appropriately modified unless they deviate from the substance of the present invention. Accordingly, the scope of the present invention is not construed as being limited to the specific examples shown below. Herein under the light emission characteristics were evaluated using a source meter (available from Keithley Instruments Corporation: 2400 series), a semiconductor parameter analyzer (available from Agilent Corporation, E5273A), an optical power meter device (available from Newport Corporation, 1930C), an optical spectroscope (available from Ocean Optics Corporation, USB2000), a spectroradiometer (available from Topcon Corporation, SR-3), and a streak camera (available from Hamamatsu Photonics K.K., Model C4334).

### Example 1

On a glass substrate on which an anode made of indium-tin oxide (ITO) having a film thickness of 50 nm was formed, the following thin films were laminated by a vacuum deposition method at a vacuum degree of 5.0×10⁻⁵ Pa to produce an organic electroluminescent device.

First, on ITO, HAT-CN was formed to have a thickness of 10 nm, then NPD was formed thereon to have a thickness of 30 nm, and further thereon, Compound 1 was formed to have a thickness of 5 nm. Next, a host material (H50), a delayed fluorescence material (T33), and a light emitting material (E1) were co-deposited from different evaporation sources to form a light emitting layer with a thickness of 35 nm. In that case, the content of the host material was 34.2% by mass, the content of the delayed fluorescent material was 65.0% by mass, and the content of the light emitting material was 0.8% by mass. Next, SF3-TRZ was formed with a thickness of 10 nm, and then, Liq and SF3-TRZ were co-deposited from different evaporation sources to form a layer with a thickness of 30 nm. The contents of Liq and SF3-TRZ in this layer were 30% by mass and 70% by mass, respectively. Further, Liq was formed with a thickness of 2 nm, and then, aluminum (Al) was vapor-deposited with a thickness of 100 nm to form a cathode, and an organic electroluminescence device was thus produced. The device was referred to as EL Device 1.

An organic electroluminescent device was produced according to the same process as above except that Comparative Compound A was used in place of Compound 1, and this was referred to as Comparative EL Device 1.

Thus produced each organic electroluminescent device was energized, and delayed fluorescence derived from the light emitting material (E1) was observed. Each organic electroluminescent device was driven at 6.3 mA/cm² to measure the initial drive voltage. The measurement results are shown in Table 3. The drive voltage in Table 3 is a relative value based on the drive voltage of Comparative EL Device 1. Each organic electroluminescent device was driven at a current density of 12.6 mA/cm², and the time taken until the emission intensity reached 95% at the start of the driving was measured (LT95). The measurement results are shown in Table 3. LT95 in Table 3 is expressed as a relative value, when LT95 of Relative EL Device 1 is defined as 1. The measurement results show that the device using a compound represented by the general formula (1) as an electron barrier material can be driven at a lower drive voltage and can have a remarkably more prolonged device lifetime, than the device using Comparative Compound A that has heretofore been used as an electron barrier material.

TABLE 3

| | Electron Barrier | | |
|---|---|---|---|
| Device Number | Material | Drive Voltage | LT95 |
| EL Device 1 | Compound 1 | −0.65 V | 2.1 times |
| Comparative EL | Comparative | Reference | 1 |
| Device 1 | Compound A | | |

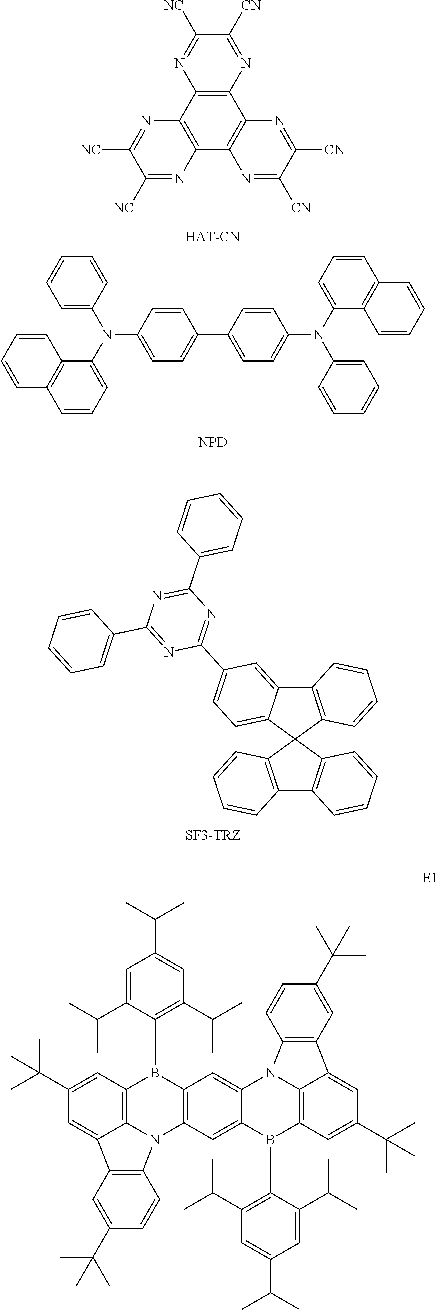 [SMILES: N#Cc1nc2c3nc(C#N)c(C#N)nc3c3nc(C#N)c(C#N)nc3c2nc1C#N ; c1ccc(N(c2ccc(-c3ccc(N(c4ccccc4)c4cccc5ccccc45)cc3)cc2)c2cccc3ccccc23)cc1 ; "NPD" ; c1ccc(-c2nc(-c3ccccc3)nc(-c3ccc4c(c3)-c3ccccc3C43c4ccccc4-c4ccccc43)n2)cc1 ; CC(C)c1cc(C(C)C)c(B2c3cc4c(cc3-n3c5ccc(C(C)(C)C)cc5c5cc(C(C)(C)C)cc2c53)B(c2c(C(C)C)cc(C(C)C)cc2C(C)C)c2cc(C(C)(C)C)cc3c5cc(C(C)(C)C)ccc5n-4c23)c(C(C)C)c1]

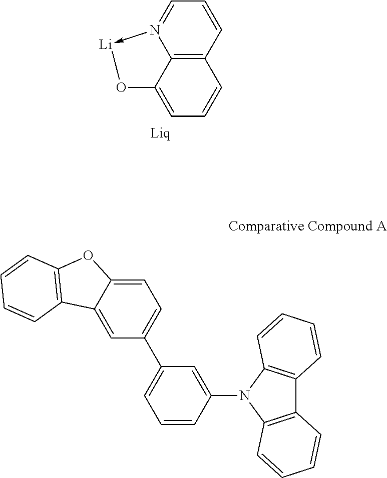

### INDUSTRIAL APPLICABILITY

The compound represented by the general formula (1) is useful as an electron barrier material, and can be effectively used in an organic semiconductor device. By using the compound of the present invention as an electron barrier layer of an organic electroluminescent device, the drive voltage can be lowered and the device lifetime can be prolonged. Accordingly, the industrial applicability of the present invention is great.

1. An electron barrier material containing a compound represented by the following general formula (1): 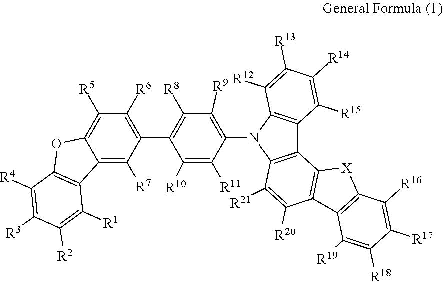
wherein R¹ to R²¹ each independently represent a hydrogen atom, a deuterium atom, or a substituent not including a cyano group;
one combination of R² and R¹³, R¹³ and R¹⁴, and R¹⁴ and R¹⁵ can bond to each other to form a benzofuro skeleton or a benzothieno skeleton;
R¹ to R¹¹, and R¹⁶ to R²¹ do not bond to the other R¹ to R¹¹, R¹⁶ to R²¹ or R¹² to R¹⁵ to form a cyclic structure; and
X represents an oxygen atom or a sulfur atom.

2. The electron barrier material according to claim 1, wherein R¹ to R²¹ do not bond to the other R¹ to R²¹ to form a cyclic structure.

3. The electron barrier material according to claim 1, wherein R¹ to R²¹ each independently represent a hydrogen atom, a deuterium atom, an optionally-deuterated alkyl group, or an optionally-deuterated phenyl group.

4. The electron barrier material according to claim 1, wherein R¹ to R¹¹, R²⁰ and R²¹ each independently represent a hydrogen atom or a deuterium atom.

5. The electron barrier material according to claim 1, wherein R¹² to R¹⁵ each independently represent a hydrogen atom or a deuterium atom.

6. The electron barrier material according to claim 1, wherein R¹⁶ to R¹⁹ each independently represent a hydrogen atom or a deuterium atom.

7. The electron barrier material according to claim 1, wherein X is an oxygen atom.

8. The electron barrier material according to claim 1, which is used in combination with a compound represented by the following general formula (G): 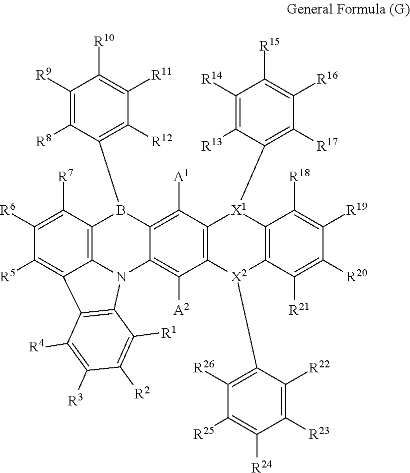
wherein one of X¹ and X² is a nitrogen atom, and the other is a boron atom;
R¹ to R²⁶, A¹ and A² each independently represent a hydrogen atom, a deuterium atom, or a substituent;
R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ can bond to each other to form a cyclic structure; and
provided that when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring.

9. An organic semiconductor device containing the electron barrier material according to claim 1.

10. The organic semiconductor device according to claim 9, wherein the organic semiconductor device is an organic electroluminescent device having an anode, a cathode, and at least two organic layers containing an electron barrier layer that contains the above electron barrier material and a light emitting layer, between the anode and the cathode.

11. The organic semiconductor device according to claim 10, wherein the light emitting layer contains a host material and a delayed fluorescent material.

12. The organic semiconductor device according to claim 10, wherein the light emitting layer contains a host material, a delayed florescent material and a fluorescence emitting material, and the amount of light emitted from the fluorescence emitting material is the largest among the light from the device.

13. The organic semiconductor device according to claim 10, wherein the light emitting layer is adjacent to the electron barrier layer.

14. The organic semiconductor device according to claim 10, wherein the light emitting layer contains the compound represented by the following general formula (G): 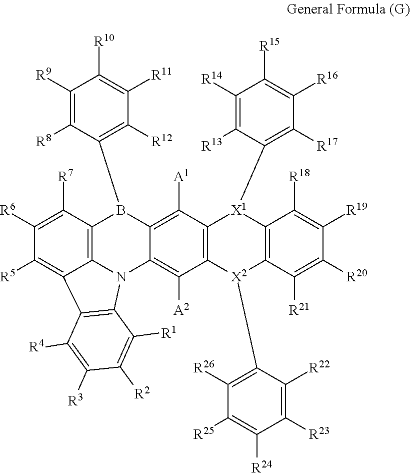
wherein one of X¹ and X² is a nitrogen atom, and the other is a boron atom;
R¹ to R²⁶, A¹ and A² each independently represent a hydrogen atom, a deuterium atom, or a substituent;
R¹ and R², R² and R³, R³ and R⁴, R⁴ and R⁵, R⁵ and R⁶, R⁶ and R⁷, R⁷ and R⁸, R⁸ and R⁹, R⁹ and R¹⁰, R¹⁰ and R¹¹, R¹¹ and R¹², R¹³ and R¹⁴, R¹⁴ and R¹⁵, R¹⁵ and R¹⁶, R¹⁶ and R¹⁷, R¹⁷ and R¹⁸, R¹⁸ and R¹⁹, R¹⁹ and R²⁰, R²⁰ and R²¹, R²¹ and R²², R²² and R²³, R²³ and R²⁴, R²⁴ and R²⁵, and R²⁵ and R²⁶ can bond to each other to form a cyclic structure;
provided that when X¹ is a nitrogen atom, R¹⁷ and R¹⁸ bond to each other to be a single bond to form a pyrrole ring, and when X² is a nitrogen atom, R²¹ and R²² bond to each other to be a single bond to form a pyrrole ring.
